(19) United States
(12) Patent Application Publication
(10) Pub. No.: US 2011/0143444 A1
(43) Pub. Date: Jun. 16, 2011

(54) METHOD OF EVALUATING FEMALE GENITAL CANCER

(75) Inventors: Takahiko Muramatsu, Kanagawa (JP); Yutaka Ihata, Kanagawa (JP); Akira Imaizumi, Kanagawa (JP); Hiroshi Yamamoto, Tokyo (JP); Etsuko Miyagi, Kanagawa (JP); Fumiki Hirahara, Kanagawa (JP)

(73) Assignee: Ajinomoto Co., Inc.

(21) Appl. No.: 12/968,578

(22) Filed: Dec. 15, 2010

Related U.S. Application Data
(63) Continuation of application No. PCT/JP2009/061348, filed on Jun. 22, 2009.

(30) Foreign Application Priority Data
Jun. 20, 2008 (JP) 2008-162612

(51) Int. Cl. G01N 33/68; G06F 19/00

(52) U.S. Cl. 436/90; 702/19

(57) ABSTRACT

According to the method of evaluating female genital cancer of the present invention, amino acid concentration data on concentration values of amino acids in blood collected from a subject to be evaluated is measured, and the state of female genital cancer including at least one of cervical cancer, endometrial cancer, and ovarian cancer in the subject is evaluated based on the concentration value of at least one of Thr, Ser, Asn, Gln, Pro, Gly, Ala, Cit, Val, Met, Ile, Leu, Tyr, Phe, His, Trp, Orn, Lys, and Arg contained in the measured amino acid concentration data of the subject.

This application is a Continuation of PCT/JP2009/061348, filed Jun. 22, 2009, which claims priority from Japanese patent application JP 2008-162612 filed Jun. 20, 2008. The contents of each of the aforementioned application are incorporated herein by reference in their entirety.

### BACKGROUND OF THE INVENTION

1. Field of the Invention

The present invention relates to a method of evaluating female genital cancer including at least one of cervical cancer, endometrial cancer, and ovarian cancer, which utilizes a concentration of an amino acid in blood (plasma).

2. Description of the Related Art

In Japan, the number of deaths from cervical cancer is 2494, the number of deaths from endometrial cancer is 1436, and the number of deaths from ovarian cancer is 4420 in 2004. With regard to the survival rate of these cancers, the 5-year survival rate of some of the cancers in an early stage (I to II stages) is above 80%, but the 5-year survival rate of the advanced cancers is extremely lowered to about 10% to 20%. Therefore, early detection is important for curing these cancers.

The diagnosis of cervical cancer is performed by cytodiagnosis, histological diagnosis, colposcopy, or HPV (human papillomavirus) examination. Cytodiagnosis and HPV examination cannot be definitive diagnosis and can be definitive diagnosis by performing histological diagnosis or colposcopy. However, histological diagnosis and colposcopy are a high-invasiveness examination so that it is not realistic to subject all patients who are suspected of having cervical cancer to them.

The diagnosis of endometrial cancer is performed mainly by endometrical cytodiagnosis. Endometrical cytodiagnosis cannot be definitive diagnosis and can be definitive diagnosis by performing curettage diagnosis. However, curettage diagnosis is a high-invasiveness examination so that it is not realistic to subject all patients of suspicious for endometrial cancer to it.

The diagnosis of ovarian cancer is performed by ultrasonotomography and tumor marker (mainly, CA125), CT (computed tomography), or MRI (magnetic resonance imaging).

These methods cannot be definitive diagnosis and can be definitive diagnosis by performing histopathological diagnosis of an ovary extracted by an operation. However, according to the report of van Nagell J R et al., that an extraction operation of eleven benign tumors (false positive) is necessary for finding one ovarian cancer (true positive) (see “van Nagell J R, DePriest P D, Reedy M B, Gallion H H, Ueland F R, Pavlik E J, Kryscio R J., The efficiency of transvaginal sonographic screening in asymptomatic women at risk for ovarian cancer., Gynecol Oncol, 2000, 77; 350-356”), the positive predictive value of ovarian cancer is as low as 8.3%.

In addition, most of these cancer diagnosing methods are invasive, as described above. The execution of these diagnosing methods involves loads, such as physical pain and mental pain, on patients, and the risk of bleeding due to examination can occur. Further, these diagnosing methods are independently performed in each state of female genital cancer and cause cost for each examination so that the economical and time loads of subjects are increased. Therefore, from the viewpoint of the physical load on patients and the cost effectiveness, desirably, subjects having a high possibility of occurrence of female genital cancer are selected by a method having less invasiveness and mental pain and by one examination at low cost, the selected subjects are diagnosed, and the subjects who have obtained definitive diagnosis are to be treated.

It is known that blood amino acid concentration is changed by occurrence of cancer. For instance, Cynober (see “Cynober, L. ed., Metabolic and therapeutic aspects of amino acids in clinical nutrition. 2nd ed., CRC Press.”) has reported that the consumption amount in cancer cells of each of glutamine mainly as an oxidative energy source, arginine as the precursor of nitrogen oxide or polyamine, and methionine subjected to activation of methionine take-in ability by cancer cells is increased. In addition, Vissers et al. (see “Vissers, Y. L J., et. al., Plasma arginine concentration are reduced in cancer patients: evidence for arginine deficiency?, The American Journal of Clinical Nutrition, 2005 81, p. 1142-1146”), Park (see “Park, K. G., et al., Arginine metabolism in benign and malignant disease of breast and colon: evidence for possible inhibition of tumor-infiltrating macrophages., Nutrition, 1991 7, p. 185-188”), Proenza et al. (see “Proenza, A. M., J. Oliver, A. Palou and P. Roca, Breast and lung cancer are associated with a decrease in blood cell amino acid content. J Nutr Biochem, 2003. 14(3): p. 133-8”), and Cascino (see “Cascino, A., M. Muscaritoli, C. Cangiano, L. Conversano, A. Laviano, S. Ariemma, M. M. Meguid and F. Rossi Fanelli, Plasma amino acid imbalance in patients with lung and breast cancer. Anticancer Res, 1995. 15(2): p. 507-10”) have reported that the plasma amino acid composition of cancer patients is different from that of healthy subjects.

WO 2004/052191 and WO 2006/098192 disclose a method for associating amino acid concentration with biological state. WO 2008/016111 discloses a method for evaluating the state of lung cancer using an amino acid concentration.

However, there is a problem that diagnosing methods and apparatuses, which use a plurality of amino acids as explanatory variables to diagnose the presence or absence of occurrence of female genital cancer, have not developed from the viewpoint of time and cost, and have not been practically used. In addition, there is a problem that even when the presence or absence of occurrence of female genital cancer is discriminated by an index formula group for discriminating lung cancer disclosed in WO 2008/016111, sufficient discriminative ability cannot be obtained due to different discriminated targets.

### SUMMARY OF THE INVENTION

It is an object of the present invention to at least partially solve the problems in the conventional technology. The present invention has been made in view of the problems described above, and an object of the present invention is to provide a method of evaluating female genital cancer, which can evaluate the state of female genital cancer accurately, by using, of blood amino acid concentrations, the amino acid concentration associated with the state of female genital cancer.

The present inventors have earnestly studied the problems to solve them, have identified amino acids useful for 2-group discrimination between female genital cancer and female genital cancer-free, have found that multivariate discriminants (index formulae or correlation equations) containing the concentrations of the identified amino acids as explanatory variables, significantly correlate with the state of female genital cancer, and have completed the present invention. Specifically, the present inventors have searched for more specific index formulae with respect to female genital cancer, have been able to obtain index formulae which are more suitable for evaluating the state of female genital cancer than the index formulae disclosed in WO 2004/052191, WO 2006/098192, and WO 2008/016111, and have completed the present invention.

To solve the problem and achieve the object described above, a method of evaluating female genital cancer according to one aspect of the present invention includes a measuring step of measuring amino acid concentration data on a concentration value of an amino acid in blood collected from a subject to be evaluated, and a concentration value criterion evaluating step of evaluating a state of female genital cancer including at least one of cervical cancer, endometrial cancer, and ovarian cancer in the subject, based on the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data of the subject measured at the measuring step.

Another aspect of the present invention is the method of evaluating female genital cancer, wherein the concentration value criterion evaluating step further includes a concentration value criterion discriminating step of discriminating (i) between the female genital cancer and a female genital cancer-free, (ii) between any one of the cervical cancer, the endometrial cancer, and the ovarian cancer and the female genital cancer-free, (iii) between any one of the cervical cancer and the endometrial cancer and any one of a cervical cancer-free and an endometrial cancer-free, (iv) between the cervical cancer and the cervical cancer-free, (v) between the endometrial cancer and the endometrial cancer-free, (vi) between the ovarian cancer and a ovarian cancer-free, (vii) between a female genital cancer suffering risk group and a healthy group, or (viii) between the cervical cancer, the endometrial cancer, and the ovarian cancer in the subject, based on the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data of the subject measured at the measuring step.

Still another aspect of the present invention is the method of evaluating female genital cancer, wherein the concentration value criterion evaluating step further includes (I) a discriminant value calculating step of calculating a discriminant value that is a value of a multivariate discriminant with a concentration of the amino acid as an explanatory variable, based on both (i) the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data of the subject measured at the measuring step and (ii) the previously established multivariate discriminant, and (II) a discriminant value criterion evaluating step of evaluating the state of female genital cancer in the subject based on the discriminant value calculated at the discriminant value calculating step. The multivariate discriminant contains at least one of Thr, Ser, Asn, Gln, Pro, Gly, Ala, Cit, Val, Met, Ile, Leu, Tyr, Phe, His, Trp, Orn, Lys, and Arg as the explanatory variable.

Still another aspect of the present invention is the method of evaluating female genital cancer, wherein the discriminant value criterion evaluating step further includes a discriminant value criterion discriminating step of discriminating (i) between the female genital cancer and a female genital cancer-free, (ii) between any one of the cervical cancer, the endometrial cancer, and the ovarian cancer and the female genital cancer-free, (iii) between any one of the cervical cancer and the endometrial cancer and any one of a cervical cancer-free and an endometrial cancer-free, (iv) between the cervical cancer and the cervical cancer-free, (v) between the endometrial cancer and the endometrial cancer-free, (vi) between the ovarian cancer and a ovarian cancer-free, (vii) between a female genital cancer suffering risk group and a healthy group, or (viii) between the cervical cancer, the endometrial cancer, and the ovarian cancer in the subject, based on the discriminant value calculated at the discriminant value calculating step.

Still another aspect of the present invention is the method of evaluating female genital cancer, wherein the multivariate discriminant is any one of a fractional expression, the sum of a plurality of the fractional expressions, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

Still another aspect of the present invention is the method of evaluating female genital cancer, wherein (I) at the discriminant value calculating step, the discriminant value is calculated based on (i) the concentration value of at least one of Thr, Ser, Asn, Gln, Pro, Ala, Val, Met, Ile, Leu, Tyr, Phe, His, Trp, Orn, and Arg contained in the amino acid concentration data of the subject measured at the measuring step and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) at the discriminant value criterion discriminating step, the discrimination between any one of the cervical cancer, the endometrial cancer, and the ovarian cancer and the female genital cancer-free in the subject is conducted based on the discriminant value calculated at the discriminant value calculating step. Still another aspect of the present invention is the method of evaluating female genital cancer, wherein the multivariate discriminant is (i) the fractional expression with Gln, His, and Arg as the explanatory variables, (ii) the fractional expression with a-ABA, His, and Met as the explanatory variables, (iii) the fractional expression with Ile, His, Cit, Arg, Tyr, and Trp as the explanatory variables, (iv) the fractional expression with a-ABA, Cit, and Met as the explanatory variables, (v) the linear discriminant with Gly, Val, His, and Arg as the explanatory variables, (vi) the linear discriminant with Gly, a-ABA, Met, and His as the explanatory variables, (vii) the linear discriminant with Ala, Ile, His, Trp, and Arg as the explanatory variables, (viii) the linear discriminant with Gly, Cit, Met, and Phe as the explanatory variables, (ix) the linear discriminant with His, Leu, Met, Cit, Ile, and Tyr as the explanatory variables, (x) the logistic regression equation with Val, Leu, His, and Arg as the explanatory variables, (xi) the logistic regression equation with a-ABA, Met, Tyr, and His as the explanatory variables, (xii) the logistic regression equation with Val, Ile, His, Trp, and Arg as the explanatory variables, (xiii) the logistic regression equation with Cit, a-ABA, Met, and Tyr as the explanatory variables, or (xiv) the logistic regression equation with His, Leu, Met, Cit, Ile, and Tyr as the explanatory variables.

Still another aspect of the present invention is the method of evaluating female genital cancer, wherein (I) at the discriminant value calculating step, the discriminant value is calculated based on (i) the concentration value of at least one of Thr, Ser, Asn, Pro, Gly, Cit, Val, Met, Ile, Leu, Phe, His, Trp, Orn, Lys, and Arg contained in the amino acid concentration data of the subject measured at the measuring step and (ii) the multivariate discriminant containing at least one of Thr, Ser, Asn, Pro, Gly, Cit, Val, Met, Ile, Leu, Phe, His, Trp, Orn, Lys, and Arg as the explanatory variable, and (II) at the discriminant value criterion discriminating step, the discrimination between any one of the cervical cancer and the endometrial cancer and any one of the cervical cancer-free and the endometrial cancer-free in the subject is conducted based on the discriminant value calculated at the discriminant value calculating step. Still another aspect of the present invention is the method of evaluating female genital cancer, wherein the multivariate discriminant is (i) the fractional expression with Lys, His, and Arg as the explanatory variables, (ii) the fractional expression with a-ABA, His, and Met as the explanatory variables, (iii) the fractional expression with Ile, His, Cit, and Arg as the explanatory variables, (iv) the linear discriminant with Gly, Val, His, and Arg as the explanatory variables, (v) the linear discriminant with Gly, Phe, His, and Arg as the explanatory variables, (vi) the linear discriminant with Cit, Ile, His, and Arg as the explanatory variables, (vii) the linear discriminant with His, Leu, Met, Cit, Ile, and Tyr as the explanatory variables, (viii) the logistic regression equation with Val, His, Lys, and Arg as the explanatory variables, (ix) the logistic regression equation with Thr, a-ABA, Met, and His as the explanatory variables, (x) the logistic regression equation with Cit, Ile, His, and Arg as the explanatory variables, or (xi) the logistic regression equation with His, Leu, Met, Cit, Ile, and Tyr as the explanatory variables.

Still another aspect of the present invention is the method of evaluating female genital cancer, wherein (I) at the discriminant value calculating step, the discriminant value is calculated based on (i) the concentration value of at least one of Asn, Val, Met, Leu, Phe, His, Trp, Orn, Lys, and Arg contained in the amino acid concentration data of the subject measured at the measuring step and (ii) the multivariate discriminant containing at least one of Asn, Val, Met, Leu, Phe, His, Trp, Orn, Lys, and Arg as the explanatory variable, and (II) at the discriminant value criterion discriminating step, the discrimination between the cervical cancer and the cervical cancer-free in the subject is conducted based on the discriminant value calculated at the discriminant value calculating step. Still another aspect of the present invention is the method of evaluating female genital cancer, wherein the multivariate discriminant is (i) the fractional expression with a-ABA, His, and Val as the explanatory variables, (ii) the fractional expression with a-ABA, Met, and Val as the explanatory variables, (iii) the fractional expression with Met, His, Cit, and Arg as the explanatory variables, (iv) the linear discriminant with Gly, Val, His, and Arg as the explanatory variables, (v) the linear discriminant with Gly, Val, Met, and Lys as the explanatory variables, (vi) the linear discriminant with Cit, Met, His, and Arg as the explanatory variables, (vii) the linear discriminant with His, Leu, Met, Ile, Tyr, and Lys as the explanatory variables, (viii) the logistic regression equation with Val, Leu, His, and Arg as the explanatory variables, (ix) the logistic regression equation with Met, His, Orn, and Arg as the explanatory variables, (x) the logistic regression equation with Val, Tyr, His, and Arg as the explanatory variables, or (xi) the logistic regression equation with His, Leu, Met, Ile, Tyr, and Lys as the explanatory variables.

Still another aspect of the present invention is the method of evaluating female genital cancer, wherein (I) at the discriminant value calculating step, the discriminant value is calculated based on (i) the concentration value of at least one of Thr, Ser, Asn, Pro, Gly, Cit, Val, Met, Ile, Leu, Phe, His, Trp, and Arg contained in the amino acid concentration data of the subject measured at the measuring step and (ii) the multivariate discriminant containing at least one of Thr, Ser, Asn, Pro, Gly, Cit, Val, Met, Ile, Leu, Phe, His, Trp, and Arg as the explanatory variable, and (II) at the discriminant value criterion discriminating step, the discrimination between the endometrial cancer and the endometrial cancer-free in the subject is conducted based on the discriminant value calculated at the discriminant value calculating step. Still another aspect of the present invention is the method of evaluating female genital cancer, wherein the multivariate discriminant is (i) the fractional expression with Lys, His, and Arg as the explanatory variables, (ii) the fractional expression with a-ABA, His, and Met as the explanatory variables, (iii) the fractional expression with Ile, His, Asn, and Cit as the explanatory variables, (iv) the linear discriminant with Gln, His, Lys, and Arg as the explanatory variables, (v) the linear discriminant with Gly, Met, Phe, and His as the explanatory variables, (vi) the linear discriminant with Cit, Ile, His, and Arg as the explanatory variables, (vii) the linear discriminant with His, Asn, Val, Pro, Cit, and Ile as the explanatory variables, (viii) the logistic regression equation with Gln, Gly, His, and Arg as the explanatory variables, (ix) the logistic regression equation with Gln, Phe, His, and Arg as the explanatory variables, (x) the logistic regression equation with Gln, Ile, His, and Arg as the explanatory variables, or (xi) the logistic regression equation with His, Asn, Val, Pro, Cit, and Ile as the explanatory variables.

Still another aspect of the present invention is the method of evaluating female genital cancer, wherein (I) at the discriminant value calculating step, the discriminant value is calculated based on (i) the concentration value of at least one of Thr, Ser, Asn, Gln, Ala, Cit, Val, Met, Ile, Leu, Tyr, Phe, His, Trp, Orn, Lys, and Arg contained in the amino acid concentration data of the subject measured at the measuring step and (ii) the multivariate discriminant containing at least one of Thr, Ser, Asn, Gln, Ala, Cit, Val, Met, Ile, Leu, Tyr, Phe, His, Trp, Orn, Lys, and Arg as the explanatory variable, and (II) at the discriminant value criterion discriminating step, the discrimination between the ovarian cancer and the ovarian cancer-free in the subject is conducted based on the discriminant value calculated at the discriminant value calculating step. Still another aspect of the present invention is the method of evaluating female genital cancer, wherein the multivariate discriminant is (i) the fractional expression with Orn, Cit, and Met as the explanatory variables, (ii) the fractional expression with Gln, Cit, and Tyr as the explanatory variables, (iii) the fractional expression with Orn, His, Phe, and Trp as the explanatory variables, (iv) the linear discriminant with Ser, Cit, Orn, and Trp as the explanatory variables, (v) the linear discriminant with Ser, Cit, Ile, and Orn as the explanatory variables, (vi) the linear discriminant with Phe, Trp, Orn, and Lys as the explanatory variables, (vii) the linear discriminant with His, Trp, Glu, Cit, Ile, and Orn as the explanatory variables, (viii) the logistic regression equation with Ser, Cit, Trp, and Orn as the explanatory variables, (ix) the logistic regression equation with Gln, Cit, Ile, and Tyr as the explanatory variables, (x) the logistic regression equation with Asn, Phe, His, and Trp as the explanatory variables, or (xi) the logistic regression equation with His, Trp, Glu, Cit, Ile, and Orn as the explanatory variables.

Still another aspect of the present invention is the method of evaluating female genital cancer, wherein (I) at the discriminant value calculating step, the discriminant value is calculated based on (i) the concentration value of at least one of Thr, Ser, Asn, Gln, Pro, Ala, Val, Met, Ile, Leu, Tyr, Phe, His, Trp, Orn, and Arg contained in the amino acid concentration data of the subject measured at the measuring step and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) at the discriminant value criterion discriminating step, the discrimination between the female genital cancer suffering risk group and the healthy group in the subject is conducted based on the discriminant value calculated at the discriminant value calculating step.

Still another aspect of the present invention is the method of evaluating female genital cancer, wherein the multivariate discriminant is the linear discriminant with Phe, His, Met, Pro, Lys, and Arg as the explanatory variables, or the logistic regression equation with Phe, His, Met, Pro, Lys, and Arg as the explanatory variables.

Still another aspect of the present invention is the method of evaluating female genital cancer, wherein (I) at the discriminant value calculating step, the discriminant value is calculated based on (i) the concentration value of at least one of Thr, Ser, Asn, Glu, Gln, Pro, Gly, Ala, Cit, ABA, Val, Met, Ile, Leu, Tyr, Phe, His, Trp, Orn, Lys, and Arg contained in the amino acid concentration data of the subject measured at the measuring step and (ii) the multivariate discriminant containing at least one of Thr, Ser, Asn, Glu, Gln, Pro, Gly, Ala, Cit, ABA, Val, Met, Ile, Leu, Tyr, Phe, His, Trp, Orn, Lys, and Arg as the explanatory variable, and (II) at the discriminant value criterion discriminating step, the discrimination between the cervical cancer, the endometrial cancer, and the ovarian cancer in the subject is conducted based on the discriminant value calculated at the discriminant value calculating step. Still another aspect of the present invention is the method of evaluating female genital cancer, wherein the multivariate discriminant is the discriminant with Cit, Met, Lys, Asn, Ala, Thr, Gln, and a-ABA as the explanatory variables which is prepared by the Mahalanobis' generalized distance method, or the discriminant prepared with His, Leu, Ser, Thr, Glu, Gln, Ala, and Lys as the explanatory variables which is prepared by the Mahalanobis' generalized distance method.

A female genital cancer-evaluating apparatus according to one aspect of the present invention includes a control unit and a memory unit to evaluate a state of female genital cancer including at least one of cervical cancer, endometrial cancer, and ovarian cancer in a subject to be evaluated. The control unit includes (I) a discriminant value-calculating unit that calculates a discriminant value that is a value of a multivariate discriminant with a concentration of an amino acid as an explanatory variable, based on both (i) a concentration value of at least one of Thr, Ser, Asn, Gln, Pro, Gly, Ala, Cit, Val, Met, Ile, Leu, Tyr, Phe, His, Trp, Orn, Lys, and Arg contained in a previously obtained amino acid concentration data of the subject on the concentration value of the amino acid and (ii) the multivariate discriminant stored in the memory unit, and (II) a discriminant value criterion-evaluating unit that evaluates the state of female genital cancer in the subject based on the discriminant value calculated by the discriminant value-calculating unit. The multivariate discriminant contains at least one of Thr, Ser, Asn, Gln, Pro, Gly, Ala, Cit, Val, Met, Ile, Leu, Tyr, Phe, His, Trp, Orn, Lys, and Arg as the explanatory variable.

Another aspect of the present invention is the female genital cancer-evaluating apparatus, wherein the discriminant value criterion-evaluating unit further includes a discriminant value criterion-discriminating unit that discriminates (i) between the female genital cancer and a female genital cancer-free, (ii) between any one of the cervical cancer, the endometrial cancer, and the ovarian cancer and the female genital cancer-free, (iii) between any one of the cervical cancer and the endometrial cancer and any one of a cervical cancer-free and an endometrial cancer-free, (iv) between the cervical cancer and the cervical cancer-free, (v) between the endometrial cancer and the endometrial cancer-free, (vi) between the ovarian cancer and a ovarian cancer-free, (vii) between a female genital cancer suffering risk group and a healthy group, or (viii) between the cervical cancer, the endometrial cancer, and the ovarian cancer in the subject, based on the discriminant value calculated by the discriminant value-calculating unit.

Still another aspect of the present invention is the female genital cancer-evaluating apparatus, wherein the multivariate discriminant is any one of a fractional expression, the sum of a plurality of the fractional expressions, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

Still another aspect of the present invention is the female genital cancer-evaluating apparatus, wherein (I) the discriminant value-calculating unit calculates the discriminant value based on (i) the concentration value of at least one of Thr, Ser, Asn, Gln, Pro, Ala, Val, Met, Ile, Leu, Tyr, Phe, His, Trp, Orn, and Arg contained in the amino acid concentration data of the subject and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) the discriminant value criterion-discriminating unit discriminates between any one of the cervical cancer, the endometrial cancer, and the ovarian cancer and the female genital cancer-free in the subject based on the discriminant value calculated by the discriminant value-calculating unit. Still another aspect of the present invention is the female genital cancer-evaluating apparatus, wherein the multivariate discriminant is (i) the fractional expression with Gln, His, and Arg as the explanatory variables, (ii) the fractional expression with a-ABA, His, and Met as the explanatory variables, (iii) the fractional expression with Ile, His, Cit, Arg, Tyr, and Trp as the explanatory variables, (iv) the fractional expression with a-ABA, Cit, and Met as the explanatory variables, (v) the linear discriminant with Gly, Val, His, and Arg as the explanatory variables, (vi) the linear discriminant with Gly, a-ABA, Met, and His as the explanatory variables, (vii) the linear discriminant with Ala, Ile, His, Trp, and Arg as the explanatory variables, (viii) the linear discriminant with Gly, Cit, Met, and Phe as the explanatory variables, (ix) the linear discriminant with His, Leu, Met, Cit, Ile, and Tyr as the explanatory variables, (x) the logistic regression equation with Val, Leu, His, and Arg as the explanatory variables, (xi) the logistic regression equation with a-ABA, Met, Tyr, and His as the explanatory variables, (xii) the logistic regression equation with Val, Ile, His, Trp, and Arg as the explanatory variables, (xiii) the logistic regression equation with Cit, a-ABA, Met, and Tyr as the explanatory variables, or (xiv) the logistic regression equation with His, Leu, Met, Cit, Ile, and Tyr as the explanatory variables.

Still another aspect of the present invention is the female genital cancer-evaluating apparatus, wherein (I) the discriminant value-calculating unit calculates the discriminant value based on (i) the concentration value of at least one of Thr, Ser, Asn, Pro, Gly, Cit, Val, Met, Ile, Leu, Phe, His, Trp, Orn, Lys, and Arg contained in the amino acid concentration data of the subject and (ii) the multivariate discriminant containing at least one of Thr, Ser, Asn, Pro, Gly, Cit, Val, Met, Ile, Leu, Phe, His, Trp, Orn, Lys, and Arg as the explanatory variable, and (II) the discriminant value criterion-discriminating unit discriminates between any one of the cervical cancer and the endometrial cancer and any one of the cervical cancer-free and the endometrial cancer-free in the subject based on the discriminant value calculated by the discriminant value-calculating unit. Still another aspect of the present invention is the female genital cancer-evaluating apparatus, wherein the multivariate discriminant is (i) the fractional expression with Lys, His, and Arg as the explanatory variables, (ii) the fractional expression with a-ABA, His, and Met as the explanatory variables, (iii) the fractional expression with Ile, His, Cit, and Arg as the explanatory variables, (iv) the linear discriminant with Gly, Val, His, and Arg as the explanatory variables, (v) the linear discriminant with Gly, Phe, His, and Arg as the explanatory variables, (vi) the linear discriminant with Cit, Ile, His, and Arg as the explanatory variables, (vii) the linear discriminant with His, Leu, Met, Cit, Ile, and Tyr as the explanatory variables, (viii) the logistic regression equation with Val, His, Lys, and Arg as the explanatory variables, (ix) the logistic regression equation with Thr, a-ABA, Met, and His as the explanatory variables, (x) the logistic regression equation with Cit, Ile, His, and Arg as the explanatory variables, or (xi) the logistic regression equation with His, Leu, Met, Cit, Ile, and Tyr as the explanatory variables.

Still another aspect of the present invention is the female genital cancer-evaluating apparatus, wherein (I) the discriminant value-calculating unit calculates the discriminant value based on (i) the concentration value of at least one of Asn, Val, Met, Leu, Phe, His, Trp, Orn, Lys, and Arg contained in the amino acid concentration data of the subject and (ii) the multivariate discriminant containing at least one of Asn, Val, Met, Leu, Phe, His, Trp, Orn, Lys, and Arg as the explanatory variable, and (II) the discriminant value criterion-discriminating unit discriminates between the cervical cancer and the cervical cancer-free in the subject based on the discriminant value calculated by the discriminant value-calculating unit. Still another aspect of the present invention is the female genital cancer-evaluating apparatus, wherein the multivariate discriminant is (i) the fractional expression with a-ABA, His, and Val as the explanatory variables, (ii) the fractional expression with a-ABA, Met, and Val as the explanatory variables, (iii) the fractional expression with Met, His, Cit, and Arg as the explanatory variables, (iv) the linear discriminant with Gly, Val, His, and Arg as the explanatory variables, (v) the linear discriminant with Gly, Val, Met, and Lys as the explanatory variables, (vi) the linear discriminant with Cit, Met, His, and Arg as the explanatory variables, (vii) the linear discriminant with His, Leu, Met, Ile, Tyr, and Lys as the explanatory variables, (viii) the logistic regression equation with Val, Leu, His, and Arg as the explanatory variables, (ix) the logistic regression equation with Met, His, Orn, and Arg as the explanatory variables, (x) the logistic regression equation with Val, Tyr, His, and Arg as the explanatory variables, or (xi) the logistic regression equation with His, Leu, Met, Ile, Tyr, and Lys as the explanatory variables.

Still another aspect of the present invention is the female genital cancer-evaluating apparatus, wherein (I) the discriminant value-calculating unit calculates the discriminant value based on (i) the concentration value of at least one of Thr, Ser, Asn, Pro, Gly, Cit, Val, Met, Ile, Leu, Phe, His, Trp, and Arg contained in the amino acid concentration data of the subject and (ii) the multivariate discriminant containing at least one of Thr, Ser, Asn, Pro, Gly, Cit, Val, Met, Ile, Leu, Phe, His, Trp, and Arg as the explanatory variable, and (II) the discriminant value criterion-discriminating unit discriminates between the endometrial cancer and the endometrial cancer-free in the subject based on the discriminant value calculated by the discriminant value-calculating unit. Still another aspect of the present invention is the female genital cancer-evaluating apparatus, wherein the multivariate discriminant is (i) the fractional expression with Lys, His, and Arg as the explanatory variables, (ii) the fractional expression with a-ABA, His, and Met as the explanatory variables, (iii) the fractional expression with Ile, His, Asn, and Cit as the explanatory variables, (iv) the linear discriminant with Gln, His, Lys, and Arg as the explanatory variables, (v) the linear discriminant with Gly, Met, Phe, and His as the explanatory variables, (vi) the linear discriminant with Cit, Ile, His, and Arg as the explanatory variables, (vii) the linear discriminant with His, Asn, Val, Pro, Cit, and Ile as the explanatory variables, (viii) the logistic regression equation with Gln, Gly, His, and Arg as the explanatory variables, (ix) the logistic regression equation with Gln, Phe, His, and Arg as the explanatory variables, (x) the logistic regression equation with Gln, Ile, His, and Arg as the explanatory variables, or (xi) the logistic regression equation with His, Asn, Val, Pro, Cit, and Ile as the explanatory variables.

Still another aspect of the present invention is the female genital cancer-evaluating apparatus, wherein (I) the discriminant value-calculating unit calculates the discriminant value based on (i) the concentration value of at least one of Thr, Ser, Asn, Gln, Ala, Cit, Val, Met, Ile, Leu, Tyr, Phe, His, Trp, Orn, Lys, and Arg contained in the amino acid concentration data of the subject and (ii) the multivariate discriminant containing at least one of Thr, Ser, Asn, Gln, Ala, Cit, Val, Met, Ile, Leu, Tyr, Phe, His, Trp, Orn, Lys, and Arg as the explanatory variable, and (II) the discriminant value criterion-discriminating unit discriminates between the ovarian cancer and the ovarian cancer-free in the subject based on the discriminant value calculated by the discriminant value-calculating unit. Still another aspect of the present invention is the female genital cancer-evaluating apparatus, wherein the multivariate discriminant is (i) the fractional expression with Orn, Cit, and Met as the explanatory variables, (ii) the fractional expression with Gln, Cit, and Tyr as the explanatory variables, (iii) the fractional expression with Orn, His, Phe, and Trp as the explanatory variables, (iv) the linear discriminant with Ser, Cit, Orn, and Trp as the explanatory variables, (v) the linear discriminant with Ser, Cit, Ile, and Orn as the explanatory variables, (vi) the linear discriminant with Phe, Trp, Orn, and Lys as the explanatory variables, (vii) the linear discriminant with His, Trp, Glu, Cit, Ile, and Orn as the explanatory variables, (viii) the logistic regression equation with Ser, Cit, Trp, and Orn as the explanatory variables, (ix) the logistic regression equation with Gln, Cit, Ile, and Tyr as the explanatory variables, (x) the logistic regression equation with Asn, Phe, His, and Trp as the explanatory variables, or (xi) the logistic regression equation with His, Trp, Glu, Cit, Ile, and Orn as the explanatory variables.

Still another aspect of the present invention is the female genital cancer-evaluating apparatus, wherein (I) the discriminant value-calculating unit calculates the discriminant value based on (i) the concentration value of at least one of Thr, Ser, Asn, Gln, Pro, Ala, Val, Met, Ile, Leu, Tyr, Phe, His, Trp, Orn, and Arg contained in the amino acid concentration data of the subject and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) the discriminant value criterion-discriminating unit discriminates between the female genital cancer suffering risk group and the healthy group in the subject based on the discriminant value calculated by the discriminant value-calculating unit. Still another aspect of the present invention is the female genital cancer-evaluating apparatus, wherein the multivariate discriminant is the linear discriminant with Phe, His, Met, Pro, Lys, and Arg as the explanatory variables, or the logistic regression equation with Phe, His, Met, Pro, Lys, and Arg as the explanatory variables.

Still another aspect of the present invention is the female genital cancer-evaluating apparatus, wherein (I) the discriminant value-calculating unit calculates the discriminant value based on (i) the concentration value of at least one of Thr, Ser, Asn, Glu, Gln, Pro, Gly, Ala, Cit, ABA, Val, Met, Ile, Leu, Tyr, Phe, His, Trp, Orn, Lys, and Arg contained in the amino acid concentration data of the subject and (ii) the multivariate discriminant containing at least one of Thr, Ser, Asn, Glu, Gln, Pro, Gly, Ala, Cit, ABA, Val, Met, Ile, Leu, Tyr, Phe, His, Trp, Orn, Lys, and Arg as the explanatory variable, and (II) the discriminant value criterion-discriminating unit discriminates between the cervical cancer, the endometrial cancer, and the ovarian cancer in the subject based on the discriminant value calculated by the discriminant value-calculating unit. Still another aspect of the present invention is the female genital cancer-evaluating apparatus, wherein the multivariate discriminant is the discriminant with Cit, Met, Lys, Asn, Ala, Thr, Gln, and a-ABA as the explanatory variables which is prepared by the Mahalanobis' generalized distance method, or the discriminant prepared with His, Leu, Ser, Thr, Glu, Gln, Ala, and Lys as the explanatory variables which is prepared by the Mahalanobis' generalized distance method.

Still another aspect of the present invention is the female genital cancer-evaluating apparatus, wherein the control unit further includes a multivariate discriminant-preparing unit that prepares the multivariate discriminant stored in the memory unit, based on female genital cancer state information containing the amino acid concentration data and female genital cancer state index data on an index for indicating the state of female genital cancer, stored in the memory unit. The multivariate discriminant-preparing unit further includes (I) a candidate multivariate discriminant-preparing unit that prepares a candidate multivariate discriminant that is a candidate of the multivariate discriminant, based on a predetermined discriminant-preparing method from the female genital cancer state information, (II) a candidate multivariate discriminant-verifying unit that verifies the candidate multivariate discriminant prepared by the candidate multivariate discriminant-preparing unit, based on a predetermined verifying method, and (III) an explanatory variable-selecting unit that selects the explanatory variable of the candidate multivariate discriminant based on a predetermined explanatory variable-selecting method from a verification result obtained by the candidate multivariate discriminant-verifying unit, thereby selecting a combination of the amino acid concentration data contained in the female genital cancer state information used in preparing the candidate multivariate discriminant. The multivariate discriminant-preparing unit prepares the multivariate discriminant by selecting the candidate multivariate discriminant used as the multivariate discriminant, from a plurality of the candidate multivariate discriminants, based on the verification results accumulated by repeatedly executing the candidate multivariate discriminant-preparing unit, the candidate multivariate discriminant-verifying unit, and the explanatory variable-selecting unit.

A female genital cancer-evaluating method according to one aspect of the present invention is a method of evaluating a state of female genital cancer including at least one of cervical cancer, endometrial cancer, and ovarian cancer in a subject to be evaluated. The method is carried out with an information processing apparatus including a control unit and a memory unit. The method includes (I) a discriminant value calculating step of calculating a discriminant value that is a value of a multivariate discriminant with a concentration of an amino acid as an explanatory variable, based on both (i) a concentration value of at least one of Thr, Ser, Asn, Gln, Pro, Gly, Ala, Cit, Val, Met, Ile, Leu, Tyr, Phe, His, Trp, Orn, Lys, and Arg contained in a previously obtained amino acid concentration data of the subject on the concentration value of the amino acid and (ii) the multivariate discriminant stored in the memory unit, and (II) a discriminant value criterion evaluating step of evaluating the state of female genital cancer in the subject based on the discriminant value calculated at the discriminant value calculating step. The multivariate discriminant contains at least one of Thr, Ser, Asn, Gln, Pro, Gly, Ala, Cit, Val, Met, Ile, Leu, Tyr, Phe, His, Trp, Orn, Lys, and Arg as the explanatory variable. The steps (I) and (II) are executed by the control unit.

Another aspect of the present invention is the female genital cancer-evaluating method, wherein the discriminant value criterion evaluating step further includes a discriminant value criterion discriminating step of discriminating (i) between the female genital cancer and a female genital cancer-free, (ii) between any one of the cervical cancer, the endometrial cancer, and the ovarian cancer and the female genital cancer-free, (iii) between any one of the cervical cancer and the endometrial cancer and any one of a cervical cancer-free and an endometrial cancer-free, (iv) between the cervical cancer and the cervical cancer-free, (v) between the endometrial cancer and the endometrial cancer-free, (vi) between the ovarian cancer and a ovarian cancer-free, (vii) between a female genital cancer suffering risk group and a healthy group, or (viii) between the cervical cancer, the endometrial cancer, and the ovarian cancer in the subject, based on the discriminant value calculated at the discriminant value calculating step.

Still another aspect of the present invention is the female genital cancer-evaluating method, wherein the multivariate discriminant is any one of a fractional expression, the sum of a plurality of the fractional expressions, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

Still another aspect of the present invention is the female genital cancer-evaluating method, wherein (I) at the discriminant value calculating step, the discriminant value is calculated based on (i) the concentration value of at least one of Thr, Ser, Asn, Gln, Pro, Ala, Val, Met, Ile, Leu, Tyr, Phe, His, Trp, Orn, and Arg contained in the amino acid concentration data of the subject and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) at the discriminant value criterion discriminating step, the discrimination between any one of the cervical cancer, the endometrial cancer, and the ovarian cancer and the female genital cancer-free in the subject is conducted based on the discriminant value calculated at the discriminant value calculating step. Still another aspect of the present invention is the female genital cancer-evaluating method, wherein the multivariate discriminant is (i) the fractional expression with Gln, His, and Arg as the explanatory variables, (ii) the fractional expression with a-ABA, His, and Met as the explanatory variables, (iii) the fractional expression with Ile, His, Cit, Arg, Tyr, and Trp as the explanatory variables, (iv) the fractional expression with a-ABA, Cit, and Met as the explanatory variables, (v) the linear discriminant with Gly, Val, His, and Arg as the explanatory variables, (vi) the linear discriminant with Gly, a-ABA, Met, and His as the explanatory variables, (vii) the linear discriminant with Ala, Ile, His, Trp, and Arg as the explanatory variables, (viii) the linear discriminant with Gly, Cit, Met, and Phe as the explanatory variables, (ix) the linear discriminant with His, Leu, Met, Cit, Ile, and Tyr as the explanatory variables, (x) the logistic regression equation with Val, Leu, His, and Arg as the explanatory variables, (xi) the logistic regression equation with a-ABA, Met, Tyr, and His as the explanatory variables, (xii) the logistic regression equation with Val, Ile, His, Trp, and Arg as the explanatory variables, (xiii) the logistic regression equation with Cit, a-ABA, Met, and Tyr as the explanatory variables, or (xiv) the logistic regression equation with His, Leu, Met, Cit, Ile, and Tyr as the explanatory variables.

Still another aspect of the present invention is the female genital cancer-evaluating method, wherein (I) at the discriminant value calculating step, the discriminant value is calculated based on (i) the concentration value of at least one of Thr, Ser, Asn, Pro, Gly, Cit, Val, Met, Ile, Leu, Phe, His, Trp, Orn, Lys, and Arg contained in the amino acid concentration data of the subject and (ii) the multivariate discriminant containing at least one of Thr, Ser, Asn, Pro, Gly, Cit, Val, Met, Ile, Leu, Phe, His, Trp, Orn, Lys, and Arg as the explanatory variable, and (II) at the discriminant value criterion discriminating step, the discrimination between any one of the cervical cancer and the endometrial cancer and any one of the cervical cancer-free and the endometrial cancer-free in the subject is conducted based on the discriminant value calculated at the discriminant value calculating step. Still another aspect of the present invention is the female genital cancer-evaluating method, wherein the multivariate discriminant is (i) the fractional expression with Lys, His, and Arg as the explanatory variables, (ii) the fractional expression with a-ABA, His, and Met as the explanatory variables, (iii) the fractional expression with Ile, His, Cit, and Arg as the explanatory variables, (iv) the linear discriminant with Gly, Val, His, and Arg as the explanatory variables, (v) the linear discriminant with Gly, Phe, His, and Arg as the explanatory variables, (vi) the linear discriminant with Cit, Ile, His, and Arg as the explanatory variables, (vii) the linear discriminant with His, Leu, Met, Cit, Ile, and Tyr as the explanatory variables, (viii) the logistic regression equation with Val, His, Lys, and Arg as the explanatory variables, (ix) the logistic regression equation with Thr, a-ABA, Met, and His as the explanatory variables, (x) the logistic regression equation with Cit, Ile, His, and Arg as the explanatory variables, or (xi) the logistic regression equation with His, Leu, Met, Cit, Ile, and Tyr as the explanatory variables.

Still another aspect of the present invention is the female genital cancer-evaluating method, wherein (I) at the discriminant value calculating step, the discriminant value is calculated based on (i) the concentration value of at least one of Asn, Val, Met, Leu, Phe, His, Trp, Orn, Lys, and Arg contained in the amino acid concentration data of the subject and (ii) the multivariate discriminant containing at least one of Asn, Val, Met, Leu, Phe, His, Trp, Orn, Lys, and Arg as the explanatory variable, and (II) at the discriminant value criterion discriminating step, the discrimination between the cervical cancer and the cervical cancer-free in the subject is conducted based on the discriminant value calculated at the discriminant value calculating step. Still another aspect of the present invention is the female genital cancer-evaluating method, wherein the multivariate discriminant is (i) the fractional expression with a-ABA, His, and Val as the explanatory variables, (ii) the fractional expression with a-ABA, Met, and Val as the explanatory variables, (iii) the fractional expression with Met, His, Cit, and Arg as the explanatory variables, (iv) the linear discriminant with Gly, Val, His, and Arg as the explanatory variables, (v) the linear discriminant with Gly, Val, Met, and Lys as the explanatory variables, (vi) the linear discriminant with Cit, Met, His, and Arg as the explanatory variables, (vii) the linear discriminant with His, Leu, Met, Ile, Tyr, and Lys as the explanatory variables, (viii) the logistic regression equation with Val, Leu, His, and Arg as the explanatory variables, (ix) the logistic regression equation with Met, His, Orn, and Arg as the explanatory variables, (x) the logistic regression equation with Val, Tyr, His, and Arg as the explanatory variables, or (xi) the logistic regression equation with His, Leu, Met, Ile, Tyr, and Lys as the explanatory variables.

Still another aspect of the present invention is the female genital cancer-evaluating method, wherein (I) at the discriminant value calculating step, the discriminant value is calculated based on (i) the concentration value of at least one of Thr, Ser, Asn, Pro, Gly, Cit, Val, Met, Ile, Leu, Phe, His, Trp, and Arg contained in the amino acid concentration data of the subject and (ii) the multivariate discriminant containing at least one of Thr, Ser, Asn, Pro, Gly, Cit, Val, Met, Ile, Leu, Phe, His, Trp, and Arg as the explanatory variable, and (II) at the discriminant value criterion discriminating step, the discrimination between the endometrial cancer and the endometrial cancer-free in the subject is conducted based on the discriminant value calculated at the discriminant value calculating step. Still another aspect of the present invention is the female genital cancer-evaluating method, wherein the multivariate discriminant is (i) the fractional expression with Lys, His, and Arg as the explanatory variables, (ii) the fractional expression with a-ABA, His, and Met as the explanatory variables, (iii) the fractional expression with Ile, His, Asn, and Cit as the explanatory variables, (iv) the linear discriminant with Gln, His, Lys, and Arg as the explanatory variables, (v) the linear discriminant with Gly, Met, Phe, and His as the explanatory variables, (vi) the linear discriminant with Cit, Ile, His, and Arg as the explanatory variables, (vii) the linear discriminant with His, Asn, Val, Pro, Cit, and Ile as the explanatory variables, (viii) the logistic regression equation with Gln, Gly, His, and Arg as the explanatory variables, (ix) the logistic regression equation with Gln, Phe, His, and Arg as the explanatory variables, (x) the logistic regression equation with Gln, Ile, His, and Arg as the explanatory variables, or (xi) the logistic regression equation with His, Asn, Val, Pro, Cit, and Ile as the explanatory variables.

Still another aspect of the present invention is the female genital cancer-evaluating method, wherein (I) at the discriminant value calculating step, the discriminant value is calculated based on (i) the concentration value of at least one of Thr, Ser, Asn, Gln, Ala, Cit, Val, Met, Ile, Leu, Tyr, Phe, His, Trp, Orn, Lys, and Arg contained in the amino acid concentration data of the subject and (ii) the multivariate discriminant containing at least one of Thr, Ser, Asn, Gln, Ala, Cit, Val, Met, Ile, Leu, Tyr, Phe, His, Trp, Orn, Lys, and Arg as the explanatory variable, and (II) at the discriminant value criterion discriminating step, the discrimination between the ovarian cancer and the ovarian cancer-free in the subject is conducted based on the discriminant value calculated at the discriminant value calculating step. Still another aspect of the present invention is the female genital cancer-evaluating method, wherein the multivariate discriminant is (i) the fractional expression with Orn, Cit, and Met as the explanatory variables, (ii) the fractional expression with Gln, Cit, and Tyr as the explanatory variables, (iii) the fractional expression with Orn, His, Phe, and Trp as the explanatory variables, (iv) the linear discriminant with Ser, Cit, Orn, and Trp as the explanatory variables, (v) the linear discriminant with Ser, Cit, Ile, and Orn as the explanatory variables, (vi) the linear discriminant with Phe, Trp, Orn, and Lys as the explanatory variables, (vii) the linear discriminant with His, Trp, Glu, Cit, Ile, and Orn as the explanatory variables, (viii) the logistic regression equation with Ser, Cit, Trp, and Orn as the explanatory variables, (ix) the logistic regression equation with Gln, Cit, Ile, and Tyr as the explanatory variables, (x) the logistic regression equation with Asn, Phe, His, and Trp as the explanatory variables, or (xi) the logistic regression equation with His, Trp, Glu, Cit, Ile, and Orn as the explanatory variables.

Still another aspect of the present invention is the female genital cancer-evaluating method, wherein (I) at the discriminant value calculating step, the discriminant value is calculated based on (i) the concentration value of at least one of Thr, Ser, Asn, Gln, Pro, Ala, Val, Met, Ile, Leu, Tyr, Phe, His, Trp, Orn, and Arg contained in the amino acid concentration data of the subject and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) at the discriminant value criterion discriminating step, the discrimination between the female genital cancer suffering risk group and the healthy group in the subject is conducted based on the discriminant value calculated at the discriminant value calculating step. Still another aspect of the present invention is the female genital cancer-evaluating method, wherein the multivariate discriminant is the linear discriminant with Phe, His, Met, Pro, Lys, and Arg as the explanatory variables, or the logistic regression equation with Phe, His, Met, Pro, Lys, and Arg as the explanatory variables.

Still another aspect of the present invention is the female genital cancer-evaluating method, wherein (I) at the discriminant value calculating step, the discriminant value is calculated based on (i) the concentration value of at least one of Thr, Ser, Asn, Glu, Gln, Pro, Gly, Ala, Cit, ABA, Val, Met, Ile, Leu, Tyr, Phe, His, Trp, Orn, Lys, and Arg contained in the amino acid concentration data of the subject and (ii) the multivariate discriminant containing at least one of Thr, Ser, Asn, Glu, Gln, Pro, Gly, Ala, Cit, ABA, Val, Met, Ile, Leu, Tyr, Phe, His, Trp, Orn, Lys, and Arg as the explanatory variable, and (II) at the discriminant value criterion discriminating step, the discrimination between the cervical cancer, the endometrial cancer, and the ovarian cancer in the subject is conducted based on the discriminant value calculated at the discriminant value calculating step. Still another aspect of the present invention is the female genital cancer-evaluating method, wherein the multivariate discriminant is the discriminant with Cit, Met, Lys, Asn, Ala, Thr, Gln, and a-ABA as the explanatory variables which is prepared by the Mahalanobis' generalized distance method, or the discriminant prepared with His, Leu, Ser, Thr, Glu, Gln, Ala, and Lys as the explanatory variables which is prepared by the Mahalanobis' generalized distance method.

Still another aspect of the present invention is the female genital cancer-evaluating method, wherein the method further includes a multivariate discriminant preparing step of preparing the multivariate discriminant stored in the memory unit, based on female genital cancer state information containing the amino acid concentration data and female genital cancer state index date on an index for indicating the state of female genital cancer, stored in the memory unit. The multivariate discriminant preparing step is executed by the control unit. The multivariate discriminant preparing step further includes (I) a candidate multivariate discriminant preparing step of preparing a candidate multivariate discriminant that is a candidate of the multivariate discriminant, based on a predetermined discriminant-preparing method from the female genital cancer state information, (II) a candidate multivariate discriminant verifying step of verifying the candidate multivariate discriminant prepared at the candidate multivariate preparing step, based on a predetermined verifying method, and (III) an explanatory variable selecting step of selecting the explanatory variable of the candidate multivariate discriminant based on a predetermined explanatory variable-selecting method from a verification result obtained at the candidate multivariate discriminant verifying step, thereby selecting a combination of the amino acid concentration data contained in the female genital cancer state information used in preparing the candidate multivariate discriminant. At the multivariate discriminant preparing step, the multivariate discriminant is prepared by selecting the candidate multivariate discriminant used as the multivariate discriminant, from a plurality of the candidate multivariate discriminants, based on the verification results accumulated by repeatedly executing the candidate multivariate discriminant preparing step, the candidate multivariate discriminant verifying step, and the explanatory variable selecting step.

A female genital cancer-evaluating system according to one aspect of the present invention includes (I) a female genital cancer-evaluating apparatus including a control unit and a memory unit to evaluate a state of female genital cancer including at least one of cervical cancer, endometrial cancer, and ovarian cancer in a subject to be evaluated, and (II) an information communication terminal apparatus that provides amino acid concentration data of the subject on a concentration value of an amino acid. The apparatuses are connected to each other communicatively via a network. The information communication terminal apparatus includes an amino acid concentration data-sending unit that transmits the amino acid concentration data of the subject to the female genital cancer-evaluating apparatus, and an evaluation result-receiving unit that receives an evaluation result of the subject on the state of female genital cancer transmitted from the female genital cancer-evaluating apparatus. The control unit of the female genital cancer-evaluating apparatus includes (I) an amino acid concentration data-receiving unit that receives the amino acid concentration data of the subject transmitted from the information communication terminal apparatus, (II) a discriminant value-calculating unit that calculates a discriminant value that is a value of a multivariate discriminant with a concentration of the amino acid as an explanatory variable, based on both (i) the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data of the subject received by the amino acid concentration data-receiving unit and (ii) the multivariate discriminant stored in the memory unit, (III) a discriminant value criterion-evaluating unit that evaluates the state of female genital cancer in the subject based on the discriminant value calculated by the discriminant value-calculating unit, and (IV) an evaluation result-sending unit that transmits the evaluation result of the subject obtained by the discriminant value criterion-evaluating unit to the information communication terminal apparatus. The multivariate discriminant contains at least one of Thr, Ser, Asn, Gln, Pro, Gly, Ala, Cit, Val, Met, Ile, Leu, Tyr, Phe, His, Trp, Orn, Lys, and Arg as the explanatory variable.

A female genital cancer-evaluating program product according to one aspect of the present invention makes an information processing apparatus including a control unit and a memory unit execute a method of evaluating a state of female genital cancer including at least one of cervical cancer, endometrial cancer, and ovarian cancer in a subject to be evaluated. The method includes (I) a discriminant value calculating step of calculating a discriminant value that is a value of a multivariate discriminant with a concentration of an amino acid as an explanatory variable, based on both (i) a concentration value of at least one of Thr, Ser, Asn, Gln, Pro, Gly, Ala, Cit, Val, Met, Ile, Leu, Tyr, Phe, His, Trp, Orn, Lys, and Arg contained in a previously obtained amino acid concentration data of the subject on the concentration value of the amino acid and (ii) the multivariate discriminant stored in the memory unit, and (II) a discriminant value criterion evaluating step of evaluating the state of female genital cancer in the subject based on the discriminant value calculated at the discriminant value calculating step. The multivariate discriminant contains at least one of Thr, Ser, Asn, Gln, Pro, Gly, Ala, Cit, Val, Met, Ile, Leu, Tyr, Phe, His, Trp, Orn, Lys, and Arg as the explanatory variable. The steps (I) and (II) are executed by the control unit.

The present invention also relates to a recording medium, the recording medium according to one aspect of the present invention includes the female genital cancer-evaluating program product described above.

According to the present invention, (I) the amino acid concentration data on the concentration value of the amino acid in blood collected from the subject is measured, and (II) the state of female genital cancer including at least one of cervical cancer, endometrial cancer, and ovarian cancer in the subject is evaluated based on the concentration value of at least one of Thr, Ser, Asn, Gln, Pro, Gly, Ala, Cit, Val, Met, Ile, Leu, Tyr, Phe, His, Trp, Orn, Lys, and Arg contained in the measured amino acid concentration data of the subject. Thus, the concentrations of the amino acids which among amino acids in blood, are related to the state of female genital cancer can be utilized to bring about the effect of enabling an accurate evaluation of the state of female genital cancer. Specifically, a subject likely to contract female genital cancer can be narrowed by one sample in a short time to bring about the effect of enabling the reduction of temporal, physical and financial burden of the subject. Specifically, whether a certain sample is with female genital cancer can be evaluated accurately by the concentrations of a plurality of the amino acids to bring about the effect of enabling to make the examination efficient and high accurate.

According to the present invention, the discrimination (i) between the female genital cancer and the female genital cancer-free, (ii) between any one of the cervical cancer, the endometrial cancer, and the ovarian cancer and the female genital cancer-free, (iii) between any one of the cervical cancer and the endometrial cancer and any one of the cervical cancer-free and the endometrial cancer-free, (iv) between the cervical cancer and the cervical cancer-free, (v) between the endometrial cancer and the endometrial cancer-free, (vi) between the ovarian cancer and the ovarian cancer-free, (vii) between the female genital cancer suffering risk group and the healthy group, or (viii) between the cervical cancer, the endometrial cancer, and the ovarian cancer in the subject is conducted based on the concentration value of at least one of Thr, Ser, Asn, Gln, Pro, Gly, Ala, Cit, Val, Met, Ile, Leu, Tyr, Phe, His, Trp, Orn, Lys, and Arg contained in the measured amino acid concentration data of the subject. Thus, the concentrations of the amino acids which among amino acids in blood, are useful for (i) the 2-group discrimination between the female genital cancer and the female genital cancer-free, (ii) the discrimination between any one of the cervical cancer, the endometrial cancer, and the ovarian cancer and the female genital cancer-free, (iii) the discrimination between any one of the cervical cancer and the endometrial cancer and any one of the cervical cancer-free and the endometrial cancer-free, (iv) the 2-group discrimination between the cervical cancer and the cervical cancer-free, (v) the 2-group discrimination between the endometrial cancer and the endometrial cancer-free, (vi) the 2-group discrimination between the ovarian cancer and the ovarian cancer-free, (vii) the 2-group discrimination between the female genital cancer suffering risk group and the healthy group, or (viii) the discrimination between the cervical cancer, the endometrial cancer, and the ovarian cancer, can be utilized to bring about the effect of enabling accurately these 2-group discriminations or these discriminations.

According to the present invention, (I) the discriminant value that is the value of the multivariate discriminant with the concentration of the amino acid as the explanatory variable is calculated based on both (i) the concentration value of at least one of Thr, Ser, Asn, Gln, Pro, Gly, Ala, Cit, Val, Met, Ile, Leu, Tyr, Phe, His, Trp, Orn, Lys, and Arg contained in the measured amino acid concentration data of the subject and (ii) the previously established multivariate discriminant containing at least one of Thr, Ser, Asn, Gln, Pro, Gly, Ala, Cit, Val, Met, Ile, Leu, Tyr, Phe, His, Trp, Orn, Lys, and Arg as the explanatory variable, and (II) the state of female genital cancer in the subject is evaluated based on the calculated discriminant value. Thus, the discriminant values obtained in the multivariate discriminants correlated significantly with the state of female genital cancer can be utilized to bring about the effect of enabling an accurate evaluation of the state of female genital cancer. Specifically, a subject likely to contract female genital cancer can be narrowed by one sample in a short time to bring about the effect of enabling the reduction of temporal, physical and financial burden of the subject. Specifically, whether a certain sample is with female genital cancer can be evaluated accurately by the concentrations of a plurality of the amino acids and the discriminants with the concentrations of the amino acids as the explanatory variables to bring about the effect of enabling to make the examination efficient and high accurate.

According to the present invention, the discrimination (i) between the female genital cancer and the female genital cancer-free, (ii) between any one of the cervical cancer, the endometrial cancer, and the ovarian cancer and the female genital cancer-free, (iii) between any one of the cervical cancer and the endometrial cancer and any one of the cervical cancer-free and the endometrial cancer-free, (iv) between the cervical cancer and the cervical cancer-free, (v) between the endometrial cancer and the endometrial cancer-free, (vi) between the ovarian cancer and the ovarian cancer-free, (vii) between the female genital cancer suffering risk group and the healthy group, or (viii) between the cervical cancer, the endometrial cancer, and the ovarian cancer in the subject is conducted based on the calculated discriminant value. Thus, the discriminant values obtained in the multivariate discriminants useful for (i) the 2-group discrimination between the female genital cancer and the female genital cancer-free, (ii) the discrimination between any one of the cervical cancer, the endometrial cancer, and the ovarian cancer and the female genital cancer-free, (iii) the discrimination between any one of the cervical cancer and the endometrial cancer and any one of the cervical cancer-free and the endometrial cancer-free, (iv) the 2-group discrimination between the cervical cancer and the cervical cancer-free, (v) the 2-group discrimination between the endometrial cancer and the endometrial cancer-free, (vi) the 2-group discrimination between the ovarian cancer and the ovarian cancer-free, (vii) the 2-group discrimination between the female genital cancer suffering risk group and the healthy group, or (viii) the discrimination between the cervical cancer, the endometrial cancer, and the ovarian cancer, can be utilized to bring about the effect of enabling accurately these 2-group discriminations or these discriminations.

According to the present invention, the multivariate discriminant is any one of a fractional expression, the sum of a plurality of the fractional expressions, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Thus, the discriminant values obtained in the multivariate discriminants useful particularly for (i) the 2-group discrimination between the female genital cancer and the female genital cancer-free, (ii) the discrimination between any one of the cervical cancer, the endometrial cancer, and the ovarian cancer and the female genital cancer-free, (iii) the discrimination between any one of the cervical cancer and the endometrial cancer and any one of the cervical cancer-free and the endometrial cancer-free, (iv) the 2-group discrimination between the cervical cancer and the cervical cancer-free, (v) the 2-group discrimination between the endometrial cancer and the endometrial cancer-free, (vi) the 2-group discrimination between the ovarian cancer and the ovarian cancer-free, (vii) the 2-group discrimination between the female genital cancer suffering risk group and the healthy group, or (viii) the discrimination between the cervical cancer, the endometrial cancer, and the ovarian cancer, can be utilized to bring about the effect of enabling more accurately these 2-group discriminations or these discriminations.

According to the present invention, (I) the discriminant value is calculated based on (i) the concentration value of at least one of Thr, Ser, Asn, Gln, Pro, Ala, Val, Met, Ile, Leu, Tyr, Phe, His, Trp, Orn, and Arg contained in the measured amino acid concentration data of the subject and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) the discrimination between any one of the cervical cancer, the endometrial cancer, and the ovarian cancer and the female genital cancer-free in the subject is conducted based on the calculated discriminant value. Thus, the discriminant values obtained in the multivariate discriminants useful particularly for the discrimination between any one of the cervical cancer, the endometrial cancer, and the ovarian cancer and the female genital cancer-free, can be utilized to bring about the effect of enabling more accurately the discrimination. According to the present invention, the multivariate discriminant is (i) the fractional expression with Gln, His, and Arg as the explanatory variables, (ii) the fractional expression with a-ABA, His, and Met as the explanatory variables, (iii) the fractional expression with Ile, His, Cit, Arg, Tyr, and Trp as the explanatory variables, (iv) the fractional expression with a-ABA, Cit, and Met as the explanatory variables, (v) the linear discriminant with Gly, Val, His, and Arg as the explanatory variables, (vi) the linear discriminant with Gly, a-ABA, Met, and His as the explanatory variables, (vii) the linear discriminant with Ala, Ile, His, Trp, and Arg as the explanatory variables, (viii) the linear discriminant with Gly, Cit, Met, and Phe as the explanatory variables, (ix) the linear discriminant with His, Leu, Met, Cit, Ile, and Tyr as the explanatory variables, (x) the logistic regression equation with Val, Leu, His, and Arg as the explanatory variables, (xi) the logistic regression equation with a-ABA, Met, Tyr, and His as the explanatory variables, (xii) the logistic regression equation with Val, Ile, His, Trp, and Arg as the explanatory variables, (xiii) the logistic regression equation with Cit, a-ABA, Met, and Tyr as the explanatory variables, or (xiv) the logistic regression equation with His, Leu, Met, Cit, Ile, and Tyr as the explanatory variables. Thus, the discriminant values obtained in the multivariate discriminants useful particularly for the discrimination between any one of the cervical cancer, the endometrial cancer, and the ovarian cancer and the female genital cancer-free, can be utilized to bring about the effect of enabling more accurately the discrimination.

According to the present invention, (I) the discriminant value is calculated based on (i) the concentration value of at least one of Thr, Ser, Asn, Pro, Gly, Cit, Val, Met, Ile, Leu, Phe, His, Trp, Orn, Lys, and Arg contained in the measured amino acid concentration data of the subject and (ii) the multivariate discriminant containing at least one of Thr, Ser, Asn, Pro, Gly, Cit, Val, Met, Ile, Leu, Phe, His, Trp, Orn, Lys, and Arg as the explanatory variable, and (II) the discrimination between any one of the cervical cancer and the endometrial cancer and any one of the cervical cancer-free and the endometrial cancer-free in the subject is conducted based on the calculated discriminant value. Thus, the discriminant values obtained in the multivariate discriminants useful particularly for the discrimination between any one of the cervical cancer and the endometrial cancer and any one of the cervical cancer-free and the endometrial cancer-free, can be utilized to bring about the effect of enabling more accurately the discrimination. According to the present invention, the multivariate discriminant is (i) the fractional expression with Lys, His, and Arg as the explanatory variables, (ii) the fractional expression with a-ABA, His, and Met as the explanatory variables, (iii) the fractional expression with Ile, His, Cit, and Arg as the explanatory variables, (iv) the linear discriminant with Gly, Val, His, and Arg as the explanatory variables, (v) the linear discriminant with Gly, Phe, His, and Arg as the explanatory variables, (vi) the linear discriminant with Cit, Ile, His, and Arg as the explanatory variables, (vii) the linear discriminant with His, Leu, Met, Cit, Ile, and Tyr as the explanatory variables, (viii) the logistic regression equation with Val, His, Lys, and Arg as the explanatory variables, (ix) the logistic regression equation with Thr, a-ABA, Met, and His as the explanatory variables, (x) the logistic regression equation with Cit, Ile, His, and Arg as the explanatory variables, or (xi) the logistic regression equation with His, Leu, Met, Cit, Ile, and Tyr as the explanatory variables. Thus, the discriminant values obtained in the multivariate discriminants useful particularly for the discrimination between any one of the cervical cancer and the endometrial cancer and any one of the cervical cancer-free and the endometrial cancer-free, can be utilized to bring about the effect of enabling more accurately the discrimination.

According to the present invention, (I) the discriminant value is calculated based on (i) the concentration value of at least one of Asn, Val, Met, Leu, Phe, His, Trp, Orn, Lys, and Arg contained in the measured amino acid concentration data of the subject and (ii) the multivariate discriminant containing at least one of Asn, Val, Met, Leu, Phe, His, Trp, Orn, Lys, and Arg as the explanatory variable, and (II) the discrimination between the cervical cancer and the cervical cancer-free in the subject is conducted based on the calculated discriminant value. Thus, the discriminant values obtained in the multivariate discriminants useful particularly for the 2-group discrimination between the cervical cancer and the cervical cancer-free, can be utilized to bring about the effect of enabling more accurately the 2-group discrimination. According to the present invention, the multivariate discriminant is (i) the fractional expression with a-ABA, His, and Val as the explanatory variables, (ii) the fractional expression with a-ABA, Met, and Val as the explanatory variables, (iii) the fractional expression with Met, His, Cit, and Arg as the explanatory variables, (iv) the linear discriminant with Gly, Val, His, and Arg as the explanatory variables, (v) the linear discriminant with Gly, Val, Met, and Lys as the explanatory variables, (vi) the linear discriminant with Cit, Met, His, and Arg as the explanatory variables, (vii) the linear discriminant with His, Leu, Met, Ile, Tyr, and Lys as the explanatory variables, (viii) the logistic regression equation with Val, Leu, His, and Arg as the explanatory variables, (ix) the logistic regression equation with Met, His, Orn, and Arg as the explanatory variables, (x) the logistic regression equation with Val, Tyr, His, and Arg as the explanatory variables, or (xi) the logistic regression equation with His, Leu, Met, Ile, Tyr, and Lys as the explanatory variables. Thus, the discriminant values obtained in the multivariate discriminants useful particularly for the 2-group discrimination between the cervical cancer and the cervical cancer-free, can be utilized to bring about the effect of enabling more accurately the 2-group discrimination.

According to the present invention, (I) the discriminant value is calculated based on (i) the concentration value of at least one of Thr, Ser, Asn, Pro, Gly, Cit, Val, Met, Ile, Leu, Phe, His, Trp, and Arg contained in the measured amino acid concentration data of the subject and (ii) the multivariate discriminant containing at least one of Thr, Ser, Asn, Pro, Gly, Cit, Val, Met, Ile, Leu, Phe, His, Trp, and Arg as the explanatory variable, and (II) the discrimination between the endometrial cancer and the endometrial cancer-free in the subject is conducted based on the calculated discriminant value. Thus, the discriminant values obtained in the multivariate discriminants useful particularly for the 2-group discrimination between the endometrial cancer and the endometrial cancer-free, can be utilized to bring about the effect of enabling more accurately the 2-group discrimination. According to the present invention, the multivariate discriminant is (i) the fractional expression with Lys, His, and Arg as the explanatory variables, (ii) the fractional expression with a-ABA, His, and Met as the explanatory variables, (iii) the fractional expression with Ile, His, Asn, and Cit as the explanatory variables, (iv) the linear discriminant with Gln, His, Lys, and Arg as the explanatory variables, (v) the linear discriminant with Gly, Met, Phe, and His as the explanatory variables, (vi) the linear discriminant with Cit, Ile, His, and Arg as the explanatory variables, (vii) the linear discriminant with His, Asn, Val, Pro, Cit, and Ile as the explanatory variables, (viii) the logistic regression equation with Gln, Gly, His, and Arg as the explanatory variables, (ix) the logistic regression equation with Gln, Phe, His, and Arg as the explanatory variables, (x) the logistic regression equation with Gln, Ile, His, and Arg as the explanatory variables, or (xi) the logistic regression equation with His, Asn, Val, Pro, Cit, and Ile as the explanatory variables. Thus, the discriminant values obtained in the multivariate discriminants useful particularly for the 2-group discrimination between the endometrial cancer and the endometrial cancer-free, can be utilized to bring about the effect of enabling more accurately the 2-group discrimination.

According to the present invention, (I) the discriminant value is calculated based on (i) the concentration value of at least one of Thr, Ser, Asn, Gln, Ala, Cit, Val, Met, Ile, Leu, Tyr, Phe, His, Trp, Orn, Lys, and Arg contained in the measured amino acid concentration data of the subject and (ii) the multivariate discriminant containing at least one of Thr, Ser, Asn, Gln, Ala, Cit, Val, Met, Ile, Leu, Tyr, Phe, His, Trp, Orn, Lys, and Arg as the explanatory variable, and (II) the discrimination between the ovarian cancer and the ovarian cancer-free in the subject is conducted based on the calculated discriminant value. Thus, the discriminant values obtained in the multivariate discriminants useful particularly for the 2-group discrimination between the ovarian cancer and the ovarian cancer-free, can be utilized to bring about the effect of enabling more accurately the 2-group discrimination. According to the present invention, the multivariate discriminant is (i) the fractional expression with Orn, Cit, and Met as the explanatory variables, (ii) the fractional expression with Gln, Cit, and Tyr as the explanatory variables, (iii) the fractional expression with Orn, His, Phe, and Trp as the explanatory variables, (iv) the linear discriminant with Ser, Cit, Orn, and Trp as the explanatory variables, (v) the linear discriminant with Ser, Cit, Ile, and Orn as the explanatory variables, (vi) the linear discriminant with Phe, Trp, Orn, and Lys as the explanatory variables, (vii) the linear discriminant with His, Trp, Glu, Cit, Ile, and Orn as the explanatory variables, (viii) the logistic regression equation with Ser, Cit, Trp, and Orn as the explanatory variables, (ix) the logistic regression equation with Gln, Cit, Ile, and Tyr as the explanatory variables, (x) the logistic regression equation with Asn, Phe, His, and Trp as the explanatory variables, or (xi) the logistic regression equation with His, Trp, Glu, Cit, Ile, and Orn as the explanatory variables. Thus, the discriminant values obtained in the multivariate discriminants useful particularly for the 2-group discrimination between the ovarian cancer and the ovarian cancer-free, can be utilized to bring about the effect of enabling more accurately the 2-group discrimination.

According to the present invention, (I) the discriminant value is calculated based on (i) the concentration value of at least one of Thr, Ser, Asn, Gln, Pro, Ala, Val, Met, Ile, Leu, Tyr, Phe, His, Trp, Orn, and Arg contained in the measured amino acid concentration data of the subject and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) the discrimination between the female genital cancer suffering risk group and the healthy group in the subject is conducted based on the calculated discriminant value. Thus, the discriminant values obtained in the multivariate discriminants useful particularly for the 2-group discrimination between the female genital cancer suffering risk group and the healthy group, can be utilized to bring about the effect of enabling more accurately the 2-group discrimination. According to the present invention, the multivariate discriminant is the linear discriminant with Phe, His, Met, Pro, Lys, and Arg as the explanatory variables, or the logistic regression equation with Phe, His, Met, Pro, Lys, and Arg as the explanatory variables. Thus, the discriminant values obtained in the multivariate discriminants useful particularly for the 2-group discrimination between the female genital cancer suffering risk group and the healthy group, can be utilized to bring about the effect of enabling more accurately the 2-group discrimination.

According to the present invention, (I) the discriminant value is calculated based on (i) the concentration value of at least one of Thr, Ser, Asn, Glu, Gln, Pro, Gly, Ala, Cit, ABA, Val, Met, Ile, Leu, Tyr, Phe, His, Trp, Orn, Lys, and Arg contained in the measured amino acid concentration data of the subject and (ii) the multivariate discriminant containing at least one of Thr, Ser, Asn, Glu, Gln, Pro, Gly, Ala, Cit, ABA, Val, Met, Ile, Leu, Tyr, Phe, His, Trp, Orn, Lys, and Arg as the explanatory variable, and (II) the discrimination between the cervical cancer, the endometrial cancer, and the ovarian cancer in the subject is conducted based on the calculated discriminant value. Thus, the discriminant values obtained in the multivariate discriminants useful particularly for the discrimination between the cervical cancer, the endometrial cancer, and the ovarian cancer, can be utilized to bring about the effect of enabling more accurately the discrimination. According to the present invention, the multivariate discriminant is the discriminant with Cit, Met, Lys, Asn, Ala, Thr, Gln, and a-ABA as the explanatory variables which is prepared by the Mahalanobis' generalized distance method, or the discriminant prepared with His, Leu, Ser, Thr, Glu, Gln, Ala, and Lys as the explanatory variables which is prepared by the Mahalanobis' generalized distance method. Thus, the discriminant values obtained in the multivariate discriminants useful particularly for the discrimination between the cervical cancer, the endometrial cancer, and the ovarian cancer, can be utilized to bring about the effect of enabling more accurately the discrimination.

According to the present invention, the multivariate discriminant stored in the memory unit is prepared based on the female genital cancer state information containing the amino acid concentration data and the female genital cancer state index data on the index for indicating the state of female genital cancer, stored in the memory unit. Specifically, (1) the candidate multivariate discriminant is prepared based on the predetermined discriminant-preparing method from the female genital cancer state information, (2) the prepared candidate multivariate discriminant is verified based on the predetermined verifying method, (3) the explanatory variables of the candidate multivariate discriminant are selected based on the predetermined explanatory variable-selecting method from the verification results, thereby selecting the combination of the amino acid concentration data contained in the female genital cancer state information used in preparing of the candidate multivariate discriminant, and (4) the candidate multivariate discriminant used as the multivariate discriminant is selected from a plurality of the candidate multivariate discriminants based on the verification results accumulated by repeatedly executing (1), (2) and (3), thereby preparing the multivariate discriminant. Thus, the effect of being able to prepare the multivariate discriminant most appropriate for evaluating the state of female genital cancer is brought about.

According to the present invention, the female genital cancer-evaluating program recorded on the recording medium is read and executed by the computer, thereby allowing the computer to execute the female genital cancer-evaluating program, thus bringing about the effect of obtaining the same effect as in the female genital cancer-evaluating program.

When the state of female genital cancer is evaluated in the present invention, concentrations of other metabolites, gene expression level, protein expression level, age and sex of the subject, presence or absence of smoking, digitalized electrocardiogram waveform, or the like may be used in addition to the amino acid concentration. When the state of female genital cancer is evaluated in the present invention, the concentrations of the other metabolites, the gene expression level, the protein expression level, the age and sex of the subject, the presence or absence of the smoking, the digitalized electrocardiogram waveform, or the like may be used as the explanatory variables in the multivariate discriminant in addition to the amino acid concentration.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a principle configurational diagram showing a basic principle of the present invention;

FIG. 2 is a flowchart showing one example of a method of evaluating female genital cancer according to a first embodiment;

FIG. 3 is a principle configurational diagram showing a basic principle of the present invention;

FIG. 4 is a diagram showing an example of an entire configuration of a present system;

FIG. 5 is a diagram showing another example of an entire configuration of the present system;

FIG. 6 is a block diagram showing an example of a configuration of a female genital cancer-evaluating apparatus 100 in the present system;

FIG. 7 is a chart showing an example of information stored in a user information file 106a;

FIG. 8 is a chart showing an example of information stored in an amino acid concentration data file 106b;

FIG. 9 is a chart showing an example of information stored in a female genital cancer state information file 106c;

FIG. 10 is a chart showing an example of information stored in a designated female genital cancer state information file 106d;

FIG. 11 is a chart showing an example of information stored in a candidate multivariable discriminant file 106e1;

FIG. 12 is a chart showing an example of information stored in a verification result file 106e2;

FIG. 13 is a chart showing an example of information stored in a selected female genital cancer state information file 106e3;

FIG. 14 is a chart showing an example of information stored in a multivariable discriminant file 106e4;

FIG. 15 is a chart showing an example of information stored in a discriminant value file 106f;

FIG. 16 is a chart showing an example of information stored in an evaluation result file 106g;

FIG. 17 is a block diagram showing a configuration of a multivariable discriminant-preparing part 102h;

FIG. 18 is a block diagram showing a configuration of a discriminant value criterion-evaluating part 102j;

FIG. 19 is a block diagram showing an example of a configuration of a client apparatus 200 in the present system;

FIG. 20 is a block diagram showing an example of a configuration of a database apparatus 400 in the present system;

FIG. 21 is a flowchart showing an example of a female genital cancer evaluation service processing performed in the present system;

FIG. 22 is a flowchart showing an example of a multivariate discriminant-preparing processing performed in the female genital cancer-evaluating apparatus 100 in the present system;

FIG. 23 is boxplots showing distributions of amino acid explanatory variables in a cancer patient group, a benign disease group, and a healthy group;

FIG. 24 is boxplots showing distributions of amino acid explanatory variables in a cervical cancer group, an endometrial cancer group, an ovarian cancer group, a benign disease group, and a healthy group;

FIG. 25 is a chart showing areas under the ROC curve of each amino acid explanatory variable in the 2-group discrimination between the groups;

FIG. 26 is a chart showing index formulae 1 to 12, and area under the ROC curve, cutoff value, sensitivity, specificity, positive predictive value, negative predictive value, and correct answer rate regarding each index formula;

FIG. 27 is a chart showing a list of index formulae having the same discrimination performance as that of the index formula 1;

FIG. 28 is a chart showing a list of index formulae having the same discrimination performance as that of the index formula 2;

FIG. 29 is a chart showing a list of index formulae having the same discrimination performance as that of the index formula 3;

FIG. 30 is a chart showing a list of index formulae having the same discrimination performance as that of the index formula 3;

FIG. 31 is a chart showing a list of index formulae having the same discrimination performance as that of the index formula 4;

FIG. 32 is a chart showing a list of index formulae having the same discrimination performance as that of the index formula 5;

FIG. 33 is a chart showing a list of index formulae having the same discrimination performance as that of the index formula 6;

FIG. 34 is a chart showing a list of index formulae having the same discrimination performance as that of the index formula 6;

FIG. 35 is a chart showing a list of index formulae having the same discrimination performance as that of the index formula 7;

FIG. 36 is a chart showing a list of index formulae having the same discrimination performance as that of the index formula 8;

FIG. 37 is a chart showing a list of index formulae having the same discrimination performance as that of the index formula 9;

FIG. 38 is a chart showing a list of index formulae having the same discrimination performance as that of the index formula 9;

FIG. 39 is a chart showing a list of index formulae having the same discrimination performance as that of the index formula 10;

FIG. 40 is a chart showing a list of index formulae having the same discrimination performance as that of the index formula 11;

FIG. 41 is a chart showing a list of index formulae having the same discrimination performance as that of the index formula 12;

FIG. 42 is a chart showing a list of index formulae having the same discrimination performance as that of the index formula 12;

FIG. 43 is a chart showing index formulae 13 to 21, and area under the ROC curve, cutoff value, sensitivity, specificity, positive predictive value, negative predictive value, and correct answer rate regarding each index formula;

FIG. 44 is a chart showing a list of index formulae having the same discrimination performance as that of the index formula 13;

FIG. 45 is a chart showing a list of index formulae having the same discrimination performance as that of the index formula 14;

FIG. 46 is a chart showing a list of index formulae having the same discrimination performance as that of the index formula 15;

FIG. 47 is a chart showing a list of index formulae having the same discrimination performance as that of the index formula 15;

FIG. 48 is a chart showing a list of index formulae having the same discrimination performance as that of the index formula 16;

FIG. 49 is a chart showing a list of index formulae having the same discrimination performance as that of the index formula 17;

FIG. 50 is a chart showing a list of index formulae having the same discrimination performance as that of the index formula 18;

FIG. 51 is a chart showing a list of index formulae having the same discrimination performance as that of the index formula 18;

FIG. 52 is a chart showing a list of index formulae having the same discrimination performance as that of the index formula 19;

FIG. 53 is a chart showing a list of index formulae having the same discrimination performance as that of the index formula 20;

FIG. 54 is a chart showing a list of index formulae having the same discrimination performance as that of the index formula 21;

FIG. 55 is a chart showing a list of index formulae having the same discrimination performance as that of the index formula 21;

FIG. 56 is a chart showing index formulae 22 to 30, and area under the ROC curve, cutoff value, sensitivity, specificity, positive predictive value, negative predictive value, and correct answer rate regarding each index formula;

FIG. 57 is a chart showing a list of index formulae having the same discrimination performance as that of the index formula 22;

FIG. 58 is a chart showing a list of index formulae having the same discrimination performance as that of the index formula 23;

FIG. 59 is a chart showing a list of index formulae having the same discrimination performance as that of the index formula 24;

FIG. 60 is a chart showing a list of index formulae having the same discrimination performance as that of the index formula 24;

FIG. 61 is a chart showing a list of index formulae having the same discrimination performance as that of the index formula 25;

FIG. 62 is a chart showing a list of index formulae having the same discrimination performance as that of the index formula 26;

FIG. 63 is a chart showing a list of index formulae having the same discrimination performance as that of the index formula 27;

FIG. 64 is a chart showing a list of index formulae having the same discrimination performance as that of the index formula 27;

FIG. 65 is a chart showing a list of index formulae having the same discrimination performance as that of the index formula 28;

FIG. 66 is a chart showing a list of index formulae having the same discrimination performance as that of the index formula 29;

FIG. 67 is a chart showing a list of index formulae having the same discrimination performance as that of the index formula 30;

FIG. 68 is a chart showing a list of index formulae having the same discrimination performance as that of the index formula 30;

FIG. 69 is a chart showing index formulae 31 to 39, and area under the ROC curve, cutoff value, sensitivity, specificity, positive predictive value, negative predictive value, and correct answer rate regarding each index formula;

FIG. 70 is a chart showing a list of index formulae having the same discrimination performance as that of the index formula 31;

FIG. 71 is a chart showing a list of index formulae having the same discrimination performance as that of the index formula 32;

FIG. 72 is a chart showing a list of index formulae having the same discrimination performance as that of the index formula 33;

FIG. 73 is a chart showing a list of index formulae having the same discrimination performance as that of the index formula 33;

FIG. 74 is a chart showing a list of index formulae having the same discrimination performance as that of the index formula 34;

FIG. 75 is a chart showing a list of index formulae having the same discrimination performance as that of the index formula 35;

FIG. 76 is a chart showing a list of index formulae having the same discrimination performance as that of the index formula 36;

FIG. 77 is a chart showing a list of index formulae having the same discrimination performance as that of the index formula 36;

FIG. 78 is a chart showing a list of index formulae having the same discrimination performance as that of the index formula 37;

FIG. 79 is a chart showing a list of index formulae having the same discrimination performance as that of the index formula 38;

FIG. 80 is a chart showing a list of index formulae having the same discrimination performance as that of the index formula 39;

FIG. 81 is a chart showing a list of index formulae having the same discrimination performance as that of the index formula 39;

FIG. 82 is a chart showing index formulae 40 to 48, and area under the ROC curve, cutoff value, sensitivity, specificity, positive predictive value, negative predictive value, and correct answer rate regarding each index formula;

FIG. 83 is a chart showing a list of index formulae having the same discrimination performance as that of the index formula 40;

FIG. 84 is a chart showing a list of index formulae having the same discrimination performance as that of the index formula 41;

FIG. 85 is a chart showing a list of index formulae having the same discrimination performance as that of the index formula 42;

FIG. 86 is a chart showing a list of index formulae having the same discrimination performance as that of the index formula 42;

FIG. 87 is a chart showing a list of index formulae having the same discrimination performance as that of the index formula 43;

FIG. 88 is a chart showing a list of index formulae having the same discrimination performance as that of the index formula 44;

FIG. 89 is a chart showing a list of index formulae having the same discrimination performance as that of the index formula 45;

FIG. 90 is a chart showing a list of index formulae having the same discrimination performance as that of the index formula 45;

FIG. 91 is a chart showing a list of index formulae having the same discrimination performance as that of the index formula 46;

FIG. 92 is a chart showing a list of index formulae having the same discrimination performance as that of the index formula 47;

FIG. 93 is a chart showing a list of index formulae having the same discrimination performance as that of the index formula 48;

FIG. 94 is a chart showing a list of index formulae having the same discrimination performance as that of the index formula 48;

FIG. 95 is a chart showing index formulae 49 and 50, and Spearman correlation coefficient and area under the ROC curve regarding each index formula;

FIG. 96 is a chart showing a list of index formulae having the same discrimination performance as that of the index formula 49;

FIG. 97 is a chart showing a list of index formulae having the same discrimination performance as that of the index formula 49;

FIG. 98 is a chart showing a list of index formulae having the same discrimination performance as that of the index formula 50;

FIG. 99 is a chart showing a list of index formulae having the same discrimination performance as that of the index formula 50;

FIG. 100 is a chart showing correct answer rate in cervical cancer, endometrial cancer, and ovarian cancer;

FIG. 101 is a chart showing a list of combinations of amino acid explanatory variable groups showing the same discrimination performance as that of an explanatory variable group 1;

FIG. 102 is a chart showing a list of combinations of amino acid explanatory variable groups showing the same discrimination performance as that of the explanatory variable group 1;

FIG. 103 is a chart showing a list of combinations of amino acid explanatory variable groups showing the same discrimination performance as that of the explanatory variable group 1;

FIG. 104 is a chart showing discriminant group having amino acid explanatory variables Asn, Pro, Cit, ABA, Val, Ile, Tyr, Phe, Trp, Orn, and Lys, and constant term, as an index formula group 1;

FIG. 105 is a chart showing correct answer rate in cervical cancer, endometrial cancer, and ovarian cancer;

FIG. 106 is a chart showing a list of combinations of amino acid explanatory variable groups showing the same discrimination performance as that of the index formula group 1;

FIG. 107 is a chart showing a list of combinations of amino acid explanatory variable groups showing the same discrimination performance as that of the index formula group 1;

FIG. 108 is a chart showing area under the ROC curve in each of the 2-group discriminations with respect to each index formula;

FIG. 109 is boxplots showing distributions of amino acid explanatory variables in a cancer patient group and a cancer-free group;

FIG. 110 is boxplots showing distributions of amino acid explanatory variables in a uterus cancer patient group and a uterus cancer-free group;

FIG. 111 is boxplots showing distributions of amino acid explanatory variables in an endometrial cancer patient group and an endometrial cancer-free group;

FIG. 112 is boxplots showing distributions of amino acid explanatory variables in a cervical cancer patient group and a cervical cancer-free group;

FIG. 113 is boxplots showing distributions of amino acid explanatory variables in an ovarian cancer patient group and an ovarian cancer-free group;

FIG. 114 is boxplots showing distributions of amino acid explanatory variables in a female genital cancer suffering risk group and a healthy group;

FIG. 115 is a chart showing area under the ROC curve regarding an index formula 51;

FIG. 116 is a chart showing a list of index formulae having the same discrimination performance as that of the index formula 51;

FIG. 117 is a chart showing a list of index formulae having the same discrimination performance as that of the index formula 51;

FIG. 118 is a chart showing a list of index formulae having the same discrimination performance as that of the index formula 51;

FIG. 119 is a chart showing a list of index formulae having the same discrimination performance as that of the index formula 51;

FIG. 120 is a chart showing area under the ROC curve regarding an index formula 52;

FIG. 121 is a chart showing a list of index formulae having the same discrimination performance as that of the index formula 52;

FIG. 122 is a chart showing a list of index formulae having the same discrimination performance as that of the index formula 52;

FIG. 123 is a chart showing a list of index formulae having the same discrimination performance as that of the index formula 52;

FIG. 124 is a chart showing a list of index formulae having the same discrimination performance as that of the index formula 52;

FIG. 125 is a chart showing a list of frequency of appearance of each amino acid;

FIG. 126 is a chart showing area under the ROC curve regarding an index formula 53;

FIG. 127 is a chart showing a list of index formulae having the same discrimination performance as that of the index formula 53;

FIG. 128 is a chart showing a list of index formulae having the same discrimination performance as that of the index formula 53;

FIG. 129 is a chart showing a list of index formulae having the same discrimination performance as that of the index formula 53;

FIG. 130 is a chart showing a list of index formulae having the same discrimination performance as that of the index formula 53;

FIG. 131 is a chart showing area under the ROC curve regarding an index formula 54;

FIG. 132 is a chart showing a list of index formulae having the same discrimination performance as that of the index formula 54;

FIG. 133 is a chart showing a list of index formulae having the same discrimination performance as that of the index formula 54;

FIG. 134 is a chart showing a list of index formulae having the same discrimination performance as that of the index formula 54;

FIG. 135 is a chart showing a list of index formulae having the same discrimination performance as that of the index formula 54;

FIG. 136 is a chart showing a list of frequency of appearance of each amino acid;

FIG. 137 is a chart showing area under the ROC curve regarding an index formula 55;

FIG. 138 is a chart showing a list of index formulae having the same discrimination performance as that of the index formula 55;

FIG. 139 is a chart showing a list of index formulae having the same discrimination performance as that of the index formula 55;

FIG. 140 is a chart showing a list of index formulae having the same discrimination performance as that of the index formula 55;

FIG. 141 is a chart showing a list of index formulae having the same discrimination performance as that of the index formula 55;

FIG. 142 is a chart showing area under the ROC curve regarding an index formula 56;

FIG. 143 is a chart showing a list of index formulae having the same discrimination performance as that of the index formula 56;

FIG. 144 is a chart showing a list of index formulae having the same discrimination performance as that of the index formula 56;

FIG. 145 is a chart showing a list of index formulae having the same discrimination performance as that of the index formula 56;

FIG. 146 is a chart showing a list of index formulae having the same discrimination performance as that of the index formula 56;

FIG. 147 is a chart showing a list of frequency of appearance of each amino acid;

FIG. 148 is a chart showing area under the ROC curve regarding an index formula 57;

FIG. 149 is a chart showing a list of index formulae having the same discrimination performance as that of the index formula 57;

FIG. 150 is a chart showing a list of index formulae having the same discrimination performance as that of the index formula 57;

FIG. 151 is a chart showing a list of index formulae having the same discrimination performance as that of the index formula 57;

FIG. 152 is a chart showing a list of index formulae having the same discrimination performance as that of the index formula 57;

FIG. 153 is a chart showing area under the ROC curve regarding an index formula 58;

FIG. 154 is a chart showing a list of index formulae having the same discrimination performance as that of the index formula 58;

FIG. 155 is a chart showing a list of index formulae having the same discrimination performance as that of the index formula 58;

FIG. 156 is a chart showing a list of index formulae having the same discrimination performance as that of the index formula 58;

FIG. 157 is a chart showing a list of index formulae having the same discrimination performance as that of the index formula 58;

FIG. 158 is a chart showing a list of frequency of appearance of each amino acid;

FIG. 159 is a chart showing area under the ROC curve regarding an index formula 59;

FIG. 160 is a chart showing a list of index formulae having the same discrimination performance as that of the index formula 59;

FIG. 161 is a chart showing a list of index formulae having the same discrimination performance as that of the index formula 59;

FIG. 162 is a chart showing a list of index formulae having the same discrimination performance as that of the index formula 59;

FIG. 163 is a chart showing a list of index formulae having the same discrimination performance as that of the index formula 59;

FIG. 164 is a chart showing area under the ROC curve regarding an index formula 60;

FIG. 165 is a chart showing a list of index formulae having the same discrimination performance as that of the index formula 60;

FIG. 166 is a chart showing a list of index formulae having the same discrimination performance as that of the index formula 60;

FIG. 167 is a chart showing a list of index formulae having the same discrimination performance as that of the index formula 60;

FIG. 168 is a chart showing a list of index formulae having the same discrimination performance as that of the index formula 60;

FIG. 169 is a chart showing a list of frequency of appearance of each amino acid;

FIG. 170 is a chart showing area under the ROC curve regarding an index formula 61;

FIG. 171 is a chart showing a list of index formulae having the same discrimination performance as that of the index formula 61;

FIG. 172 is a chart showing a list of index formulae having the same discrimination performance as that of the index formula 61;

FIG. 173 is a chart showing a list of index formulae having the same discrimination performance as that of the index formula 61;

FIG. 174 is a chart showing a list of index formulae having the same discrimination performance as that of the index formula 61;

FIG. 175 is a chart showing area under the ROC curve regarding an index formula 62;

FIG. 176 is a chart showing a list of index formulae having the same discrimination performance as that of the index formula 62;

FIG. 177 is a chart showing a list of index formulae having the same discrimination performance as that of the index formula 62;

FIG. 178 is a chart showing a list of index formulae having the same discrimination performance as that of the index formula 62;

FIG. 179 is a chart showing a list of index formulae having the same discrimination performance as that of the index formula 62;

FIG. 180 is a chart showing a list of frequency of appearance of each amino acid;

FIG. 181 is a chart showing a list of index formulae having the same discrimination performance as that of the index formula 63;

FIG. 182 is a chart showing a list of index formulae having the same discrimination performance as that of the index formula 63;

FIG. 183 is a chart showing a list of combinations of amino acid explanatory variable groups showing the same discrimination performance as that of an explanatory variable group 1;

FIG. 184 is a chart showing a list of combinations of amino acid explanatory variable groups showing the same discrimination performance as that of the explanatory variable group 1;

FIG. 185 is a chart showing a list of combinations of amino acid explanatory variable groups included in linear discriminant groups having the same discrimination performance as that of a linear discriminant group 1; and

FIG. 186 is a chart showing a list of combinations of amino acid explanatory variable groups included in linear discriminant groups having the same discrimination performance as that of the linear discriminant group 1.

### DETAILED DESCRIPTION OF THE PREFERRED EMBODIMENTS

Hereinafter, an embodiment (first embodiment) of the method of evaluating female genital cancer of the present invention and an embodiment (second embodiment) of the female genital cancer-evaluating apparatus, the female genital cancer-evaluating method, the female genital cancer-evaluating system, the female genital cancer-evaluating program and the recording medium of the present invention are described in detail with reference to the drawings. The present invention is not limited to these embodiments.

### First Embodiment

### 1-1. Outline of the Invention

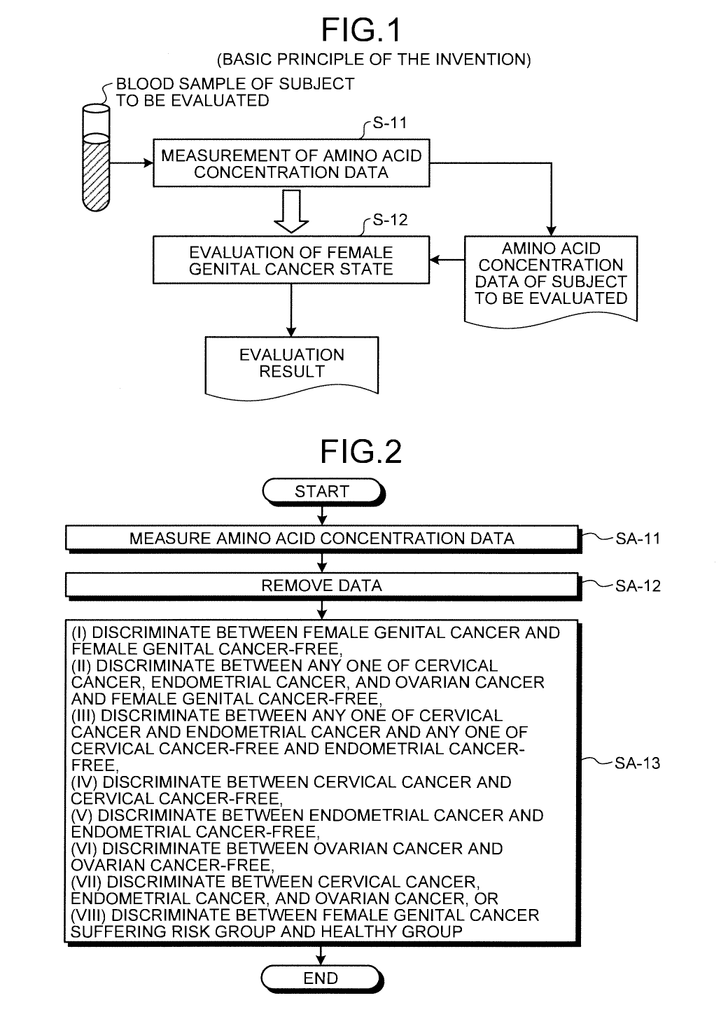

Here, an outline of the method of evaluating female genital cancer of the present invention will be described with reference to FIG. 1. FIG. 1 is a principle configurational diagram showing a basic principle of the present invention.

In the present invention, amino acid concentration data on a concentration value of an amino acid in blood collected from a subject (for example, an individual such as animal or human) to be evaluated is first measured (step S-11). Concentrations of amino acids in blood are analyzed in the following manner. A blood sample is collected in a heparin-treated tube, and then the blood plasma is separated by centrifugation of the collected blood sample. All blood plasma samples separated are frozen and stored at −70° C. before a measurement of amino acid concentrations. Before the measurement of amino acid concentrations, the blood plasma samples are deproteinized by adding sulfosalicylic acid to a concentration of 3%. An amino acid analyzer by high-performance liquid chromatography (HPLC) by using ninhydrin reaction in the post column is used for the measurement of amino acid concentrations. The unit of the amino acid concentration may be for example molar concentration, weight concentration, or these concentrations which are subjected to addition, subtraction, multiplication or division by an arbitrary constant.

In the present invention, the state of female genital cancer including at least one of cervical cancer, endometrial cancer, and ovarian cancer in the subject is evaluated based on the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data of the subject measured in step S-11 (step S-12).

According to the present invention described above, (I) the amino acid concentration data on the concentration value of the amino acid in blood collected from the subject is measured, and (II) the state of female genital cancer in the subject is evaluated based on the concentration value of at least one of Thr, Ser, Asn, Gln, Pro, Gly, Ala, Cit, Val, Met, Ile, Leu, Tyr, Phe, His, Trp, Orn, Lys, and Arg contained in the measured amino acid concentration data of the subject. Thus, the concentrations of the amino acids which among amino acids in blood, are related to the state of female genital cancer can be utilized to bring about the effect of enabling an accurate evaluation of the state of female genital cancer. Specifically, a subject likely to contract female genital cancer can be narrowed by one sample in a short time to bring about the effect of enabling the reduction of temporal, physical and financial burden of the subject. Specifically, whether a certain sample is with female genital cancer can be evaluated accurately by the concentrations of a plurality of the amino acids to bring about the effect of enabling to make the examination efficient and high accurate.

Before step S-12 is executed, data such as defective and outliers may be removed from the amino acid concentration data of the subject measured in step S-11. Thereby, the state of female genital cancer can be more accurately evaluated.

In step S-12, the discrimination (i) between the female genital cancer and the female genital cancer-free, (ii) between any one of the cervical cancer, the endometrial cancer, and the ovarian cancer and the female genital cancer-free, (iii) between any one of the cervical cancer and the endometrial cancer and any one of the cervical cancer-free and the endometrial cancer-free, (iv) between the cervical cancer and the cervical cancer-free, (v) between the endometrial cancer and the endometrial cancer-free, (vi) between the ovarian cancer and the ovarian cancer-free, (vii) between the female genital cancer suffering risk group and the healthy group, or (viii) between the cervical cancer, the endometrial cancer, and the ovarian cancer in the subject may be conducted based on the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data of the subject measured in step S-11. Thus, the concentrations of the amino acids which among amino acids in blood, are useful for (i) the 2-group discrimination between the female genital cancer and the female genital cancer-free, (ii) the discrimination between any one of the cervical cancer, the endometrial cancer, and the ovarian cancer and the female genital cancer-free, (iii) the discrimination between any one of the cervical cancer and the endometrial cancer and any one of the cervical cancer-free and the endometrial cancer-free, (iv) the 2-group discrimination between the cervical cancer and the cervical cancer-free, (v) the 2-group discrimination between the endometrial cancer and the endometrial cancer-free, (vi) the 2-group discrimination between the ovarian cancer and the ovarian cancer-free, (vii) the 2-group discrimination between the female genital cancer suffering risk group and the healthy group, or (viii) the discrimination between the cervical cancer, the endometrial cancer, and the ovarian cancer, can be utilized to bring about the effect of enabling accurately these 2-group discriminations or these discriminations.

In step S-12, (I) a discriminant value that is a value of a multivariate discriminant with a concentration of the amino acid as an explanatory variable may be calculated based on both (i) the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data of the subject measured in step S-11 and (ii) the previously established multivariate discriminant containing at least one of Thr, Ser, Asn, Gln, Pro, Gly, Ala, Cit, Val, Met, Ile, Leu, Tyr, Phe, His, Trp, Orn, Lys, and Arg as the explanatory variable, and (II) the state of female genital cancer in the subject may be evaluated based on the calculated discriminant value. Thus, the discriminant values obtained in the multivariate discriminants correlated significantly with the state of female genital cancer can be utilized to bring about the effect of enabling an accurate evaluation of the state of female genital cancer. Specifically, a subject likely to contract female genital cancer can be narrowed by one sample in a short time to bring about the effect of enabling the reduction of temporal, physical and financial burden of the subject. Specifically, whether a certain sample is with female genital cancer can be evaluated accurately by the concentrations of a plurality of the amino acids and the discriminants with the concentrations of the amino acids as the explanatory variables to bring about the effect of enabling to make the examination efficient and high accurate.

In step S-12, the discrimination (i) between the female genital cancer and the female genital cancer-free, (ii) between any one of the cervical cancer, the endometrial cancer, and the ovarian cancer and the female genital cancer-free, (iii) between any one of the cervical cancer and the endometrial cancer and any one of the cervical cancer-free and the endometrial cancer-free, (iv) between the cervical cancer and the cervical cancer-free, (v) between the endometrial cancer and the endometrial cancer-free, (vi) between the ovarian cancer and the ovarian cancer-free, (vii) between the female genital cancer suffering risk group and the healthy group, or (viii) between the cervical cancer, the endometrial cancer, and the ovarian cancer in the subject may be conducted based on the calculated discriminant value. Specifically, the discriminant value may be compared with a previously established threshold (cutoff value), thereby discriminating (i) between the female genital cancer and the female genital cancer-free, (ii) between any one of the cervical cancer, the endometrial cancer, and the ovarian cancer and the female genital cancer-free, (iii) between any one of the cervical cancer and the endometrial cancer and any one of the cervical cancer-free and the endometrial cancer-free, (iv) between the cervical cancer and the cervical cancer-free, (v) between the endometrial cancer and the endometrial cancer-free, (vi) between the ovarian cancer and the ovarian cancer-free, (vii) between the female genital cancer suffering risk group and the healthy group, or (viii) between the cervical cancer, the endometrial cancer, and the ovarian cancer in the subject. Thus, the discriminant values obtained in the multivariate discriminants useful for (i) the 2-group discrimination between the female genital cancer and the female genital cancer-free, (ii) the discrimination between any one of the cervical cancer, the endometrial cancer, and the ovarian cancer and the female genital cancer-free, (iii) the discrimination between any one of the cervical cancer and the endometrial cancer and any one of the cervical cancer-free and the endometrial cancer-free, (iv) the 2-group discrimination between the cervical cancer and the cervical cancer-free, (v) the 2-group discrimination between the endometrial cancer and the endometrial cancer-free, (vi) the 2-group discrimination between the ovarian cancer and the ovarian cancer-free, (vii) the 2-group discrimination between the female genital cancer suffering risk group and the healthy group, or (viii) the discrimination between the cervical cancer, the endometrial cancer, and the ovarian cancer, can be utilized to bring about the effect of enabling accurately these 2-group discriminations or these discriminations.

The multivariate discriminant may be any one of a fractional expression, the sum of a plurality of the fractional expressions, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Thus, the discriminant values obtained in the multivariate discriminants useful particularly for (i) the 2-group discrimination between the female genital cancer and the female genital cancer-free, (ii) the discrimination between any one of the cervical cancer, the endometrial cancer, and the ovarian cancer and the female genital cancer-free, (iii) the discrimination between any one of the cervical cancer and the endometrial cancer and any one of the cervical cancer-free and the endometrial cancer-free, (iv) the 2-group discrimination between the cervical cancer and the cervical cancer-free, (v) the 2-group discrimination between the endometrial cancer and the endometrial cancer-free, (vi) the 2-group discrimination between the ovarian cancer and the ovarian cancer-free, (vii) the 2-group discrimination between the female genital cancer suffering risk group and the healthy group, or (viii) the discrimination between the cervical cancer, the endometrial cancer, and the ovarian cancer, can be utilized to bring about the effect of enabling more accurately these 2-group discriminations or these discriminations.

In step S-12, (I) the discriminant value may be calculated based on both (i) the concentration value of at least one of Thr, Ser, Asn, Gln, Pro, Ala, Val, Met, Ile, Leu, Tyr, Phe, His, Trp, Orn, and Arg contained in the amino acid concentration data of the subject measured in step S-11 and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) the discrimination between any one of the cervical cancer, the endometrial cancer, and the ovarian cancer and the female genital cancer-free in the subject may be conducted based on the calculated discriminant value. Thus, the discriminant values obtained in the multivariate discriminants useful particularly for the discrimination between any one of the cervical cancer, the endometrial cancer, and the ovarian cancer and the female genital cancer-free, can be utilized to bring about the effect of enabling more accurately the discrimination. The multivariate discriminant to be used in this case may be (i) the fractional expression with Gln, His, and Arg as the explanatory variables, (ii) the fractional expression with a-ABA, His, and Met as the explanatory variables, (iii) the fractional expression with Ile, His, Cit, Arg, Tyr, and Trp as the explanatory variables, (iv) the fractional expression with a-ABA, Cit, and Met as the explanatory variables, (v) the linear discriminant with Gly, Val, His, and Arg as the explanatory variables, (vi) the linear discriminant with Gly, a-ABA, Met, and His as the explanatory variables, (vii) the linear discriminant with Ala, Ile, His, Trp, and Arg as the explanatory variables, (viii) the linear discriminant with Gly, Cit, Met, and Phe as the explanatory variables, (ix) the linear discriminant with His, Leu, Met, Cit, Ile, and Tyr as the explanatory variables, (x) the logistic regression equation with Val, Leu, His, and Arg as the explanatory variables, (xi) the logistic regression equation with a-ABA, Met, Tyr, and His as the explanatory variables, (xii) the logistic regression equation with Val, Ile, His, Trp, and Arg as the explanatory variables, (xiii) the logistic regression equation with Cit, a-ABA, Met, and Tyr as the explanatory variables, or (xiv) the logistic regression equation with His, Leu, Met, Cit, Ile, and Tyr as the explanatory variables. Thus, the discriminant values obtained in the multivariate discriminants useful particularly for the discrimination between any one of the cervical cancer, the endometrial cancer, and the ovarian cancer and the female genital cancer-free, can be utilized to bring about the effect of enabling more accurately the discrimination.

In step S-12, (I) the discriminant value may be calculated based on both (i) the concentration value of at least one of Thr, Ser, Asn, Pro, Gly, Cit, Val, Met, Ile, Leu, Phe, His, Trp, Orn, Lys, and Arg contained in the amino acid concentration data of the subject measured in step S-11 and (ii) the multivariate discriminant containing at least one of Thr, Ser, Asn, Pro, Gly, Cit, Val, Met, Ile, Leu, Phe, His, Trp, Orn, Lys, and Arg as the explanatory variable, and (II) the discrimination between any one of the cervical cancer and the endometrial cancer and any one of the cervical cancer-free and the endometrial cancer-free in the subject may be conducted based on the calculated discriminant value. Thus, the discriminant values obtained in the multivariate discriminants useful particularly for the discrimination between any one of the cervical cancer and the endometrial cancer and any one of the cervical cancer-free and the endometrial cancer-free, can be utilized to bring about the effect of enabling more accurately the discrimination. The multivariate discriminant to be used in this case may be (i) the fractional expression with Lys, His, and Arg as the explanatory variables, (ii) the fractional expression with a-ABA, His, and Met as the explanatory variables, (iii) the fractional expression with Ile, His, Cit, and Arg as the explanatory variables, (iv) the linear discriminant with Gly, Val, His, and Arg as the explanatory variables, (v) the linear discriminant with Gly, Phe, His, and Arg as the explanatory variables, (vi) the linear discriminant with Cit, Ile, His, and Arg as the explanatory variables, (vii) the linear discriminant with His, Leu, Met, Cit, Ile, and Tyr as the explanatory variables, (viii) the logistic regression equation with Val, His, Lys, and Arg as the explanatory variables, (ix) the logistic regression equation with Thr, a-ABA, Met, and His as the explanatory variables, (x) the logistic regression equation with Cit, Ile, His, and Arg as the explanatory variables, or (xi) the logistic regression equation with His, Leu, Met, Cit, Ile, and Tyr as the explanatory variables. Thus, the discriminant values obtained in the multivariate discriminants useful particularly for the discrimination between any one of the cervical cancer and the endometrial cancer and any one of the cervical cancer-free and the endometrial cancer-free, can be utilized to bring about the effect of enabling more accurately the discrimination.

In step S-12, (I) the discriminant value may be calculated based on both (i) the concentration value of at least one of Asn, Val, Met, Leu, Phe, His, Trp, Orn, Lys, and Arg contained in the amino acid concentration data of the subject measured in step S-11 and (ii) the multivariate discriminant containing at least one of Asn, Val, Met, Leu, Phe, His, Trp, Orn, Lys, and Arg as the explanatory variable, and (II) the discrimination between the cervical cancer and the cervical cancer-free in the subject may be conducted based on the calculated discriminant value. Thus, the discriminant values obtained in the multivariate discriminants useful particularly for the 2-group discrimination between the cervical cancer and the cervical cancer-free, can be utilized to bring about the effect of enabling more accurately the 2-group discrimination. The multivariate discriminant to be used in this case may be (i) the fractional expression with a-ABA, His, and Val as the explanatory variables, (ii) the fractional expression with a-ABA, Met, and Val as the explanatory variables, (iii) the fractional expression with Met, His, Cit, and Arg as the explanatory variables, (iv) the linear discriminant with Gly, Val, His, and Arg as the explanatory variables, (v) the linear discriminant with Gly, Val, Met, and Lys as the explanatory variables, (vi) the linear discriminant with Cit, Met, His, and Arg as the explanatory variables, (vii) the linear discriminant with His, Leu, Met, Ile, Tyr, and Lys as the explanatory variables, (viii) the logistic regression equation with Val, Leu, His, and Arg as the explanatory variables, (ix) the logistic regression equation with Met, His, Orn, and Arg as the explanatory variables, (x) the logistic regression equation with Val, Tyr, His, and Arg as the explanatory variables, or (xi) the logistic regression equation with His, Leu, Met, Ile, Tyr, and Lys as the explanatory variables. Thus, the discriminant values obtained in the multivariate discriminants useful particularly for the 2-group discrimination between the cervical cancer and the cervical cancer-free, can be utilized to bring about the effect of enabling more accurately the 2-group discrimination.

In step S-12, (I) the discriminant value may be calculated based on both (i) the concentration value of at least one of Thr, Ser, Asn, Pro, Gly, Cit, Val, Met, Ile, Leu, Phe, His, Trp, and Arg contained in the amino acid concentration data of the subject measured in step S-11 and (ii) the multivariate discriminant containing at least one of Thr, Ser, Asn, Pro, Gly, Cit, Val, Met, Ile, Leu, Phe, His, Trp, and Arg as the explanatory variable, and (II) the discrimination between the endometrial cancer and the endometrial cancer-free in the subject may be conducted based on the calculated discriminant value. Thus, the discriminant values obtained in the multivariate discriminants useful particularly for the 2-group discrimination between the endometrial cancer and the endometrial cancer-free, can be utilized to bring about the effect of enabling more accurately the 2-group discrimination. The multivariate discriminant to be used in this case may be (i) the fractional expression with Lys, His, and Arg as the explanatory variables, (ii) the fractional expression with a-ABA, His, and Met as the explanatory variables, (iii) the fractional expression with Ile, His, Asn, and Cit as the explanatory variables, (iv) the linear discriminant with Gln, His, Lys, and Arg as the explanatory variables, (v) the linear discriminant with Gly, Met, Phe, and His as the explanatory variables, (vi) the linear discriminant with Cit, Ile, His, and Arg as the explanatory variables, (vii) the linear discriminant with His, Asn, Val, Pro, Cit, and Ile as the explanatory variables, (viii) the logistic regression equation with Gln, Gly, His, and Arg as the explanatory variables, (ix) the logistic regression equation with Gln, Phe, His, and Arg as the explanatory variables, (x) the logistic regression equation with Gln, Ile, His, and Arg as the explanatory variables, or (xi) the logistic regression equation with His, Asn, Val, Pro, Cit, and Ile as the explanatory variables. Thus, the discriminant values obtained in the multivariate discriminants useful particularly for the 2-group discrimination between the endometrial cancer and the endometrial cancer-free, can be utilized to bring about the effect of enabling more accurately the 2-group discrimination.

In step S-12, (I) the discriminant value may be calculated based on both (i) the concentration value of at least one of Thr, Ser, Asn, Gln, Ala, Cit, Val, Met, Ile, Leu, Tyr, Phe, His, Trp, Orn, Lys, and Arg contained in the amino acid concentration data of the subject measured in step S-11 and (ii) the multivariate discriminant containing at least one of Thr, Ser, Asn, Gln, Ala, Cit, Val, Met, Ile, Leu, Tyr, Phe, His, Trp, Orn, Lys, and Arg as the explanatory variable, and (II) the discrimination between the ovarian cancer and the ovarian cancer-free in the subject may be conducted based on the calculated discriminant value. Thus, the discriminant values obtained in the multivariate discriminants useful particularly for the 2-group discrimination between the ovarian cancer and the ovarian cancer-free, can be utilized to bring about the effect of enabling more accurately the 2-group discrimination. The multivariate discriminant to be used in this case may be (i) the fractional expression with Orn, Cit, and Met as the explanatory variables, (ii) the fractional expression with Gln, Cit, and Tyr as the explanatory variables, (iii) the fractional expression with Orn, His, Phe, and Trp as the explanatory variables, (iv) the linear discriminant with Ser, Cit, Orn, and Trp as the explanatory variables, (v) the linear discriminant with Ser, Cit, Ile, and Orn as the explanatory variables, (vi) the linear discriminant with Phe, Trp, Orn, and Lys as the explanatory variables, (vii) the linear discriminant with His, Trp, Glu, Cit, Ile, and Orn as the explanatory variables, (viii) the logistic regression equation with Ser, Cit, Trp, and Orn as the explanatory variables, (ix) the logistic regression equation with Gln, Cit, Ile, and Tyr as the explanatory variables, (x) the logistic regression equation with Asn, Phe, His, and Trp as the explanatory variables, or (xi) the logistic regression equation with His, Trp, Glu, Cit, Ile, and Orn as the explanatory variables. Thus, the discriminant values obtained in the multivariate discriminants useful particularly for the 2-group discrimination between the ovarian cancer and the ovarian cancer-free, can be utilized to bring about the effect of enabling more accurately the 2-group discrimination.

In step S-12, (I) the discriminant value may be calculated based on both (i) the concentration value of at least one of Thr, Ser, Asn, Gln, Pro, Ala, Val, Met, Ile, Leu, Tyr, Phe, His, Trp, Orn, and Arg contained in the amino acid concentration data of the subject measured in step S-11 and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) the discrimination between the female genital cancer suffering risk group and the healthy group in the subject may be conducted based on the calculated discriminant value. Thus, the discriminant values obtained in the multivariate discriminants useful particularly for the 2-group discrimination between the female genital cancer suffering risk group and the healthy group, can be utilized to bring about the effect of enabling more accurately the 2-group discrimination. The multivariate discriminant to be used in this case may be the linear discriminant with Phe, His, Met, Pro, Lys, and Arg as the explanatory variables, or the logistic regression equation with Phe, His, Met, Pro, Lys, and Arg as the explanatory variables. Thus, the discriminant values obtained in the multivariate discriminants useful particularly for the 2-group discrimination between the female genital cancer suffering risk group and the healthy group, can be utilized to bring about the effect of enabling more accurately the 2-group discrimination.

In step S-12, (I) the discriminant value may be calculated based on both (i) the concentration value of at least one of Thr, Ser, Asn, Glu, Gln, Pro, Gly, Ala, Cit, ABA, Val, Met, Ile, Leu, Tyr, Phe, His, Trp, Orn, Lys, and Arg contained in the amino acid concentration data of the subject measured in step S-11 and (ii) the multivariate discriminant containing at least one of Thr, Ser, Asn, Glu, Gln, Pro, Gly, Ala, Cit, ABA, Val, Met, Ile, Leu, Tyr, Phe, His, Trp, Orn, Lys, and Arg as the explanatory variable, and (II) the discrimination between the cervical cancer, the endometrial cancer, and the ovarian cancer in the subject may be conducted based on the calculated discriminant value. Thus, the discriminant values obtained in the multivariate discriminants useful particularly for the discrimination between the cervical cancer, the endometrial cancer, and the ovarian cancer, can be utilized to bring about the effect of enabling more accurately the discrimination. The multivariate discriminant to be used in this case may be the discriminant with Cit, Met, Lys, Asn, Ala, Thr, Gln, and a-ABA as the explanatory variables which is prepared by the Mahalanobis' generalized distance method, or the discriminant prepared with His, Leu, Ser, Thr, Glu, Gln, Ala, and Lys as the explanatory variables which is prepared by the Mahalanobis' generalized distance method. Thus, the discriminant values obtained in the multivariate discriminants useful particularly for the discrimination between the cervical cancer, the endometrial cancer, and the ovarian cancer, can be utilized to bring about the effect of enabling more accurately the discrimination.

The multivariate discriminant described above can be prepared by a method described in International Publication WO 2004/052191 that is an international application filed by the present applicant or by a method (multivariate discriminant-preparing processing described in the second embodiment described later) described in International Publication WO 2006/098192 that is an international application filed by the present applicant. Any multivariate discriminants obtained by these methods can be preferably used in the evaluation of the state of female genital cancer, regardless of the unit of the amino acid concentration in the amino acid concentration data as input data.

The multivariate discriminant refers to a form of equation used generally in multivariate analysis and includes, for example, multiple regression equation, multiple logistic regression equation, linear discriminant function, Mahalanobis' generalized distance, canonical discriminant function, support vector machine, and decision tree. The multivariate discriminant also includes an equation shown by the sum of different forms of multivariate discriminants. In the multiple regression equation, multiple logistic regression equation and canonical discriminant function, a coefficient and constant term are added to each explanatory variable, and the coefficient and constant term in this case are preferably real numbers, more preferably values in the range of 99% confidence interval for the coefficient and constant term obtained from data for discrimination, more preferably in the range of 95% confidence interval for the coefficient and constant term obtained from data for discrimination. The value of each coefficient and the confidence interval thereof may be those multiplied by a real number, and the value of each constant term and the confidence interval thereof may be those having an arbitrary actual constant added or subtracted or those multiplied or divided by an arbitrary actual constant.

In the fractional expression, the numerator of the fractional expression is expressed by the sum of the amino acids A, B, C etc. and the denominator of the fractional expression is expressed by the sum of the amino acids a, b, c etc. The fractional expression also includes the sum of the fractional expressions α, β, γ etc. (for example, α+β) having such constitution. The fractional expression also includes divided fractional expressions. The amino acids used in the numerator or denominator may have suitable coefficients respectively. The amino acids used in the numerator or denominator may appear repeatedly. Each fractional expression may have a suitable coefficient. A value of a coefficient for each explanatory variable and a value for a constant term may be any real numbers. In combinations where explanatory variables in the numerator and explanatory variables in the denominator in the fractional expression are switched with each other, the positive (or negative) sign is generally reversed in correlation with objective explanatory variables, but because their correlation is maintained, such combinations can be assumed to be equivalent to one another in discrimination, and thus the fractional expression also includes combinations where explanatory variables in the numerator and explanatory variables in the denominator in the fractional expression are switched with each other.

When the state of female genital cancer is evaluated in the present invention, the concentrations of the other metabolites, the gene expression level, the protein expression level, the age and sex of the subject, the presence or absence of the smoking, the digitalized electrocardiogram waveform, or the like may be used in addition to the amino acid concentration. When the state of female genital cancer is evaluated in the present invention, the concentrations of the other metabolites, the gene expression level, the protein expression level, the age and sex of the subject, the presence or absence of the smoking, the digitalized electrocardiogram waveform, or the like may be used as the explanatory variables in the multivariate discriminant in addition to the amino acid concentration.

### 1-2. Method of Evaluating Female Genital Cancer in Accordance with the First Embodiment

Herein, the method of evaluating female genital cancer according to the first embodiment is described with reference to FIG. 2. FIG. 2 is a flowchart showing one example of the method of evaluating female genital cancer according to the first embodiment.

The amino acid concentration data on the concentration values of the amino acids is measured from blood collected from an individual such as animal or human (step SA-11). The measurement of the concentration values of the amino acids is conducted by the method described above.

Data such as defective and outliers is then removed from the amino acid concentration data of the individual measured in step SA-11 (step SA-12).

Then, any one of the discriminations described in the following 11. to 18. is conducted in the individual, based on the amino acid concentration data of the individual from which the data such as the defective and the outliers have been removed in step SA-12 or the previously established multivariate discriminant with the concentration of the amino acid as the explanatory variable (the multivariate discriminant is any one of a fractional expression, the sum of a plurality of the fractional expressions, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.) (step SA-13).

11. Discrimination Between Female Genital Cancer and Female Genital Cancer-Free

(A) the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data is compared with a previously established threshold (cutoff value), thereby discriminating between the female genital cancer and the female genital cancer-free in the individual, or (B) (I) the discriminant value is calculated based on both (i) the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data and (ii) the multivariate discriminant containing at least one of Thr, Ser, Asn, Gln, Pro, Gly, Ala, Cit, Val, Met, Ile, Leu, Tyr, Phe, His, Trp, Orn, Lys, and Arg as the explanatory variable, and (II) the calculated discriminant value is compared with a previously established threshold (cutoff value), thereby discriminating between the female genital cancer and the female genital cancer-free in the individual.

12. Discrimination Between any One of the Cervical Cancer, the Endometrial Cancer, and the Ovarian Cancer and the Female Genital Cancer-Free

(A) the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data is compared with a previously established threshold (cutoff value), thereby discriminating between any one of the cervical cancer, the endometrial cancer, and the ovarian cancer and the female genital cancer-free in the individual, or (B) (I) the discriminant value is calculated based on both (i) the concentration value of at least one of Thr, Ser, Asn, Gln, Pro, Ala, Val, Met, Ile, Leu, Tyr, Phe, His, Trp, Orn, and Arg contained in the amino acid concentration data and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) the calculated discriminant value is compared with a previously established threshold (cutoff value), thereby discriminating between any one of the cervical cancer, the endometrial cancer, and the ovarian cancer and the female genital cancer-free in the individual.

13. Discrimination Between any One of the Cervical Cancer and the Endometrial Cancer and any One of the Cervical Cancer-Free and the Endometrial Cancer-Free

(A) the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data is compared with a previously established threshold (cutoff value), thereby discriminating between any one of the cervical cancer and the endometrial cancer and any one of the cervical cancer-free and the endometrial cancer-free in the individual, or (B) (I) the discriminant value is calculated based on both (i) the concentration value of at least one of Thr, Ser, Asn, Pro, Gly, Cit, Val, Met, Ile, Leu, Phe, His, Trp, Orn, Lys, and Arg contained in the amino acid concentration data and (ii) the multivariate discriminant containing at least one of Thr, Ser, Asn, Pro, Gly, Cit, Val, Met, Ile, Leu, Phe, His, Trp, Orn, Lys, and Arg as the explanatory variable, and (II) the calculated discriminant value is compared with a previously established threshold (cutoff value), thereby discriminating between any one of the cervical cancer and the endometrial cancer and any one of the cervical cancer-free and the endometrial cancer-free in the individual.

14. Discrimination Between the Cervical Cancer and the Cervical Cancer-Free

(A) the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data is compared with a previously established threshold (cutoff value), thereby discriminating between the cervical cancer and the cervical cancer-free in the individual, or (B) (I) the discriminant value is calculated based on both (i) the concentration value of at least one of Asn, Val, Met, Leu, Phe, His, Trp, Orn, Lys, and Arg contained in the amino acid concentration data and (ii) the multivariate discriminant containing at least one of Asn, Val, Met, Leu, Phe, His, Trp, Orn, Lys, and Arg as the explanatory variable, and (II) the calculated discriminant value is compared with a previously established threshold (cutoff value), thereby discriminating between the cervical cancer and the cervical cancer-free in the individual.

15. Discrimination Between the Endometrial Cancer and the Endometrial Cancer-Free

(A) the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data is compared with a previously established threshold (cutoff value), thereby discriminating between the endometrial cancer and the endometrial cancer-free in the individual, or (B) (I) the discriminant value is calculated based on both (i) the concentration value of at least one of Thr, Ser, Asn, Pro, Gly, Cit, Val, Met, Ile, Leu, Phe, His, Trp, and Arg contained in the amino acid concentration data and (ii) the multivariate discriminant containing at least one of Thr, Ser, Asn, Pro, Gly, Cit, Val, Met, Ile, Leu, Phe, His, Trp, and Arg as the explanatory variable, and (II) the calculated discriminant value is compared with a previously established threshold (cutoff value), thereby discriminating between the endometrial cancer and the endometrial cancer-free in the individual.

16. Discrimination Between the Ovarian Cancer and the Ovarian Cancer-Free

(A) the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data is compared with a previously established threshold (cutoff value), thereby discriminating between the ovarian cancer and the ovarian cancer-free in the individual, or (B) (I) the discriminant value is calculated based on both (i) the concentration value of at least one of Thr, Ser, Asn, Gln, Ala, Cit, Val, Met, Ile, Leu, Tyr, Phe, His, Trp, Orn, Lys, and Arg contained in the amino acid concentration data and (ii) the multivariate discriminant containing at least one of Thr, Ser, Asn, Gln, Ala, Cit, Val, Met, Ile, Leu, Tyr, Phe, His, Trp, Orn, Lys, and Arg as the explanatory variable, and (II) the calculated discriminant value is compared with a previously established threshold (cutoff value), thereby discriminating between the ovarian cancer and the ovarian cancer-free in the individual.

17. Discrimination Between the Cervical Cancer, the Endometrial Cancer, and the Ovarian Cancer

(A) the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data is compared with a previously established threshold (cutoff value), thereby discriminating between the cervical cancer, the endometrial cancer, and the ovarian cancer in the individual, or (B) (I) the discriminant value is calculated based on both (i) the concentration value of at least one of Thr, Ser, Asn, Glu, Gln, Pro, Gly, Ala, Cit, ABA, Val, Met, Ile, Leu, Tyr, Phe, His, Trp, Orn, Lys, and Arg contained in the amino acid concentration data and (ii) the multivariate discriminant containing at least one of Thr, Ser, Asn, Glu, Gln, Pro, Gly, Ala, Cit, ABA, Val, Met, Ile, Leu, Tyr, Phe, His, Trp, Orn, Lys, and Arg as the explanatory variable, and (II) the calculated discriminant value is compared with a previously established threshold (cutoff value), thereby discriminating between the cervical cancer, the endometrial cancer, and the ovarian cancer in the individual.

18. Discrimination Between the Female Genital Cancer Suffering Risk Group and the Healthy Group

(A) the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data is compared with a previously established threshold (cutoff value), thereby discriminating between the female genital cancer suffering risk group and the healthy group in the individual, or (B) (I) the discriminant value is calculated based on both (i) the concentration value of at least one of Thr, Ser, Asn, Gln, Pro, Ala, Val, Met, Ile, Leu, Tyr, Phe, His, Trp, Orn, and Arg contained in the amino acid concentration data and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) the calculated discriminant value is compared with a previously established threshold (cutoff value), thereby discriminating between the female genital cancer suffering risk group and the healthy group in the individual.

### 1-3. Summary of the First Embodiment and Other Embodiments

In the method of evaluating female genital cancer as described above in detail, (1) the amino acid concentration data is measured from blood collected from the individual, (2) the data such as the defective and the outliers is removed from the measured amino acid concentration data of the individual, and (3) any one of the discriminations described in 11. to 18. above is conducted based on (i) the amino acid concentration data of the individual from which the data such as the defective and the outliers have been removed or (ii) the previously established multivariate discriminant with the concentration of the amino acid as the explanatory variable. Thus, the concentrations of the amino acids which among amino acids in blood, are useful for (i) the 2-group discrimination between the female genital cancer and the female genital cancer-free, (ii) the discrimination between any one of the cervical cancer, the endometrial cancer, and the ovarian cancer and the female genital cancer-free, (iii) the discrimination between any one of the cervical cancer and the endometrial cancer and any one of the cervical cancer-free and the endometrial cancer-free, (iv) the 2-group discrimination between the cervical cancer and the cervical cancer-free, (v) the 2-group discrimination between the endometrial cancer and the endometrial cancer-free, (vi) the 2-group discrimination between the ovarian cancer and the ovarian cancer-free, (vii) the 2-group discrimination between the female genital cancer suffering risk group and the healthy group, or (viii) the discrimination between the cervical cancer, the endometrial cancer, and the ovarian cancer, can be utilized to bring about the effect of enabling accurately these 2-group discriminations or these discriminations. The discriminant values obtained in the multivariate discriminants useful for (i) the 2-group discrimination between the female genital cancer and the female genital cancer-free, (ii) the discrimination between any one of the cervical cancer, the endometrial cancer, and the ovarian cancer and the female genital cancer-free, (iii) the discrimination between any one of the cervical cancer and the endometrial cancer and any one of the cervical cancer-free and the endometrial cancer-free, (iv) the 2-group discrimination between the cervical cancer and the cervical cancer-free, (v) the 2-group discrimination between the endometrial cancer and the endometrial cancer-free, (vi) the 2-group discrimination between the ovarian cancer and the ovarian cancer-free, (vii) the 2-group discrimination between the female genital cancer suffering risk group and the healthy group, or (viii) the discrimination between the cervical cancer, the endometrial cancer, and the ovarian cancer, can be utilized to bring about the effect of enabling accurately these 2-group discriminations or these discriminations.

When the discrimination described in 12. above is conducted in step SA-13, the multivariate discriminant may be (i) the fractional expression with Gln, His, and Arg as the explanatory variables, (ii) the fractional expression with a-ABA, His, and Met as the explanatory variables, (iii) the fractional expression with Ile, His, Cit, Arg, Tyr, and Trp as the explanatory variables, (iv) the fractional expression with a-ABA, Cit, and Met as the explanatory variables, (v) the linear discriminant with Gly, Val, His, and Arg as the explanatory variables, (vi) the linear discriminant with Gly, a-ABA, Met, and His as the explanatory variables, (vii) the linear discriminant with Ala, Ile, His, Trp, and Arg as the explanatory variables, (viii) the linear discriminant with Gly, Cit, Met, and Phe as the explanatory variables, (ix) the linear discriminant with His, Leu, Met, Cit, Ile, and Tyr as the explanatory variables, (x) the logistic regression equation with Val, Leu, His, and Arg as the explanatory variables, (xi) the logistic regression equation with a-ABA, Met, Tyr, and His as the explanatory variables, (xii) the logistic regression equation with Val, Ile, His, Trp, and Arg as the explanatory variables, (xiii) the logistic regression equation with Cit, a-ABA, Met, and Tyr as the explanatory variables, or (xiv) the logistic regression equation with His, Leu, Met, Cit, Ile, and Tyr as the explanatory variables. Thus, the discriminant values obtained in the multivariate discriminants useful particularly for the discrimination between any one of the cervical cancer, the endometrial cancer, and the ovarian cancer and the female genital cancer-free, can be utilized to bring about the effect of enabling more accurately the discrimination.

When the discrimination described in 13. above is conducted in step SA-13, the multivariate discriminant may be (i) the fractional expression with Lys, His, and Arg as the explanatory variables, (ii) the fractional expression with a-ABA, His, and Met as the explanatory variables, (iii) the fractional expression with Ile, His, Cit, and Arg as the explanatory variables, (iv) the linear discriminant with Gly, Val, His, and Arg as the explanatory variables, (v) the linear discriminant with Gly, Phe, His, and Arg as the explanatory variables, (vi) the linear discriminant with Cit, Ile, His, and Arg as the explanatory variables, (vii) the linear discriminant with His, Leu, Met, Cit, Ile, and Tyr as the explanatory variables, (viii) the logistic regression equation with Val, His, Lys, and Arg as the explanatory variables, (ix) the logistic regression equation with Thr, a-ABA, Met, and His as the explanatory variables, (x) the logistic regression equation with Cit, Ile, His, and Arg as the explanatory variables, or (xi) the logistic regression equation with His, Leu, Met, Cit, Ile, and Tyr as the explanatory variables. Thus, the discriminant values obtained in the multivariate discriminants useful particularly for the discrimination between any one of the cervical cancer and the endometrial cancer and any one of the cervical cancer-free and the endometrial cancer-free, can be utilized to bring about the effect of enabling more accurately the discrimination.

When the discrimination described in 14. above is conducted in step SA-13, the multivariate discriminant may be (i) the fractional expression with a-ABA, His, and Val as the explanatory variables, (ii) the fractional expression with a-ABA, Met, and Val as the explanatory variables, (iii) the fractional expression with Met, His, Cit, and Arg as the explanatory variables, (iv) the linear discriminant with Gly, Val, His, and Arg as the explanatory variables, (v) the linear discriminant with Gly, Val, Met, and Lys as the explanatory variables, (vi) the linear discriminant with Cit, Met, His, and Arg as the explanatory variables, (vii) the linear discriminant with His, Leu, Met, Ile, Tyr, and Lys as the explanatory variables, (viii) the logistic regression equation with Val, Leu, His, and Arg as the explanatory variables, (ix) the logistic regression equation with Met, His, Orn, and Arg as the explanatory variables, (x) the logistic regression equation with Val, Tyr, His, and Arg as the explanatory variables, or (xi) the logistic regression equation with His, Leu, Met, Ile, Tyr, and Lys as the explanatory variables. Thus, the discriminant values obtained in the multivariate discriminants useful particularly for the 2-group discrimination between the cervical cancer and the cervical cancer-free, can be utilized to bring about the effect of enabling more accurately the 2-group discrimination.

When the discrimination described in 15. above is conducted in step SA-13, the multivariate discriminant may be (i) the fractional expression with Lys, His, and Arg as the explanatory variables, (ii) the fractional expression with a-ABA, His, and Met as the explanatory variables, (iii) the fractional expression with Ile, His, Asn, and Cit as the explanatory variables, (iv) the linear discriminant with Gln, His, Lys, and Arg as the explanatory variables, (v) the linear discriminant with Gly, Met, Phe, and His as the explanatory variables, (vi) the linear discriminant with Cit, Ile, His, and Arg as the explanatory variables, (vii) the linear discriminant with His, Asn, Val, Pro, Cit, and Ile as the explanatory variables, (viii) the logistic regression equation with Gln, Gly, His, and Arg as the explanatory variables, (ix) the logistic regression equation with Gln, Phe, His, and Arg as the explanatory variables, (x) the logistic regression equation with Gln, Ile, His, and Arg as the explanatory variables, or (xi) the logistic regression equation with His, Asn, Val, Pro, Cit, and Ile as the explanatory variables. Thus, the discriminant values obtained in the multivariate discriminants useful particularly for the 2-group discrimination between the endometrial cancer and the endometrial cancer-free, can be utilized to bring about the effect of enabling more accurately the 2-group discrimination.

When the discrimination described in 16. above is conducted in step SA-13, the multivariate discriminant may be (i) the fractional expression with Orn, Cit, and Met as the explanatory variables, (ii) the fractional expression with Gln, Cit, and Tyr as the explanatory variables, (iii) the fractional expression with Orn, His, Phe, and Trp as the explanatory variables, (iv) the linear discriminant with Ser, Cit, Orn, and Trp as the explanatory variables, (v) the linear discriminant with Ser, Cit, Ile, and Orn as the explanatory variables, (vi) the linear discriminant with Phe, Trp, Orn, and Lys as the explanatory variables, (vii) the linear discriminant with His, Trp, Glu, Cit, Ile, and Orn as the explanatory variables, (viii) the logistic regression equation with Ser, Cit, Trp, and Orn as the explanatory variables, (ix) the logistic regression equation with Gln, Cit, Ile, and Tyr as the explanatory variables, (x) the logistic regression equation with Asn, Phe, His, and Trp as the explanatory variables, or (xi) the logistic regression equation with His, Trp, Glu, Cit, Ile, and Orn as the explanatory variables. Thus, the discriminant values obtained in the multivariate discriminants useful particularly for the 2-group discrimination between the ovarian cancer and the ovarian cancer-free, can be utilized to bring about the effect of enabling more accurately the 2-group discrimination.

When the discrimination described in 17. above is conducted in step SA-13, the multivariate discriminant may be the discriminant with Cit, Met, Lys, Asn, Ala, Thr, Gln, and a-ABA as the explanatory variables which is prepared by the Mahalanobis' generalized distance method, or the discriminant prepared with His, Leu, Ser, Thr, Glu, Gln, Ala, and Lys as the explanatory variables which is prepared by the Mahalanobis' generalized distance method. Thus, the discriminant values obtained in the multivariate discriminants useful particularly for the discrimination between the cervical cancer, the endometrial cancer, and the ovarian cancer, can be utilized to bring about the effect of enabling more accurately the discrimination.

When the discrimination described in 18. above is conducted in step SA-13, the multivariate discriminant may be the linear discriminant with Phe, His, Met, Pro, Lys, and Arg as the explanatory variables, or the logistic regression equation with Phe, His, Met, Pro, Lys, and Arg as the explanatory variables. Thus, the discriminant values obtained in the multivariate discriminants useful particularly for the 2-group discrimination between the female genital cancer suffering risk group and the healthy group, can be utilized to bring about the effect of enabling more accurately the 2-group discrimination.

The multivariate discriminant described above can be prepared by a method described in International Publication WO 2004/052191 that is an international application filed by the present applicant or by a method (multivariate discriminant-preparing processing described in the second embodiment described later) described in International Publication WO 2006/098192 that is an international application filed by the present applicant. Any multivariate discriminants obtained by these methods can be preferably used in the evaluation of the state of female genital cancer, regardless of the unit of the amino acid concentration in the amino acid concentration data as input data.

### Second Embodiment

### 2-1. Outline of the Invention

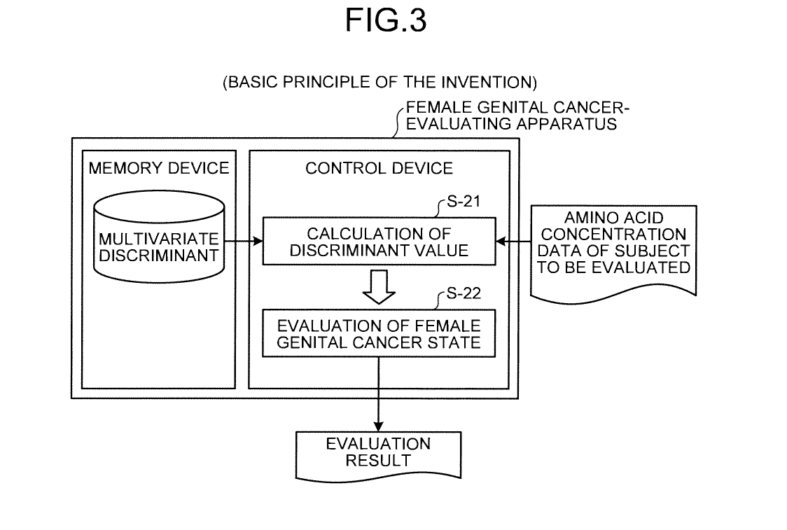

Herein, an outline of the female genital cancer-evaluating apparatus, the female genital cancer-evaluating method, the female genital cancer-evaluating system, the female genital cancer-evaluating program and the recording medium of the present invention are described in detail with reference to FIG. 3. FIG. 3 is a principle configurational diagram showing a basic principle of the present invention.

In the present invention, a discriminant value that is a value of a multivariate discriminant with a concentration of an amino acid as an explanatory variable is calculated in a control device, based on both (i) a concentration value of at least one of Arg, Asn, Cit, Gly, His, Leu, Met, Lys, Phe, Thr, Trp, Tyr, and Val contained in previously obtained amino acid concentration data on the concentration value of the amino acid of a subject (for example, an individual such as animal or human) to be evaluated and (ii) the multivariate discriminant containing at least one of Arg, Asn, Cit, Gly, His, Leu, Met, Lys, Phe, Thr, Trp, Tyr, and Val as the explanatory variable, stored in a memory device (step S-21).

In the present invention, the state of female genital cancer including at least one of cervical cancer, endometrial cancer, and ovarian cancer in the subject is evaluated in the control device based on the discriminant value calculated in step S-21 (step S-22).

According to the present invention described above, (I) the discriminant value that is the value of the multivariate discriminant with the concentration of the amino acid as the explanatory variable is calculated based on both (i) the concentration value of at least one of Thr, Ser, Asn, Gln, Pro, Gly, Ala, Cit, Val, Met, Ile, Leu, Tyr, Phe, His, Trp, Orn, Lys, and Arg contained in the previously obtained amino acid concentration data on the concentration value of the amino acid of the subject and (ii) the multivariate discriminant stored in the memory device containing at least one of Thr, Ser, Asn, Gln, Pro, Gly, Ala, Cit, Val, Met, Ile, Leu, Tyr, Phe, His, Trp, Orn, Lys, and Arg as the explanatory variable, and (II) the state of female genital cancer in the subject is evaluated based on the calculated discriminant value. Thus, the discriminant values obtained in the multivariate discriminants correlated significantly with the state of female genital cancer can be utilized to bring about the effect of enabling an accurate evaluation of the state of female genital cancer. Specifically, a subject likely to contract female genital cancer can be narrowed by one sample in a short time to bring about the effect of enabling the reduction of temporal, physical and financial burden of the subject. Specifically, whether a certain sample is with female genital cancer can be evaluated accurately by the concentrations of a plurality of the amino acids and the discriminants with the concentrations of the amino acids as the explanatory variables to bring about the effect of enabling to make the examination efficient and high accurate.

In step S-22, the discrimination (i) between the female genital cancer and the female genital cancer-free, (ii) between any one of the cervical cancer, the endometrial cancer, and the ovarian cancer and the female genital cancer-free, (iii) between any one of the cervical cancer and the endometrial cancer and any one of the cervical cancer-free and the endometrial cancer-free, (iv) between the cervical cancer and the cervical cancer-free, (v) between the endometrial cancer and the endometrial cancer-free, (vi) between the ovarian cancer and the ovarian cancer-free, (vii) between the female genital cancer suffering risk group and the healthy group, or (viii) between the cervical cancer, the endometrial cancer, and the ovarian cancer in the subject may be conducted based on the discriminant value calculated in step S-21. Thus, the discriminant values obtained in the multivariate discriminants useful for (i) the 2-group discrimination between the female genital cancer and the female genital cancer-free, (ii) the discrimination between any one of the cervical cancer, the endometrial cancer, and the ovarian cancer and the female genital cancer-free, (iii) the discrimination between any one of the cervical cancer and the endometrial cancer and any one of the cervical cancer-free and the endometrial cancer-free, (iv) the 2-group discrimination between the cervical cancer and the cervical cancer-free, (v) the 2-group discrimination between the endometrial cancer and the endometrial cancer-free, (vi) the 2-group discrimination between the ovarian cancer and the ovarian cancer-free, (vii) the 2-group discrimination between the female genital cancer suffering risk group and the healthy group, or (viii) the discrimination between the cervical cancer, the endometrial cancer, and the ovarian cancer, can be utilized to bring about the effect of enabling accurately these 2-group discriminations or these discriminations.

The multivariate discriminant may be any one of a fractional expression, the sum of a plurality of the fractional expressions, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Thus, the discriminant values obtained in the multivariate discriminants useful particularly for (i) the 2-group discrimination between the female genital cancer and the female genital cancer-free, (ii) the discrimination between any one of the cervical cancer, the endometrial cancer, and the ovarian cancer and the female genital cancer-free, (iii) the discrimination between any one of the cervical cancer and the endometrial cancer and any one of the cervical cancer-free and the endometrial cancer-free, (iv) the 2-group discrimination between the cervical cancer and the cervical cancer-free, (v) the 2-group discrimination between the endometrial cancer and the endometrial cancer-free, (vi) the 2-group discrimination between the ovarian cancer and the ovarian cancer-free, (vii) the 2-group discrimination between the female genital cancer suffering risk group and the healthy group, or (viii) the discrimination between the cervical cancer, the endometrial cancer, and the ovarian cancer, can be utilized to bring about the effect of enabling more accurately these 2-group discriminations or these discriminations.

(I) In step S-21, the discriminant value may be calculated based on both (i) the concentration value of at least one of Thr, Ser, Asn, Gln, Pro, Ala, Val, Met, Ile, Leu, Tyr, Phe, His, Trp, Orn, and Arg contained in the amino acid concentration data and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) in step S-22, the discrimination between any one of the cervical cancer, the endometrial cancer, and the ovarian cancer and the female genital cancer-free in the subject may be conducted based on the calculated discriminant value. Thus, the discriminant values obtained in the multivariate discriminants useful particularly for the discrimination between any one of the cervical cancer, the endometrial cancer, and the ovarian cancer and the female genital cancer-free, can be utilized to bring about the effect of enabling more accurately the discrimination. The multivariate discriminant to be used in this case may be (i) the fractional expression with Gln, His, and Arg as the explanatory variables, (ii) the fractional expression with a-ABA, His, and Met as the explanatory variables, (iii) the fractional expression with Ile, His, Cit, Arg, Tyr, and Trp as the explanatory variables, (iv) the fractional expression with a-ABA, Cit, and Met as the explanatory variables, (v) the linear discriminant with Gly, Val, His, and Arg as the explanatory variables, (vi) the linear discriminant with Gly, a-ABA, Met, and His as the explanatory variables, (vii) the linear discriminant with Ala, Ile, His, Trp, and Arg as the explanatory variables, (viii) the linear discriminant with Gly, Cit, Met, and Phe as the explanatory variables, (ix) the linear discriminant with His, Leu, Met, Cit, Ile, and Tyr as the explanatory variables, (x) the logistic regression equation with Val, Leu, His, and Arg as the explanatory variables, (xi) the logistic regression equation with a-ABA, Met, Tyr, and His as the explanatory variables, (xii) the logistic regression equation with Val, Ile, His, Trp, and Arg as the explanatory variables, (xiii) the logistic regression equation with Cit, a-ABA, Met, and Tyr as the explanatory variables, or (xiv) the logistic regression equation with His, Leu, Met, Cit, Ile, and Tyr as the explanatory variables. Thus, the discriminant values obtained in the multivariate discriminants useful particularly for the discrimination between any one of the cervical cancer, the endometrial cancer, and the ovarian cancer and the female genital cancer-free, can be utilized to bring about the effect of enabling more accurately the discrimination.

(I) In step S-21, the discriminant value may be calculated based on both (i) the concentration value of at least one of Thr, Ser, Asn, Pro, Gly, Cit, Val, Met, Ile, Leu, Phe, His, Trp, Orn, Lys, and Arg contained in the amino acid concentration data and (ii) the multivariate discriminant containing at least one of Thr, Ser, Asn, Pro, Gly, Cit, Val, Met, Ile, Leu, Phe, His, Trp, Orn, Lys, and Arg as the explanatory variable, and (II) in step S-22, the discrimination between any one of the cervical cancer and the endometrial cancer and any one of the cervical cancer-free and the endometrial cancer-free in the subject may be conducted based on the calculated discriminant value. Thus, the discriminant values obtained in the multivariate discriminants useful particularly for the discrimination between any one of the cervical cancer and the endometrial cancer and any one of the cervical cancer-free and the endometrial cancer-free, can be utilized to bring about the effect of enabling more accurately the discrimination. The multivariate discriminant to be used in this case may be (i) the fractional expression with Lys, His, and Arg as the explanatory variables, (ii) the fractional expression with a-ABA, His, and Met as the explanatory variables, (iii) the fractional expression with Ile, His, Cit, and Arg as the explanatory variables, (iv) the linear discriminant with Gly, Val, His, and Arg as the explanatory variables, (v) the linear discriminant with Gly, Phe, His, and Arg as the explanatory variables, (vi) the linear discriminant with Cit, Ile, His, and Arg as the explanatory variables, (vii) the linear discriminant with His, Leu, Met, Cit, Ile, and Tyr as the explanatory variables, (viii) the logistic regression equation with Val, His, Lys, and Arg as the explanatory variables, (ix) the logistic regression equation with Thr, a-ABA, Met, and His as the explanatory variables, (x) the logistic regression equation with Cit, Ile, His, and Arg as the explanatory variables, or (xi) the logistic regression equation with His, Leu, Met, Cit, Ile, and Tyr as the explanatory variables. Thus, the discriminant values obtained in the multivariate discriminants useful particularly for the discrimination between any one of the cervical cancer and the endometrial cancer and any one of the cervical cancer-free and the endometrial cancer-free, can be utilized to bring about the effect of enabling more accurately the discrimination.

(I) In step S-21, the discriminant value may be calculated based on both (i) the concentration value of at least one of Asn, Val, Met, Leu, Phe, His, Trp, Orn, Lys, and Arg contained in the amino acid concentration data and (ii) the multivariate discriminant containing at least one of Asn, Val, Met, Leu, Phe, His, Trp, Orn, Lys, and Arg as the explanatory variable, and (II) in step S-22, the discrimination between the cervical cancer and the cervical cancer-free in the subject may be conducted based on the calculated discriminant value. Thus, the discriminant values obtained in the multivariate discriminants useful particularly for the 2-group discrimination between the cervical cancer and the cervical cancer-free, can be utilized to bring about the effect of enabling more accurately the 2-group discrimination. The multivariate discriminant to be used in this case may be (i) the fractional expression with a-ABA, His, and Val as the explanatory variables, (ii) the fractional expression with a-ABA, Met, and Val as the explanatory variables, (iii) the fractional expression with Met, His, Cit, and Arg as the explanatory variables, (iv) the linear discriminant with Gly, Val, His, and Arg as the explanatory variables, (v) the linear discriminant with Gly, Val, Met, and Lys as the explanatory variables, (vi) the linear discriminant with Cit, Met, His, and Arg as the explanatory variables, (vii) the linear discriminant with His, Leu, Met, Ile, Tyr, and Lys as the explanatory variables, (viii) the logistic regression equation with Val, Leu, His, and Arg as the explanatory variables, (ix) the logistic regression equation with Met, His, Orn, and Arg as the explanatory variables, (x) the logistic regression equation with Val, Tyr, His, and Arg as the explanatory variables, or (xi) the logistic regression equation with His, Leu, Met, Ile, Tyr, and Lys as the explanatory variables. Thus, the discriminant values obtained in the multivariate discriminants useful particularly for the 2-group discrimination between the cervical cancer and the cervical cancer-free, can be utilized to bring about the effect of enabling more accurately the 2-group discrimination.

(I) In step S-21, the discriminant value may be calculated based on both (i) the concentration value of at least one of Thr, Ser, Asn, Pro, Gly, Cit, Val, Met, Ile, Leu, Phe, His, Trp, and Arg contained in the amino acid concentration data and (ii) the multivariate discriminant containing at least one of Thr, Ser, Asn, Pro, Gly, Cit, Val, Met, Ile, Leu, Phe, His, Trp, and Arg as the explanatory variable, and (II) in step S-22, the discrimination between the endometrial cancer and the endometrial cancer-free in the subject may be conducted based on the calculated discriminant value. Thus, the discriminant values obtained in the multivariate discriminants useful particularly for the 2-group discrimination between the endometrial cancer and the endometrial cancer-free, can be utilized to bring about the effect of enabling more accurately the 2-group discrimination. The multivariate discriminant to be used in this case may be (i) the fractional expression with Lys, His, and Arg as the explanatory variables, (ii) the fractional expression with a-ABA, His, and Met as the explanatory variables, (iii) the fractional expression with Ile, His, Asn, and Cit as the explanatory variables, (iv) the linear discriminant with Gln, His, Lys, and Arg as the explanatory variables, (v) the linear discriminant with Gly, Met, Phe, and His as the explanatory variables, (vi) the linear discriminant with Cit, Ile, His, and Arg as the explanatory variables, (vii) the linear discriminant with His, Asn, Val, Pro, Cit, and Ile as the explanatory variables, (viii) the logistic regression equation with Gln, Gly, His, and Arg as the explanatory variables, (ix) the logistic regression equation with Gln, Phe, His, and Arg as the explanatory variables, (x) the logistic regression equation with Gln, Ile, His, and Arg as the explanatory variables, or (xi) the logistic regression equation with His, Asn, Val, Pro, Cit, and Ile as the explanatory variables. Thus, the discriminant values obtained in the multivariate discriminants useful particularly for the 2-group discrimination between the endometrial cancer and the endometrial cancer-free, can be utilized to bring about the effect of enabling more accurately the 2-group discrimination.

(I) In step S-21, the discriminant value may be calculated based on both (i) the concentration value of at least one of Thr, Ser, Asn, Gln, Ala, Cit, Val, Met, Ile, Leu, Tyr, Phe, His, Trp, Orn, Lys, and Arg contained in the amino acid concentration data and (ii) the multivariate discriminant containing at least one of Thr, Ser, Asn, Gln, Ala, Cit, Val, Met, Ile, Leu, Tyr, Phe, His, Trp, Orn, Lys, and Arg as the explanatory variable, and (II) in step S-22, the discrimination between the ovarian cancer and the ovarian cancer-free in the subject may be conducted based on the calculated discriminant value. Thus, the discriminant values obtained in the multivariate discriminants useful particularly for the 2-group discrimination between the ovarian cancer and the ovarian cancer-free, can be utilized to bring about the effect of enabling more accurately the 2-group discrimination. The multivariate discriminant to be used in this case may be (i) the fractional expression with Orn, Cit, and Met as the explanatory variables, (ii) the fractional expression with Gln, Cit, and Tyr as the explanatory variables, (iii) the fractional expression with Orn, His, Phe, and Trp as the explanatory variables, (iv) the linear discriminant with Ser, Cit, Orn, and Trp as the explanatory variables, (v) the linear discriminant with Ser, Cit, Ile, and Orn as the explanatory variables, (vi) the linear discriminant with Phe, Trp, Orn, and Lys as the explanatory variables, (vii) the linear discriminant with His, Trp, Glu, Cit, Ile, and Orn as the explanatory variables, (viii) the logistic regression equation with Ser, Cit, Trp, and Orn as the explanatory variables, (ix) the logistic regression equation with Gln, Cit, Ile, and Tyr as the explanatory variables, (x) the logistic regression equation with Asn, Phe, His, and Trp as the explanatory variables, or (xi) the logistic regression equation with His, Trp, Glu, Cit, Ile, and Orn as the explanatory variables. Thus, the discriminant values obtained in the multivariate discriminants useful particularly for the 2-group discrimination between the ovarian cancer and the ovarian cancer-free, can be utilized to bring about the effect of enabling more accurately the 2-group discrimination.

(I) In step S-21, the discriminant value may be calculated based on both (i) the concentration value of at least one of Thr, Ser, Asn, Gln, Pro, Ala, Val, Met, Ile, Leu, Tyr, Phe, His, Trp, Orn, and Arg contained in the amino acid concentration data and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) in step S-22, the discrimination between the female genital cancer suffering risk group and the healthy group in the subject may be conducted based on the calculated discriminant value. Thus, the discriminant values obtained in the multivariate discriminants useful particularly for the 2-group discrimination between the female genital cancer suffering risk group and the healthy group, can be utilized to bring about the effect of enabling more accurately the 2-group discrimination. The multivariate discriminant to be used in this case may be the linear discriminant with Phe, His, Met, Pro, Lys, and Arg as the explanatory variables, or the logistic regression equation with Phe, His, Met, Pro, Lys, and Arg as the explanatory variables. Thus, the discriminant values obtained in the multivariate discriminants useful particularly for the 2-group discrimination between the female genital cancer suffering risk group and the healthy group, can be utilized to bring about the effect of enabling more accurately the 2-group discrimination.

(I) In step S-21, the discriminant value may be calculated based on both (i) the concentration value of at least one of Thr, Ser, Asn, Glu, Gln, Pro, Gly, Ala, Cit, ABA, Val, Met, Ile, Leu, Tyr, Phe, His, Trp, Orn, Lys, and Arg contained in the amino acid concentration data and (ii) the multivariate discriminant containing at least one of Thr, Ser, Asn, Glu, Gln, Pro, Gly, Ala, Cit, ABA, Val, Met, Ile, Leu, Tyr, Phe, His, Trp, Orn, Lys, and Arg as the explanatory variable, and (II) in step S-22, the discrimination between the cervical cancer, the endometrial cancer, and the ovarian cancer in the subject may be conducted based on the calculated discriminant value. Thus, the discriminant values obtained in the multivariate discriminants useful particularly for the discrimination between the cervical cancer, the endometrial cancer, and the ovarian cancer, can be utilized to bring about the effect of enabling more accurately the discrimination. The multivariate discriminant to be used in this case may be the discriminant with Cit, Met, Lys, Asn, Ala, Thr, Gln, and a-ABA as the explanatory variables which is prepared by the Mahalanobis' generalized distance method, or the discriminant prepared with His, Leu, Ser, Thr, Glu, Gln, Ala, and Lys as the explanatory variables which is prepared by the Mahalanobis' generalized distance method. Thus, the discriminant values obtained in the multivariate discriminants useful particularly for the discrimination between the cervical cancer, the endometrial cancer, and the ovarian cancer, can be utilized to bring about the effect of enabling more accurately the discrimination.

The multivariate discriminant described above can be prepared by a method described in International Publication WO 2004/052191 that is an international application filed by the present applicant or by a method (multivariate discriminant-preparing processing described later) described in International Publication WO 2006/098192 that is an international application filed by the present applicant. Any multivariate discriminants obtained by these methods can be preferably used in the evaluation of the state of female genital cancer, regardless of the unit of the amino acid concentration in the amino acid concentration data as input data.

The multivariate discriminant refers to a form of equation used generally in multivariate analysis and includes, for example, multiple regression equation, multiple logistic regression equation, linear discriminant function, Mahalanobis' generalized distance, canonical discriminant function, support vector machine, and decision tree. The multivariate discriminant also includes an equation shown by the sum of different forms of the multivariate discriminants. In the multiple regression equation, multiple logistic regression equation and canonical discriminant function, a coefficient and constant term are added to each explanatory variable, and the coefficient and constant term in this case are preferably real numbers, more preferably values in the range of 99% confidence interval for the coefficient and constant term obtained from data for discrimination, more preferably in the range of 95% confidence interval for the coefficient and constant term obtained from data for discrimination. The value of each coefficient and the confidence interval thereof may be those multiplied by a real number, and the value of each constant term and the confidence interval thereof may be those having an arbitrary actual constant added or subtracted or those multiplied or divided by an arbitrary actual constant.

In the fractional expression, the numerator of the fractional expression is expressed by the sum of the amino acids A, B, C etc. and the denominator of the fractional expression is expressed by the sum of the amino acids a, b, c etc. The fractional expression also includes the sum of the fractional expressions α, β, γ etc. (for example, a+β) having such constitution. The fractional expression also includes divided fractional expressions. The amino acids used in the numerator or denominator may have suitable coefficients respectively. The amino acids used in the numerator or denominator may appear repeatedly. Each fractional expression may have a suitable coefficient. A value of a coefficient for each explanatory variable and a value for a constant term may be any real numbers. In combinations where explanatory variables in the numerator and explanatory variables in the denominator in the fractional expression are switched with each other, the positive (or negative) sign is generally reversed in correlation with objective explanatory variables, but because their correlation is maintained, such combinations can be assumed to be equivalent to one another in discrimination, and thus the fractional expression also includes combinations where explanatory variables in the numerator and explanatory variables in the denominator in the fractional expression are switched with each other.

When the state of female genital cancer is evaluated in the present invention, the concentrations of the other metabolites, the gene expression level, the protein expression level, the age and sex of the subject, the presence or absence of the smoking, the digitalized electrocardiogram waveform, or the like may be used in addition to the amino acid concentration. When the state of female genital cancer is evaluated in the present invention, the concentrations of the other metabolites, the gene expression level, the protein expression level, the age and sex of the subject, the presence or absence of the smoking, the digitalized electrocardiogram waveform, or the like may be used as the explanatory variables in the multivariate discriminant in addition to the amino acid concentration.

Here, the summary of the multivariate discriminant-preparing processing (steps 1 to 4) is described in detail.

First, a candidate multivariate discriminant (e.g., y=a₁x₁+a₂x₂+ . . . +aₙxₙ, y: female genital cancer state index data, xᵢ: amino acid concentration data, aᵢ: constant, i=1, 2, . . . , n) that is a candidate for the multivariate discriminant is prepared in the control device based on a predetermined discriminant-preparing method from female genital cancer state information stored in the memory device containing the amino acid concentration data and female genital cancer state index data on an index for indicating the state of female genital cancer (step 1). Data containing defective and outliers may be removed in advance from the female genital cancer state information.

In step 1, a plurality of the candidate multivariate discriminants may be prepared from the female genital cancer state information by using a plurality of the different discriminant-preparing methods (including those for multivariate analysis such as principal component analysis, discriminant analysis, support vector machine, multiple regression analysis, logistic regression analysis, k-means method, cluster analysis, and decision tree). Specifically, a plurality of the candidate multivariate discriminants may be prepared simultaneously and concurrently by using a plurality of different algorithms with the female genital cancer state information which is multivariate data composed of the amino acid concentration data and the female genital cancer state index data obtained by analyzing blood samples from a large number of healthy subjects and female genital cancer patients. For example, the two different candidate multivariate discriminants may be formed by performing discriminant analysis and logistic regression analysis simultaneously with the different algorithms. Alternatively, the candidate multivariate discriminant may be formed by converting the female genital cancer state information with the candidate multivariate discriminant prepared by performing principal component analysis and then performing discriminant analysis of the converted female genital cancer state information. In this way, it is possible to finally prepare the multivariate discriminant suitable for diagnostic condition.

The candidate multivariate discriminant prepared by principal component analysis is a linear expression consisting of amino acid explanatory variables maximizing the variance of all amino acid concentration data. The candidate multivariate discriminant prepared by discriminant analysis is a high-powered expression (including exponential and logarithmic expressions) consisting of amino acid explanatory variables minimizing the ratio of the sum of the variances in respective groups to the variance of all amino acid concentration data. The candidate multivariate discriminant prepared by using support vector machine is a high-powered expression (including kernel function) consisting of amino acid explanatory variables maximizing the boundary between groups. The candidate multivariate discriminant prepared by multiple regression analysis is a high-powered expression consisting of amino acid explanatory variables minimizing the sum of the distances from all amino acid concentration data. The candidate multivariate discriminant prepared by logistic regression analysis is a fraction expression having, as a component, the natural logarithm having a linear expression consisting of amino acid explanatory variables maximizing the likelihood as the exponent. The k-means method is a method of searching k pieces of neighboring amino acid concentration data in various groups, designating the group containing the greatest number of the neighboring points as its data-belonging group, and selecting the amino acid explanatory variable that makes the group to which input amino acid concentration data belong agree well with the designated group. The cluster analysis is a method of clustering (grouping) the points closest in entire amino acid concentration data. The decision tree is a method of ordering amino acid explanatory variables and predicting the group of amino acid concentration data from the pattern possibly held by the higher-ordered amino acid explanatory variable.

Returning to the description of the multivariate discriminant-preparing processing, the candidate multivariate discriminant prepared in step 1 is verified (mutually verified) in the control device based on a particular verifying method (step 2). The verification of the candidate multivariate discriminant is performed on each other to each candidate multivariate discriminant prepared in step 1.

In step 2, at least one of discrimination rate, sensitivity, specificity, information criterion, and the like of the candidate multivariate discriminant may be verified by at least one of the bootstrap method, holdout method, leave-one-out method, and the like. In this way, it is possible to prepare the candidate multivariate discriminant higher in predictability or reliability, by taking the female genital cancer state information and the diagnostic condition into consideration.

The discrimination rate is the rate of the female genital cancer states judged correct according to the present invention in all input data. The sensitivity is the rate of the female genital cancer states judged correct according to the present invention in the female genital cancer states declared female genital cancer in the input data. The specificity is the rate of the female genital cancer states judged correct according to the present invention in the female genital cancer states declared healthy in the input data. The information criterion is the sum of the number of the amino acid explanatory variables in the candidate multivariate discriminant prepared in step 1 and the difference in number between the female genital cancer states evaluated according to the present invention and those declared in input data. The predictability is the average of the discrimination rate, sensitivity, or specificity obtained by repeating verification of the candidate multivariate discriminant. Alternatively, the reliability is the variance of the discrimination rate, sensitivity, or specificity obtained by repeating verification of the candidate multivariate discriminant.

Returning to the description of the multivariate discriminant-preparing processing, a combination of the amino acid concentration data contained in the female genital cancer state information used in preparing the candidate multivariate discriminant is selected by selecting the explanatory variable of the candidate multivariate discriminant in the control device based on a predetermined explanatory variable-selecting method from the verification result obtained in step 2 (step 3). The selection of the amino acid explanatory variable is performed on each candidate multivariate discriminant prepared in step 1. In this way, it is possible to select the amino acid explanatory variable of the candidate multivariate discriminant properly. The step 1 is executed once again by using the female genital cancer state information including the amino acid concentration data selected in step 3.

In step 3, the amino acid explanatory variable of the candidate multivariate discriminant may be selected based on at least one of the stepwise method, best path method, local search method, and genetic algorithm from the verification result obtained in step 2.

The best path method is a method of selecting an amino acid explanatory variable by optimizing an evaluation index of the candidate multivariate discriminant while eliminating the amino acid explanatory variables contained in the candidate multivariate discriminant one by one.

Returning to the description of the multivariate discriminant-preparing processing, the steps 1, 2 and 3 are repeatedly performed in the control device, and based on verification results thus accumulated, the candidate multivariate discriminant used as the multivariate discriminant is selected from a plurality of the candidate multivariate discriminants, thereby preparing the multivariate discriminant (step 4). In the selection of the candidate multivariate discriminant, there are cases where the optimum multivariate discriminant is selected from the candidate multivariate discriminants prepared in the same discriminant-preparing method or the optimum multivariate discriminant is selected from all candidate multivariate discriminants.

As described above, in the multivariate discriminant-preparing processing, the processing for the preparation of the candidate multivariate discriminants, the verification of the candidate multivariate discriminants, and the selection of the explanatory variables in the candidate multivariate discriminants are performed based on the female genital cancer state information in a series of operations in a systematized manner, whereby the multivariate discriminant most appropriate for evaluating each female genital cancer state can be prepared.

### 2-2. System Configuration

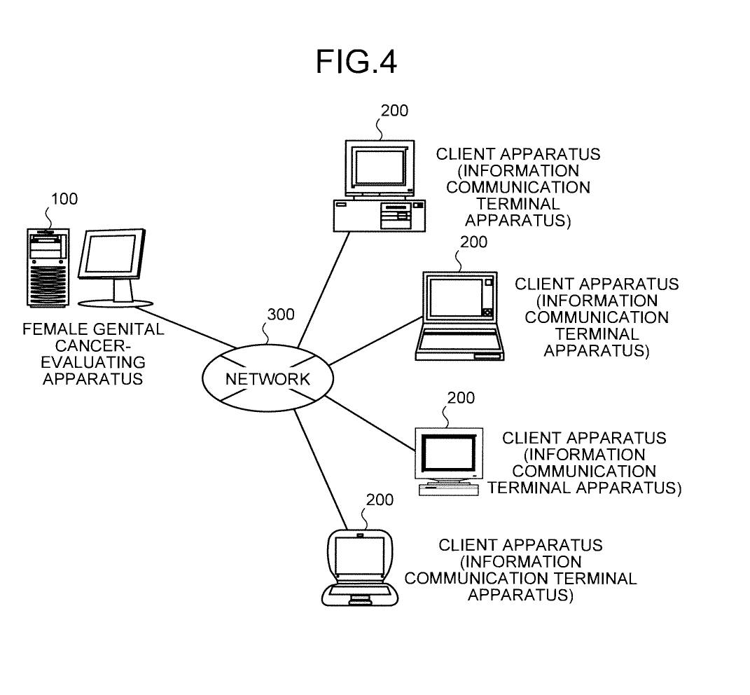

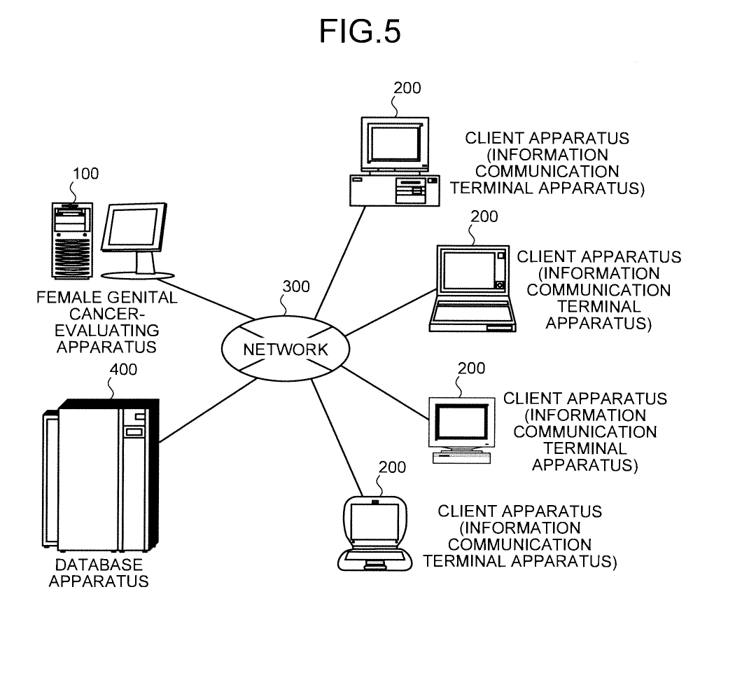

Hereinafter, the configuration of the female genital cancer-evaluating system according to the second embodiment (hereinafter referred to sometimes as the present system) will be described with reference to FIGS. 4 to 20. This system is merely one example, and the present invention is not limited thereto.

First, an entire configuration of the present system will be described with reference to FIGS. 4 and 5. FIG. 4 is a diagram showing an example of the entire configuration of the present system. FIG. 5 is a diagram showing another example of the entire configuration of the present system. As shown in FIG. 4, the present system is constituted in which the female genital cancer-evaluating apparatus 100 that evaluates the state of female genital cancer in the subject, and the client apparatus 200 (corresponding to the information communication terminal apparatus of the present invention) that provides the amino acid concentration data of the subject on the concentration values of the amino acids, are communicatively connected to each other via a network 300.

In the present system as shown in FIG. 5, in addition to the female genital cancer-evaluating apparatus 100 and the client apparatus 200, the database apparatus 400 storing, for example, the female genital cancer state information used in preparing the multivariate discriminant and the multivariate discriminant used in evaluating the state of female genital cancer in the female genital cancer-evaluating apparatus 100, may be communicatively connected via the network 300. In this configuration, the information on the state of female genital cancer etc. are provided via the network 300 from the female genital cancer-evaluating apparatus 100 to the client apparatuses 200 and the database apparatus 400, or from the client apparatuses 200 and the database apparatus 400 to the female genital cancer-evaluating apparatus 100. The information on the state of female genital cancer is information on the measured values of particular items of the state of female genital cancer of human. The information on the state of female genital cancer is generated in the female genital cancer-evaluating apparatus 100, client apparatus 200, or other apparatuses (e.g., various measuring apparatuses) and stored mainly in the database apparatus 400.

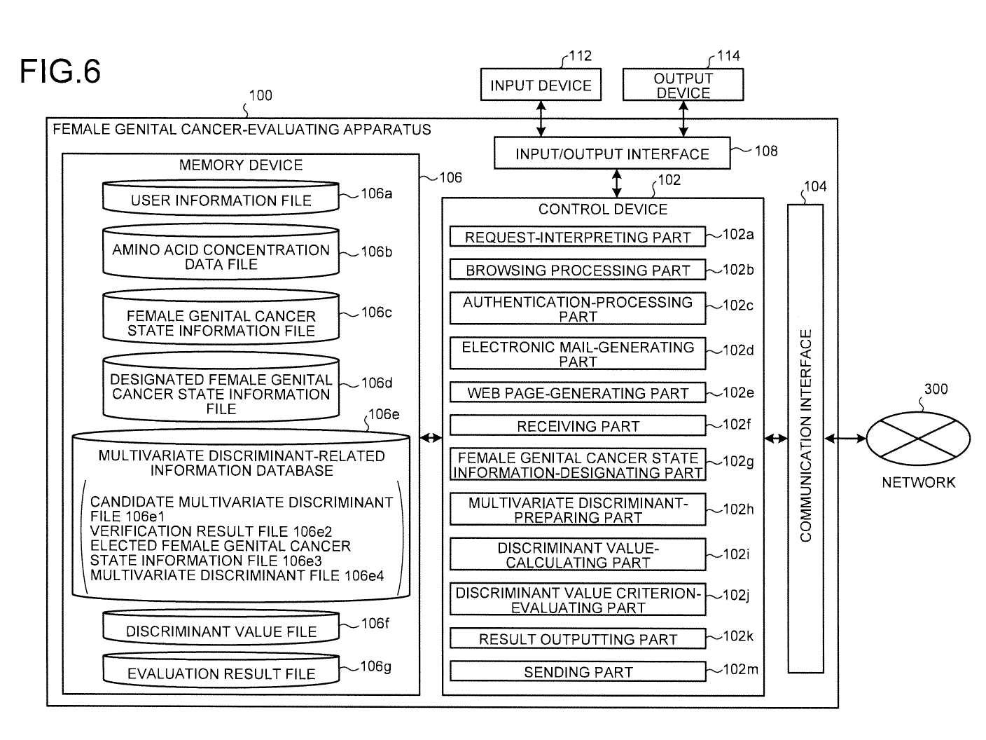

Now, the configuration of the female genital cancer-evaluating apparatus 100 in the present system will be described with reference to FIGS. 6 to 18. FIG. 6 is a block diagram showing an example of the configuration of the female genital cancer-evaluating apparatus 100 in the present system, showing conceptually only the region relevant to the present invention.

The female genital cancer-evaluating apparatus 100 includes (a) a control device 102, such as CPU (Central Processing Unit), that integrally controls the female genital cancer-evaluating apparatus 100, (b) a communication interface 104 that connects the female genital cancer-evaluating apparatus 100 to the network 300 communicatively via communication apparatuses such as a router and wired or wireless communication lines such as a private line, (c) a memory device 106 that stores various databases, tables, files and others, and (d) an input/output interface 108 connected to an input device 112 and an output device 114, and these parts are connected to each other communicatively via any communication channel. The female genital cancer-evaluating apparatus 100 may be present together with various analyzers (e.g., amino acid analyzer) in a same housing. A typical configuration of disintegration/integration of the female genital cancer-evaluating apparatus 100 is not limited to that shown in the figure, and all or a part of it may be disintegrated or integrated functionally or physically in any unit, for example, according to various loads applied. For example, a part of the processing may be performed via CGI (Common Gateway Interface).

The memory device 106 is a storage means, and examples thereof include memory apparatuses such as RAM (Random Access Memory) and ROM (Read Only Memory), fixed disk drives such as a hard disk, a flexible disk, an optical disk, and the like. The memory device 106 stores computer programs giving instructions to the CPU for various processings, together with OS (Operating System). As shown in the figure, the memory device 106 stores the user information file 106a, the amino acid concentration data file 106b, the female genital cancer state information file 106c, the designated female genital cancer state information file 106d, a multivariate discriminant-related information database 106e, the discriminant value file 106f, and the evaluation result file 106g.

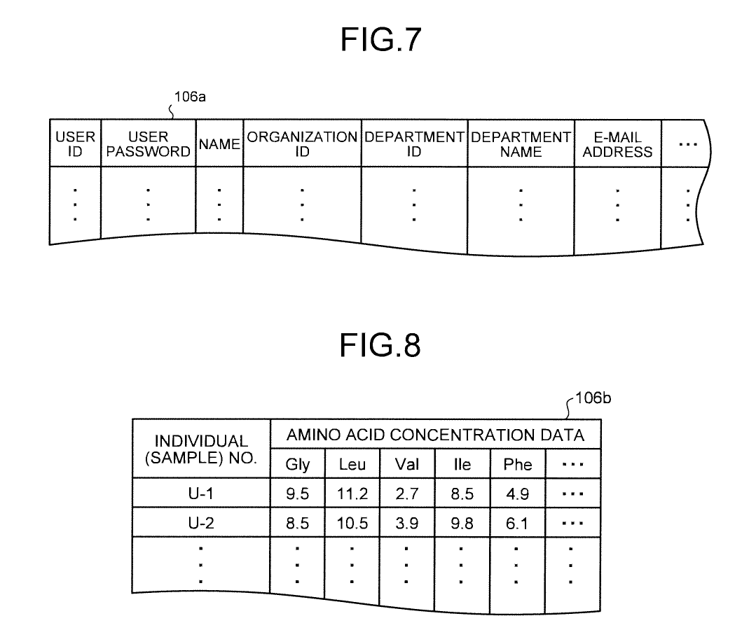

The user information file 106a stores user information on users. FIG. 7 is a chart showing an example of information stored in the user information file 106a. As shown in FIG. 7, the information stored in the user information file 106a includes user ID (identification) for identifying a user uniquely, user password for authentication of the user, user name, organization ID for uniquely identifying an organization of the user, department ID for uniquely identifying a department of the user organization, department name, and electronic mail address of the user that are correlated to one another.

Returning to FIG. 6, the amino acid concentration data file 106b stores the amino acid concentration data on the concentration values of the amino acids. FIG. 8 is a chart showing an example of information stored in the amino acid concentration data file 106b. As shown in FIG. 8, the information stored in the amino acid concentration data file 106b includes individual number for uniquely identifying an individual (sample) as a subject to be evaluated and amino acid concentration data that are correlated to one another. In FIG. 8, the amino acid concentration data is assumed to be numerical values, i.e., on a continuous scale, but the amino acid concentration data may be expressed on a nominal scale or an ordinal scale. In the case of the nominal or ordinal scale, any number may be allocated to each state for analysis. The amino acid concentration data may be combined with other biological information (e.g., the concentrations of metabolites other than the amino acids, the gene expression level, the protein expression level, the age and sex of the subject, the presence or absence of the smoking, and the digitalized electrocardiogram waveform).

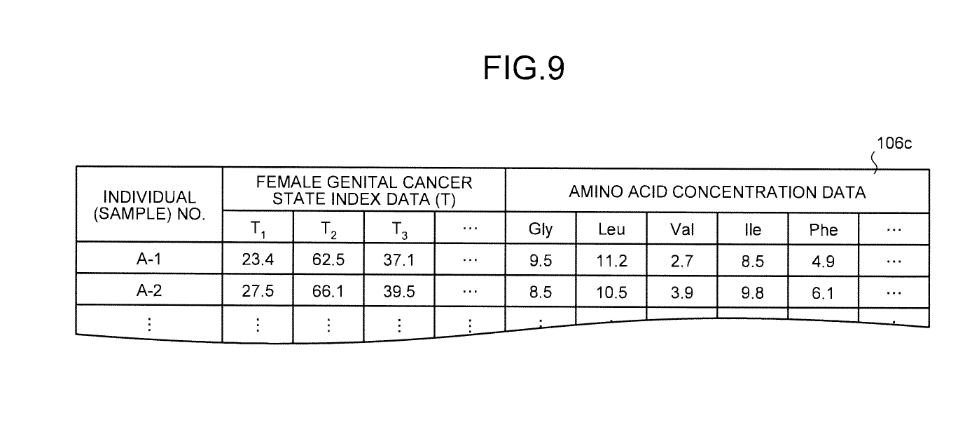

Returning to FIG. 6, the female genital cancer state information file 106c stores the female genital cancer state information used in preparing the multivariate discriminant. FIG. 9 is a chart showing an example of information stored in the female genital cancer state information file 106c. As shown in FIG. 9, the information stored in the female genital cancer state information file 106c includes individual (sample) number, female genital cancer state index data (T) corresponding to female genital cancer state index (index T₁, index T₂, index T₃ . . . ), and amino acid concentration data that are correlated to one another. In FIG. 9, the female genital cancer state index data and the amino acid concentration data are assumed to be numerical values, i.e., on a continuous scale, but the female genital cancer state index data and the amino acid concentration data may be expressed on a nominal scale or an ordinal scale. In the case of the nominal or ordinal scale, any number may be allocated to each state for analysis. The female genital cancer state index data is a single known condition index serving as a marker of the state of female genital cancer, and numerical data may be used.

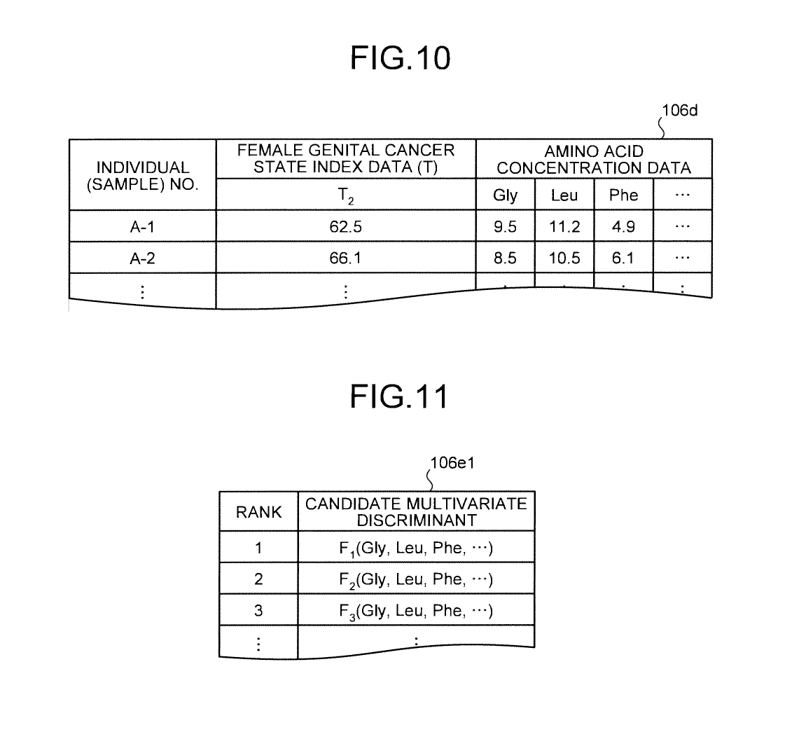

Returning to FIG. 6, the designated female genital cancer state information file 106d stores the female genital cancer state information designated in a female genital cancer state information-designating part 102g described below. FIG. 10 is a chart showing an example of information stored in the designated female genital cancer state information file 106d. As shown in FIG. 10, the information stored in the designated female genital cancer state information file 106d includes individual number, designated female genital cancer state index data, and designated amino acid concentration data that are correlated to one another.

Returning to FIG. 6, the multivariate discriminant-related information database 106e is composed of (i) the candidate multivariate discriminant file 106e1 storing the candidate multivariate discriminant prepared in a candidate multivariate discriminant-preparing part 102h1 described below, (ii) the verification result file 106e2 storing the verification results obtained in a candidate multivariate discriminant-verifying part 102h2 described below, (iii) the selected female genital cancer state information file 106e3 storing the female genital cancer state information containing the combination of the amino acid concentration data selected in an explanatory variable-selecting part 102h3 described below, and (iv) the multivariate discriminant file 106e4 storing the multivariate discriminant prepared in the multivariate discriminant-preparing part 102h described below.

The candidate multivariate discriminant file 106e1 stores the candidate multivariate discriminants prepared in the candidate multivariate discriminant-preparing part 102h1 described below. FIG. 11 is a chart showing an example of information stored in the candidate multivariate discriminant file 106e1. As shown in FIG. 11, the information stored in the candidate multivariate discriminant file 106e1 includes rank, and candidate multivariate discriminant (e.g., F₁ (Gly, Leu, Phe, . . . ), F₂ (Gly, Leu, Phe, . . . ), or F₃ (Gly, Leu, Phe, . . . ) in FIG. 11) that are correlated to each other.

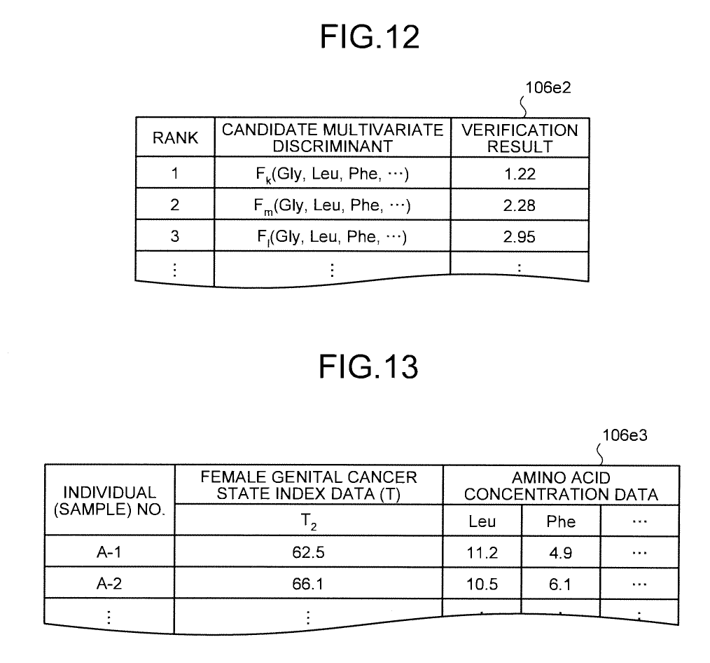

Returning to FIG. 6, the verification result file 106e2 stores the verification results obtained in the candidate multivariate discriminant-verifying part 102h2 described below. FIG. 12 is a chart showing an example of information stored in the verification result file 106e2. As shown in FIG. 12, the information stored in the verification result file 106e2 includes rank, candidate multivariate discriminant (e.g., Fₖ (Gly, Leu, Phe, . . . ), Fₘ (Gly, Leu, Phe, . . . ), F₁ (Gly, Leu, Phe, . . . ) in FIG. 12), and verification result of each candidate multivariate discriminant (e.g., evaluation value of each candidate multivariate discriminant) that are correlated to one another.

Returning to FIG. 6, the selected female genital cancer state information file 106e3 stores the female genital cancer state information including the combination of the amino acid concentration data corresponding to the explanatory variables selected in the explanatory variable-selecting part 102h3 described below. FIG. 13 is a chart showing an example of information stored in the selected female genital cancer state information file 106e3. As shown in FIG. 13, the information stored in the selected female genital cancer state information file 106e3 includes individual number, female genital cancer state index data designated in the female genital cancer state information-designating part 102g described below, and amino acid concentration data selected in the explanatory variable-selecting part 102h3 described below that are correlated to one another.

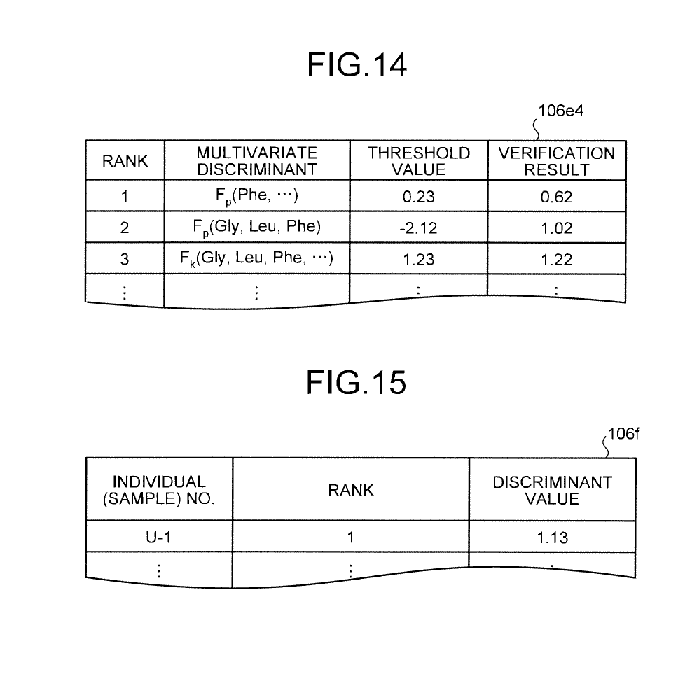

Returning to FIG. 6, the multivariate discriminant file 106e4 stores the multivariate discriminants prepared in the multivariate discriminant-preparing part 102h described below. FIG. 14 is a chart showing an example of information stored in the multivariate discriminant file 106e4. As shown in FIG. 14, the information stored in the multivariate discriminant file 106e4 includes rank, multivariate discriminant (e.g., Fₚ (Phe, . . . ), Fₚ (Gly, Leu, Phe), Fₖ (Gly, Leu, Phe, . . . ) in FIG. 14), a threshold corresponding to each discriminant-preparing method, and verification result of each multivariate discriminant (e.g., evaluation value of each multivariate discriminant) that are correlated to one another.

Returning to FIG. 6, the discriminant value file 106f stores the discriminant value calculated in a discriminant value-calculating part 102i described below. FIG. 15 is a chart showing an example of information stored in the discriminant value file 106f. As shown in FIG. 15, the information stored in the discriminant value file 106f includes individual number for uniquely identifying the individual (sample) as the subject, rank (number for uniquely identifying the multivariate discriminant), and discriminant value that are correlated to one another.

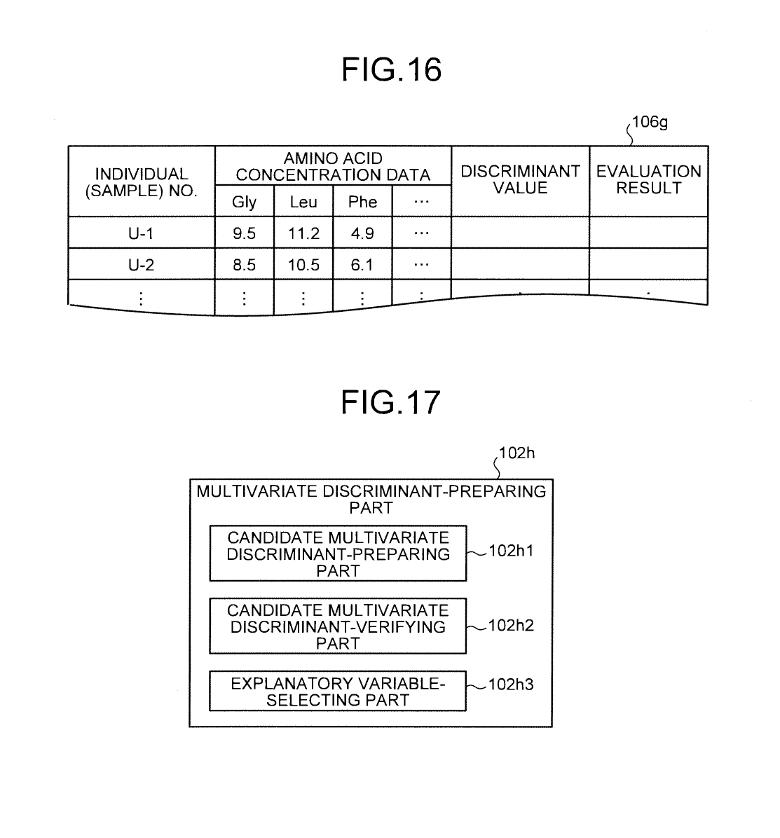

Returning to FIG. 6, the evaluation result file 106g stores the evaluation results obtained in the discriminant value criterion-evaluating part 102j described below (specifically the discrimination results obtained in a discriminant value criterion-discriminating part 102j1 described below). FIG. 16 is a chart showing an example of information stored in the evaluation result file 106g. The information stored in the evaluation result file 106g includes individual number for uniquely identifying the individual (sample) as the subject, previously obtained amino acid concentration data of the subject, discriminant value calculated by multivariate discriminant, and evaluation result on the state of female genital cancer that are correlated to one another.

Returning to FIG. 6, the memory device 106 stores various Web data for providing the client apparatuses 200 with web site information, CGI programs, and others as information other than the information described above. The Web data include data for displaying the Web pages described below and others, and the data are generated as, for example, a HTML (HyperText Markup Language) or XML (Extensible Markup Language) text file. Files for components and files for operation for generation of the Web data, and other temporary files, and the like are also stored in the memory device 106. In addition, the memory device 106 may store as needed sound files of sounds for transmission to the client apparatuses 200 in WAVE format or AIFF (Audio Interchange File Format) format and image files of still images or motion pictures in JPEG (Joint Photographic Experts Group) format or MPEG2 (Moving Picture Experts Group phase 2) format.

The communication interface 104 allows communication between the female genital cancer-evaluating apparatus 100 and the network 300 (or communication apparatus such as a router). Thus, the communication interface 104 has a function to communicate data via a communication line with other terminals.

The input/output interface 108 is connected to the input device 112 and the output device 114. A monitor (including a home television), a speaker, or a printer may be used as the output device 114 (hereinafter, the output device 114 may be described as a monitor 114). A keyboard, a mouse, a microphone, or a monitor functioning as a pointing device together with a mouse may be used as the input device 112.

The control device 102 has an internal memory storing control programs such as OS (Operating System), programs for various processing procedures, and other needed data, and performs various information processings according to these programs. As shown in the figure, the control device 102 includes mainly a request-interpreting part 102a, a browsing processing part 102b, an authentication-processing part 102c, an electronic mail-generating part 102d, a Web page-generating part 102e, a receiving part 102f, the female genital cancer state information-designating part 102g, the multivariate discriminant-preparing part 102h, the discriminant value-calculating part 102i, the discriminant value criterion-evaluating part 102j, a result outputting part 102k, and a sending part 102m. The control device 102 performs data processings such as removal of data including defective, removal of data including many outliers, and removal of explanatory variables for the defective-including data in the female genital cancer state information transmitted from the database apparatus 400 and in the amino acid concentration data transmitted from the client apparatus 200.

The request-interpreting part 102a interprets the requests transmitted from the client apparatus 200 or the database apparatus 400 and sends the requests to other parts in the control device 102 according to results of interpreting the requests. Upon receiving browsing requests for various screens transmitted from the client apparatus 200, the browsing processing part 102b generates and transmits web data for these screens. Upon receiving authentication requests transmitted from the client apparatus 200 or the database apparatus 400, the authentication-processing part 102c performs authentication. The electronic mail-generating part 102d generates electronic mails including various kinds of information. The Web page-generating part 102e generates Web pages for users to browse with the client apparatus 200.

The receiving part 102f receives, via the network 300, information (specifically, the amino acid concentration data, the female genital cancer state information, the multivariate discriminant etc.) transmitted from the client apparatus 200 and the database apparatus 400. The female genital cancer state information-designating part 102g designates objective female genital cancer state index data and objective amino acid concentration data in preparing the multivariate discriminant.

The multivariate discriminant-preparing part 102h generates the multivariate discriminants based on the female genital cancer state information received in the receiving part 102f and the female genital cancer state information designated in the female genital cancer state information-designating part 102g. Specifically, the multivariate discriminant-preparing part 102h generates the multivariate discriminant by selecting the candidate multivariate discriminant used as the multivariate discriminant from a plurality of the candidate multivariate discriminants, based on verification results accumulated by repeating processings in the candidate multivariate discriminant-preparing part 102h1, the candidate multivariate discriminant-verifying part 102h2, and the explanatory variable-selecting part 102h3 from the female genital cancer state information.

If the multivariate discriminants are stored previously in a predetermined region of the memory device 106, the multivariate discriminant-preparing part 102h may generate the multivariate discriminant by selecting the desired multivariate discriminant out of the memory device 106. Alternatively, the multivariate discriminant-preparing part 102h may generate the multivariate discriminant by selecting and downloading the desired multivariate discriminant from the multivariate discriminants previously stored in another computer apparatus (e.g., the database apparatus 400).

Hereinafter, a configuration of the multivariate discriminant-preparing part 102h will be described with reference to FIG. 17. FIG. 17 is a block diagram showing the configuration of the multivariate discriminant-preparing part 102h, and only a part in the configuration related to the present invention is shown conceptually. The multivariate discriminant-preparing part 102h has the candidate multivariate discriminant-preparing part 102h1, the candidate multivariate discriminant-verifying part 102h2, and the explanatory variable-selecting part 102h3, additionally. The candidate multivariate discriminant-preparing part 102h1 generates the candidate multivariate discriminant that is a candidate of the multivariate discriminant, from the female genital cancer state information based on a predetermined discriminant-preparing method. The candidate multivariate discriminant-preparing part 102h1 may generate a plurality of the candidate multivariate discriminants from the female genital cancer state information, by using a plurality of the different discriminant-preparing methods. The candidate multivariate discriminant-verifying part 102h2 verifies the candidate multivariate discriminant prepared in the candidate multivariate discriminant-preparing part 102h1 based on a particular verifying method. The candidate multivariate discriminant-verifying part 102h2 may verify at least one of the discrimination rate, sensitivity, specificity, and information criterion of the candidate multivariate discriminants based on at least one of the bootstrap method, holdout method, and leave-one-out method. The explanatory variable-selecting part 102h3 selects the combination of the amino acid concentration data contained in the female genital cancer state information used in preparing the candidate multivariate discriminant, by selecting the explanatory variables of the candidate multivariate discriminant based on a particular explanatory variable-selecting method from the verification results obtained in the candidate multivariate discriminant-verifying part 102h2. The explanatory variable-selecting part 102h3 may select the explanatory variables of the candidate multivariate discriminant based on at least one of the stepwise method, best path method, local search method, and genetic algorithm from the verification results.

Returning to FIG. 6, the discriminant value-calculating part 102i calculates the discriminant value that is the value of the multivariate discriminant, based on both (i) the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data of the subject received in the receiving part 102f and (ii) the multivariate discriminant containing at least one of Thr, Ser, Asn, Gln, Pro, Gly, Ala, Cit, Val, Met, Ile, Leu, Tyr, Phe, His, Trp, Orn, Lys, and Arg as the explanatory variable prepared in the multivariate discriminant-preparing part 102h.

The multivariate discriminant may be any one of a fractional expression, the sum of a plurality of the fractional expressions, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

When the discriminant value criterion-discriminating part 102j1 discriminates between any one of the cervical cancer, the endometrial cancer, and the ovarian cancer and the female genital cancer-free, the discriminant value-calculating part 102i may calculate the discriminant value based on both (i) the concentration value of at least one of Thr, Ser, Asn, Gln, Pro, Ala, Val, Met, Ile, Leu, Tyr, Phe, His, Trp, Orn, and Arg contained in the amino acid concentration data of the subject received in the receiving part 102f and (ii) the multivariate discriminant containing at least one of Thr, Ser, Asn, Gln, Pro, Ala, Val, Met, Ile, Leu, Tyr, Phe, His, Trp, Orn, and Arg as the explanatory variable prepared in the multivariate discriminant-preparing part 102h. The multivariate discriminant to be used in this case may be (i) the fractional expression with Gln, His, and Arg as the explanatory variables, (ii) the fractional expression with a-ABA, His, and Met as the explanatory variables, (iii) the fractional expression with Ile, His, Cit, Arg, Tyr, and Trp as the explanatory variables, (iv) the fractional expression with a-ABA, Cit, and Met as the explanatory variables, (v) the linear discriminant with Gly, Val, His, and Arg as the explanatory variables, (vi) the linear discriminant with Gly, a-ABA, Met, and His as the explanatory variables, (vii) the linear discriminant with Ala, Ile, His, Trp, and Arg as the explanatory variables, (viii) the linear discriminant with Gly, Cit, Met, and Phe as the explanatory variables, (ix) the linear discriminant with His, Leu, Met, Cit, Ile, and Tyr as the explanatory variables, (x) the logistic regression equation with Val, Leu, His, and Arg as the explanatory variables, (xi) the logistic regression equation with a-ABA, Met, Tyr, and His as the explanatory variables, (xii) the logistic regression equation with Val, Ile, His, Trp, and Arg as the explanatory variables, (xiii) the logistic regression equation with Cit, a-ABA, Met, and Tyr as the explanatory variables, or (xiv) the logistic regression equation with His, Leu, Met, Cit, Ile, and Tyr as the explanatory variables.

When the discriminant value criterion-discriminating part 102j1 discriminates between any one of the cervical cancer and the endometrial cancer and any one of the cervical cancer-free and the endometrial cancer-free, the discriminant value-calculating part 102i may calculate the discriminant value based on both (i) the concentration value of at least one of Thr, Ser, Asn, Pro, Gly, Cit, Val, Met, Ile, Leu, Phe, His, Trp, Orn, Lys, and Arg contained in the amino acid concentration data of the subject received in the receiving part 102f and (ii) the multivariate discriminant containing at least one of Thr, Ser, Asn, Pro, Gly, Cit, Val, Met, Ile, Leu, Phe, His, Trp, Orn, Lys, and Arg as the explanatory variable prepared in the multivariate discriminant-preparing part 102h. The multivariate discriminant to be used in this case may be (i) the fractional expression with Lys, His, and Arg as the explanatory variables, (ii) the fractional expression with a-ABA, His, and Met as the explanatory variables, (iii) the fractional expression with Ile, His, Cit, and Arg as the explanatory variables, (iv) the linear discriminant with Gly, Val, His, and Arg as the explanatory variables, (v) the linear discriminant with Gly, Phe, His, and Arg as the explanatory variables, (vi) the linear discriminant with Cit, Ile, His, and Arg as the explanatory variables, (vii) the linear discriminant with His, Leu, Met, Cit, Ile, and Tyr as the explanatory variables, (viii) the logistic regression equation with Val, His, Lys, and Arg as the explanatory variables, (ix) the logistic regression equation with Thr, a-ABA, Met, and His as the explanatory variables, (x) the logistic regression equation with Cit, Ile, His, and Arg as the explanatory variables, or (xi) the logistic regression equation with His, Leu, Met, Cit, Ile, and Tyr as the explanatory variables.

When the discriminant value criterion-discriminating part 102j1 discriminates between the cervical cancer and the cervical cancer-free, the discriminant value-calculating part 102i may calculate the discriminant value based on both (i) the concentration value of at least one of Asn, Val, Met, Leu, Phe, His, Trp, Orn, Lys, and Arg contained in the amino acid concentration data of the subject received in the receiving part 102f and (ii) the multivariate discriminant containing at least one of Asn, Val, Met, Leu, Phe, His, Trp, Orn, Lys, and Arg as the explanatory variable prepared in the multivariate discriminant-preparing part 102h. The multivariate discriminant to be used in this case may be (i) the fractional expression with a-ABA, His, and Val as the explanatory variables, (ii) the fractional expression with a-ABA, Met, and Val as the explanatory variables, (iii) the fractional expression with Met, His, Cit, and Arg as the explanatory variables, (iv) the linear discriminant with Gly, Val, His, and Arg as the explanatory variables, (v) the linear discriminant with Gly, Val, Met, and Lys as the explanatory variables, (vi) the linear discriminant with Cit, Met, His, and Arg as the explanatory variables, (vii) the linear discriminant with His, Leu, Met, Ile, Tyr, and Lys as the explanatory variables, (viii) the logistic regression equation with Val, Leu, His, and Arg as the explanatory variables, (ix) the logistic regression equation with Met, His, Orn, and Arg as the explanatory variables, (x) the logistic regression equation with Val, Tyr, His, and Arg as the explanatory variables, or (xi) the logistic regression equation with His, Leu, Met, Ile, Tyr, and Lys as the explanatory variables.

When the discriminant value criterion-discriminating part 102j1 discriminates between the endometrial cancer and the endometrial cancer-free, the discriminant value-calculating part 102i may calculate the discriminant value based on both (i) the concentration value of at least one of Thr, Ser, Asn, Pro, Gly, Cit, Val, Met, Ile, Leu, Phe, His, Trp, and Arg contained in the amino acid concentration data of the subject received in the receiving part 102f and (ii) the multivariate discriminant containing at least one of Thr, Ser, Asn, Pro, Gly, Cit, Val, Met, Ile, Leu, Phe, His, Trp, and Arg as the explanatory variable prepared in the multivariate discriminant-preparing part 102h. The multivariate discriminant to be used in this case may be (i) the fractional expression with Lys, His, and Arg as the explanatory variables, (ii) the fractional expression with a-ABA, His, and Met as the explanatory variables, (iii) the fractional expression with Ile, His, Asn, and Cit as the explanatory variables, (iv) the linear discriminant with Gln, His, Lys, and Arg as the explanatory variables, (v) the linear discriminant with Gly, Met, Phe, and His as the explanatory variables, (vi) the linear discriminant with Cit, Ile, His, and Arg as the explanatory variables, (vii) the linear discriminant with His, Asn, Val, Pro, Cit, and Ile as the explanatory variables, (viii) the logistic regression equation with Gln, Gly, His, and Arg as the explanatory variables, (ix) the logistic regression equation with Gln, Phe, His, and Arg as the explanatory variables, (x) the logistic regression equation with Gln, Ile, His, and Arg as the explanatory variables, or (xi) the logistic regression equation with His, Asn, Val, Pro, Cit, and Ile as the explanatory variables.

When the discriminant value criterion-discriminating part 102j1 discriminates between the ovarian cancer and the ovarian cancer-free, the discriminant value-calculating part 102i may calculate the discriminant value based on both (i) the concentration value of at least one of Thr, Ser, Asn, Gln, Ala, Cit, Val, Met, Ile, Leu, Tyr, Phe, His, Trp, Orn, Lys, and Arg contained in the amino acid concentration data of the subject received in the receiving part 102f and (ii) the multivariate discriminant containing at least one of Thr, Ser, Asn, Gln, Ala, Cit, Val, Met, Ile, Leu, Tyr, Phe, His, Trp, Orn, Lys, and Arg as the explanatory variable prepared in the multivariate discriminant-preparing part 102h. The multivariate discriminant to be used in this case may be (i) the fractional expression with Orn, Cit, and Met as the explanatory variables, (ii) the fractional expression with Gln, Cit, and Tyr as the explanatory variables, (iii) the fractional expression with Orn, His, Phe, and Trp as the explanatory variables, (iv) the linear discriminant with Ser, Cit, Orn, and Trp as the explanatory variables, (v) the linear discriminant with Ser, Cit, Ile, and Orn as the explanatory variables, (vi) the linear discriminant with Phe, Trp, Orn, and Lys as the explanatory variables, (vii) the linear discriminant with His, Trp, Glu, Cit, Ile, and Orn as the explanatory variables, (viii) the logistic regression equation with Ser, Cit, Trp, and Orn as the explanatory variables, (ix) the logistic regression equation with Gln, Cit, Ile, and Tyr as the explanatory variables, (x) the logistic regression equation with Asn, Phe, His, and Trp as the explanatory variables, or (xi) the logistic regression equation with His, Trp, Glu, Cit, Ile, and Orn as the explanatory variables.

When the discriminant value criterion-discriminating part 102j1 discriminates between the female genital cancer suffering risk group and the healthy group, the discriminant value-calculating part 102i may calculate the discriminant value based on both (i) the concentration value of at least one of Thr, Ser, Asn, Gln, Pro, Ala, Val, Met, Ile, Leu, Tyr, Phe, His, Trp, Orn, and Arg contained in the amino acid concentration data of the subject received in the receiving part 102f and (ii) the multivariate discriminant containing at least one of Thr, Ser, Asn, Gln, Pro, Ala, Val, Met, Ile, Leu, Tyr, Phe, His, Trp, Orn, and Arg as the explanatory variable prepared in the multivariate discriminant-preparing part 102h. The multivariate discriminant to be used in this case may be the linear discriminant with Phe, His, Met, Pro, Lys, and Arg as the explanatory variables, or the logistic regression equation with Phe, His, Met, Pro, Lys, and Arg as the explanatory variables.

When the discriminant value criterion-discriminating part 102j1 discriminates between the cervical cancer, the endometrial cancer, and the ovarian cancer, the discriminant value-calculating part 102i may calculate the discriminant value based on both (i) the concentration value of at least one of Thr, Ser, Asn, Glu, Gln, Pro, Gly, Ala, Cit, ABA, Val, Met, Ile, Leu, Tyr, Phe, His, Trp, Orn, Lys, and Arg contained in the amino acid concentration data of the subject received in the receiving part 102f and (ii) the multivariate discriminant containing at least one of Thr, Ser, Asn, Glu, Gln, Pro, Gly, Ala, Cit, ABA, Val, Met, Ile, Leu, Tyr, Phe, His, Trp, Orn, Lys, and Arg as the explanatory variable prepared in the multivariate discriminant-preparing part 102h. The multivariate discriminant to be used in this case may be the discriminant with Cit, Met, Lys, Asn, Ala, Thr, Gln, and a-ABA as the explanatory variables which is prepared by the Mahalanobis' generalized distance method, or the discriminant prepared with His, Leu, Ser, Thr, Glu, Gln, Ala, and Lys as the explanatory variables which is prepared by the Mahalanobis' generalized distance method.

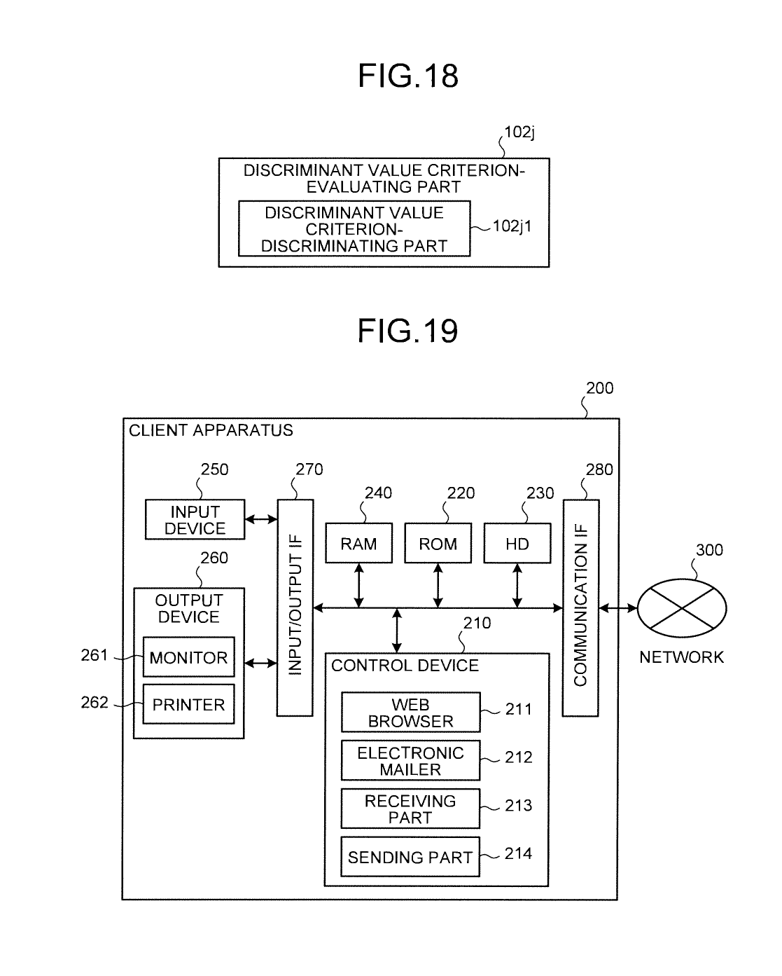

The discriminant value criterion-evaluating part 102j evaluates the state of female genital cancer in the subject based on the discriminant value calculated in the discriminant value-calculating part 102i. The discriminant value criterion-evaluating part 102j further includes the discriminant value criterion-discriminating part 102j1. Now, the configuration of the discriminant value criterion-evaluating part 102j will be described with reference to FIG. 18. FIG. 18 is a block diagram showing the configuration of the discriminant value criterion-evaluating part 102j, and only a part in the configuration related to the present invention is shown conceptually. The discriminant value criterion-discriminating part 102j1 discriminates (i) between the female genital cancer and the female genital cancer-free, (ii) between any one of the cervical cancer, the endometrial cancer, and the ovarian cancer and the female genital cancer-free, (iii) between any one of the cervical cancer and the endometrial cancer and any one of the cervical cancer-free and the endometrial cancer-free, (iv) between the cervical cancer and the cervical cancer-free, (v) between the endometrial cancer and the endometrial cancer-free, (vi) between the ovarian cancer and the ovarian cancer-free, (vii) between the female genital cancer suffering risk group and the healthy group, or (viii) between the cervical cancer, the endometrial cancer, and the ovarian cancer in the subject, based on the discriminant value calculated in the discriminant value-calculating part 102i.

Returning to FIG. 6, the result outputting part 102k outputs, into the output device 114, the processing results in each processing part in the control device 102 (the evaluation results obtained in the discriminant value criterion-evaluating part 102j (specifically, the discrimination results obtained in the discriminant value criterion-discriminating part 102j1)) etc.

The sending part 102m transmits the evaluation results to the client apparatus 200 that is a sender of the amino acid concentration data of the subject, and transmits the multivariate discriminant prepared in the female genital cancer-evaluating apparatus 100 and the evaluation results to the database apparatus 400.

Hereinafter, a configuration of the client apparatus 200 in the present system will be described with reference to FIG. 19. FIG. 19 is a block diagram showing an example of the configuration of the client apparatus 200 in the present system, and only the part in the configuration relevant to the present invention is shown conceptually.

The client apparatus 200 includes a control device 210, ROM 220, HD (Hard Disk) 230, RAM 240, an input device 250, an output device 260, an input/output IF 270, and a communication IF 280 that are connected communicatively to one another through a communication channel.

The control device 210 has a Web browser 211, an electronic mailer 212, a receiving part 213, and a sending part 214. The Web browser 211 performs browsing processings of interpreting Web data and displaying the interpreted Web data on a monitor 261 described below. The Web browser 211 may have various plug-in softwares, such as stream player, having functions to receive, display and feedback streaming screen images. The electronic mailer 212 sends and receives electronic mails using a particular protocol (e.g., SMTP (Simple Mail Transfer Protocol) or POPS (Post Office Protocol version 3)). The receiving part 213 receives various kinds of information, such as the evaluation results transmitted from the female genital cancer-evaluating apparatus 100, via the communication IF 280. The sending part 214 sends various kinds of information such as the amino acid concentration data of the subject, via the communication IF 280, to the female genital cancer-evaluating apparatus 100.

The input device 250 is for example a keyboard, a mouse or a microphone. The monitor 261 described below also functions as a pointing device together with a mouse. The output device 260 is an output means for outputting information received via the communication IF 280, and includes the monitor 261 (including home television) and a printer 262. In addition, the output device 260 may have a speaker or the like additionally. The input/output IF 270 is connected to the input device 250 and the output device 260.

The communication IF 280 connects the client apparatus 200 to the network 300 (or communication apparatus such as a router) communicatively. In other words, the client apparatuses 200 are connected to the network 300 via a communication apparatus such as a modem, TA (Terminal Adapter) or a router, and a telephone line, or a private line. In this way, the client apparatuses 200 can access to the female genital cancer-evaluating apparatus 100 by using a particular protocol.

The client apparatus 200 may be realized by installing softwares (including programs, data and others) for a Web data-browsing function and an electronic mail-processing function to an information processing apparatus (for example, an information processing terminal such as a known personal computer, a workstation, a family computer, Internet TV (Television), PHS (Personal Handyphone System) terminal, a mobile phone terminal, a mobile unit communication terminal or PDA (Personal Digital Assistants)) connected as needed with peripheral devices such as a printer, a monitor, and an image scanner.

All or a part of processings of the control device 210 in the client apparatus 200 may be performed by CPU and programs read and executed by the CPU. Computer programs for giving instructions to the CPU and executing various processings together with the OS (Operating System) are recorded in the ROM 220 or HD 230. The computer programs, which are executed as they are loaded in the RAM 240, constitute the control device 210 with the CPU. The computer programs may be stored in application program servers connected via any network to the client apparatus 200, and the client apparatus 200 may download all or a part of them as needed. All or any part of processings of the control device 210 may be realized by hardware such as wired-logic.

Hereinafter, the network 300 in the present system will be described with reference to FIGS. 4 and 5. The network 300 has a function to connect the female genital cancer-evaluating apparatus 100, the client apparatuses 200, and the database apparatus 400 mutually, communicatively to one another, and is for example the Internet, an intranet, or LAN (Local Area Network (both wired/wireless)). The network 300 may be VAN (Value Added Network), a personal computer communication network, a public telephone network (including both analog and digital), a leased line network (including both analog and digital), CATV (Community Antenna Television) network, a portable switched network or a portable packet-switched network (including IMT2000 (International Mobile Telecommunication 2000) system, GSM (Global System for Mobile Communications) system, or PDC (Personal Digital Cellular)/PDC-P system), a wireless calling network, a local wireless network such as Bluetooth (registered trademark), PHS network, a satellite communication network (including CS (Communication Satellite), BS (Broadcasting Satellite), ISDB (Integrated Services Digital Broadcasting), and the like), or the like.

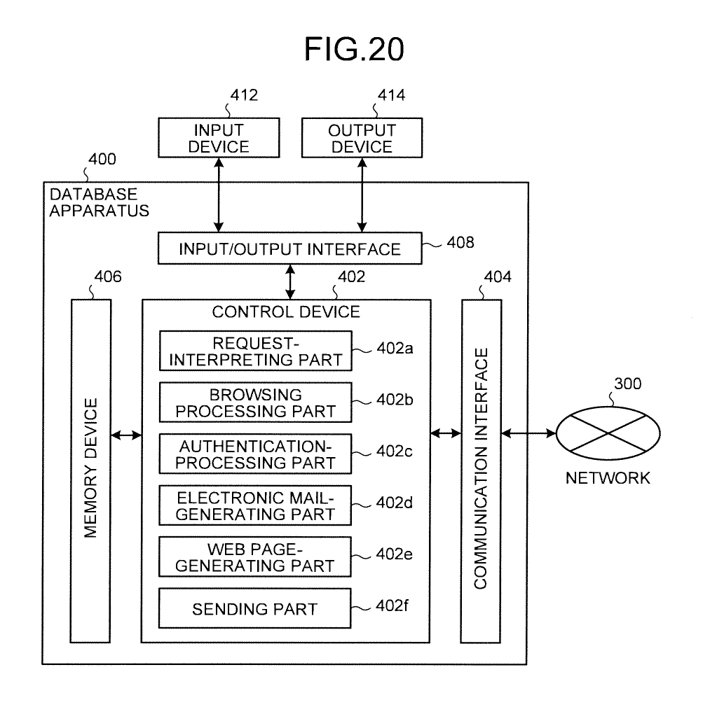

Hereinafter, the configuration of the database apparatus 400 in the present system will be described with reference to FIG. 20. FIG. 20 is a block diagram showing an example of the configuration of the database apparatus 400 in the present system, showing conceptually only the region relevant to the present invention.

The database apparatus 400 has functions to store, for example, the female genital cancer state information used in preparing the multivariate discriminants in the female genital cancer-evaluating apparatus 100 or in the database apparatus 400, the multivariate discriminants prepared in the female genital cancer-evaluating apparatus 100, and the evaluation results obtained in the female genital cancer-evaluating apparatus 100. As shown in FIG. 20, the database apparatus 400 includes (a) a control device 402, such as CPU, which integrally controls the entire database apparatus 400, (b) a communication interface 404 connecting the database apparatus to the network 300 communicatively via a communication apparatus such as a router and via wired or wireless communication circuits such as a private line, (c) a memory device 406 storing various databases, tables and files (for example, files for Web pages), and (d) an input/output interface 408 connected to an input device 412 and an output device 414, and these parts are connected communicatively to each other via any communication channel.

The memory device 406 is a storage means, and may be, for example, memory apparatus such as RAM or ROM, a fixed disk drive such as a hard disk, a flexible disk, an optical disk, and the like. The memory device 406 stores, for example, various programs used in various processings. The communication interface 404 allows communication between the database apparatus 400 and the network 300 (or a communication apparatus such as a router). Thus, the communication interface 404 has a function to communicate data via a communication line with other terminals. The input/output interface 408 is connected to the input device 412 and the output device 414. A monitor (including a home television), a speaker, or a printer may be used as the output device 414 (hereinafter, the output device 414 may be described as a monitor 414). A keyboard, a mouse, a microphone, or a monitor functioning as a pointing device together with a mouse may be used as the input device 412.

The control device 402 has an internal memory storing control programs such as OS (Operating System), programs for various processing procedures, and other needed data, and performs various information processings according to these programs. As shown in the figure, the control device 402 includes mainly a request-interpreting part 402a, a browsing processing part 402b, an authentication-processing part 402c, an electronic mail-generating part 402d, a Web page-generating part 402e, and a sending part 402f.

The request-interpreting part 402a interprets the requests transmitted from the female genital cancer-evaluating apparatus 100 and sends the requests to other parts in the control device 402 according to results of interpreting the requests. Upon receiving browsing requests for various screens transmitted from the female genital cancer-evaluating apparatus 100, the browsing processing part 402b generates and transmits web data for these screens. Upon receiving authentication requests transmitted from the female genital cancer-evaluating apparatus 100, the authentication-processing part 402c performs authentication. The electronic mail-generating part 402d generates electronic mails including various kinds of information. The Web page-generating part 402e generates Web pages for users to browse with the client apparatus 200. The sending part 402f transmits various kinds of information such as the female genital cancer state information and the multivariate discriminants to the female genital cancer-evaluating apparatus 100.

### 2-3. Processing in the Present System

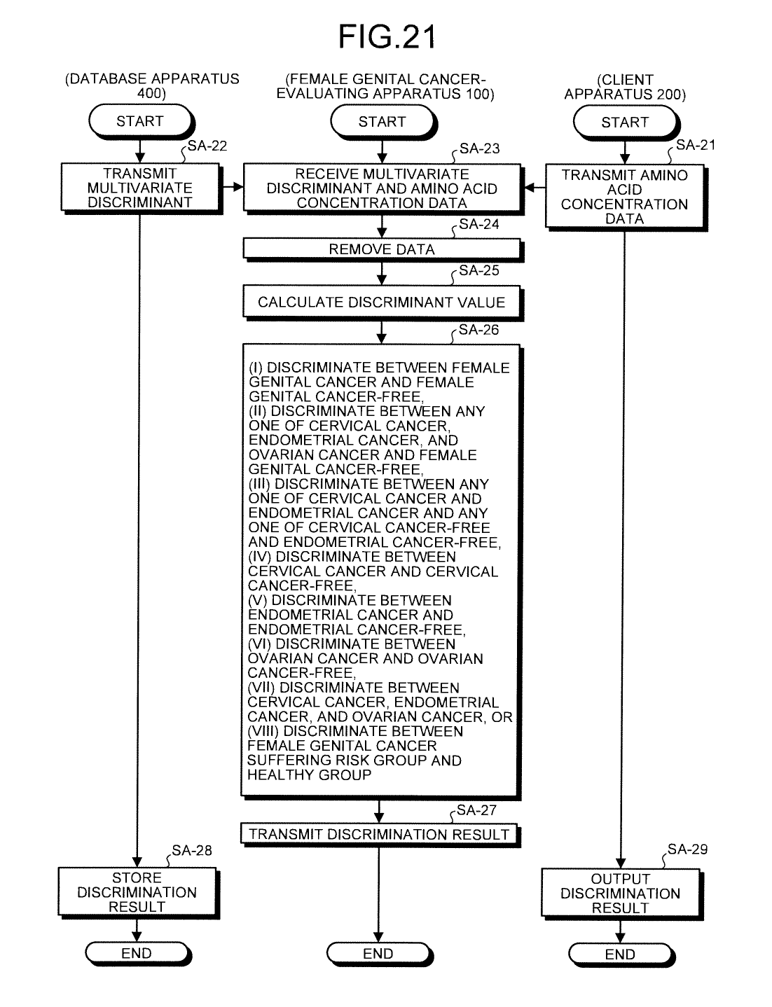

Here, an example of a female genital cancer evaluation service processing performed in the present system constituted as described above will be described with reference to FIG. 21. FIG. 21 is a flowchart showing the example of the female genital cancer evaluation service processing.

The amino acid concentration data used in the present processing is data concerning the concentration values of amino acids obtained by analyzing blood previously collected from an individual. Hereinafter, the method of analyzing blood amino acid will be described briefly. First, a blood sample is collected in a heparin-treated tube, and then the blood plasma is separated by centrifugation of the tube. All blood plasma samples separated are frozen and stored at −70° C. before a measurement of an amino acid concentration. Before the measurement of the amino acid concentration, the blood plasma samples are deproteinized by adding sulfosalicylic acid to a concentration of 3%. An amino acid analyzer by high-performance liquid chromatography (HPLC) by using ninhydrin reaction in the post column is used for the measurement of the amino acid concentration.

First, the client apparatus 200 accesses the female genital cancer-evaluating apparatus 100 when the user specifies the Web site address (such as URL) provided from the female genital cancer-evaluating apparatus 100, via the input device 250 on the screen displaying the Web browser 211. Specifically, when the user instructs update of the Web browser 211 screen on the client apparatus 200, the Web browser 211 sends the Web site address provided from the female genital cancer-evaluating apparatus 100 by a particular protocol to the female genital cancer-evaluating apparatus 100, thereby transmitting requests demanding a transmission of Web page corresponding to an amino acid concentration data transmission screen to the female genital cancer-evaluating apparatus 100 based on a routing of the address.

Then, upon receipt of the request transmitted from the client apparatus 200, the request-interpreting part 102a in the female genital cancer-evaluating apparatus 100 analyzes the transmitted requests and sends the requests to other parts in the control device 102 according to analytical results. Specifically, when the transmitted requests are requests to send the Web page corresponding to the amino acid concentration data transmission screen, mainly the browsing processing part 102b in the female genital cancer-evaluating apparatus 100 obtains the Web data for display of the Web page stored in a predetermined region of the memory device 106 and sends the obtained Web data to the client apparatus 200. More specifically, upon receiving the requests to transmit the Web page corresponding to the amino acid concentration data transmission screen by the user, the control device 102 in the female genital cancer-evaluating apparatus 100 demands inputs of user ID and user password from the user. If the user ID and password are input, the authentication-processing part 102c in the female genital cancer-evaluating apparatus 100 examines the input user ID and password by comparing them with the user ID and user password stored in the user information file 106a for authentication. Only when the user is authenticated, the browsing processing part 102b in the female genital cancer-evaluating apparatus 100 sends the Web data for displaying the Web page corresponding to the amino acid concentration data transmission screen to the client apparatus 200. The client apparatus 200 is identified with the IP (Internet Protocol) address transmitted from the client apparatus 200 together with the transmission requests.

Then, the client apparatus 200 receives, in the receiving part 213, the Web data (for displaying the Web page corresponding to the amino acid concentration data transmission screen) transmitted from the female genital cancer-evaluating apparatus 100, interprets the received Web data with the Web browser 211, and displays the amino acid concentration data transmission screen on the monitor 261.

When the user inputs and selects, via the input device 250, for example the amino acid concentration data of the individual on the amino acid concentration data transmission screen displayed on the monitor 261, the sending part 214 of the client apparatus 200 transmits an identifier for identifying input information and selected items to the female genital cancer-evaluating apparatus 100, thereby transmitting the amino acid concentration data of the individual as the subject to the female genital cancer-evaluating apparatus 100 (step SA-21). In step SA-21, the transmission of the amino acid concentration data may be realized for example by using an existing file transfer technology such as FTP (File Transfer Protocol).

Then, the request-interpreting part 102a of the female genital cancer-evaluating apparatus 100 interprets the identifier transmitted from the client apparatus 200 thereby interpreting the requests from the client apparatus 200, and requests the database apparatus 400 to send the multivariate discriminant for the evaluation of the state of female genital cancer.

Then, the request-interpreting part 402a in the database apparatus 400 interprets the transmission requests from the female genital cancer-evaluating apparatus 100 and transmits, to the female genital cancer-evaluating apparatus 100, the multivariate discriminant containing at least one of Thr, Ser, Asn, Gln, Pro, Gly, Ala, Cit, Val, Met, Ile, Leu, Tyr, Phe, His, Trp, Orn, Lys, and Arg as the explanatory variables stored in a predetermined region of the memory device 406 (for example, the multivariate discriminant is the updated newest multivariate discriminant. the multivariate discriminant is any one of a fractional expression, the sum of a plurality of the fractional expressions, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.) (step SA-22).

When the discrimination between any one of the cervical cancer, the endometrial cancer, and the ovarian cancer and the female genital cancer-free is conducted in step SA-26 described below, in step SA-22, the multivariate discriminant transmitted to the female genital cancer-evaluating evaluating apparatus 100 may be the multivariate discriminant containing at least one of Thr, Ser, Asn, Gln, Pro, Ala, Val, Met, Ile, Leu, Tyr, Phe, His, Trp, Orn, and Arg as the explanatory variable. Specifically, the multivariate discriminant may be (i) the fractional expression with Gln, His, and Arg as the explanatory variables, (ii) the fractional expression with a-ABA, His, and Met as the explanatory variables, (iii) the fractional expression with Ile, His, Cit, Arg, Tyr, and Trp as the explanatory variables, (iv) the fractional expression with a-ABA, Cit, and Met as the explanatory variables, (v) the linear discriminant with Gly, Val, His, and Arg as the explanatory variables, (vi) the linear discriminant with Gly, a-ABA, Met, and His as the explanatory variables, (vii) the linear discriminant with Ala, Ile, His, Trp, and Arg as the explanatory variables, (viii) the linear discriminant with Gly, Cit, Met, and Phe as the explanatory variables, (ix) the linear discriminant with His, Leu, Met, Cit, Ile, and Tyr as the explanatory variables, (x) the logistic regression equation with Val, Leu, His, and Arg as the explanatory variables, (xi) the logistic regression equation with a-ABA, Met, Tyr, and His as the explanatory variables, (xii) the logistic regression equation with Val, Ile, His, Trp, and Arg as the explanatory variables, (xiii) the logistic regression equation with Cit, a-ABA, Met, and Tyr as the explanatory variables, or (xiv) the logistic regression equation with His, Leu, Met, Cit, Ile, and Tyr as the explanatory variables.

When the discrimination between any one of the cervical cancer and the endometrial cancer and any one of the cervical cancer-free and the endometrial cancer-free is conducted in step SA-26 described below, in step SA-22, the multivariate discriminant transmitted to the female genital cancer-evaluating apparatus 100 may be the multivariate discriminant containing at least one of Thr, Ser, Asn, Pro, Gly, Cit, Val, Met, Ile, Leu, Phe, His, Trp, Orn, Lys, and Arg as the explanatory variable. Specifically, the multivariate discriminant may be (i) the fractional expression with Lys, His, and Arg as the explanatory variables, (ii) the fractional expression with a-ABA, His, and Met as the explanatory variables, (iii) the fractional expression with Ile, His, Cit, and Arg as the explanatory variables, (iv) the linear discriminant with Gly, Val, His, and Arg as the explanatory variables, (v) the linear discriminant with Gly, Phe, His, and Arg as the explanatory variables, (vi) the linear discriminant with Cit, Ile, His, and Arg as the explanatory variables, (vii) the linear discriminant with His, Leu, Met, Cit, Ile, and Tyr as the explanatory variables, (viii) the logistic regression equation with Val, His, Lys, and Arg as the explanatory variables, (ix) the logistic regression equation with Thr, a-ABA, Met, and His as the explanatory variables, (x) the logistic regression equation with Cit, Ile, His, and Arg as the explanatory variables, or (xi) the logistic regression equation with His, Leu, Met, Cit, Ile, and Tyr as the explanatory variables.

When the discrimination between the cervical cancer and the cervical cancer-free is conducted in step SA-26 described below, in step SA-22, the multivariate discriminant transmitted to the female genital cancer-evaluating apparatus 100 may be the multivariate discriminant containing at least one of Asn, Val, Met, Leu, Phe, His, Trp, Orn, Lys, and Arg as the explanatory variable. Specifically, the multivariate discriminant may be (i) the fractional expression with a-ABA, His, and Val as the explanatory variables, (ii) the fractional expression with a-ABA, Met, and Val as the explanatory variables, (iii) the fractional expression with Met, His, Cit, and Arg as the explanatory variables, (iv) the linear discriminant with Gly, Val, His, and Arg as the explanatory variables, (v) the linear discriminant with Gly, Val, Met, and Lys as the explanatory variables, (vi) the linear discriminant with Cit, Met, His, and Arg as the explanatory variables, (vii) the linear discriminant with His, Leu, Met, Ile, Tyr, and Lys as the explanatory variables, (viii) the logistic regression equation with Val, Leu, His, and Arg as the explanatory variables, (ix) the logistic regression equation with Met, His, Orn, and Arg as the explanatory variables, (x) the logistic regression equation with Val, Tyr, His, and Arg as the explanatory variables, or (xi) the logistic regression equation with His, Leu, Met, Ile, Tyr, and Lys as the explanatory variables.

When the discrimination between the endometrial cancer and the endometrial cancer-free is conducted in step SA-26 described below, in step SA-22, the multivariate discriminant transmitted to the female genital cancer-evaluating apparatus 100 may be the multivariate discriminant containing at least one of Thr, Ser, Asn, Pro, Gly, Cit, Val, Met, Ile, Leu, Phe, His, Trp, and Arg as the explanatory variable. Specifically, the multivariate discriminant may be (i) the fractional expression with Lys, His, and Arg as the explanatory variables, (ii) the fractional expression with a-ABA, His, and Met as the explanatory variables, (iii) the fractional expression with Ile, His, Asn, and Cit as the explanatory variables, (iv) the linear discriminant with Gln, His, Lys, and Arg as the explanatory variables, (v) the linear discriminant with Gly, Met, Phe, and His as the explanatory variables, (vi) the linear discriminant with Cit, Ile, His, and Arg as the explanatory variables, (vii) the linear discriminant with His, Asn, Val, Pro, Cit, and Ile as the explanatory variables, (viii) the logistic regression equation with Gln, Gly, His, and Arg as the explanatory variables, (ix) the logistic regression equation with Gln, Phe, His, and Arg as the explanatory variables, (x) the logistic regression equation with Gln, Ile, His, and Arg as the explanatory variables, or (xi) the logistic regression equation with His, Asn, Val, Pro, Cit, and Ile as the explanatory variables.

When the discrimination between the ovarian cancer and the ovarian cancer-free is conducted in step SA-26 described below, in step SA-22, the multivariate discriminant transmitted to the female genital cancer-evaluating apparatus 100 may be the multivariate discriminant containing at least one of Thr, Ser, Asn, Gln, Ala, Cit, Val, Met, Ile, Leu, Tyr, Phe, His, Trp, Orn, Lys, and Arg as the explanatory variable. Specifically, the multivariate discriminant may be (i) the fractional expression with Orn, Cit, and Met as the explanatory variables, (ii) the fractional expression with Gln, Cit, and Tyr as the explanatory variables, (iii) the fractional expression with Orn, His, Phe, and Trp as the explanatory variables, (iv) the linear discriminant with Ser, Cit, Orn, and Trp as the explanatory variables, (v) the linear discriminant with Ser, Cit, Ile, and Orn as the explanatory variables, (vi) the linear discriminant with Phe, Trp, Orn, and Lys as the explanatory variables, (vii) the linear discriminant with His, Trp, Glu, Cit, Ile, and Orn as the explanatory variables, (viii) the logistic regression equation with Ser, Cit, Trp, and Orn as the explanatory variables, (ix) the logistic regression equation with Gln, Cit, Ile, and Tyr as the explanatory variables, (x) the logistic regression equation with Asn, Phe, His, and Trp as the explanatory variables, or (xi) the logistic regression equation with His, Trp, Glu, Cit, Ile, and Orn as the explanatory variables.

When the discrimination between the female genital cancer suffering risk group and the healthy group is conducted in step SA-26 described below, in step SA-22, the multivariate discriminant transmitted to the female genital cancer-evaluating apparatus 100 may be the multivariate discriminant containing at least one of Thr, Ser, Asn, Gln, Pro, Ala, Val, Met, Ile, Leu, Tyr, Phe, His, Trp, Orn, and Arg as the explanatory variable. Specifically, the multivariate discriminant may be the linear discriminant with Phe, His, Met, Pro, Lys, and Arg as the explanatory variables, or the logistic regression equation with Phe, His, Met, Pro, Lys, and Arg as the explanatory variables.

When the discrimination between the cervical cancer, the endometrial cancer, and the ovarian cancer is conducted in step SA-26 described below, in step SA-22, the multivariate discriminant transmitted to the female genital cancer-evaluating apparatus 100 may be the multivariate discriminant containing at least one of Thr, Ser, Asn, Glu, Gln, Pro, Gly, Ala, Cit, ABA, Val, Met, Ile, Leu, Tyr, Phe, His, Trp, Orn, Lys, and Arg as the explanatory variable. Specifically, the multivariate discriminant may be the discriminant with Cit, Met, Lys, Asn, Ala, Thr, Gln, and a-ABA as the explanatory variables which is prepared by the Mahalanobis' generalized distance method, or the discriminant prepared with His, Leu, Ser, Thr, Glu, Gln, Ala, and Lys as the explanatory variables which is prepared by the Mahalanobis' generalized distance method.

Returning to FIG. 21, The female genital cancer-evaluating apparatus 100 receives, in the receiving part 102f, the amino acid concentration data of the individual transmitted from the client apparatuses 200 and the multivariate discriminant transmitted from the database apparatus 400, and stores the received amino acid concentration data in a predetermined memory region of the amino acid concentration data file 106b and the received multivariate discriminant in a predetermined memory region of the multivariate discriminant file 106e4 (step SA-23).

Then, the control device 102 in the female genital cancer-evaluating apparatus 100 removes data such as defective and outliers from the amino acid concentration data of the individual received in step SA-23 (step SA-24).

Then, the female genital cancer-evaluating apparatus 100 calculates, in the discriminant value-calculating part 102i, the discriminant value that is the value of the multivariate discriminant, based on both (i) the amino acid concentration data of the individual from which the data such as the defective and outliers have been removed in step SA-24 and (ii) the multivariate discriminant received in step SA-23 (step SA-25), compares, in the discriminant value criterion-discriminating part 102j1, the discriminant value calculated in step SA-25 with a previously established threshold (cutoff value), thereby conducting any one of the discriminations described in the following 21. to 28. in the individual, and stores the discrimination results in a predetermined memory region of the evaluation result file 106g (step SA-26).

21. Discrimination Between the Female Genital Cancer and the Female Genital Cancer-Free

(I) In step SA-25, the discriminant value is calculated based on both (i) the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data of the individual and (ii) the multivariate discriminant containing at least one of Thr, Ser, Asn, Gln, Pro, Gly, Ala, Cit, Val, Met, Ile, Leu, Tyr, Phe, His, Trp, Orn, Lys, and Arg as the explanatory variable, and (II) in step SA-26, the calculated discriminant value is compared with a previously established threshold (cutoff value), thereby discriminating between the female genital cancer and the female genital cancer-free in the individual.

22. Discrimination Between any One of the Cervical Cancer, the Endometrial Cancer, and the Ovarian Cancer and the Female Genital Cancer-Free

(I) In step SA-25, the discriminant value is calculated based on both (i) the concentration value of at least one of Thr, Ser, Asn, Gln, Pro, Ala, Val, Met, Ile, Leu, Tyr, Phe, His, Trp, Orn, and Arg contained in the amino acid concentration data of the individual and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) in step SA-26, the calculated discriminant value is compared with a previously established threshold (cutoff value), thereby discriminating between any one of the cervical cancer, the endometrial cancer, and the ovarian cancer and the female genital cancer-free in the individual.

23. Discrimination Between any One of the Cervical Cancer and the Endometrial Cancer and any One of the Cervical Cancer-Free and the Endometrial Cancer-Free

(I) In step SA-25, the discriminant value is calculated based on both (i) the concentration value of at least one of Thr, Ser, Asn, Pro, Gly, Cit, Val, Met, Ile, Leu, Phe, His, Trp, Orn, Lys, and Arg contained in the amino acid concentration data of the individual and (ii) the multivariate discriminant containing at least one of Thr, Ser, Asn, Pro, Gly, Cit, Val, Met, Ile, Leu, Phe, His, Trp, Orn, Lys, and Arg as the explanatory variable, and (II) in step SA-26, the calculated discriminant value is compared with a previously established threshold (cutoff value), thereby discriminating between any one of the cervical cancer and the endometrial cancer and any one of the cervical cancer-free and the endometrial cancer-free in the individual.

24. Discrimination Between the Cervical Cancer and the Cervical Cancer-Free

(I) In step SA-25, the discriminant value is calculated based on both (i) the concentration value of at least one of Asn, Val, Met, Leu, Phe, His, Trp, Orn, Lys, and Arg contained in the amino acid concentration data of the individual and (ii) the multivariate discriminant containing at least one of Asn, Val, Met, Leu, Phe, His, Trp, Orn, Lys, and Arg as the explanatory variable, and (II) in step SA-26, the calculated discriminant value is compared with a previously established threshold (cutoff value), thereby discriminating between the cervical cancer and the cervical cancer-free in the individual.

25. Discrimination Between the Endometrial Cancer and the Endometrial Cancer-Free

(I) In step SA-25, the discriminant value is calculated based on both (i) the concentration value of at least one of Thr, Ser, Asn, Pro, Gly, Cit, Val, Met, Ile, Leu, Phe, His, Trp, and Arg contained in the amino acid concentration data of the individual and (ii) the multivariate discriminant containing at least one of Thr, Ser, Asn, Pro, Gly, Cit, Val, Met, Ile, Leu, Phe, His, Trp, and Arg as the explanatory variable, and (II) in step SA-26, the calculated discriminant value is compared with a previously established threshold (cutoff value), thereby discriminating between the endometrial cancer and the endometrial cancer-free in the individual.

26. Discrimination Between the Ovarian Cancer and the Ovarian Cancer-Free

(I) In step SA-25, the discriminant value is calculated based on both (i) the concentration value of at least one of Thr, Ser, Asn, Gln, Ala, Cit, Val, Met, Ile, Leu, Tyr, Phe, His, Trp, Orn, Lys, and Arg contained in the amino acid concentration data of the individual and (ii) the multivariate discriminant containing at least one of Thr, Ser, Asn, Gln, Ala, Cit, Val, Met, Ile, Leu, Tyr, Phe, His, Trp, Orn, Lys, and Arg as the explanatory variable, and (II) in step SA-26, the calculated discriminant value is compared with a previously established threshold (cutoff value), thereby discriminating between the ovarian cancer and the ovarian cancer-free in the individual.

27. Discrimination Between the Cervical Cancer, the Endometrial Cancer, and the Ovarian Cancer

(I) In step SA-25, the discriminant value is calculated based on both (i) the concentration value of at least one of Thr, Ser, Asn, Glu, Gln, Pro, Gly, Ala, Cit, ABA, Val, Met, Ile, Leu, Tyr, Phe, His, Trp, Orn, Lys, and Arg contained in the amino acid concentration data of the individual and (ii) the multivariate discriminant containing at least one of Thr, Ser, Asn, Glu, Gln, Pro, Gly, Ala, Cit, ABA, Val, Met, Ile, Leu, Tyr, Phe, His, Trp, Orn, Lys, and Arg as the explanatory variable, and (II) in step SA-26, the calculated discriminant value is compared with a previously established threshold (cutoff value), thereby discriminating between the cervical cancer, the endometrial cancer, and the ovarian cancer in the individual.

28. Discrimination Between the Female Genital Cancer Suffering Risk Group and the Healthy Group

(I) In step SA-25, the discriminant value is calculated based on both (i) the concentration value of at least one of Thr, Ser, Asn, Gln, Pro, Ala, Val, Met, Ile, Leu, Tyr, Phe, His, Trp, Orn, and Arg contained in the amino acid concentration data of the individual and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) in step SA-26, the discrimination between the female genital cancer suffering risk group and the healthy group in the individual is conducted based on the calculated discriminant value.

Returning to FIG. 21, the sending part 102m in the female genital cancer-evaluating apparatus 100 sends, to the client apparatus 200 that has sent the amino acid concentration data and to the database apparatus 400, the discrimination results obtained in step SA-26 (step SA-27). Specifically, the female genital cancer-evaluating apparatus 100 first generates a Web page for displaying the discrimination results in the Web page-generating part 102e and stores the Web data corresponding to the generated Web page in a predetermined memory region of the memory device 106. Then, the user is authenticated as described above by inputting a predetermined URL (Uniform Resource Locator) into the Web browser 211 of the client apparatus 200 via the input device 250, and the client apparatus 200 sends a Web page browsing request to the female genital cancer-evaluating apparatus 100. The female genital cancer-evaluating apparatus 100 then interprets the browsing request transmitted from the client apparatus 200 in the browsing processing part 102b and reads the Web data corresponding to the Web page for displaying the discrimination results, out of the predetermined memory region of the memory device 106. The sending part 102m in the female genital cancer-evaluating apparatus 100 then sends the read-out Web data to the client apparatus 200 and simultaneously sends the Web data or the discrimination results to the database apparatus 400.

In step SA-27, the control device 102 in the female genital cancer-evaluating apparatus 100 may notify the discrimination results to the user client apparatus 200 by electronic mail. Specifically, the electronic mail-generating part 102d in the female genital cancer-evaluating apparatus 100 first acquires the user electronic mail address by referencing the user information stored in the user information file 106a based on the user ID and the like at the transmission timing. The electronic mail-generating part 102d in the female genital cancer-evaluating apparatus 100 then generates electronic mail data with the acquired electronic mail address as its mail address, including the user name and the discrimination results. The sending part 102m in the female genital cancer-evaluating apparatus 100 then sends the generated electronic mail data to the user client apparatus 200.

Also in step SA-27, the female genital cancer-evaluating apparatus 100 may send the discrimination results to the user client apparatus 200 by using, for example, an existing file transfer technology such as FTP.

Returning to FIG. 21, the control device 402 in the database apparatus 400 receives the discrimination results or the Web data transmitted from the female genital cancer-evaluating apparatus 100 and stores (accumulates) the received discrimination results or the received Web data in a predetermined memory region of the memory device 406 (step SA-28).

The receiving part 213 of the client apparatus 200 receives the Web data transmitted from the female genital cancer-evaluating apparatus 100, and the received Web data is interpreted with the Web browser 211, to display on the monitor 261 the Web page screen displaying the discrimination result of the individual (step SA-29). When the discrimination results are sent from the female genital cancer-evaluating apparatus 100 by electronic mail, the electronic mail transmitted from the female genital cancer-evaluating apparatus 100 is received at any timing, and the received electronic mail is displayed on the monitor 261 with the known function of the electronic mailer 212 in the client apparatus 200.

In this way, the user can confirm the discrimination results on female genital cancer of the individual, by browsing the Web page displayed on the monitor 261. The user may print out the content of the Web page displayed on the monitor 261 by the printer 262.

When the discrimination results are transmitted by electronic mail from the female genital cancer-evaluating apparatus 100, the user reads the electronic mail displayed on the monitor 261, whereby the user can confirm the discrimination results on female genital cancer of the individual. The user may print out the content of the electronic mail displayed on the monitor 261 by the printer 262.

Given the foregoing description, the explanation of the female genital cancer evaluation service processing is finished.

### 2-4. Summary of the Second Embodiment and Other Embodiments

According to the female genital cancer-evaluating system described above in detail, the client apparatus 200 sends the amino acid concentration data of the individual to the female genital cancer-evaluating apparatus 100. Upon receiving the requests from the female genital cancer-evaluating apparatus 100, the database apparatus 400 transmits the multivariate discriminant for the discrimination of female genital cancer to the female genital cancer-evaluating apparatus 100. By the female genital cancer-evaluating apparatus 100, (1) the amino acid concentration data is received from the client apparatus 200, and the multivariate discriminant is received from the database apparatus 400 simultaneously, (2) the discriminant value is calculated based on both the received amino acid concentration data and the received multivariate discriminant, (3) the calculated discriminant value is compared with the previously established threshold, thereby conducting any one of the discriminations described in 21. to 28. above in the individual, and (4) the discrimination results are transmitted to the client apparatus 200 and database apparatus 400. Then, the client apparatus 200 receives and displays the discrimination results transmitted from the female genital cancer-evaluating apparatus 100, and the database apparatus 400 receives and stores the discrimination results transmitted from the female genital cancer-evaluating apparatus 100. Thus, the discriminant values obtained in the multivariate discriminants useful particularly for (i) the 2-group discrimination between the female genital cancer and the female genital cancer-free, (ii) the discrimination between any one of the cervical cancer, the endometrial cancer, and the ovarian cancer and the female genital cancer-free, (iii) the discrimination between any one of the cervical cancer and the endometrial cancer and any one of the cervical cancer-free and the endometrial cancer-free, (iv) the 2-group discrimination between the cervical cancer and the cervical cancer-free, (v) the 2-group discrimination between the endometrial cancer and the endometrial cancer-free, (vi) the 2-group discrimination between the ovarian cancer and the ovarian cancer-free, (vii) the 2-group discrimination between the female genital cancer suffering risk group and the healthy group, or (viii) the discrimination between the cervical cancer, the endometrial cancer, and the ovarian cancer, can be utilized to bring about the effect of enabling more accurately these 2-group discriminations or these discriminations.

When the discrimination described in 22. above is conducted in step SA-26, the multivariate discriminant may be (i) the fractional expression with Gln, His, and Arg as the explanatory variables, (ii) the fractional expression with a-ABA, His, and Met as the explanatory variables, (iii) the fractional expression with Ile, His, Cit, Arg, Tyr, and Trp as the explanatory variables, (iv) the fractional expression with a-ABA, Cit, and Met as the explanatory variables, (v) the linear discriminant with Gly, Val, His, and Arg as the explanatory variables, (vi) the linear discriminant with Gly, a-ABA, Met, and His as the explanatory variables, (vii) the linear discriminant with Ala, Ile, His, Trp, and Arg as the explanatory variables, (viii) the linear discriminant with Gly, Cit, Met, and Phe as the explanatory variables, (ix) the linear discriminant with His, Leu, Met, Cit, Ile, and Tyr as the explanatory variables, (x) the logistic regression equation with Val, Leu, His, and Arg as the explanatory variables, (xi) the logistic regression equation with a-ABA, Met, Tyr, and His as the explanatory variables, (xii) the logistic regression equation with Val, Ile, His, Trp, and Arg as the explanatory variables, (xiii) the logistic regression equation with Cit, a-ABA, Met, and Tyr as the explanatory variables, or (xiv) the logistic regression equation with His, Leu, Met, Cit, Ile, and Tyr as the explanatory variables. Thus, the discriminant values obtained in the multivariate discriminants useful particularly for the discrimination between any one of the cervical cancer, the endometrial cancer, and the ovarian cancer and the female genital cancer-free, can be utilized to bring about the effect of enabling more accurately the discrimination.

When the discrimination described in 23. above is conducted in step SA-26, the multivariate discriminant may be (i) the fractional expression with Lys, His, and Arg as the explanatory variables, (ii) the fractional expression with a-ABA, His, and Met as the explanatory variables, (iii) the fractional expression with Ile, His, Cit, and Arg as the explanatory variables, (iv) the linear discriminant with Gly, Val, His, and Arg as the explanatory variables, (v) the linear discriminant with Gly, Phe, His, and Arg as the explanatory variables, (vi) the linear discriminant with Cit, Ile, His, and Arg as the explanatory variables, (vii) the linear discriminant with His, Leu, Met, Cit, Ile, and Tyr as the explanatory variables, (viii) the logistic regression equation with Val, His, Lys, and Arg as the explanatory variables, (ix) the logistic regression equation with Thr, a-ABA, Met, and His as the explanatory variables, (x) the logistic regression equation with Cit, Ile, His, and Arg as the explanatory variables, or (xi) the logistic regression equation with His, Leu, Met, Cit, Ile, and Tyr as the explanatory variables. Thus, the discriminant values obtained in the multivariate discriminants useful particularly for the discrimination between any one of the cervical cancer and the endometrial cancer and any one of the cervical cancer-free and the endometrial cancer-free, can be utilized to bring about the effect of enabling more accurately the discrimination.

When the discrimination described in 24. above is conducted in step SA-26, the multivariate discriminant may be (i) the fractional expression with a-ABA, His, and Val as the explanatory variables, (ii) the fractional expression with a-ABA, Met, and Val as the explanatory variables, (iii) the fractional expression with Met, His, Cit, and Arg as the explanatory variables, (iv) the linear discriminant with Gly, Val, His, and Arg as the explanatory variables, (v) the linear discriminant with Gly, Val, Met, and Lys as the explanatory variables, (vi) the linear discriminant with Cit, Met, His, and Arg as the explanatory variables, (vii) the linear discriminant with His, Leu, Met, Ile, Tyr, and Lys as the explanatory variables, (viii) the logistic regression equation with Val, Leu, His, and Arg as the explanatory variables, (ix) the logistic regression equation with Met, His, Orn, and Arg as the explanatory variables, (x) the logistic regression equation with Val, Tyr, His, and Arg as the explanatory variables, or (xi) the logistic regression equation with His, Leu, Met, Ile, Tyr, and Lys as the explanatory variables. Thus, the discriminant values obtained in the multivariate discriminants useful particularly for the 2-group discrimination between the cervical cancer and the cervical cancer-free, can be utilized to bring about the effect of enabling more accurately the 2-group discrimination.

When the discrimination described in 25. above is conducted in step SA-26, the multivariate discriminant may be (i) the fractional expression with Lys, His, and Arg as the explanatory variables, (ii) the fractional expression with a-ABA, His, and Met as the explanatory variables, (iii) the fractional expression with Ile, His, Asn, and Cit as the explanatory variables, (iv) the linear discriminant with Gln, His, Lys, and Arg as the explanatory variables, (v) the linear discriminant with Gly, Met, Phe, and His as the explanatory variables, (vi) the linear discriminant with Cit, Ile, His, and Arg as the explanatory variables, (vii) the linear discriminant with His, Asn, Val, Pro, Cit, and Ile as the explanatory variables, (viii) the logistic regression equation with Gln, Gly, His, and Arg as the explanatory variables, (ix) the logistic regression equation with Gln, Phe, His, and Arg as the explanatory variables, (x) the logistic regression equation with Gln, Ile, His, and Arg as the explanatory variables, or (xi) the logistic regression equation with His, Asn, Val, Pro, Cit, and Ile as the explanatory variables. Thus, the discriminant values obtained in the multivariate discriminants useful particularly for the 2-group discrimination between the endometrial cancer and the endometrial cancer-free, can be utilized to bring about the effect of enabling more accurately the 2-group discrimination.

When the discrimination described in 26. above is conducted in step SA-26, the multivariate discriminant may be (i) the fractional expression with Orn, Cit, and Met as the explanatory variables, (ii) the fractional expression with Gln, Cit, and Tyr as the explanatory variables, (iii) the fractional expression with Orn, His, Phe, and Trp as the explanatory variables, (iv) the linear discriminant with Ser, Cit, Orn, and Trp as the explanatory variables, (v) the linear discriminant with Ser, Cit, Ile, and Orn as the explanatory variables, (vi) the linear discriminant with Phe, Trp, Orn, and Lys as the explanatory variables, (vii) the linear discriminant with His, Trp, Glu, Cit, Ile, and Orn as the explanatory variables, (viii) the logistic regression equation with Ser, Cit, Trp, and Orn as the explanatory variables, (ix) the logistic regression equation with Gln, Cit, Ile, and Tyr as the explanatory variables, (x) the logistic regression equation with Asn, Phe, His, and Trp as the explanatory variables, or (xi) the logistic regression equation with His, Trp, Glu, Cit, Ile, and Orn as the explanatory variables. Thus, the discriminant values obtained in the multivariate discriminants useful particularly for the 2-group discrimination between the ovarian cancer and the ovarian cancer-free, can be utilized to bring about the effect of enabling more accurately the 2-group discrimination.

When the discrimination described in 27. above is conducted in step SA-26, the multivariate discriminant may be the discriminant with Cit, Met, Lys, Asn, Ala, Thr, Gln, and a-ABA as the explanatory variables which is prepared by the Mahalanobis' generalized distance method, or the discriminant prepared with His, Leu, Ser, Thr, Glu, Gln, Ala, and Lys as the explanatory variables which is prepared by the Mahalanobis' generalized distance method. Thus, the discriminant values obtained in the multivariate discriminants useful particularly for the discrimination between the cervical cancer, the endometrial cancer, and the ovarian cancer, can be utilized to bring about the effect of enabling more accurately the discrimination.

When the discrimination described in 28. above is conducted in step SA-26, the multivariate discriminant may be the linear discriminant with Phe, His, Met, Pro, Lys, and Arg as the explanatory variables, or the logistic regression equation with Phe, His, Met, Pro, Lys, and Arg as the explanatory variables. Thus, the discriminant values obtained in the multivariate discriminants useful particularly for the 2-group discrimination between the female genital cancer suffering risk group and the healthy group, can be utilized to bring about the effect of enabling more accurately the 2-group discrimination.

The multivariate discriminant described above can be prepared by a method described in International Publication WO 2004/052191 that is an international application filed by the present applicant or by a method (multivariate discriminant-preparing processing described later) described in International Publication WO 2006/098192 that is an international application filed by the present applicant. Any multivariate discriminants obtained by these methods can be preferably used in the evaluation of the state of female genital cancer, regardless of the unit of the amino acid concentration in the amino acid concentration data as input data.

In addition to the second embodiment described above, the female genital cancer-evaluating apparatus, the female genital cancer-evaluating method, the female genital cancer-evaluating system, the female genital cancer-evaluating program product and the recording medium according to the present invention can be practiced in various different embodiments within the technological scope of the claims. For example, among the processings described in the second embodiment above, all or a part of the processings described above as performed automatically may be performed manually, and all or a part of the manually conducted processings may be performed automatically by known methods. In addition, the processing procedure, control procedure, specific name, various registered data, information including parameters such as retrieval condition, screen, and database configuration shown in the description above or drawings may be modified arbitrarily, unless specified otherwise. For example, the components of the female genital cancer-evaluating apparatus 100 shown in the figures are conceptual and functional and may not be the same physically as those shown in the figure. In addition, all or an arbitrary part of the operational function of each component and each device in the female genital cancer-evaluating apparatus 100 (in particular, the operational functions executed in the control device 102) may be executed by the CPU (Central Processing Unit) or the programs executed by the CPU, and may be realized as wired-logic hardware.

The “program” is a data processing method written in any language or by any description method and may be of any format such as source code or binary code. The “program” may not be limited to a program configured singly, and may include a program configured decentrally as a plurality of modules or libraries, and a program to achieve the function together with a different program such as OS (Operating System). The program is stored on a recording medium and read mechanically as needed by the female genital cancer-evaluating apparatus 100. Any well-known configuration or procedure may be used as specific configuration, reading procedure, installation procedure after reading, and the like for reading the programs recorded on the recording medium in each apparatus.

The “recording media” includes any “portable physical media”, “fixed physical media”, and “communication media”. Examples of the “portable physical media” include flexible disk, magnetic optical disk, ROM, EPROM (Erasable Programmable Read Only Memory), EEPROM (Electronically Erasable and Programmable Read Only Memory), CD-ROM (Compact Disk Read Only Memory), MO (Magneto-Optical disk), DVD (Digital Versatile Disk), and the like. Examples of the “fixed physical media” include ROM, RAM, HD, and the like which are installed in various computer systems. The “communication media” for example stores the program for a short period of time such as communication line and carrier wave when the program is transmitted via a network such as LAN (Local Area Network), WAN (Wide Area Network), or the Internet.

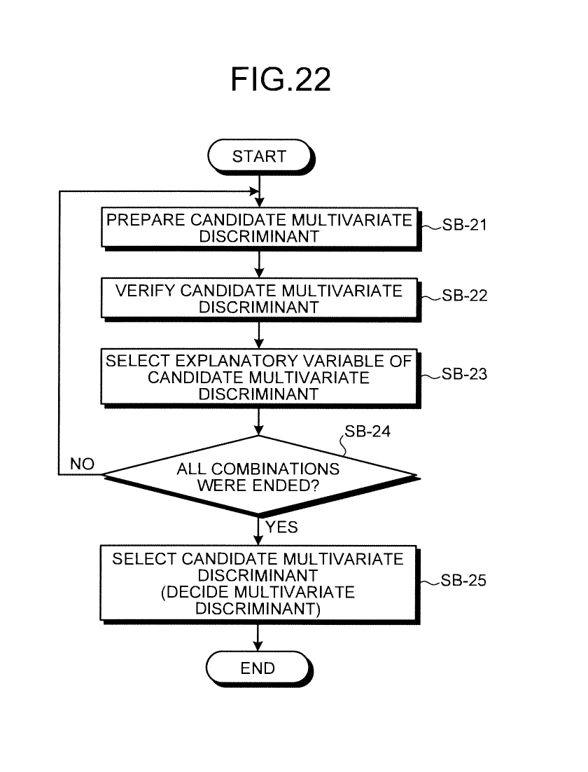

Finally, an example of the multivariate discriminant-preparing processing performed in the female genital cancer-evaluating apparatus 100 is described in detail with reference to FIG. 22. FIG. 22 is a flowchart showing an example of the multivariate discriminant-preparing processing. The multivariate discriminant-preparing processing may be performed in the database apparatus 400 handling the female genital cancer state information.

In the present description, the female genital cancer-evaluating apparatus 100 stores the female genital cancer state information previously obtained from the database apparatus 400 in a predetermined memory region of the female genital cancer state information file 106c. The female genital cancer-evaluating apparatus 100 shall store, in a predetermined memory region of the designated female genital cancer state information file 106d, the female genital cancer state information including the female genital cancer state index data and amino acid concentration data designated previously in the female genital cancer state information-designating part 102g.

The candidate multivariate discriminant-preparing part 102h1 in the multivariate discriminant-preparing part 102h first prepares the candidate multivariate discriminants according to a predetermined discriminant-preparing method from the female genital cancer state information stored in a predetermine memory region of the designated female genital cancer state information file 106d, and stores the prepared candidate multivariate discriminants in a predetermined memory region of the candidate multivariate discriminant file 106e1 (step SB-21). Specifically, the candidate multivariate discriminant-preparing part 102h1 in the multivariate discriminant-preparing part 102h first selects a desired method out of a plurality of different discriminant-preparing methods (including those for multivariate analysis such as principal component analysis, discriminant analysis, support vector machine, multiple regression analysis, logistic regression analysis, k-means method, cluster analysis, and decision tree) and determines the form of the candidate multivariate discriminant to be prepared based on the selected discriminant-preparing method. The candidate multivariate discriminant-preparing part 102h1 in the multivariate discriminant-preparing part 102h then performs various calculation corresponding to the selected function-selecting method (e.g., average or variance), based on the female genital cancer state information. The candidate multivariate discriminant-preparing part 102h1 in the multivariate discriminant-preparing part 102h then determines the parameters for the calculation result and the determined candidate multivariate discriminant. In this way, the candidate multivariate discriminant is generated based on the selected discriminant-preparing method. When the candidate multivariate discriminants are generated simultaneously and concurrently (in parallel) by using a plurality of different discriminant-preparing methods in combination, the processings described above may be executed concurrently for each selected discriminant-preparing method. Alternatively when the candidate multivariate discriminants are generated in series by using a plurality of different discriminant-preparing methods in combination, for example, the candidate multivariate discriminants may be generated by converting the female genital cancer state information with the candidate multivariate discriminants prepared by performing principal component analysis and performing discriminant analysis of the converted female genital cancer state information.

The candidate multivariate discriminant-verifying part 102h2 in the multivariate discriminant-preparing part 102h verifies (mutually verifies) the candidate multivariate discriminant prepared in step SB-21 according to a particular verifying method and stores the verification result in a predetermined memory region of the verification result file 106e2 (step SB-22). Specifically, the candidate multivariate discriminant-verifying part 102h2 in the multivariate discriminant-preparing part 102h first generates the verification data to be used in verification of the candidate multivariate discriminant, based on the female genital cancer state information stored in a predetermined memory region of the designated female genital cancer state information file 106d, and verifies the candidate multivariate discriminant according to the generated verification data. If a plurality of the candidate multivariate discriminants is generated by using a plurality of different discriminant-preparing methods in step SB-21, the candidate multivariate discriminant-verifying part 102h2 in the multivariate discriminant-preparing part 102h verifies each candidate multivariate discriminant corresponding to each discriminant-preparing method according to a particular verifying method. Here in step SB-22, at least one of the discrimination rate, sensitivity, specificity, information criterion, and the like of the candidate multivariate discriminant may be verified based on at least one method of the bootstrap method, holdout method, leave-one-out method, and the like. Thus, it is possible to select the candidate multivariate discriminant higher in predictability or reliability, by taking the female genital cancer state information and diagnostic condition into consideration.

Then, the explanatory variable-selecting part 102h3 in the multivariate discriminant-preparing part 102h selects the combination of the amino acid concentration data contained in the female genital cancer state information used in preparing the candidate multivariate discriminant by selecting the explanatory variable of the candidate multivariate discriminant from the verification result obtained in step SB-22 according to a predetermined explanatory variable-selecting method, and stores the female genital cancer state information including the selected combination of the amino acid concentration data in a predetermined memory region of the selected female genital cancer state information file 106e3 (step SB-23). When a plurality of the candidate multivariate discriminants is generated by using a plurality of different discriminant-preparing methods in step SB-21 and each candidate multivariate discriminant corresponding to each discriminant-preparing method is verified according to a predetermined verifying method in step SB-22, the explanatory variable-selecting part 102h3 in the multivariate discriminant-preparing part 102h selects the explanatory variable of the candidate multivariate discriminant for each candidate multivariate discriminant corresponding to the verification result obtained in step SB-22, according to a predetermined explanatory variable-selecting method in step SB-23. Here in step SB-23, the explanatory variable of the candidate multivariate discriminant may be selected from the verification results according to at least one of the stepwise method, best path method, local search method, and genetic algorithm. The best path method is a method of selecting an explanatory variable by optimizing an evaluation index of the candidate multivariate discriminant while eliminating the explanatory variables contained in the candidate multivariate discriminant one by one. In step SB-23, the explanatory variable-selecting part 102h3 in the multivariate discriminant-preparing part 102h may select the combination of the amino acid concentration data based on the female genital cancer state information stored in a predetermined memory region of the designated female genital cancer state information file 106d.

The multivariate discriminant-preparing part 102h then judges whether all combinations of the amino acid concentration data contained in the female genital cancer state information stored in a predetermined memory region of the designated female genital cancer state information file 106d are processed, and if the judgment result is “End” (Yes in step SB-24), the processing advances to the next step (step SB-25), and if the judgment result is not “End” (No in step SB-24), it returns to step SB-21. The multivariate discriminant-preparing part 102h may judge whether the processing is performed a predetermined number of times, and if the judgment result is “End” (Yes in step SB-24), the processing may advance to the next step (step SB-25), and if the judgment result is not “End” (No in step SB-24), it may return to step SB-21. The multivariate discriminant-preparing part 102h may judge whether the combination of the amino acid concentration data selected in step SB-23 is the same as the combination of the amino acid concentration data contained in the female genital cancer state information stored in a predetermined memory region of the designated female genital cancer state information file 106d or the combination of the amino acid concentration data selected in the previous step SB-23, and if the judgment result is “the same” (Yes in step SB-24), the processing may advance to the next step (step SB-25) and if the judgment result is not “the same” (No in step SB-24), it may return to step SB-21. If the verification result is specifically the evaluation value for each multivariate discriminant, the multivariate discriminant-preparing part 102h may advance to step SB-25 or return to step SB-21, based on the comparison of the evaluation value with a particular threshold corresponding to each discriminant-preparing method.

Then, the multivariate discriminant-preparing part 102h determines the multivariate discriminant by selecting the candidate multivariate discriminant used as the multivariate discriminant based on the verification results from a plurality of the candidate multivariate discriminants, and stores the determined multivariate discriminant (the selected candidate multivariate discriminant) in particular memory region of the multivariate discriminant file 106e4 (step SB-25). Here, in step SB-25, for example, there are cases where the optimal multivariate discriminant is selected from the candidate multivariate discriminants prepared in the same discriminant-preparing method or the optimal multivariate discriminant is selected from all candidate multivariate discriminants.

Given the foregoing description, the explanation of the multivariate discriminant-preparing processing is finished.

### Example 1

Blood amino acid concentrations are measured from the blood samples of a cervical cancer patient group subjected to cervical cancer definitive diagnosis, an endometrial cancer patient group subjected to endometrial cancer definitive diagnosis, and an ovarian cancer patient group subjected to ovarian cancer definitive diagnosis and the blood samples of a cervical cancer-free group, an endometrial cancer-free group, and an ovarian cancer-free group by the amino acid analysis method. Here, in Example 1 and examples thereafter, the cervical cancer patient group, the endometrial cancer patient group, and the ovarian cancer patient group can be generically expressed as a cancer patient group, and the cervical cancer-free group, the endometrial cancer-free group, and the ovarian cancer-free group can be generically expressed as a cancer-free group. In addition, in the cancer-free group, a group suffering from benign disease such as a myoma of a uterus can be expressed as a benign disease group, and a group other than that can be expressed as a healthy group. Further, a group including the benign disease group and the cancer patient group can be expressed as a female genital cancer suffering risk group.

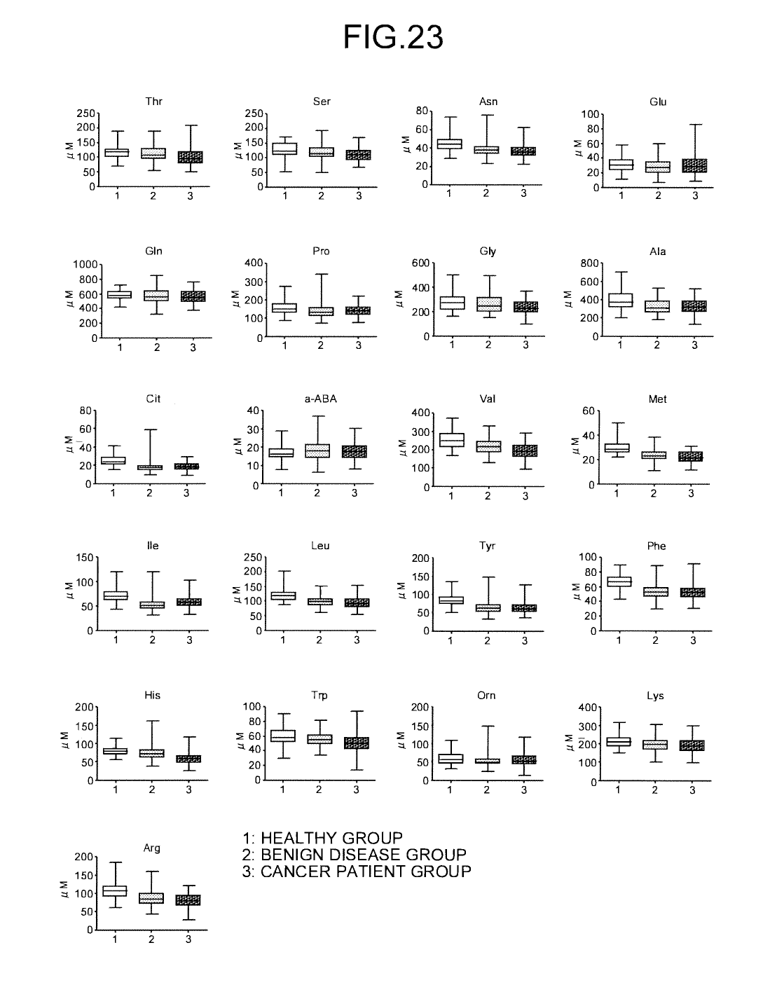

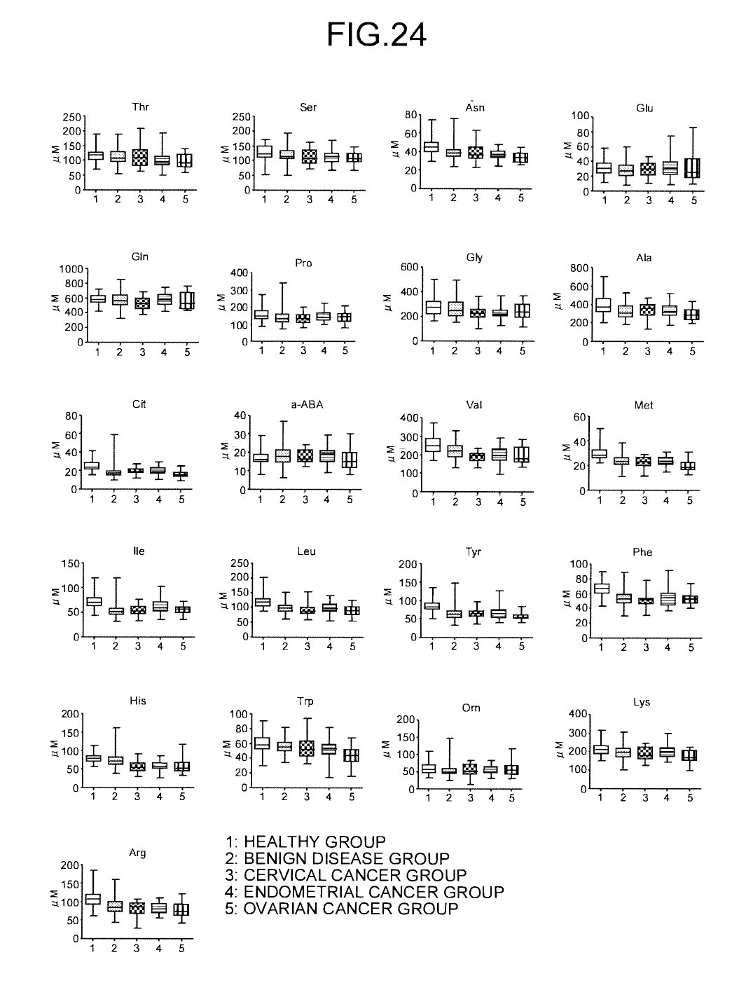

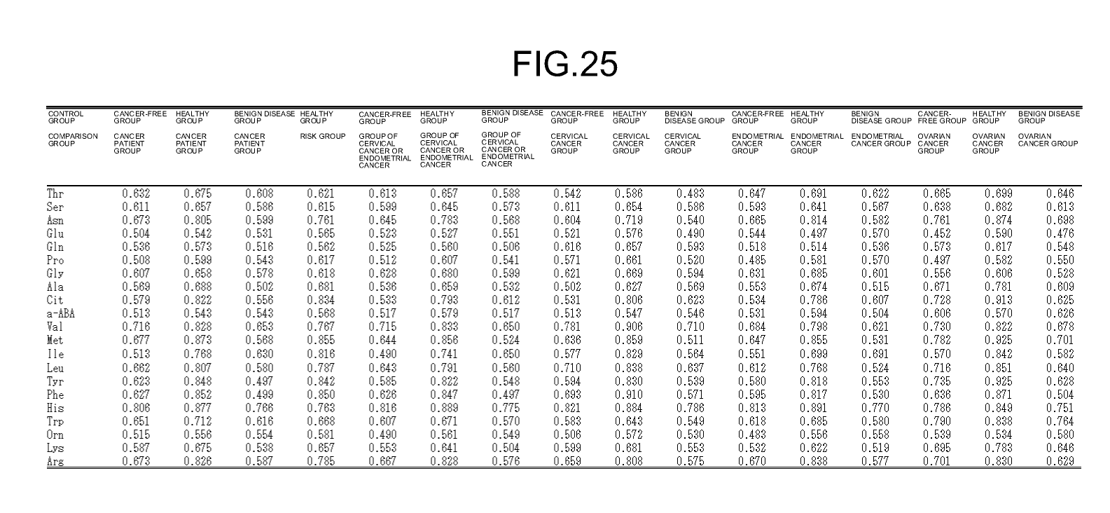

FIG. 23 is boxplots of the distribution of the amino acid explanatory variables of the cancer patient group, the benign disease group, and the healthy group. FIG. 24 is boxplots of the distribution of the amino acid explanatory variables of the cervical cancer group, the endometrial cancer group, the ovarian cancer group, the benign disease group, and the healthy group. FIG. 25 is a chart of results obtained by calculating the areas under the ROC curve of the amino acid explanatory variables in 2-group discrimination between the groups.

As shown in FIGS. 23, 24, and 25, it is found that many amino acid concentrations are different among the healthy group, the benign disease group, and the cancer patient group. In particular, in 2-group discrimination between the cancer-free group, the benign disease group, or the healthy group and the cancer patient group and in 2-group discrimination between the healthy group and the female genital cancer suffering risk group, it is found that Asn, Val, Met, Leu, His, Trp, and Arg are in the top 12 having a high ROC AUC value at all times. In addition, in 2-group discrimination between the cancer-free group, the benign disease group, or the healthy group and the cervical cancer group, it is found that Gly, Val, Leu, Phe, His, Lys, and Arg are in the top 12 having a high ROC AUC value at all times. Further, in 2-group discrimination between the cancer-free group, the benign disease group, or the healthy group and the endometrial cancer group, it is found that Thr, Asn, Gly, Val, His, Trp, and Arg are in the top 12 having a high ROC AUC value at all times. Furthermore, in 2-group discrimination between the cancer-free group, the benign disease group, or the healthy group and the ovarian cancer group, it is found that Asn, Cit, Val, Met, Leu, Tyr, His, Trp, Lys, and Arg are in the top 12 having a high ROC AUC value at all times. From this, these amino acids are found to contribute to cervical cancer, endometrial cancer, or ovarian cancer.

### Example 2

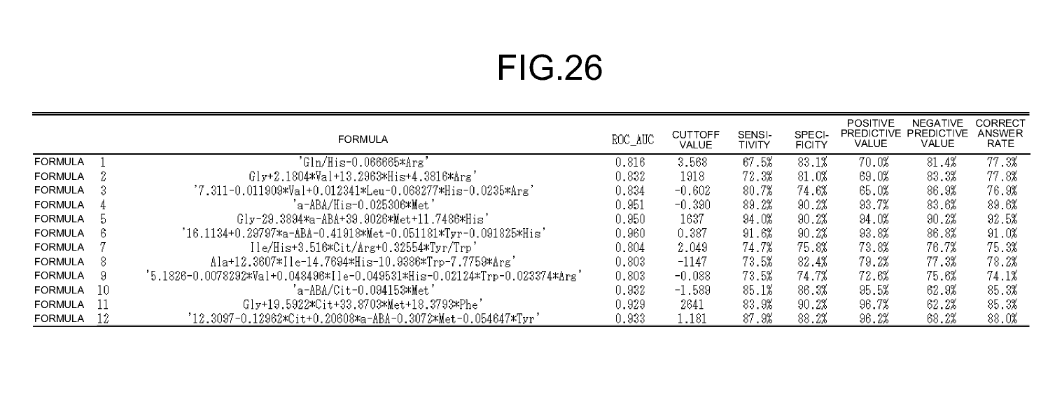

The sample data used in Example 1 is used. Indexes which maximize 2-group discriminative ability between the cancer patient group and the cancer-free group with respect to the discrimination of the cervical cancer group, the endometrial cancer group, and the ovarian cancer group are earnestly searched for, by using the method described in International publication WO 2004/052191 which is an international application by the present applicant. As a result, index formula 1 (see FIG. 26) is obtained among the index formulae having equivalent ability. Indexes which maximize 2-group discriminative ability between the cancer patient group and the cancer-free group with respect to the discrimination of the cervical cancer group, the endometrial cancer group, and the ovarian cancer group are searched for, by linear discriminant analysis (explanatory variable coverage method according to the minimum AIC (Akaike information criteria)). As a result, index formula 2 (see FIG. 26) is obtained among the index formulae having equivalent ability. Indexes which maximize 2-group discriminative ability between the cancer patient group and the cancer-free group with respect to the discrimination of the cervical cancer group, the endometrial cancer group, and the ovarian cancer group are searched for, by logistic regression analysis (explanatory variable coverage method according to the minimum AIC). As a result, index formula 3 (see FIG. 26) is obtained among the index formulae having equivalent ability. The value of each coefficient shown in index formulae 1, 2, and 3 may be multiplied by a real number, and the value of each constant term may be subjected to addition, subtraction, multiplication, and division with an arbitrary real constant.

Indexes which maximize 2-group discriminative ability between the cancer patient group and the healthy group with respect to the discrimination of the cervical cancer group, the endometrial cancer group, and the ovarian cancer group are earnestly searched for, by using the method described in International publication WO 2004/052191 which is an international application by the present applicant. As a result, index formula 4 (see FIG. 26) is obtained among the index formulae having equivalent ability. Indexes which maximize 2-group discriminative ability between the cancer patient group and the healthy group with respect to the discrimination of the cervical cancer group, the endometrial cancer group, and the ovarian cancer group are searched for, by linear discriminant analysis (explanatory variable coverage method according to the minimum AIC). As a result, index formula 5 (see FIG. 26) is obtained among the index formulae having equivalent ability. Indexes which maximize 2-group discriminative ability between the cancer patient group and the healthy group with respect to the discrimination of the cervical cancer group, the endometrial cancer group, and the ovarian cancer group are searched for, by logistic regression analysis (explanatory variable coverage method according to the minimum AIC). As a result, index formula 6 (see FIG. 26) is obtained among the index formulae having equivalent ability. The value of each coefficient shown in index formulae 4, 5, and 6 may be multiplied by a real number, and the value of each constant term may be subjected to addition, subtraction, multiplication, and division with an arbitrary real constant.

Indexes which maximize 2-group discriminative ability between the cancer patient group and the benign disease group with respect to the discrimination of the cervical cancer group, the endometrial cancer group, and the ovarian cancer group are earnestly searched for, by using the method described in International publication WO 2004/052191 which is an international application by the present applicant. As a result, index formula 7 (see FIG. 26) is obtained among the index formulae having equivalent ability. Indexes which maximize 2-group discriminative ability between the cancer patient group and the benign disease group with respect to the discrimination of the cervical cancer group, the endometrial cancer group, and the ovarian cancer group are searched for, by linear discriminant analysis (explanatory variable coverage method according to the minimum AIC). As a result, index formula 8 (see FIG. 26) is obtained among the index formulae having equivalent ability. Indexes which maximize 2-group discriminative ability between the cancer patient group and the benign disease group with respect to the discrimination of the cervical cancer group, the endometrial cancer group, and the ovarian cancer group are searched for, by logistic regression analysis (explanatory variable coverage method according to the minimum AIC). As a result, index formula 9 (see FIG. 26) is obtained among the index formulae having equivalent ability. The value of each coefficient shown in index formulae 7, 8, and 9 may be multiplied by a real number, and the value of each constant term may be subjected to addition, subtraction, multiplication, and division with an arbitrary real constant.

Indexes which maximize 2-group discriminative ability between the healthy group and the female genital cancer suffering risk group with respect to the discrimination of the cervical cancer group, the endometrial cancer group, and the ovarian cancer group are earnestly searched for, by using the method described in International publication WO 2004/052191 which is an international application by the present applicant. As a result, index formula 10 (see FIG. 26) is obtained among the index formulae having equivalent ability. Indexes which maximize 2-group discriminative ability between the healthy group and the female genital cancer suffering risk group with respect to the discrimination of the cervical cancer group, the endometrial cancer group, and the ovarian cancer group are searched for, by linear discriminant analysis (explanatory variable coverage method according to the minimum AIC). As a result, index formula 11 (see FIG. 26) is obtained among the index formulae having equivalent ability. Indexes which maximize 2-group discriminative ability between the healthy group and the female genital cancer suffering risk group with respect to the discrimination of the cervical cancer group, the endometrial cancer group, and the ovarian cancer group are searched for, by logistic regression analysis (explanatory variable coverage method according to the minimum AIC). As a result, index formula 12 (see FIG. 26) is obtained among the index formulae having equivalent ability. The value of each coefficient shown in index formulae 10, 11, and 12 may be multiplied by a real number, and the value of each constant term may be subjected to addition, subtraction, multiplication, and division with an arbitrary real constant.

To examine the diagnostic ability using index formulae 1 to 3 in the discrimination of the cervical cancer group, the endometrial cancer group, and the ovarian cancer group, 2-group discrimination between the cancer patient group and the cancer-free group is evaluated by the ROC curve. As a result, the diagnostic ability as shown in FIG. 26 is obtained so that it is found that these index formulae are useful, with high diagnostic ability. As shown in FIG. 26, with regard to these index formulae, an optimum cutoff value, and the sensitivity, specificity, positive predictive value, negative predictive value, and correct answer rate of the used data are calculated.

To examine the diagnostic ability using index formulae 4 to 6 in the discrimination of the cervical cancer group, the endometrial cancer group, and the ovarian cancer group, 2-group discrimination between the cancer patient group and the healthy group is evaluated by the ROC curve. As a result, the diagnostic ability as shown in FIG. 26 is obtained so that it is found that these index formulae are useful, with high diagnostic ability. As shown in FIG. 26, with regard to these index formulae, an optimum cutoff value, and the sensitivity, specificity, positive predictive value, negative predictive value, and correct answer rate of the used data are calculated.

To examine the diagnostic ability using index formulae 7 to 9 in the discrimination of the cervical cancer group, the endometrial cancer group, and the ovarian cancer group, 2-group discrimination between the cancer patient group and the benign disease group is evaluated by the ROC curve. As a result, the diagnostic ability as shown in FIG. 26 is obtained so that it is found that these index formulae are useful, with high diagnostic ability. As shown in FIG. 26, with regard to these index formulae, an optimum cutoff value, and the sensitivity, specificity, positive predictive value, negative predictive value, and correct answer rate of the used data are calculated.

To examine the diagnostic ability using index formulae 10 to 12 in the discrimination of the cervical cancer group, the endometrial cancer group, and the ovarian cancer group, 2-group discrimination between the healthy group and the female genital cancer suffering risk group is evaluated by the ROC curve. As a result, the diagnostic ability as shown in FIG. 26 is obtained so that it is found that these index formulae are useful, with high diagnostic ability. As shown in FIG. 26, with regard to these index formulae, an optimum cutoff value, and the sensitivity, specificity, positive predictive value, negative predictive value, and correct answer rate of the used data are calculated.

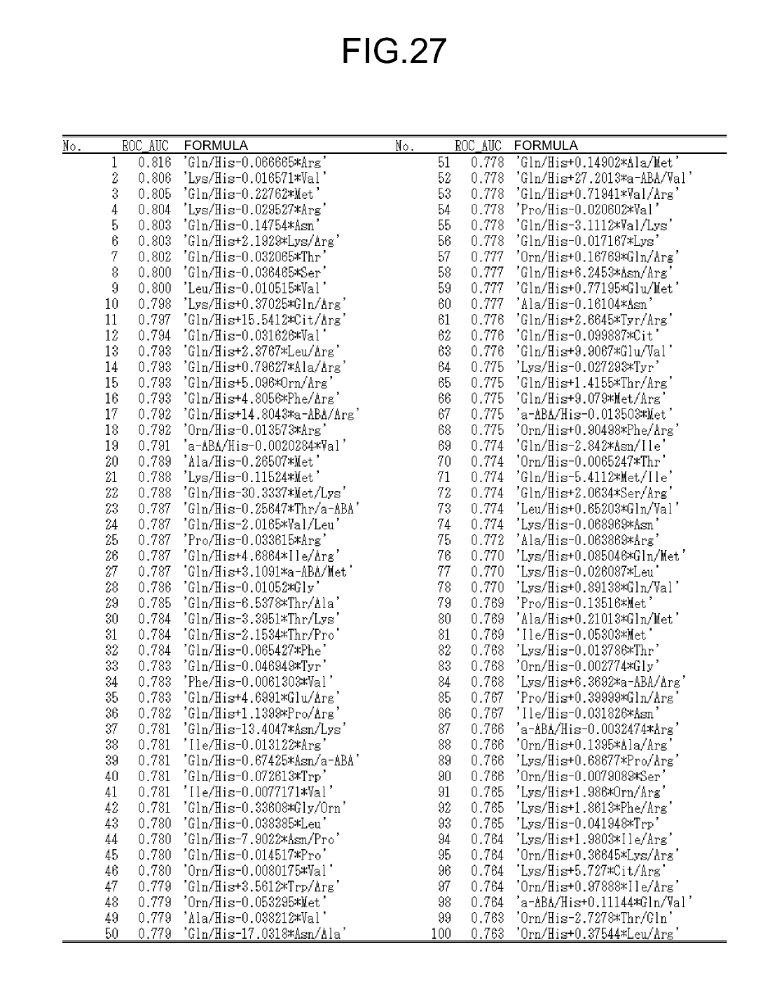

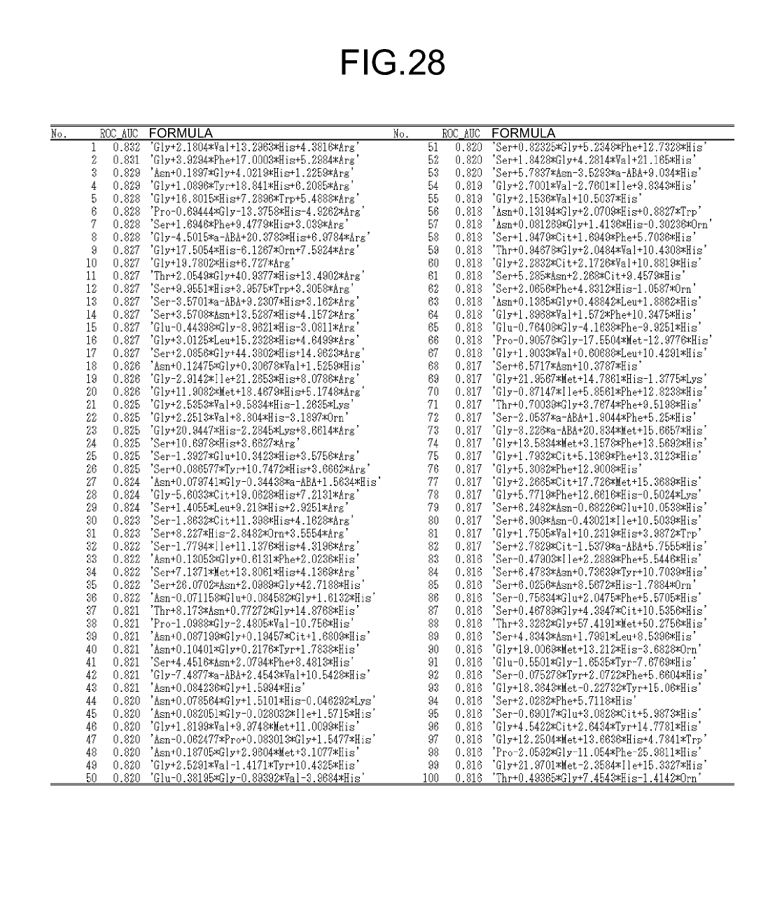

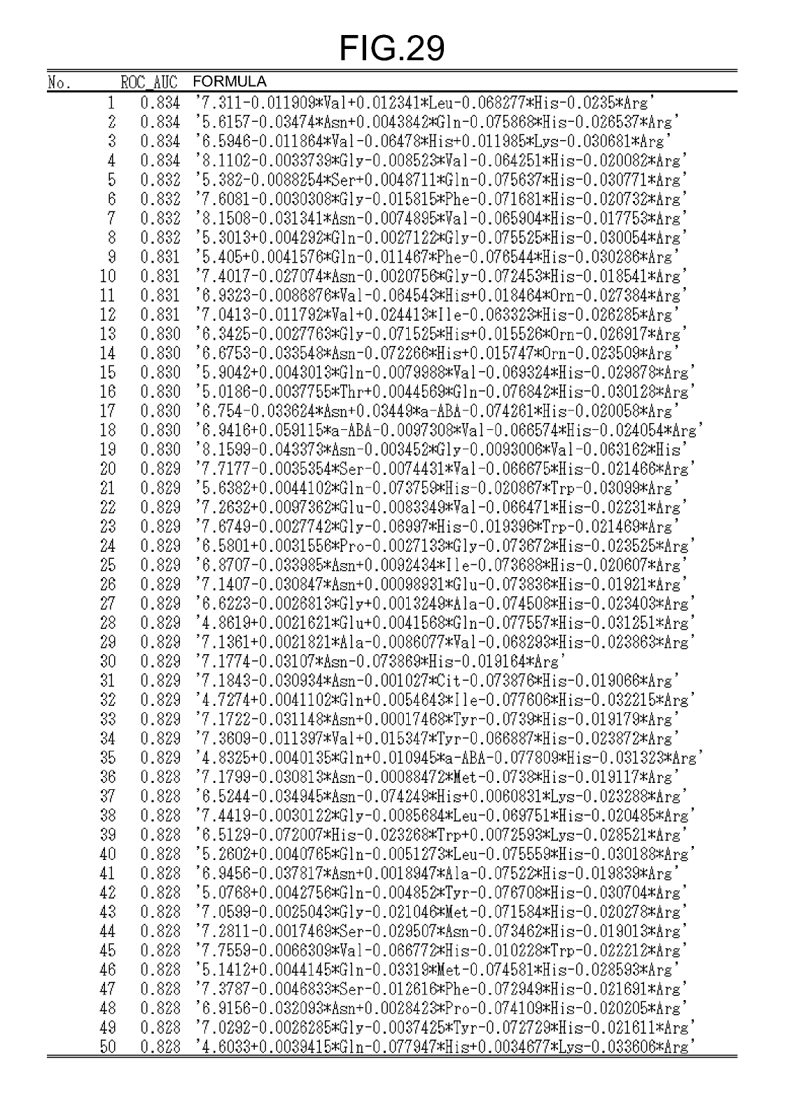

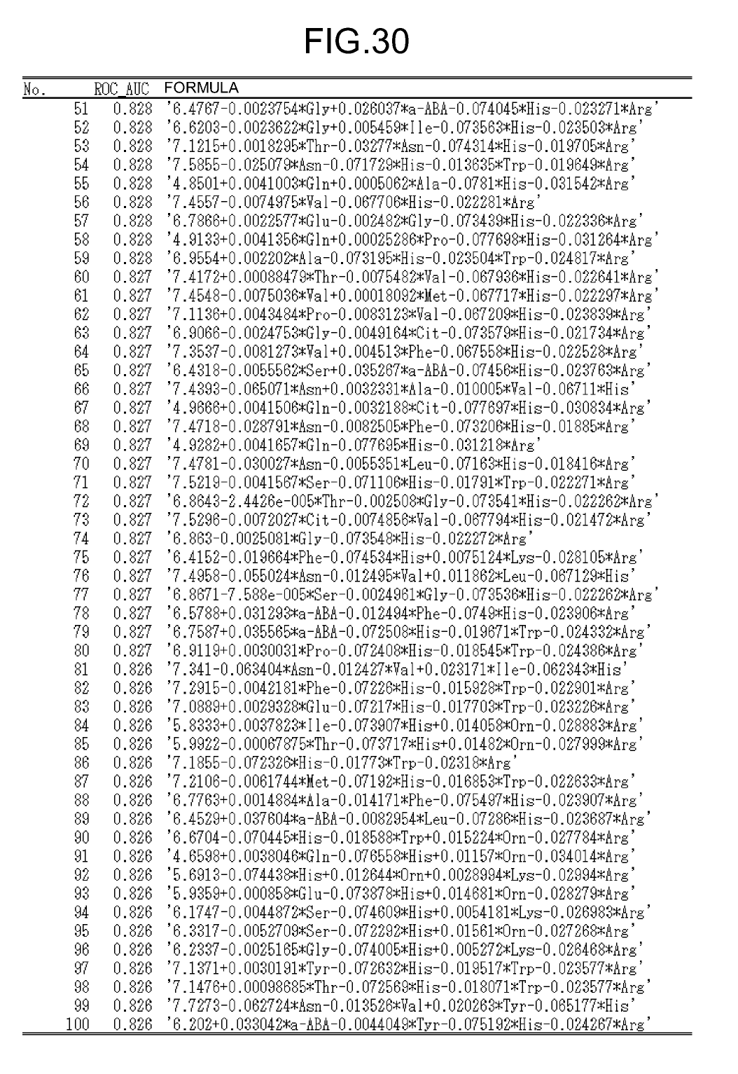

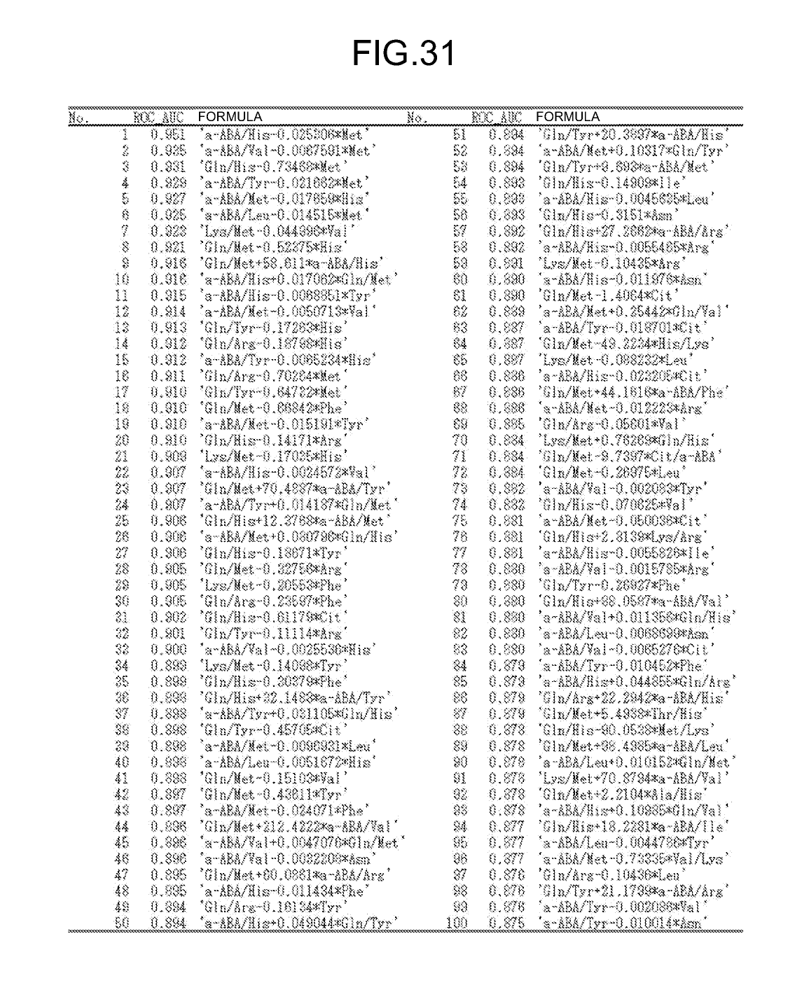

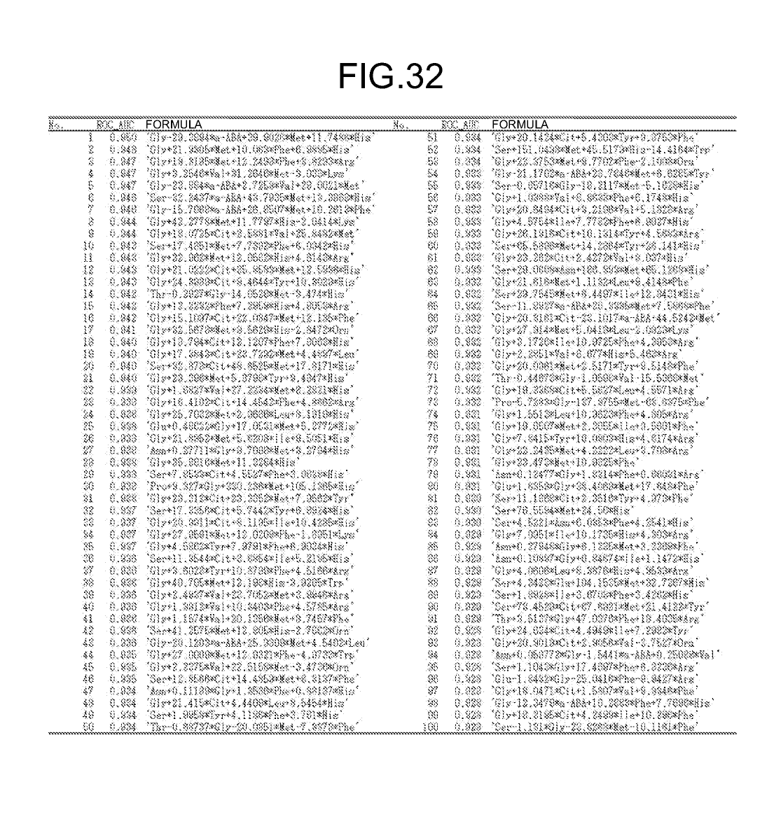

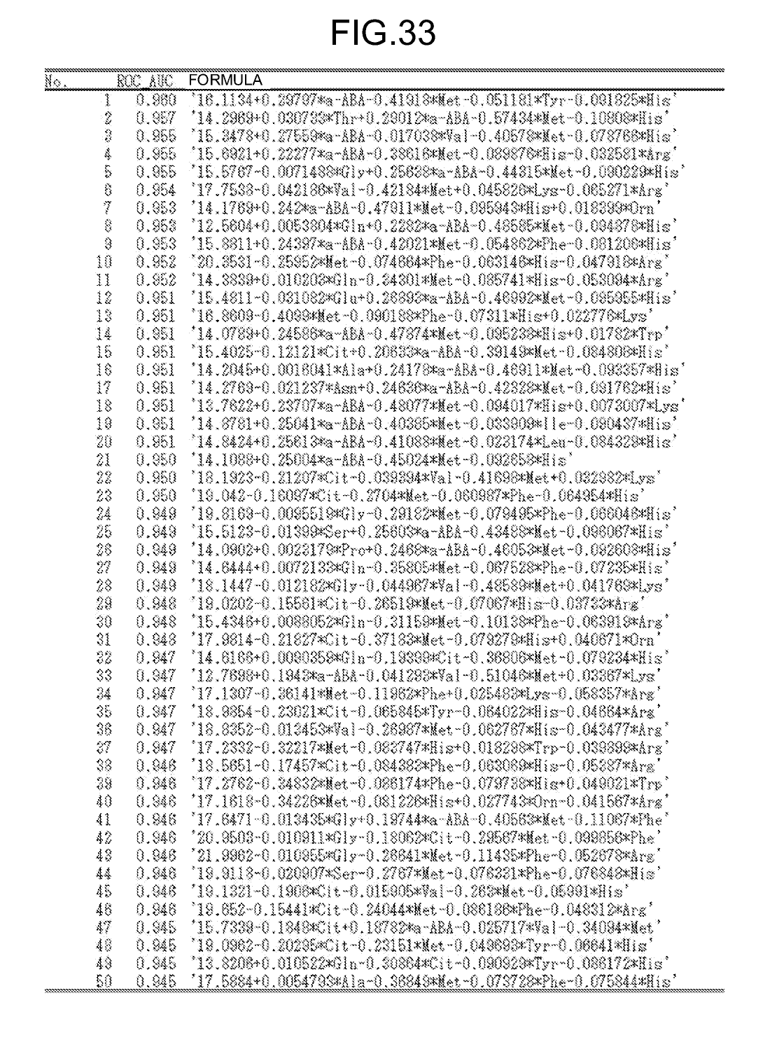

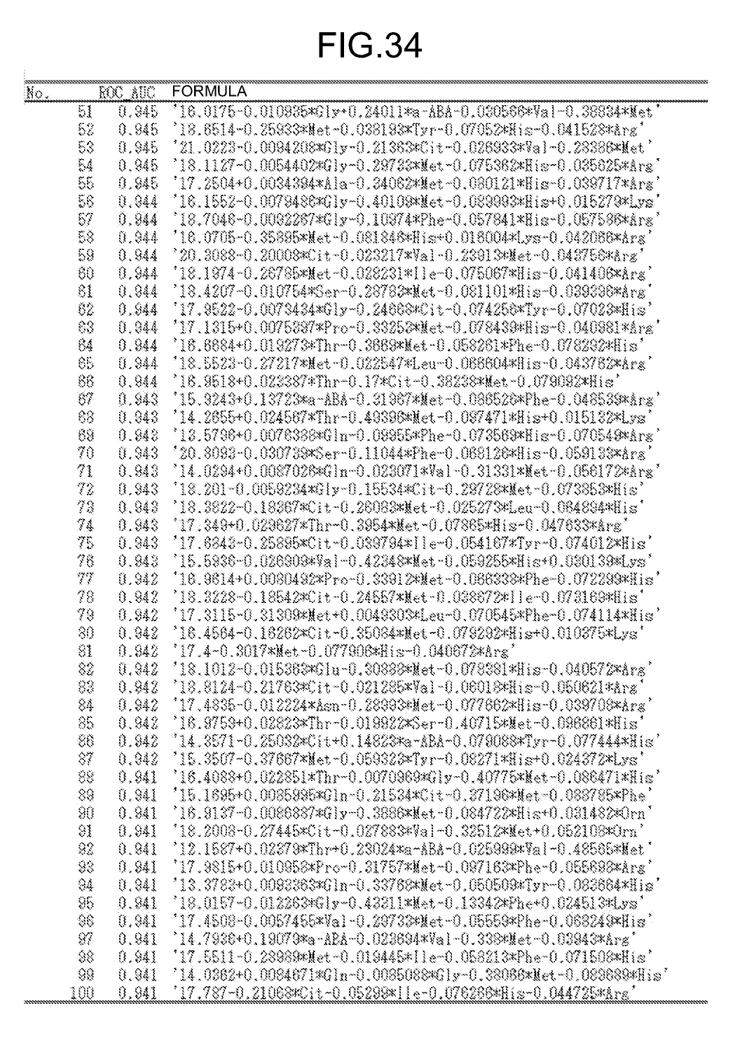

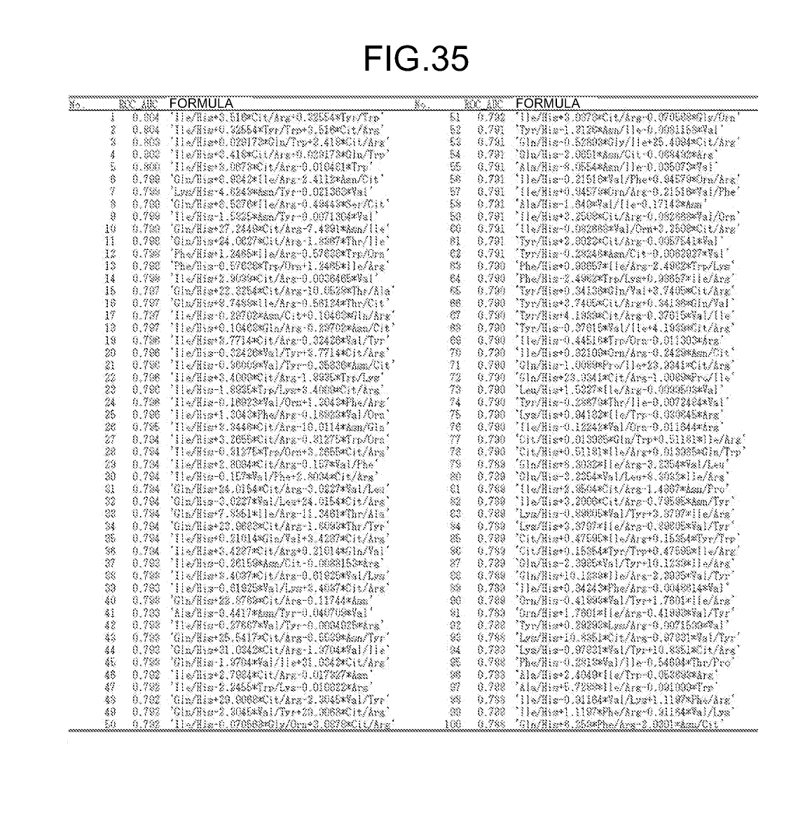

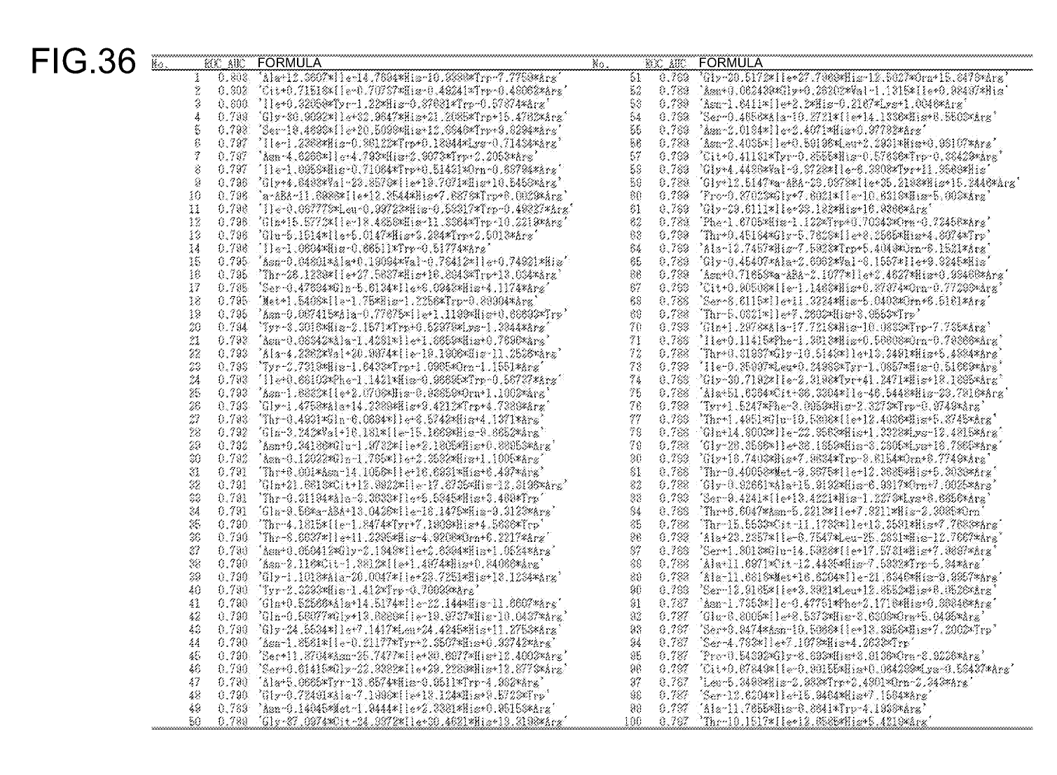

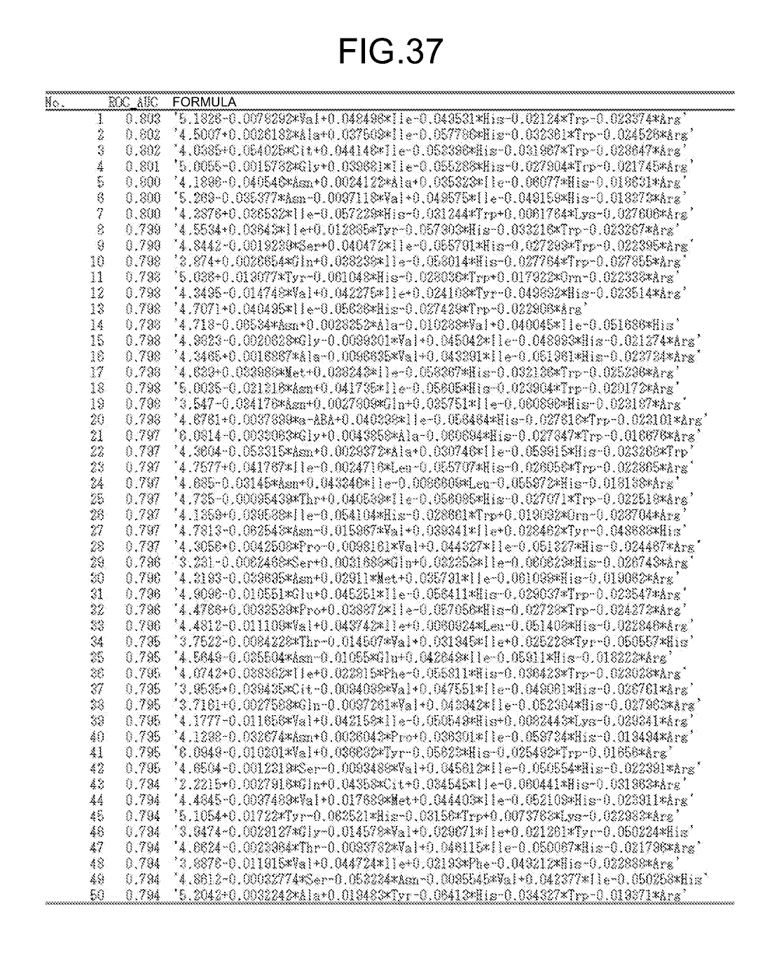

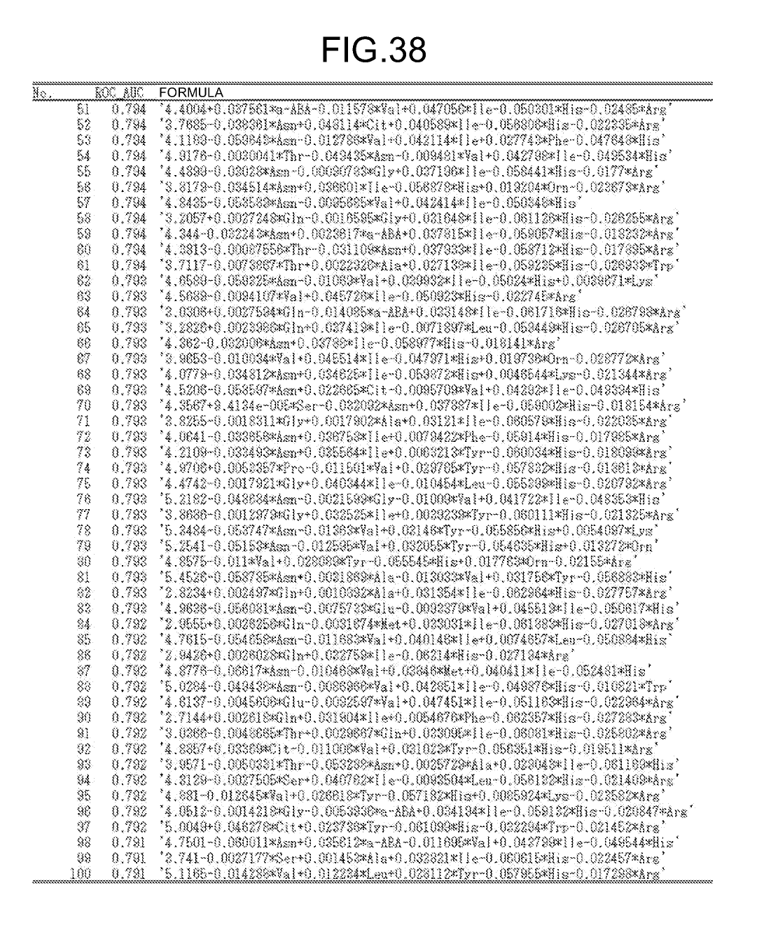

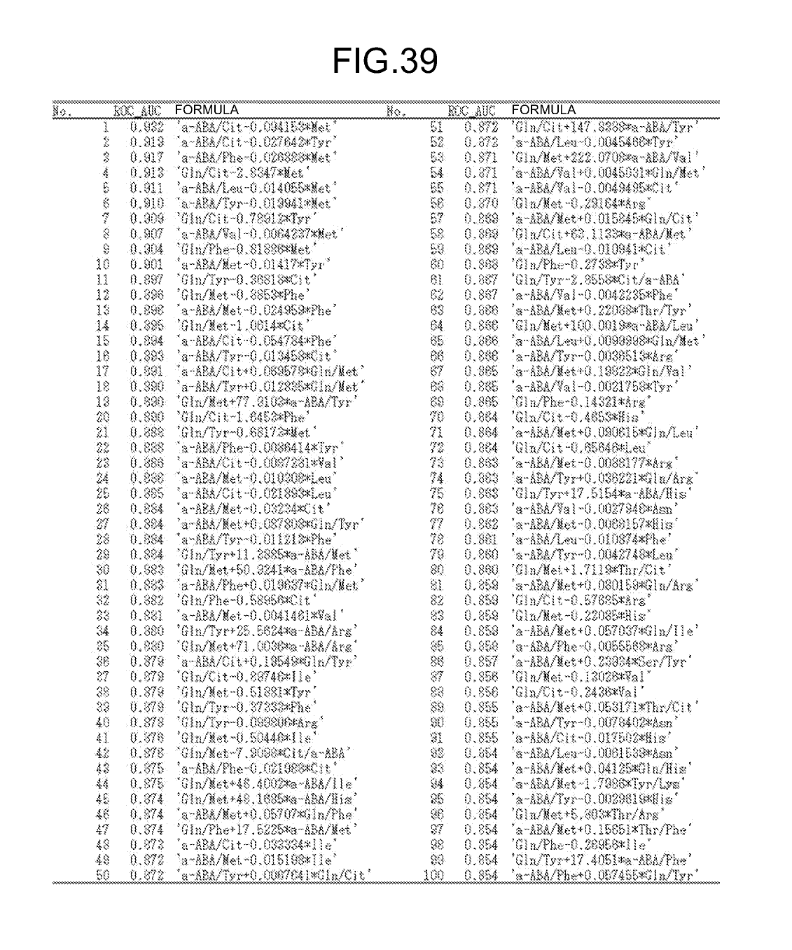

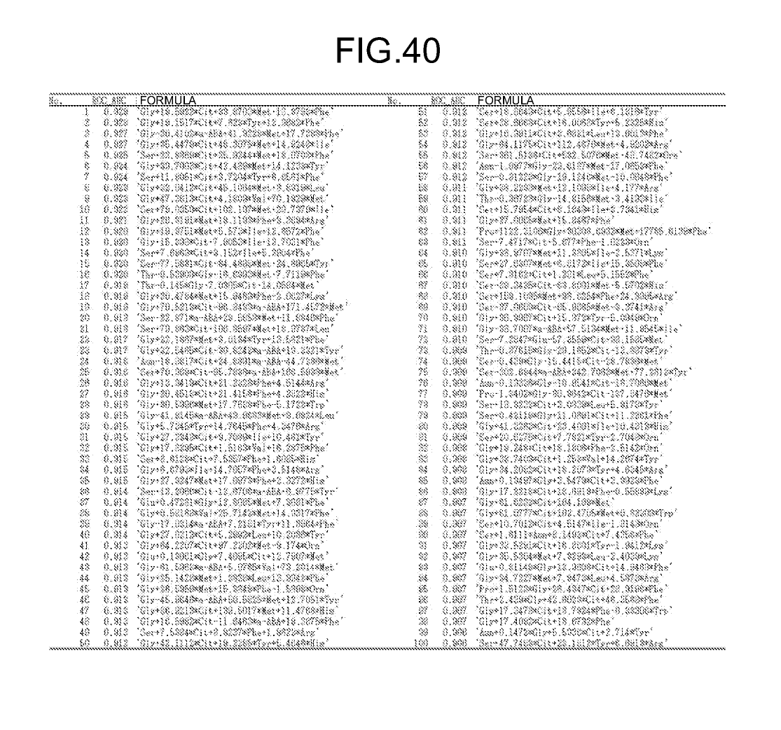

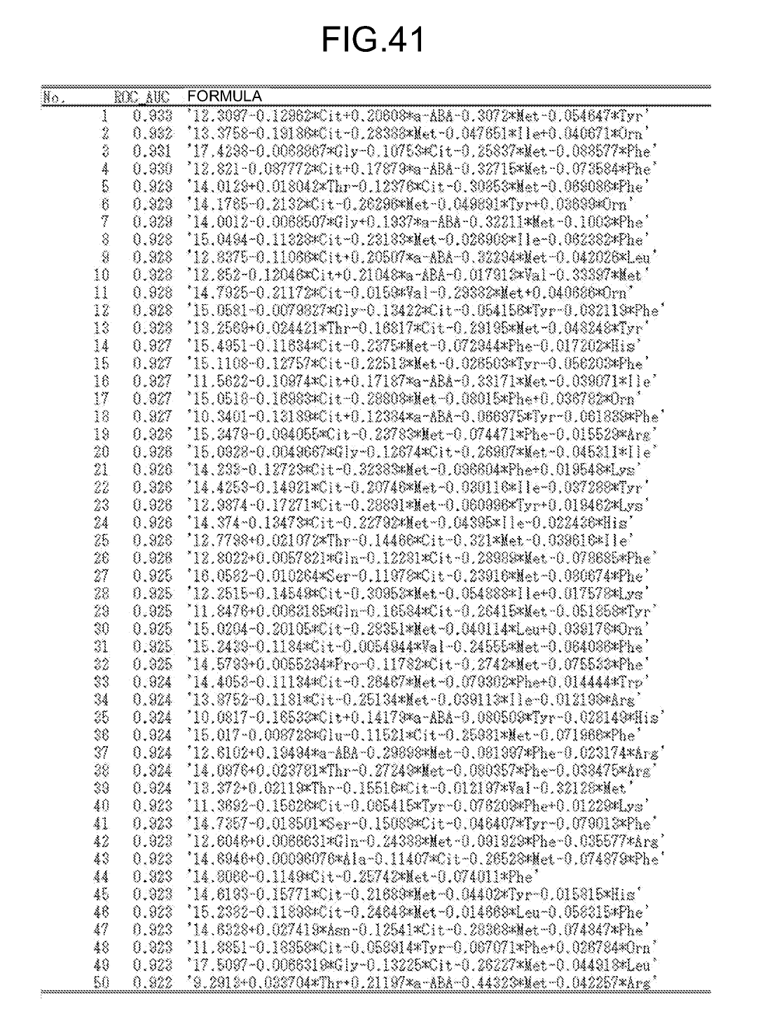

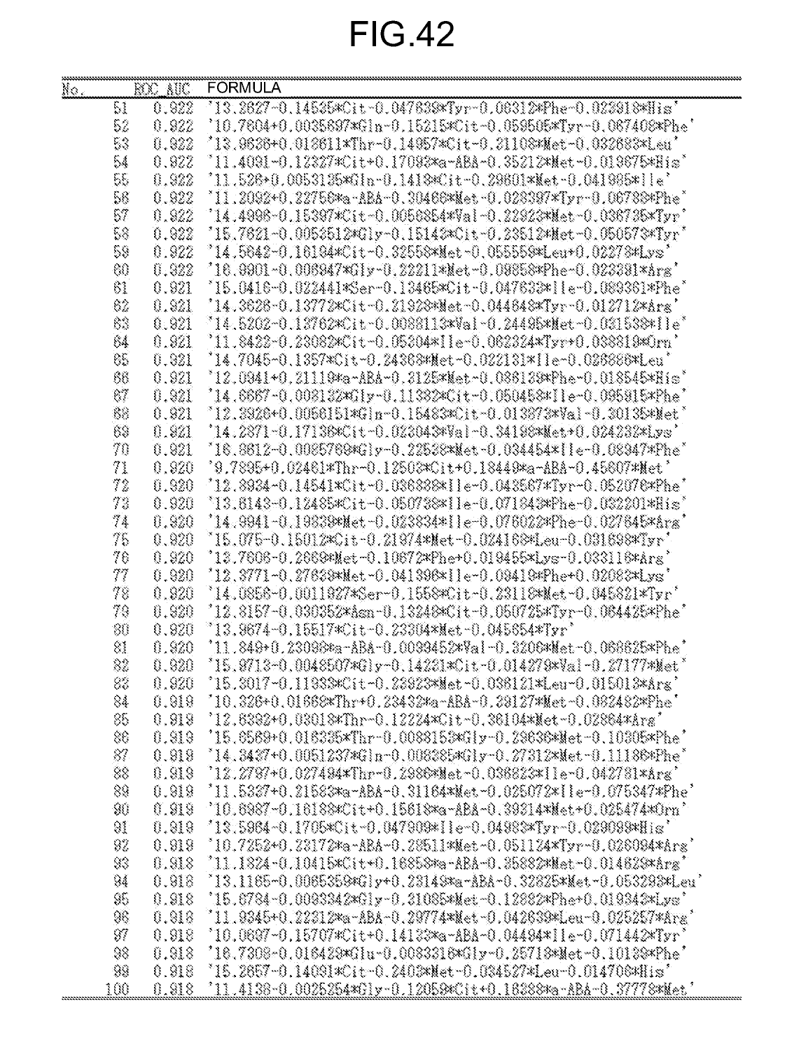

As successively shown in FIGS. 27 to 42, with respect to index formulae 1 to 12, the index formulae having equivalent discriminative ability are obtained. The value of each coefficient in the formulae shown in FIGS. 27 to 42 may be multiplied by a real number, and the value of each constant term may be subjected to addition, subtraction, multiplication, and division with an arbitrary real constant.

### Example 3

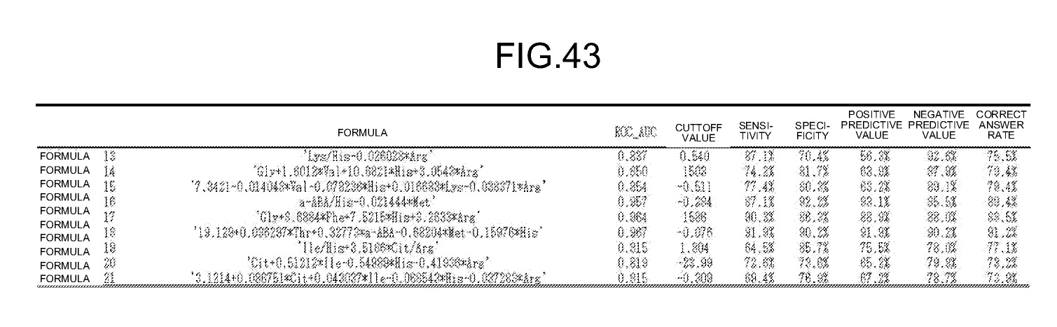

Of the sample data used in Example 1, the data of the cervical cancer group, the endometrial cancer group, and the cancer-free group are used. Indexes which maximize 2-group discriminative ability between (i) the cervical cancer group and the endometrial cancer group and (ii) the cancer-free group with respect to the discrimination of the cervical cancer group, the endometrial cancer group, and the ovarian cancer group are earnestly searched for, by using the method described in International publication WO 2004/052191 which is an international application by the present applicant. As a result, index formula 13 (see FIG. 43) is obtained among the index formulae having equivalent ability. Indexes which maximize 2-group discriminative ability between (i) the cervical cancer group and the endometrial cancer group and (ii) the cancer-free group with respect to the discrimination of the cervical cancer group, the endometrial cancer group, and the ovarian cancer group are searched for, by linear discriminant analysis (explanatory variable coverage method according to the minimum AIC). As a result, index formula 14 (see FIG. 43) is obtained among the index formulae having equivalent ability. Indexes which maximize 2-group discriminative ability between (i) the cervical cancer group and the endometrial cancer group and (ii) the cancer-free group with respect to the discrimination of the cervical cancer group, the endometrial cancer group, and the ovarian cancer group are searched for, by logistic regression analysis (explanatory variable coverage method according to the minimum AIC). As a result, index formula 15 (see FIG. 43) is obtained among the index formulae having equivalent ability. The value of each coefficient shown in index formulae 13, 14, and 15 may be multiplied by a real number, and the value of each constant term may be subjected to addition, subtraction, multiplication, and division with an arbitrary real constant.

Indexes which maximize 2-group discriminative ability between (i) the cervical cancer group and the endometrial cancer group and (ii) the healthy group with respect to the discrimination of the cervical cancer group, the endometrial cancer group, and the ovarian cancer group are earnestly searched for, by using the method described in International publication WO 2004/052191 which is an international application by the present applicant. As a result, index formula 16 (see FIG. 43) is obtained among the index formulae having equivalent ability. Indexes which maximize 2-group discriminative ability between (i) the cervical cancer group and the endometrial cancer group and (ii) the healthy group with respect to the discrimination of the cervical cancer group, the endometrial cancer group, and the ovarian cancer group are searched for, by linear discriminant analysis (explanatory variable coverage method according to the minimum AIC). As a result, index formula 17 (see FIG. 43) is obtained among the index formulae having equivalent ability. Indexes which maximize 2-group discriminative ability between (i) the cervical cancer group and the endometrial cancer group and (ii) the healthy group with respect to the discrimination of the cervical cancer group, the endometrial cancer group, and the ovarian cancer group are searched for, by logistic regression analysis (explanatory variable coverage method according to the minimum AIC). As a result, index formula 18 (see FIG. 43) is obtained among the index formulae having equivalent ability. The value of each coefficient shown in index formulae 16, 17, and 18 may be multiplied by a real number, and the value of each constant term may be subjected to addition, subtraction, multiplication, and division with an arbitrary real constant.

Indexes which maximize 2-group discriminative ability between (i) the cervical cancer group and the endometrial cancer group and (ii) the benign disease group with respect to the discrimination of the cervical cancer group, the endometrial cancer group, and the ovarian cancer group are earnestly searched for, by using the method described in International publication WO 2004/052191 which is an international application by the present applicant. As a result, index formula 19 (see FIG. 43) is obtained among the index formulae having equivalent ability. Indexes which maximize 2-group discriminative ability between (i) the cervical cancer group and the endometrial cancer group and (ii) the benign disease group with respect to the discrimination of the cervical cancer group, the endometrial cancer group, and the ovarian cancer group are searched for, by linear discriminant analysis (explanatory variable coverage method according to the minimum AIC). As a result, index formula 20 (see FIG. 43) is obtained among the index formulae having equivalent ability. Indexes which maximize 2-group discriminative ability between (i) the cervical cancer group and the endometrial cancer group and (ii) the benign disease group with respect to the discrimination of the cervical cancer group, the endometrial cancer group, and the ovarian cancer group are searched for, by logistic regression analysis (explanatory variable coverage method according to the minimum AIC). As a result, index formula 21 (see FIG. 43) is obtained among the index formulae having equivalent ability. The value of each coefficient shown in index formulae 19, 20, and 21 may be multiplied by a real number, and the value of each constant term may be subjected to addition, subtraction, multiplication, and division with an arbitrary real constant.

To examine the diagnostic ability using index formulae 13 to 15 in the discrimination of the cervical cancer group and the endometrial cancer group, 2-group discrimination between (i) the cervical cancer group and the endometrial cancer group and (ii) the cancer-free group is evaluated by the ROC curve. As a result, the diagnostic ability as shown in FIG. 43 is obtained so that it is found that these index formulae are useful, with high diagnostic ability. As shown in FIG. 43, with regard to these index formulae, an optimum cutoff value, and the sensitivity, specificity, positive predictive value, negative predictive value, and correct answer rate of the used data are calculated.

To examine the diagnostic ability using index formulae 16 to 18 in the discrimination of the cervical cancer group and the endometrial cancer group, 2-group discrimination between (i) the cervical cancer group and the endometrial cancer group and (ii) the healthy group is evaluated by the ROC curve. As a result, the diagnostic ability as shown in FIG. 43 is obtained so that it is found that these index formulae are useful, with high diagnostic ability. As shown in FIG. 43, with regard to these index formulae, an optimum cutoff value, and the sensitivity, specificity, positive predictive value, negative predictive value, and correct answer rate of the used data are calculated.

To examine the diagnostic ability using index formulae 19 to 21 in the discrimination of the cervical cancer group and the endometrial cancer group, 2-group discrimination between (i) the cervical cancer group and the endometrial cancer group and (ii) the benign disease group is evaluated by the ROC curve. As a result, the diagnostic ability as shown in FIG. 43 is obtained so that it is found that these index formulae are useful, with high diagnostic ability. As shown in FIG. 43, with regard to these index formulae, an optimum cutoff value, and the sensitivity, specificity, positive predictive value, negative predictive value, and correct answer rate of the used data are calculated.

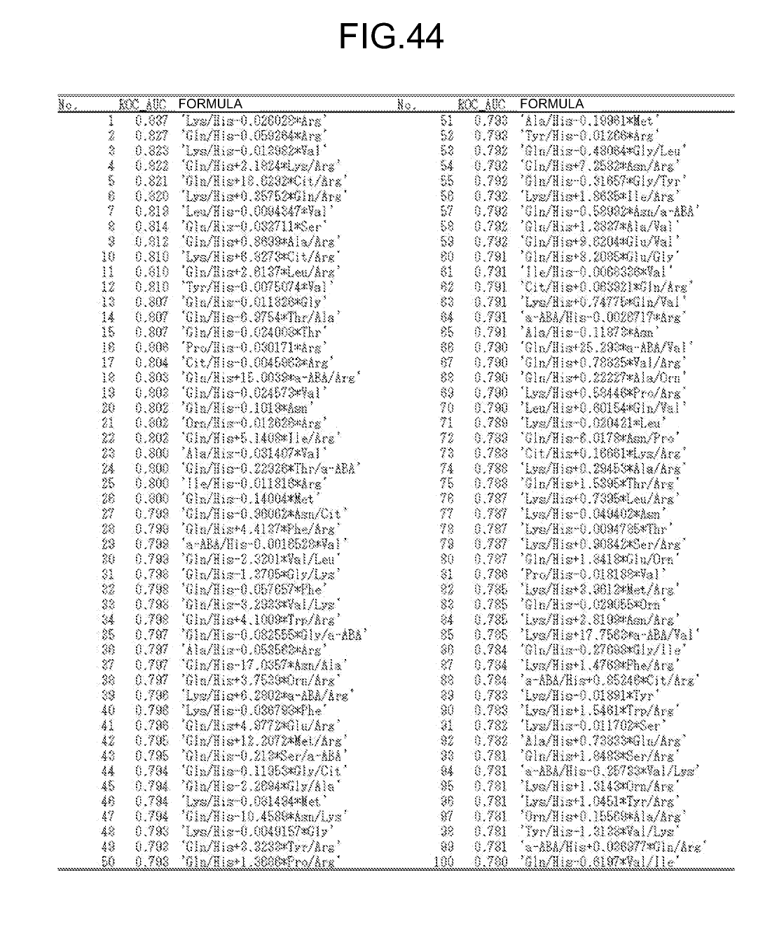

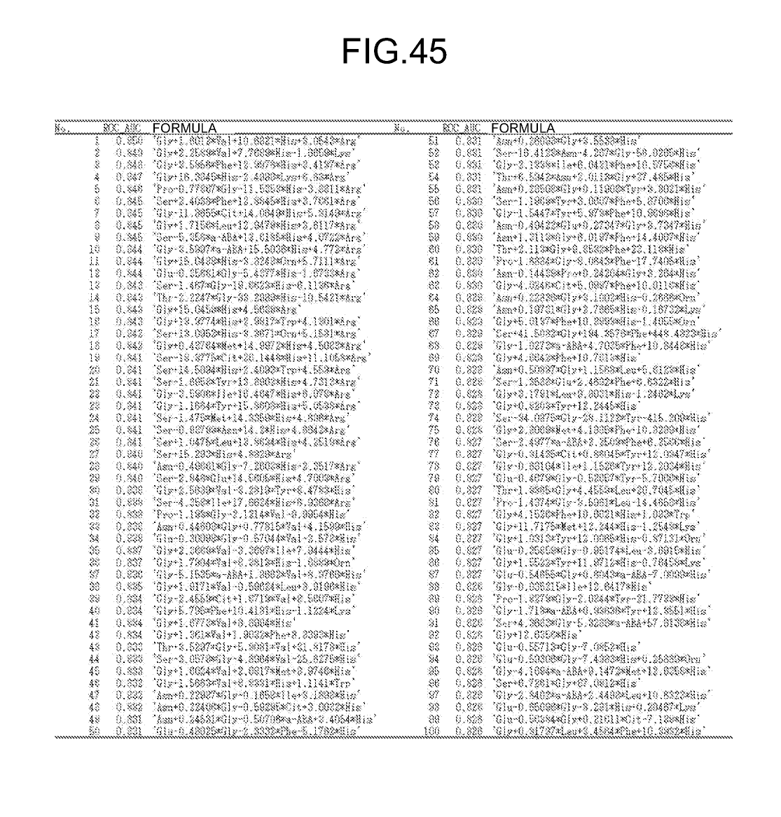

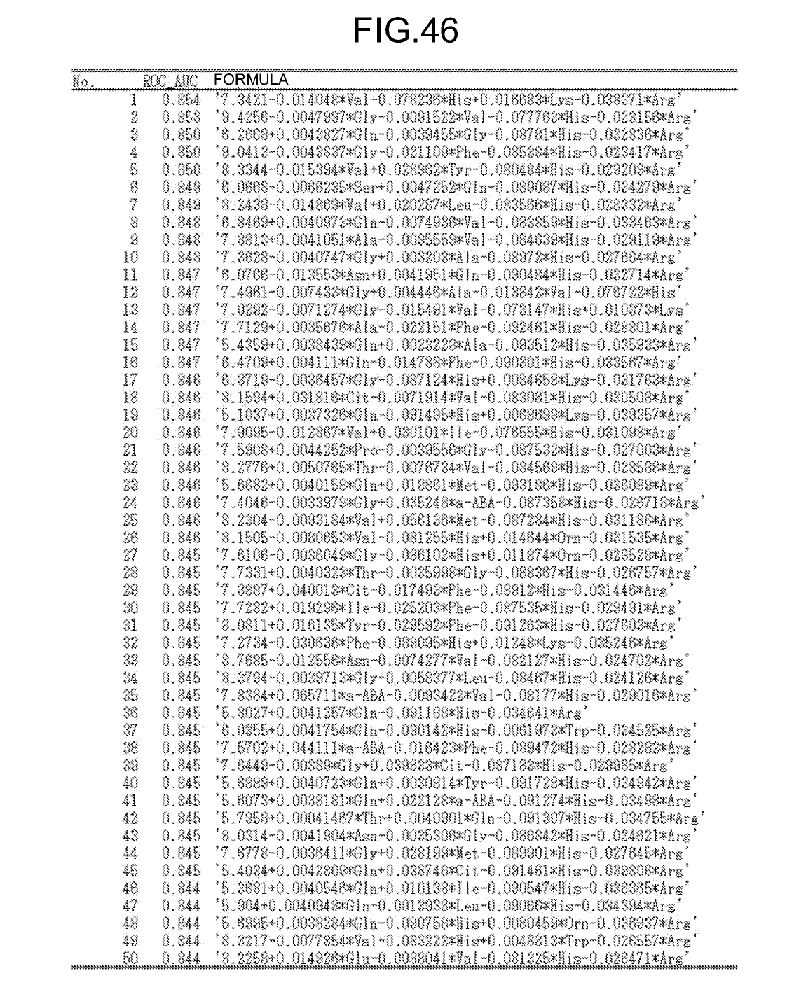

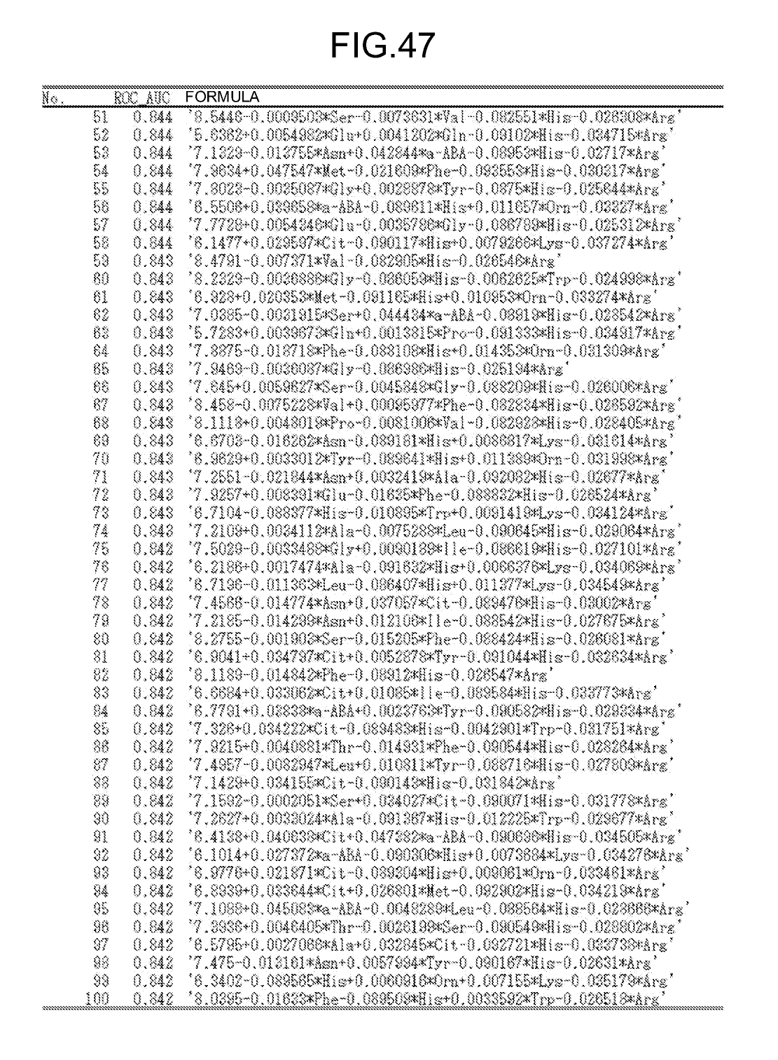

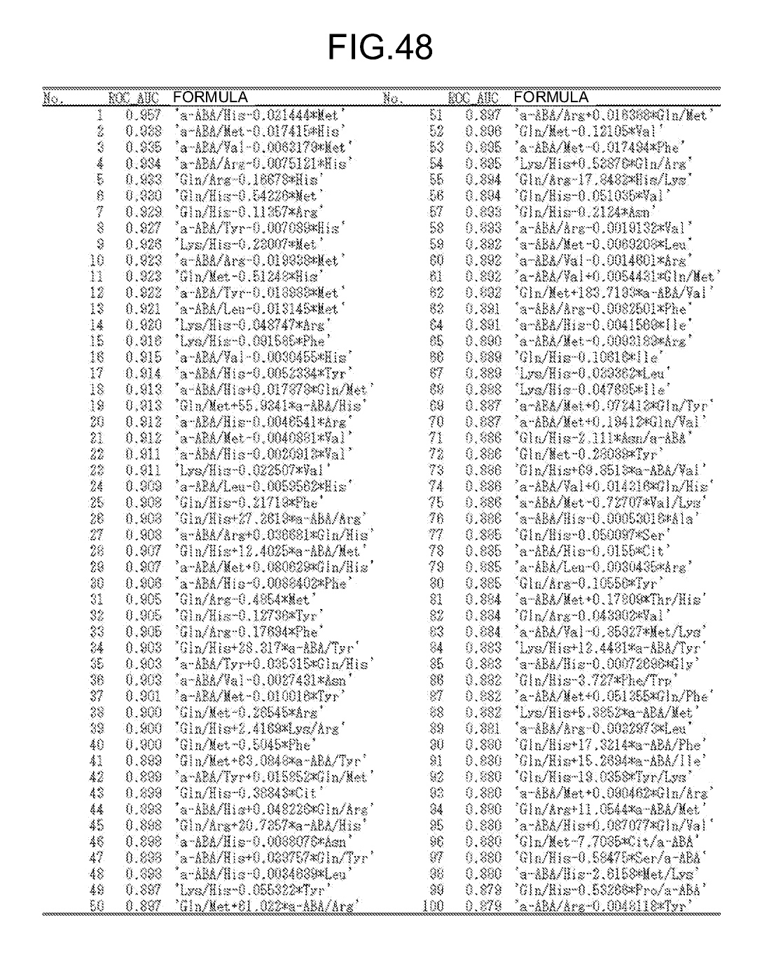

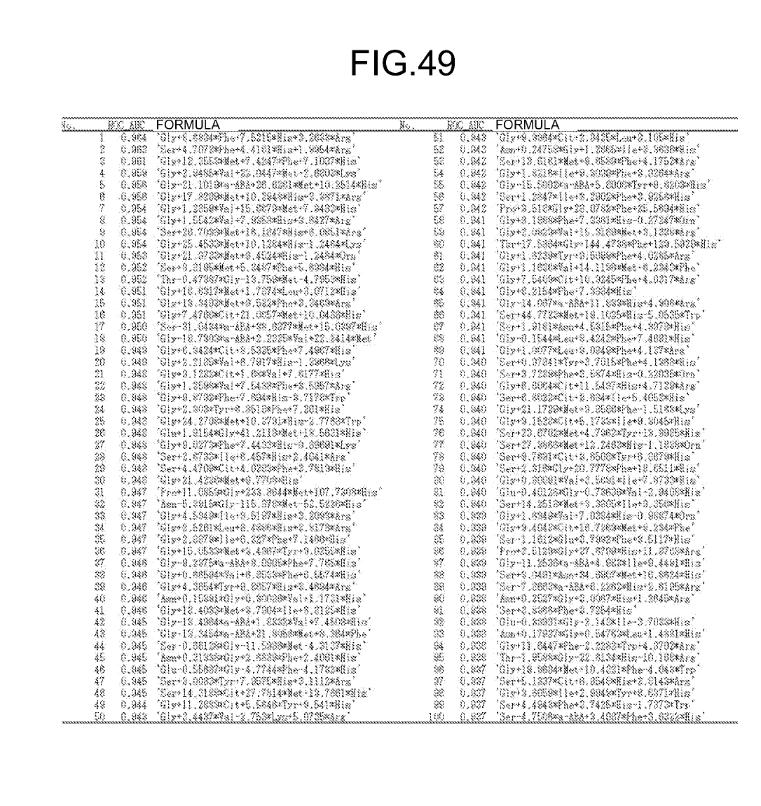

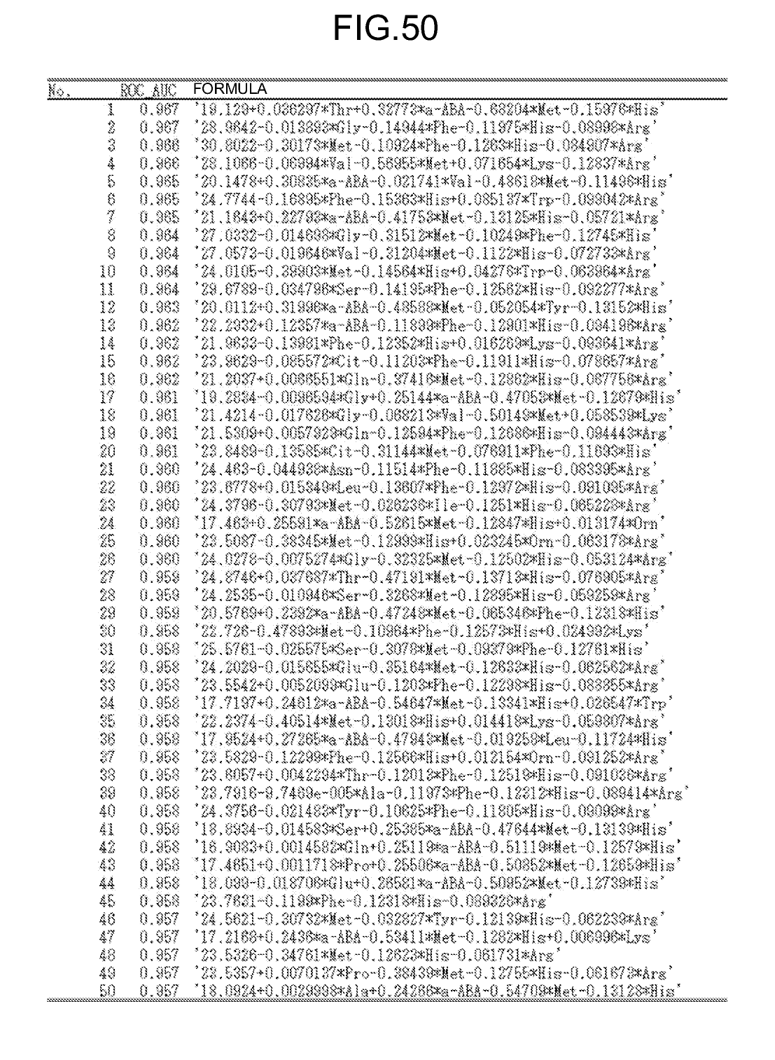

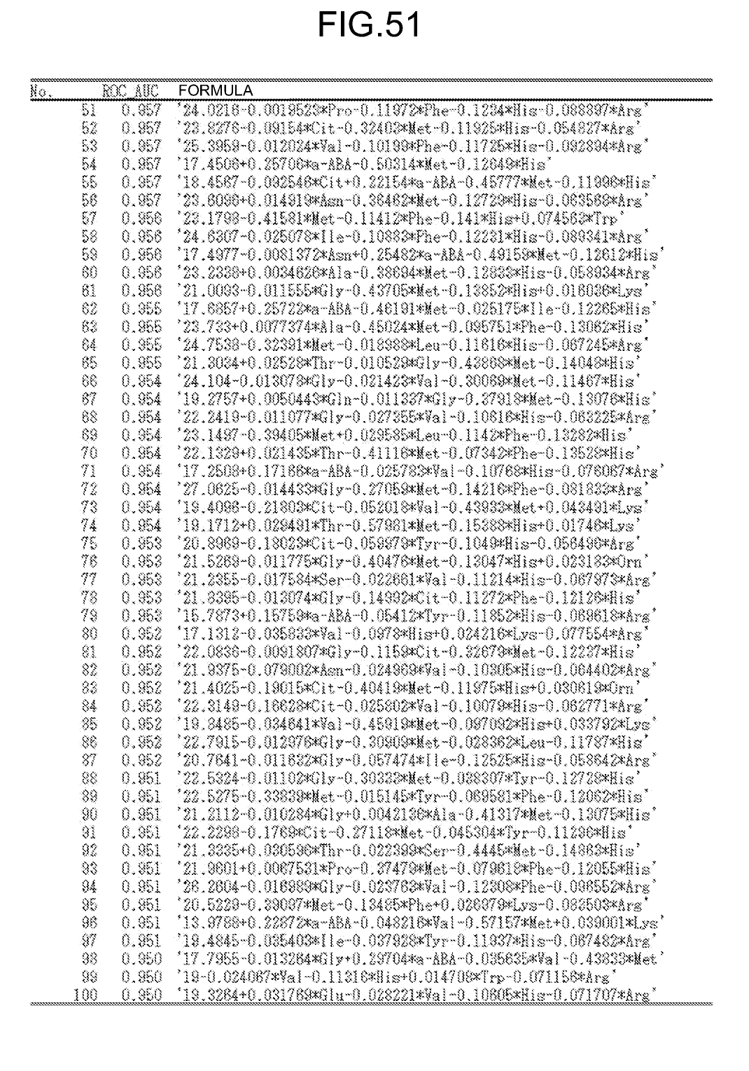

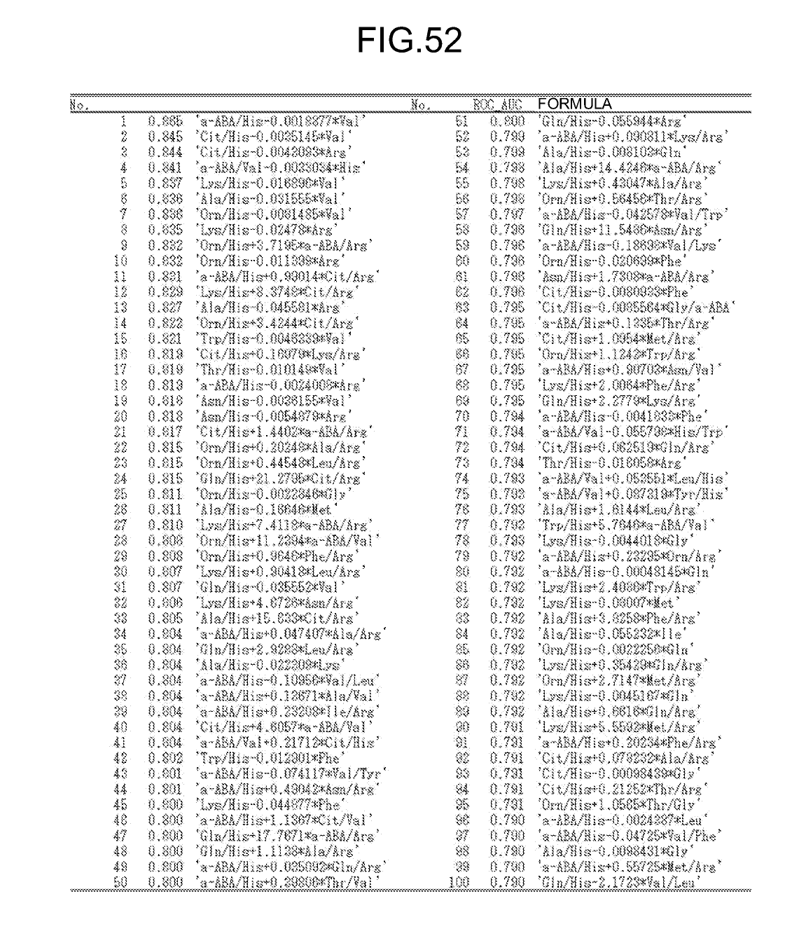

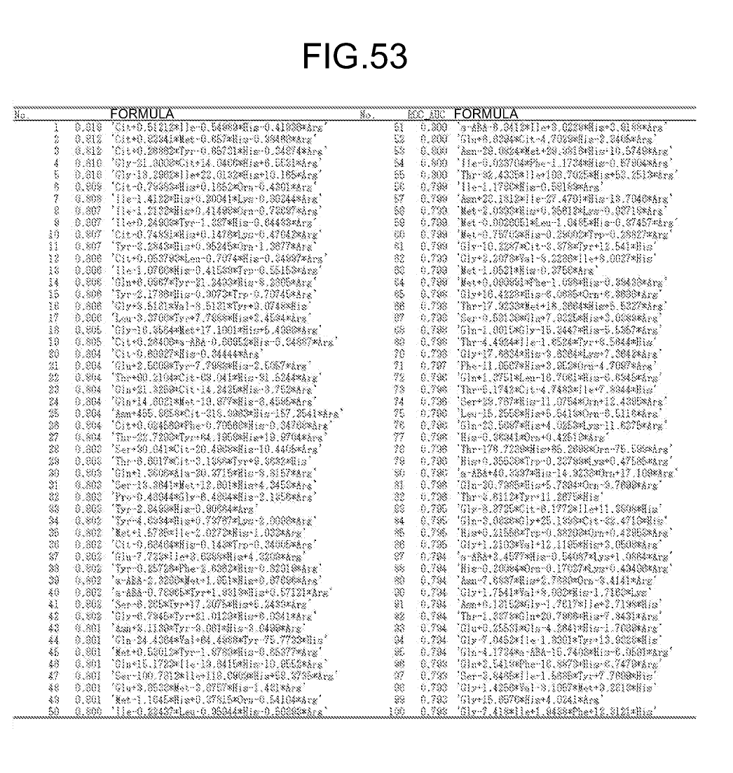

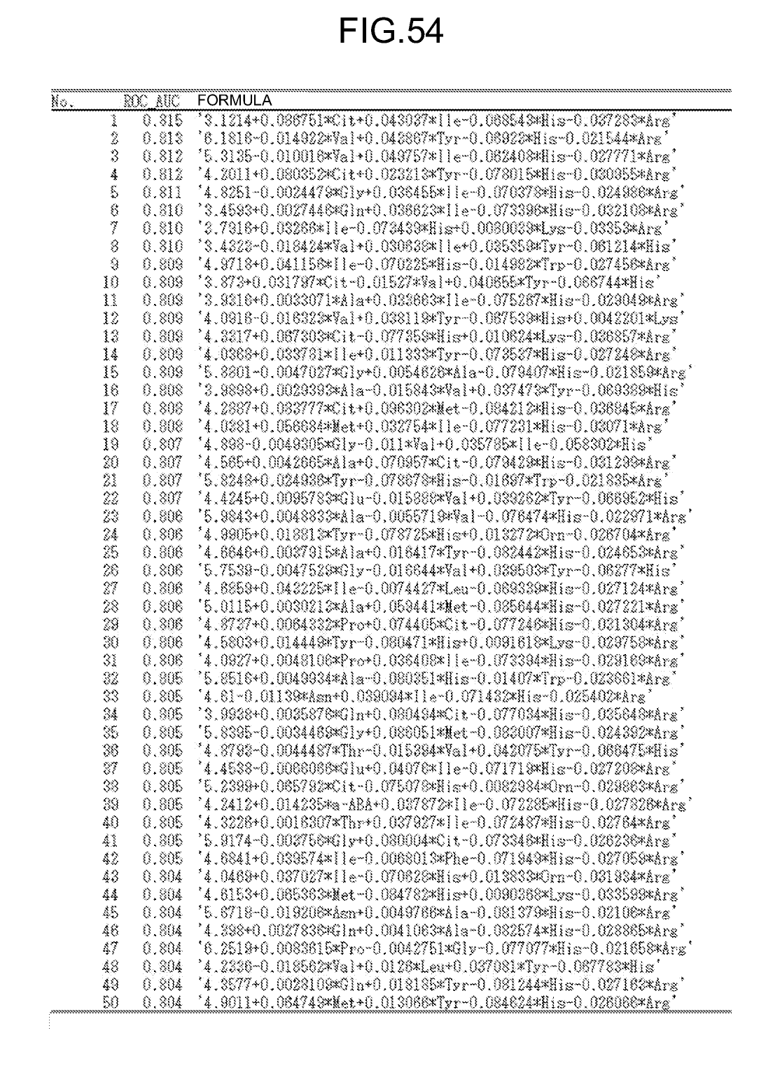

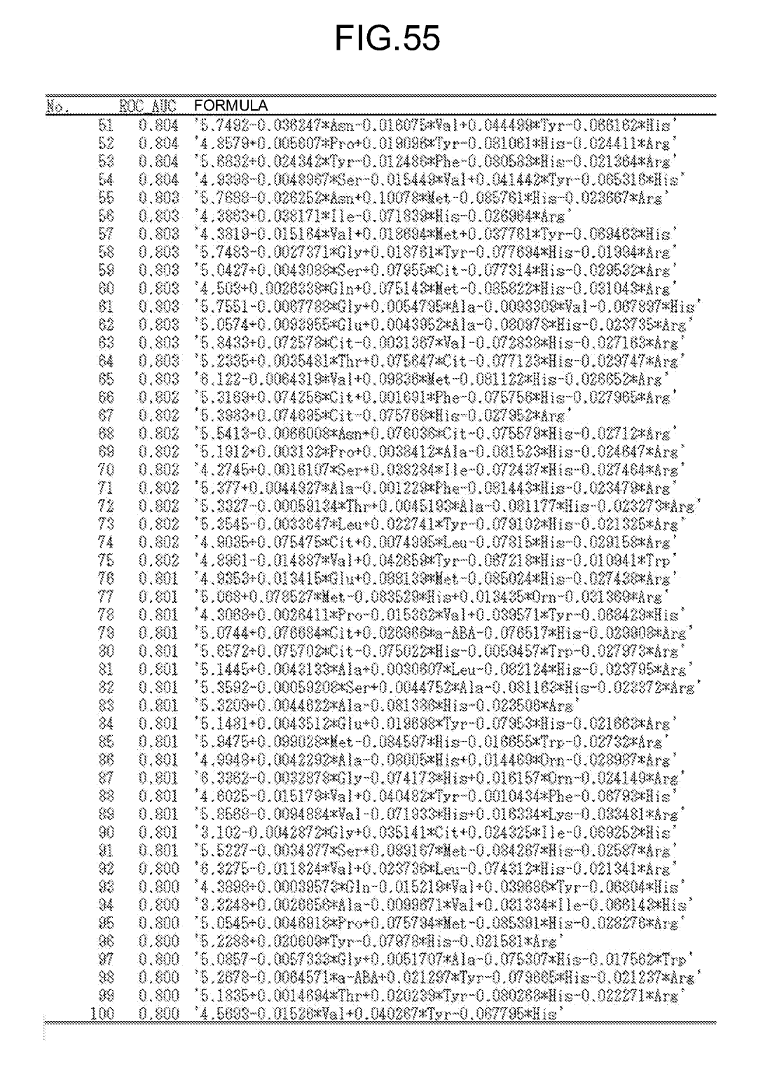

As successively shown in FIGS. 44 to 55, with respect to index formulae 13 to 21, the index formulae having equivalent discriminative ability are obtained. The value of each coefficient in the formulae shown in FIGS. 44 to 55 may be multiplied by a real number, and the value of each constant term may be subjected to addition, subtraction, multiplication, and division with an arbitrary real constant.

### Example 4

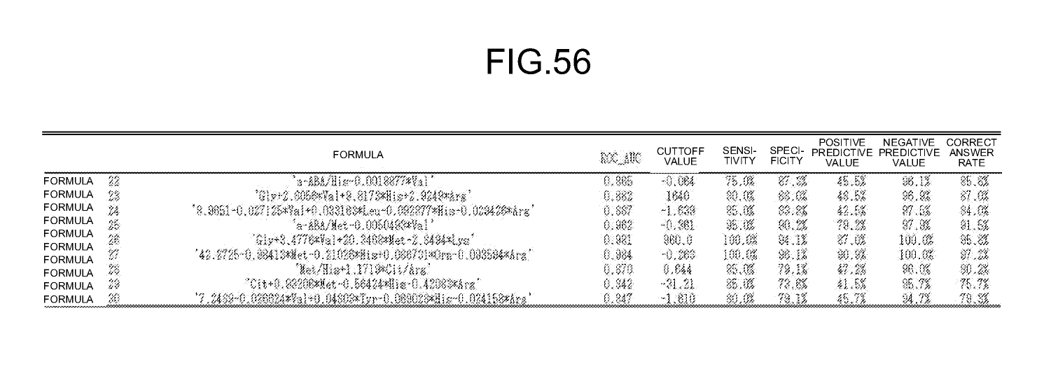

Of the sample data used in Example 1, the data of the cervical cancer group and the cancer-free group are used. Indexes which maximize 2-group discriminative ability between the cervical cancer group and the cancer-free group with respect to the discrimination of the cervical cancer group, the endometrial cancer group, and the ovarian cancer group are earnestly searched for, by using the method described in International publication WO 2004/052191 which is an international application by the present applicant. As a result, index formula 22 (see FIG. 56) is obtained among the index formulae having equivalent ability. Indexes which maximize 2-group discriminative ability between the cervical cancer group and the cancer-free group with respect to the discrimination of the cervical cancer group, the endometrial cancer group, and the ovarian cancer group are searched for, by linear discriminant analysis (explanatory variable coverage method according to the minimum AIC). As a result, index formula 23 (see FIG. 56) is obtained among the index formulae having equivalent ability. Indexes which maximize 2-group discriminative ability between the cervical cancer group and the cancer-free group with respect to the discrimination of the cervical cancer group, the endometrial cancer group, and the ovarian cancer group are searched for, by logistic regression analysis (explanatory variable coverage method according to the minimum AIC). As a result, index formula 24 (see FIG. 56) is obtained among the index formulae having equivalent ability. The value of each coefficient shown in index formulae 22, 23, and 24 may be multiplied by a real number, and the value of each constant term may be subjected to addition, subtraction, multiplication, and division with an arbitrary real constant.

Indexes which maximize 2-group discriminative ability between the cervical cancer group and the healthy group with respect to the discrimination of the cervical cancer group, the endometrial cancer group, and the ovarian cancer group are earnestly searched for, by using the method described in International publication WO 2004/052191 which is an international application by the present applicant. As a result, index formula 25 (see FIG. 56) is obtained among the index formulae having equivalent ability. Indexes which maximize 2-group discriminative ability between the cervical cancer group and the healthy group with respect to the discrimination of the cervical cancer group, the endometrial cancer group, and the ovarian cancer group are searched for, by linear discriminant analysis (explanatory variable coverage method according to the minimum AIC). As a result, index formula 26 (see FIG. 56) is obtained among the index formulae having equivalent ability. Indexes which maximize 2-group discriminative ability between the cervical cancer group and the healthy group with respect to the discrimination of the cervical cancer group, the endometrial cancer group, and the ovarian cancer group are searched for, by logistic regression analysis (explanatory variable coverage method according to the minimum AIC). As a result, index formula 27 (see FIG. 56) is obtained among the index formulae having equivalent ability. The value of each coefficient shown in index formulae 25, 26, and 27 may be multiplied by a real number, and the value of each constant term may be subjected to addition, subtraction, multiplication, and division with an arbitrary real constant.

Indexes which maximize 2-group discriminative ability between the cervical cancer group and the benign disease group with respect to the discrimination of the cervical cancer group, the endometrial cancer group, and the ovarian cancer group are earnestly searched for, by using the method described in International publication WO 2004/052191 which is an international application by the present applicant. As a result, index formula 28 (see FIG. 56) is obtained among the index formulae having equivalent ability. Indexes which maximize 2-group discriminative ability between the cervical cancer group and the benign disease group with respect to the discrimination of the cervical cancer group, the endometrial cancer group, and the ovarian cancer group are searched for, by linear discriminant analysis (explanatory variable coverage method according to the minimum AIC). As a result, index formula 29 (see FIG. 56) is obtained among the index formulae having equivalent ability. Indexes which maximize 2-group discriminative ability between the cervical cancer group and the benign disease group with respect to the discrimination of the cervical cancer group, the endometrial cancer group, and the ovarian cancer group are searched for, by logistic regression analysis (explanatory variable coverage method according to the minimum AIC). As a result, index formula 30 (see FIG. 56) is obtained among the index formulae having equivalent ability. The value of each coefficient in index formulae 28, 29, and 30 may be multiplied by a real number, and the value of each constant term may be subjected to addition, subtraction, multiplication, and division with an arbitrary real constant.

To examine the diagnostic ability using index formulae 22 to 25 in the discrimination of the cervical cancer group, 2-group discrimination between the cervical cancer group and the cancer-free group is evaluated by the ROC curve. As a result, the diagnostic ability as shown in FIG. 56 is obtained so that it is found that these index formulae are useful, with high diagnostic ability. As shown in FIG. 56, with regard to these index formulae, an optimum cutoff value, and the sensitivity, specificity, positive predictive value, negative predictive value, and correct answer rate of the used data are calculated.

To examine the diagnostic ability using index formulae 25 to 27 in the discrimination of the cervical cancer group, 2-group discrimination between the cervical cancer group and the healthy group is evaluated by the ROC curve. As a result, the diagnostic ability as shown in FIG. 56 is obtained so that it is found that these index formulae are useful, with high diagnostic ability. As shown in FIG. 56, with regard to these index formulae, an optimum cutoff value, and the sensitivity, specificity, positive predictive value, negative predictive value, and correct answer rate of the used data are calculated.

To examine the diagnostic ability using index formulae 28 to 30 in the discrimination of the cervical cancer group, 2-group discrimination between the cervical cancer group and the benign disease group is evaluated by the ROC curve. As a result, the diagnostic ability as shown in FIG. 56 is obtained so that it is found that these index formulae are useful, with high diagnostic ability. As shown in FIG. 56, with regard to these index formulae, an optimum cutoff value, and the sensitivity, specificity, positive predictive value, negative predictive value, and correct answer rate of the used data are calculated.

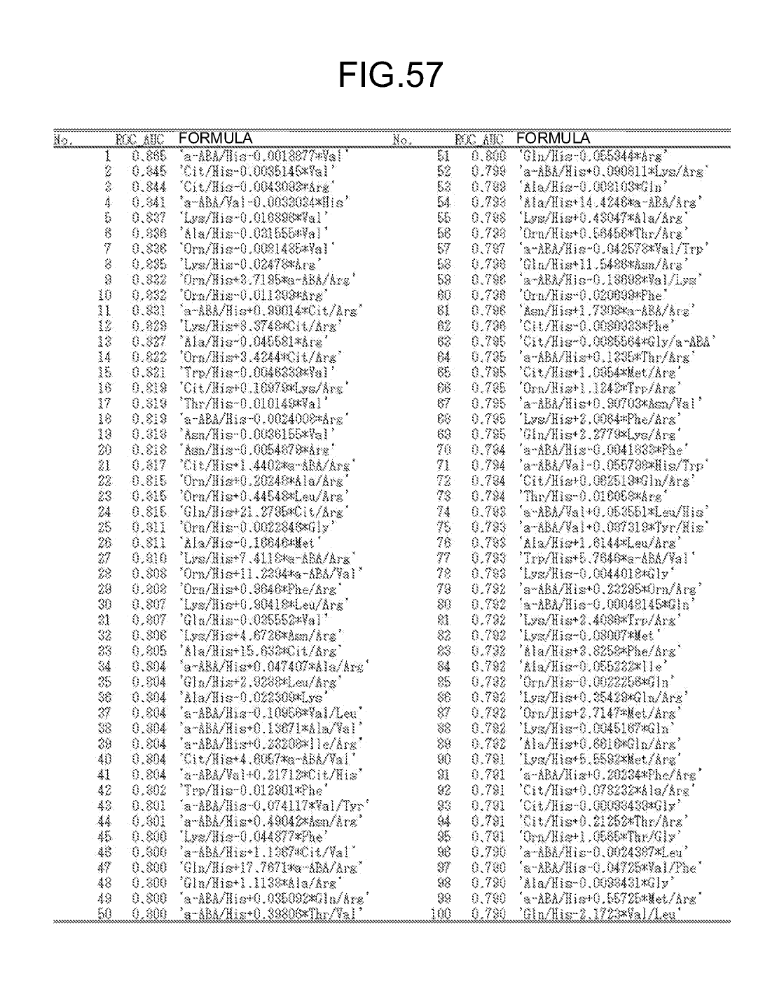

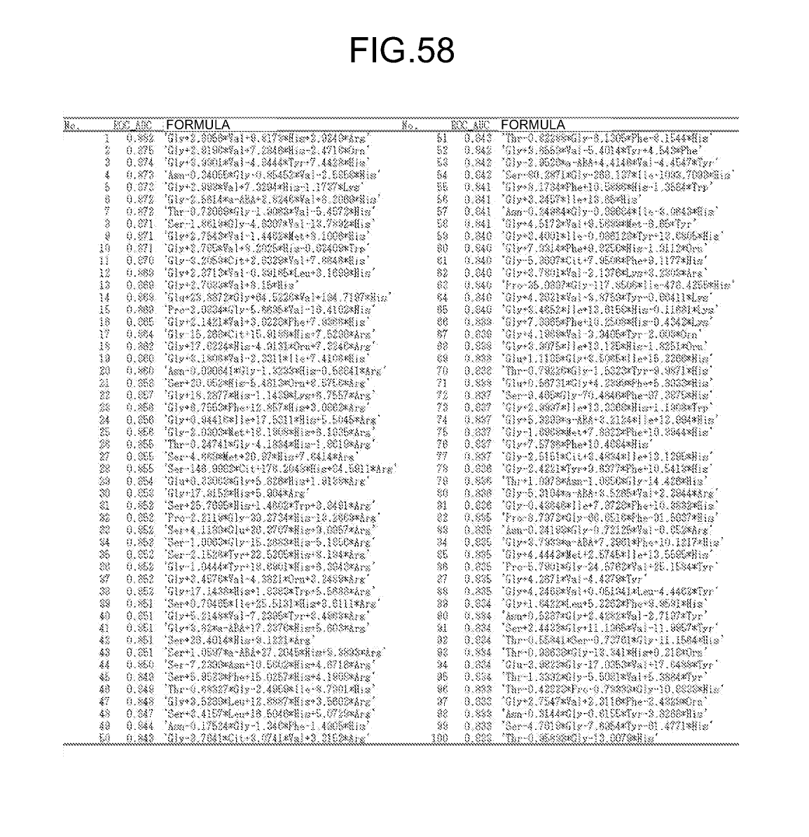

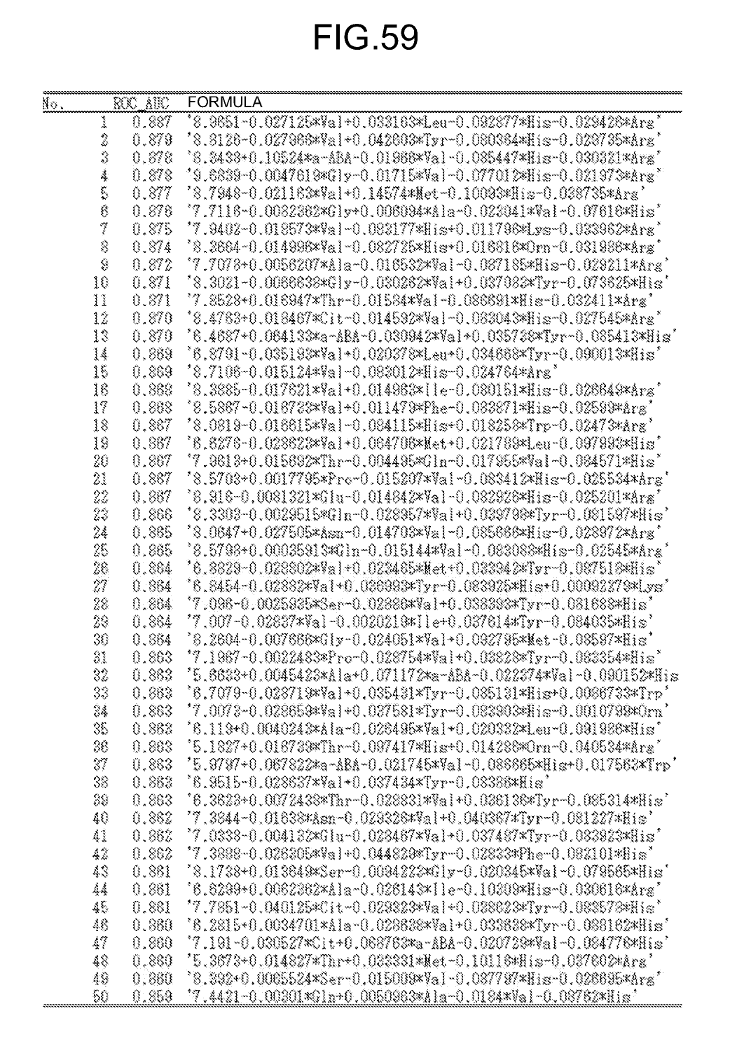

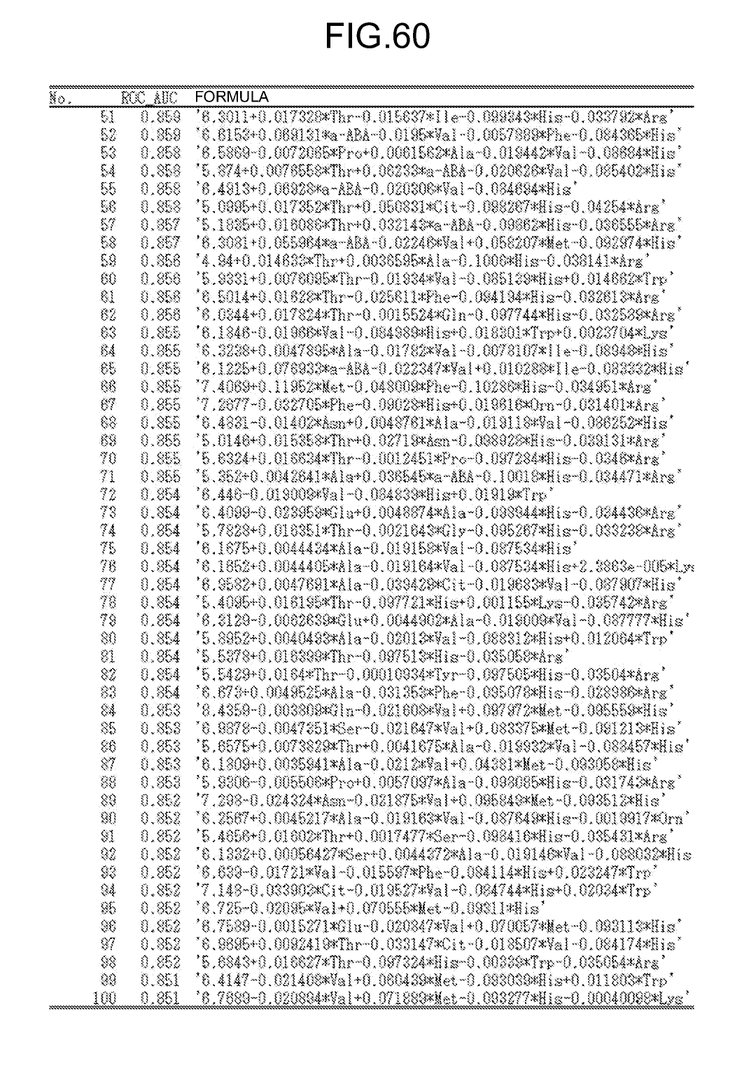

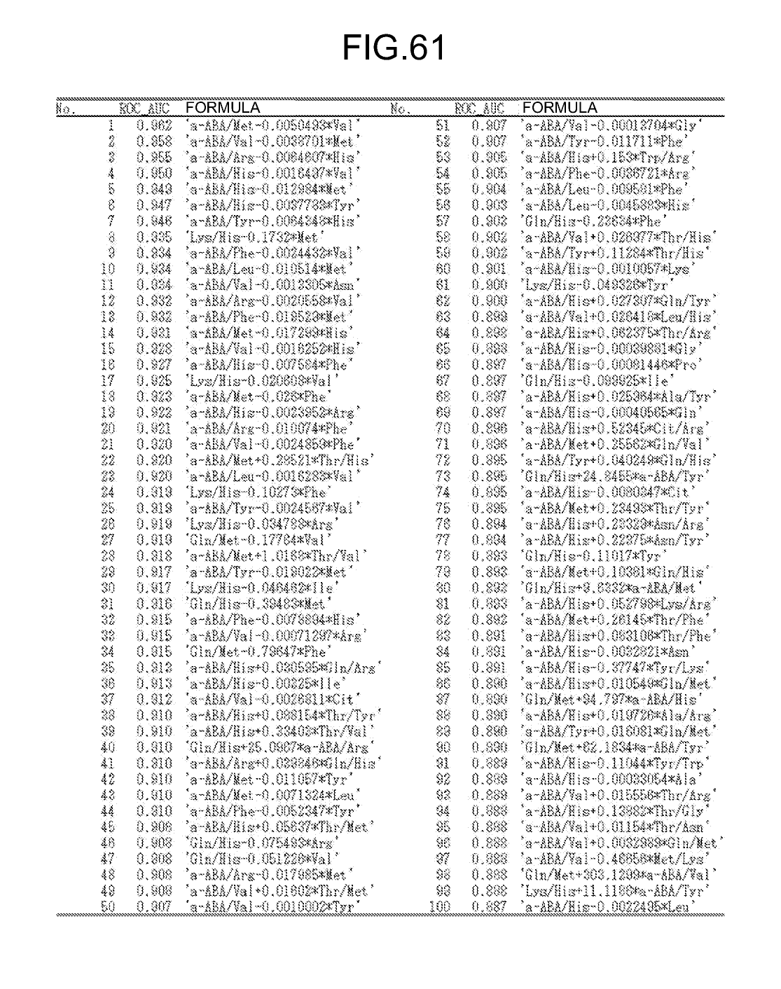

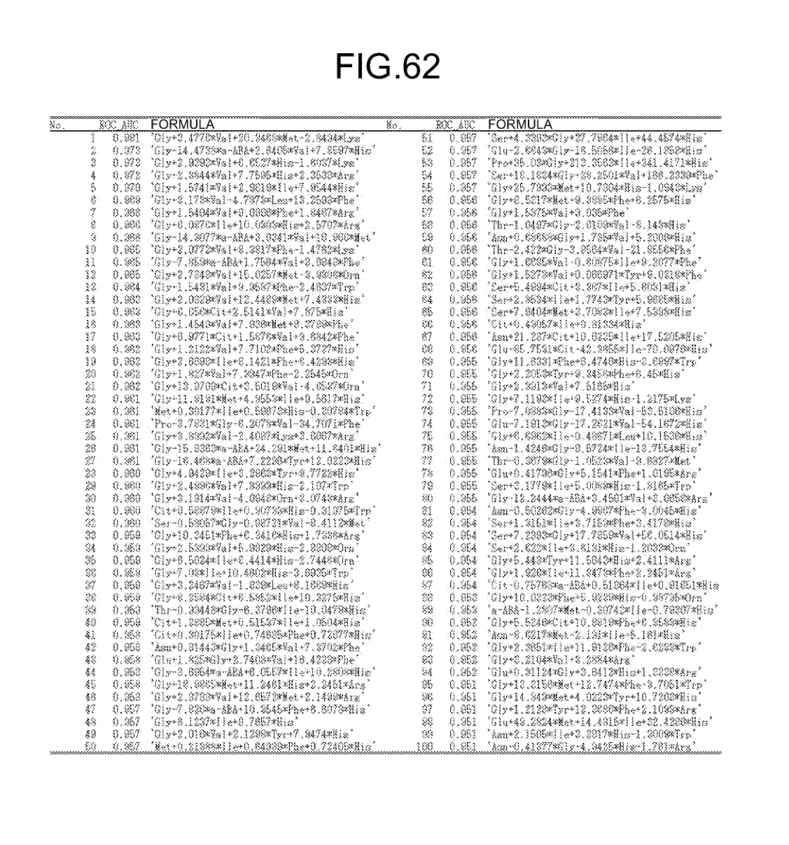

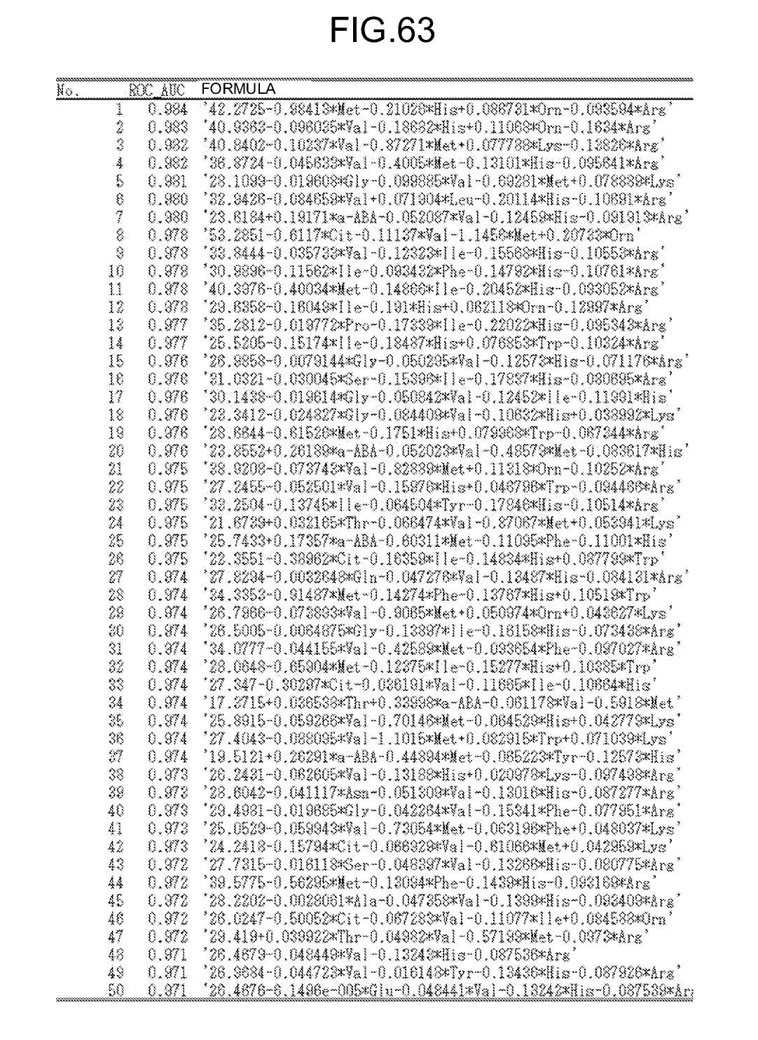

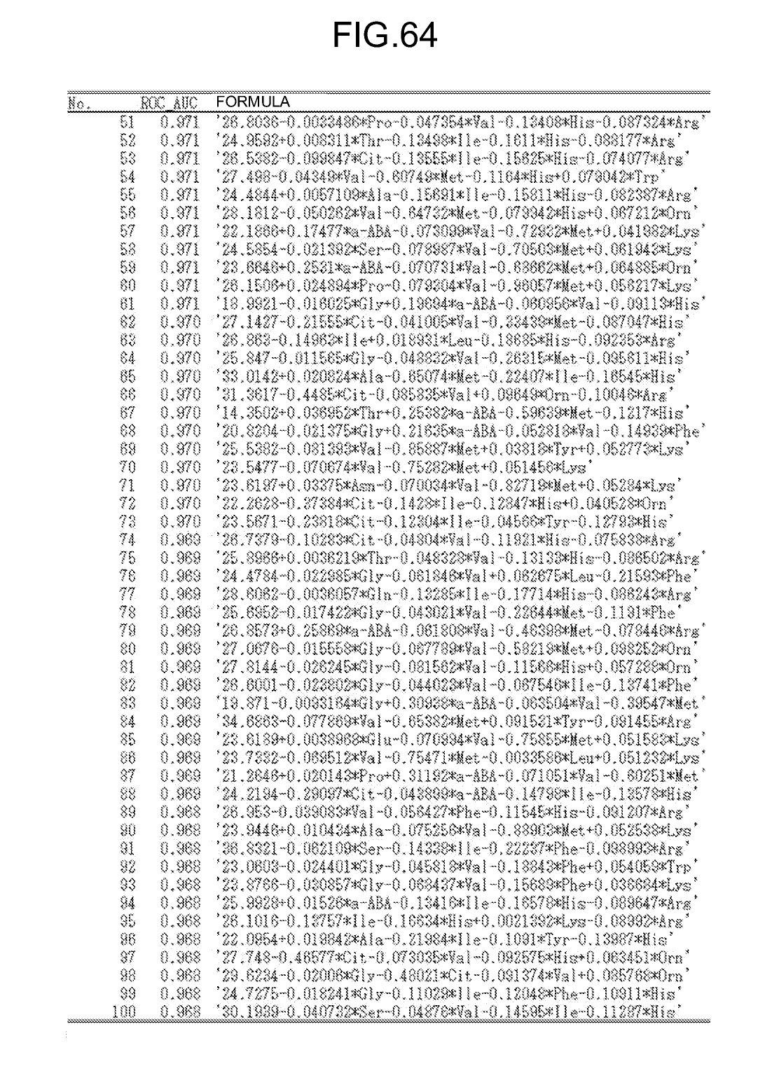

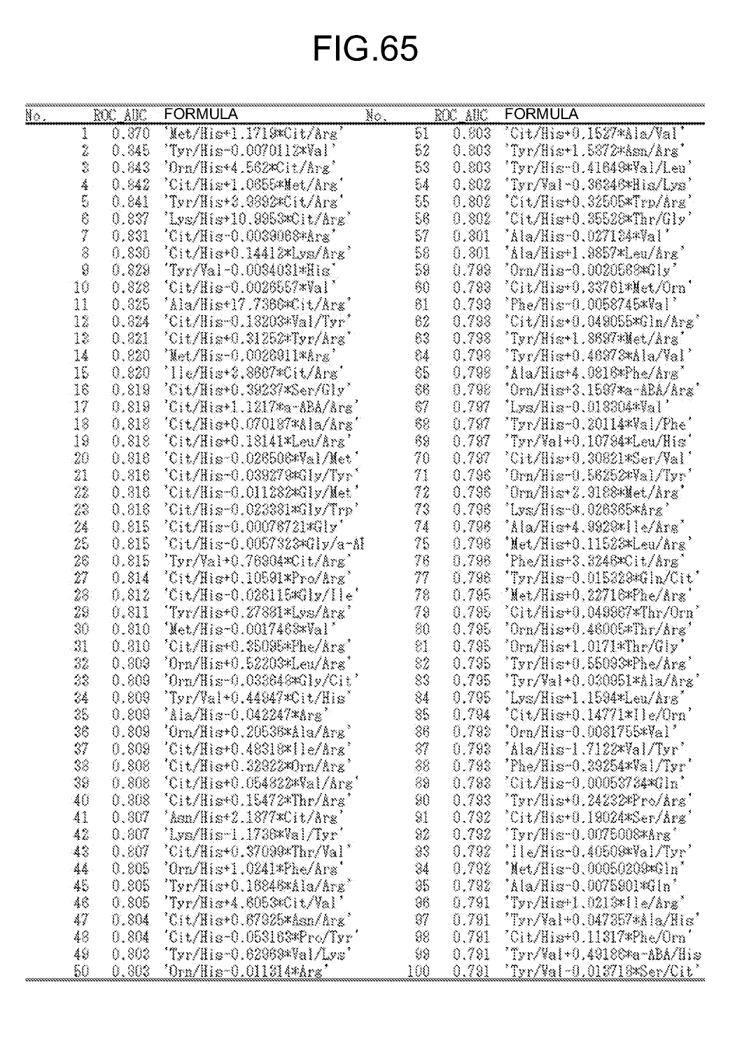

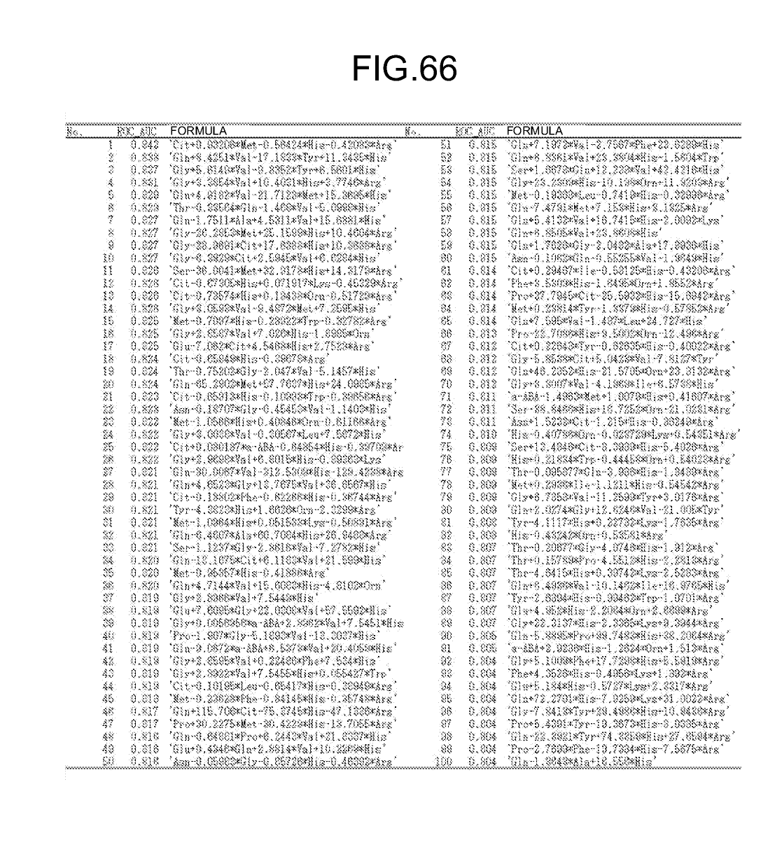

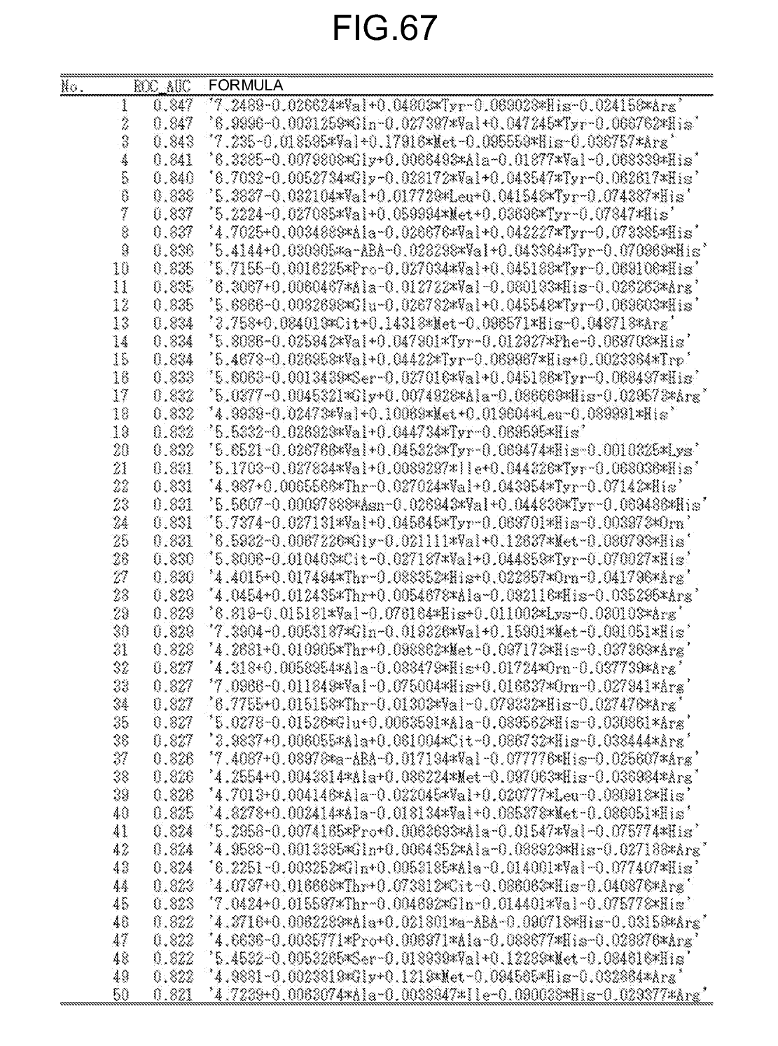

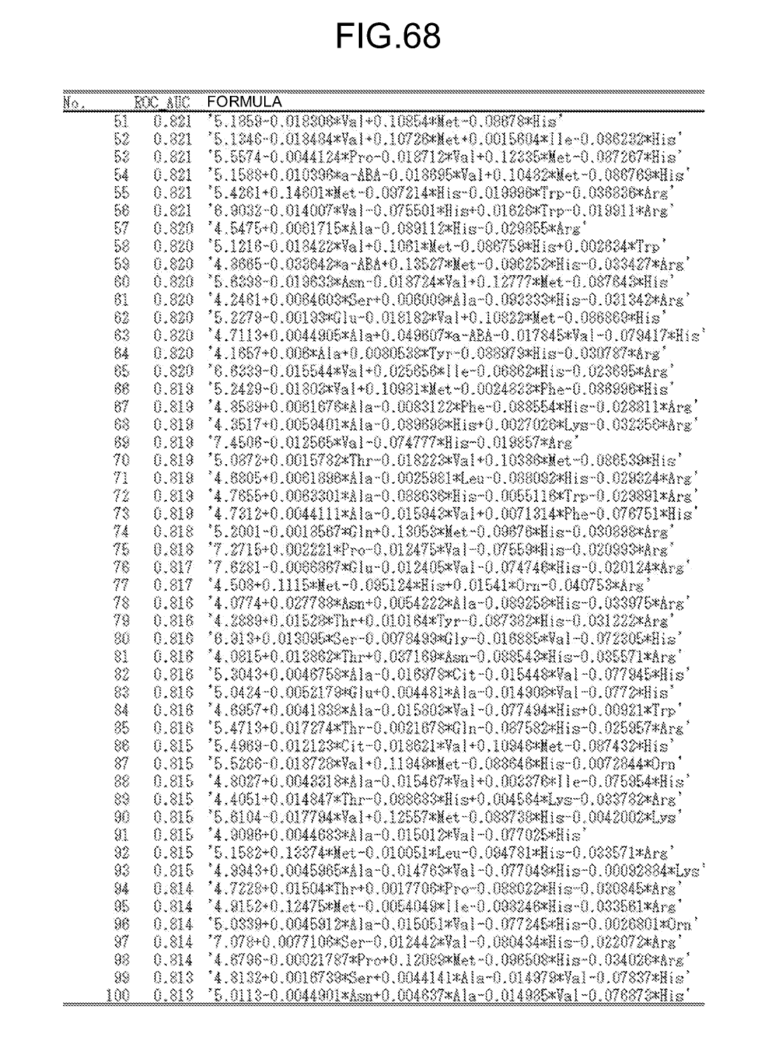

As successively shown in FIGS. 57 to 68, with respect to index formulae 22 to 30, the index formulae having equivalent discriminative ability are obtained. The value of each coefficient in the formulae shown in FIGS. 57 to 68 may be multiplied by a real number, and the value of each constant term may be subjected to addition, subtraction, multiplication, and division with an arbitrary real constant.

### Example 5

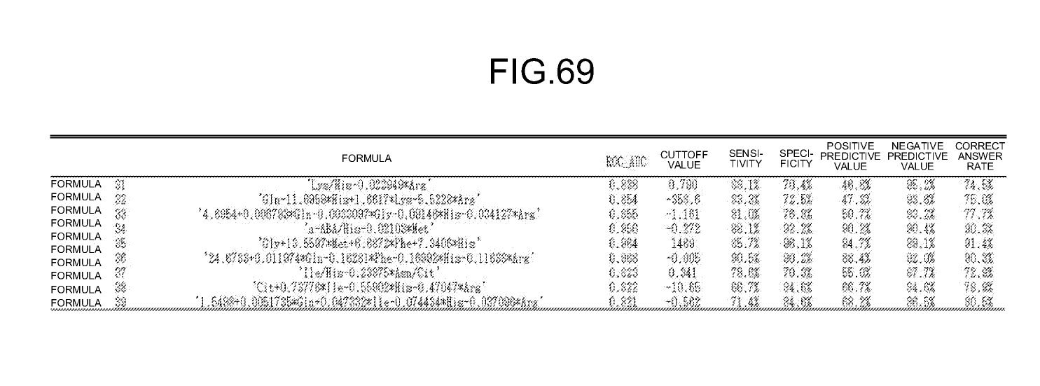

Of the sample data used in Example 1, the data of the endometrial cancer group and the cancer-free group are used. Indexes which maximize 2-group discriminative ability between the endometrial cancer group and the cancer-free group with respect to the discrimination in the cervical cancer group, the endometrial cancer group, and the ovarian cancer group are earnestly searched for, by using the method described in International publication WO 2004/052191 which is an international application by the present applicants. As a result, index formula 31 (see FIG. 69) is obtained among the index formulae having equivalent ability. Indexes which maximize 2-group discriminative ability between the endometrial cancer group and the cancer-free group with respect to the discrimination in the cervical cancer group, the endometrial cancer group, and the ovarian cancer group are searched for, by linear discriminant analysis (explanatory variable coverage method according to the minimum AIC). As a result, index formula 32 (see FIG. 69) is obtained among the index formulae having equivalent ability. Indexes which maximize 2-group discriminative ability between the endometrial cancer group and the cancer-free group with respect to the discrimination in the cervical cancer group, the endometrial cancer group, and the ovarian cancer group are searched for, by logistic regression analysis (explanatory variable coverage method according to the minimum AIC). As a result, index formula 33 (see FIG. 69) is obtained among the index formulae having equivalent ability. The value of each coefficient in index formulae 31, 32, and 33 may be multiplied by a real number, and the value of each constant term may be subjected to addition, subtraction, multiplication, and division with an arbitrary real constant.

Indexes which maximize 2-group discriminative ability between the endometrial cancer group and the healthy group with respect to the discrimination in the cervical cancer group, the endometrial cancer group, and the ovarian cancer group are earnestly searched for, by using the method described in International publication WO 2004/052191 which is an international application by the present applicant. As a result, index formula 34 (see FIG. 69) is obtained among the index formulae having equivalent ability. Indexes which maximize 2-group discriminative ability between the endometrial cancer group and the healthy group with respect to the discrimination in the cervical cancer group, the endometrial cancer group, and the ovarian cancer group are searched for, by linear discriminant analysis (explanatory variable coverage method according to the minimum AIC). As a result, index formula 35 (see FIG. 69) is obtained among the index formulae having equivalent ability. Indexes which maximize 2-group discriminative ability between the endometrial cancer group and the healthy group with respect to the discrimination in the cervical cancer group, the endometrial cancer group, and the ovarian cancer group are searched for, by logistic regression analysis (explanatory variable coverage method according to the minimum AIC). As a result, index formula 36 (see FIG. 69) is obtained among the index formulae having equivalent ability. The value of each coefficient in index formulae 34, 35, and 36 may be multiplied by a real number, and the value of each constant term may be subjected to addition, subtraction, multiplication, and division with an arbitrary real constant.

Indexes which maximize 2-group discriminative ability between the endometrial cancer group and the benign disease group with respect to the discrimination in the cervical cancer group, the endometrial cancer group, and the ovarian cancer group are earnestly searched for, by using the method described in International publication WO 2004/052191 which is an international application by the present applicant. As a result, index formula 37 (see FIG. 69) is obtained among the index formulae having equivalent ability. Indexes which maximize 2-group discriminative ability between the endometrial cancer group and the benign disease group with respect to the discrimination in the cervical cancer group, the endometrial cancer group, and the ovarian cancer group are searched for, by linear discriminant analysis (explanatory variable coverage method according to the minimum AIC). As a result, index formula 38 (see FIG. 69) is obtained among the index formulae having equivalent ability. Indexes which maximize 2-group discriminative ability between the endometrial cancer group and the benign disease group with respect to the discrimination in the cervical cancer group, the endometrial cancer group, and the ovarian cancer group are searched for, by logistic regression analysis (explanatory variable coverage method according to the minimum AIC). As a result, index formula 39 (see FIG. 69) is obtained among the index formulae having equivalent ability. The value of each coefficient in index formulae 37, 38, and 39 may be multiplied by a real number, and the value of each constant term may be subjected to addition, subtraction, multiplication, and division with an arbitrary real constant.

To examine the diagnostic ability using index formulae 31 to 33 in the discrimination of the endometrial cancer group, 2-group discrimination between the endometrial cancer group and the cancer-free group is evaluated by the ROC curve. As a result, the diagnostic ability as shown in FIG. 69 is obtained so that it is found that these index formulae are useful, with high diagnostic ability. As shown in FIG. 69, with regard to these index formulae, an optimum cutoff value, and the sensitivity, specificity, positive predictive value, negative predictive value, and correct answer rate of the used data are calculated.

To examine the diagnostic ability using index formulae 34 to 36 in the discrimination of the endometrial cancer group, 2-group discrimination between the endometrial cancer group and the healthy group is evaluated by the ROC curve. As a result, the diagnostic ability as shown in FIG. 69 is obtained so that it is found that these index formulae are useful, with high diagnostic ability. As shown in FIG. 69, with regard to these index formulae, an optimum cutoff value, and the sensitivity, specificity, positive predictive value, negative predictive value, and correct answer rate of the used data are calculated.

To examine the diagnostic ability using index formulae 37 to 39 in the discrimination of the endometrial cancer group, 2-group discrimination between the endometrial cancer group and the benign disease group is evaluated by the ROC curve. As a result, the diagnostic ability as shown in FIG. 69 is obtained so that it is found that these index formulae are useful, with high diagnostic ability. As shown in FIG. 69, with regard to these index formulae, an optimum cutoff value, and the sensitivity, specificity, positive predictive value, negative predictive value, and correct answer rate of the used data are calculated.

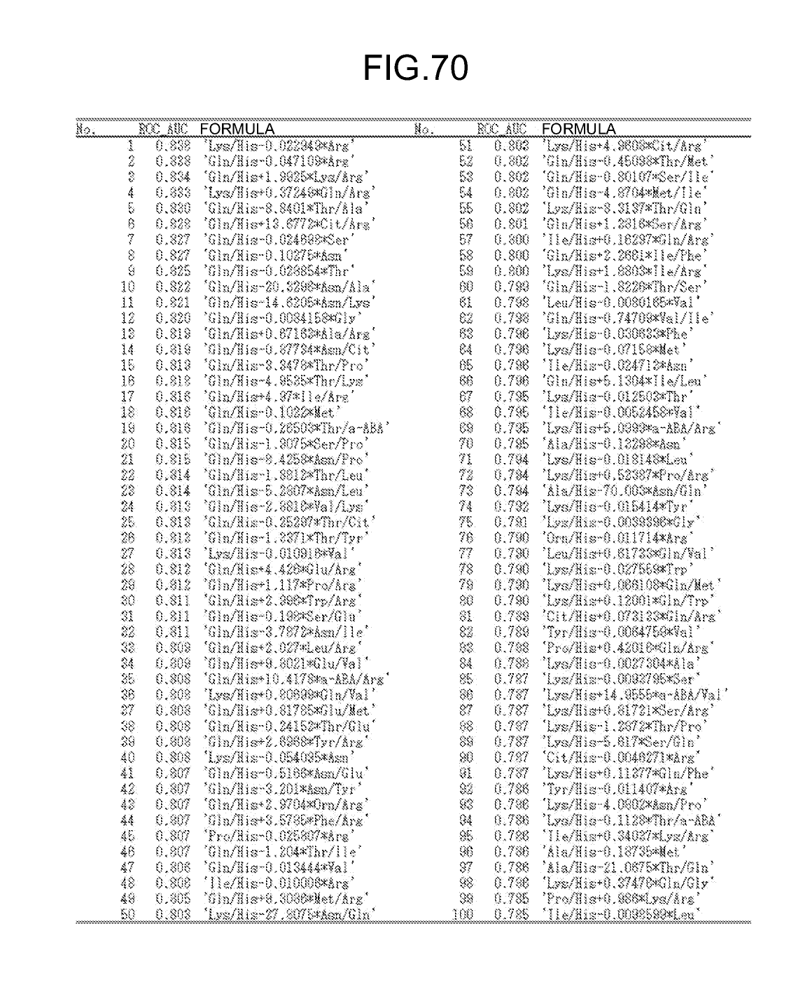

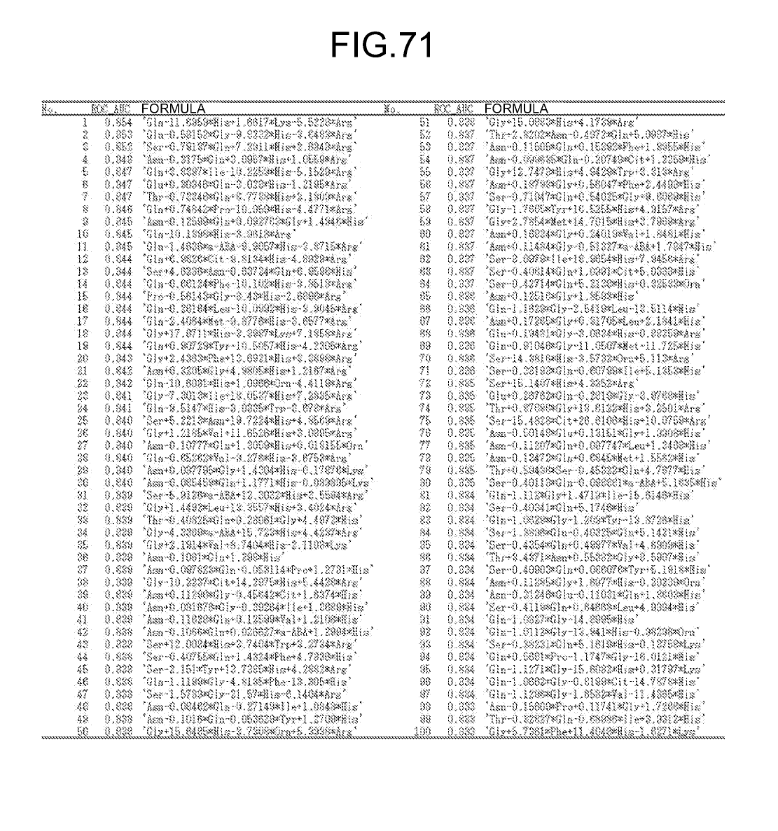

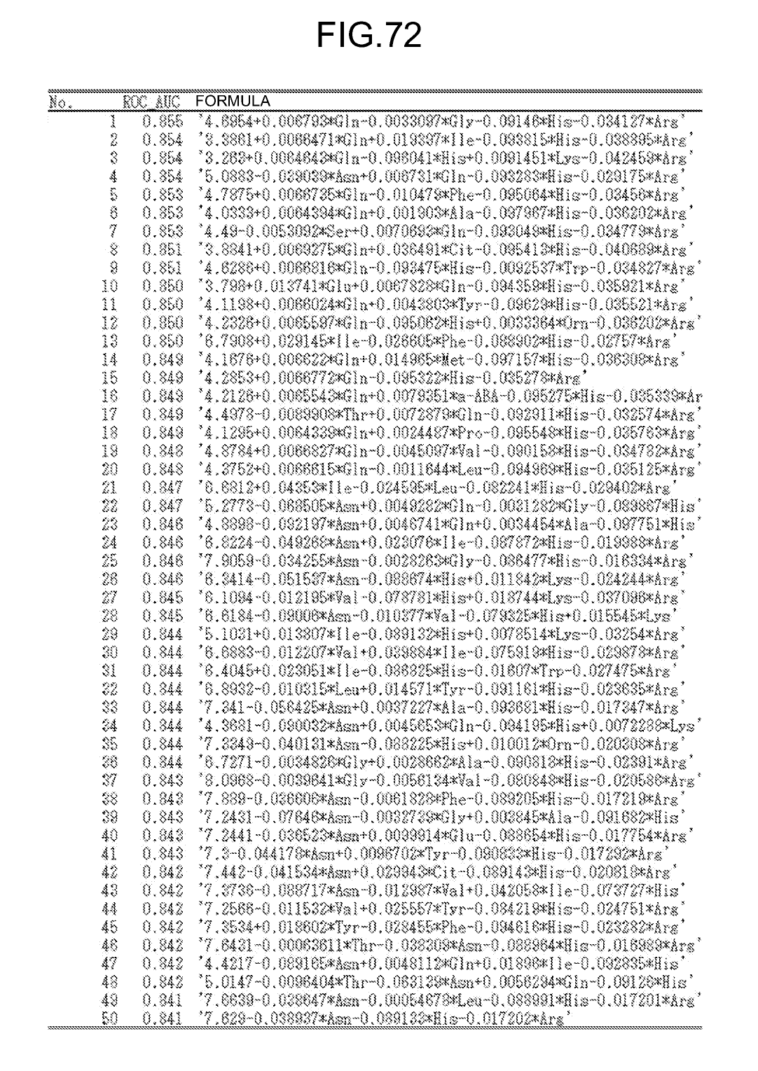

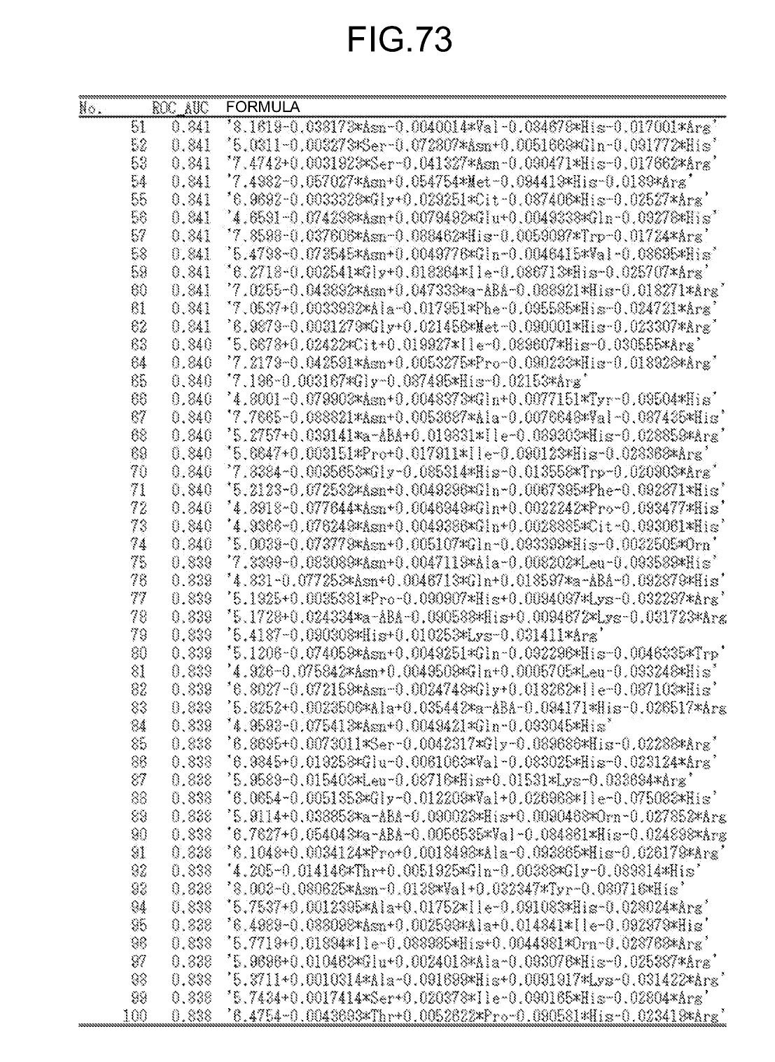

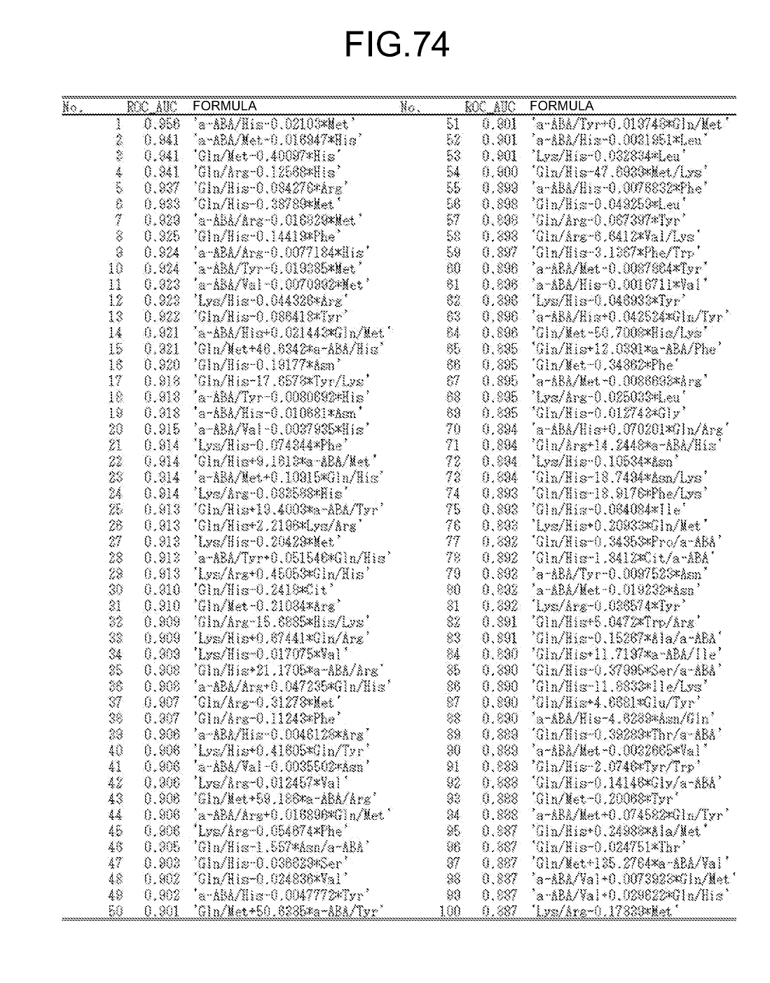

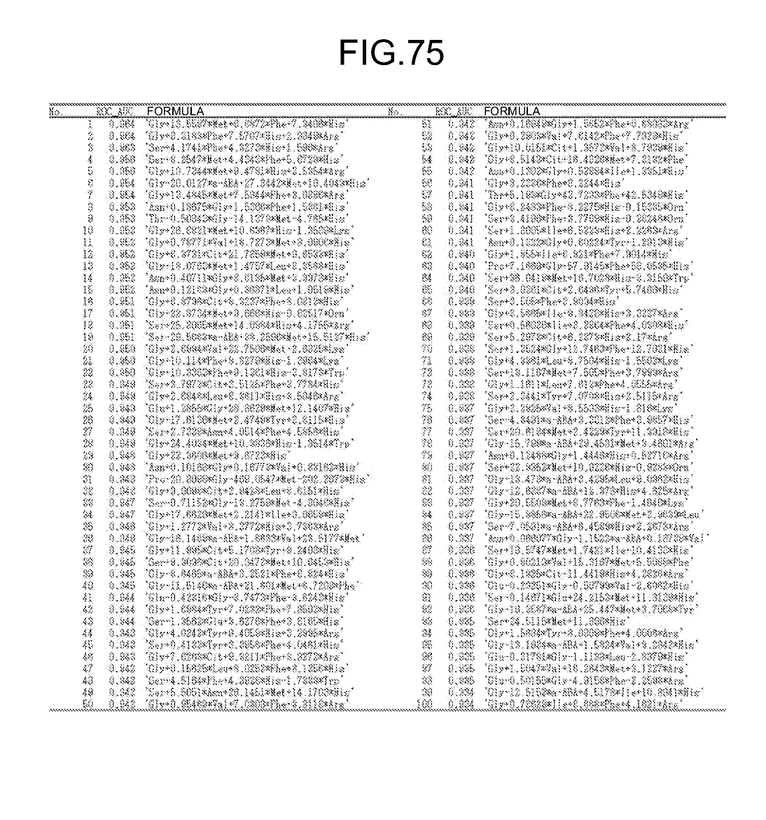

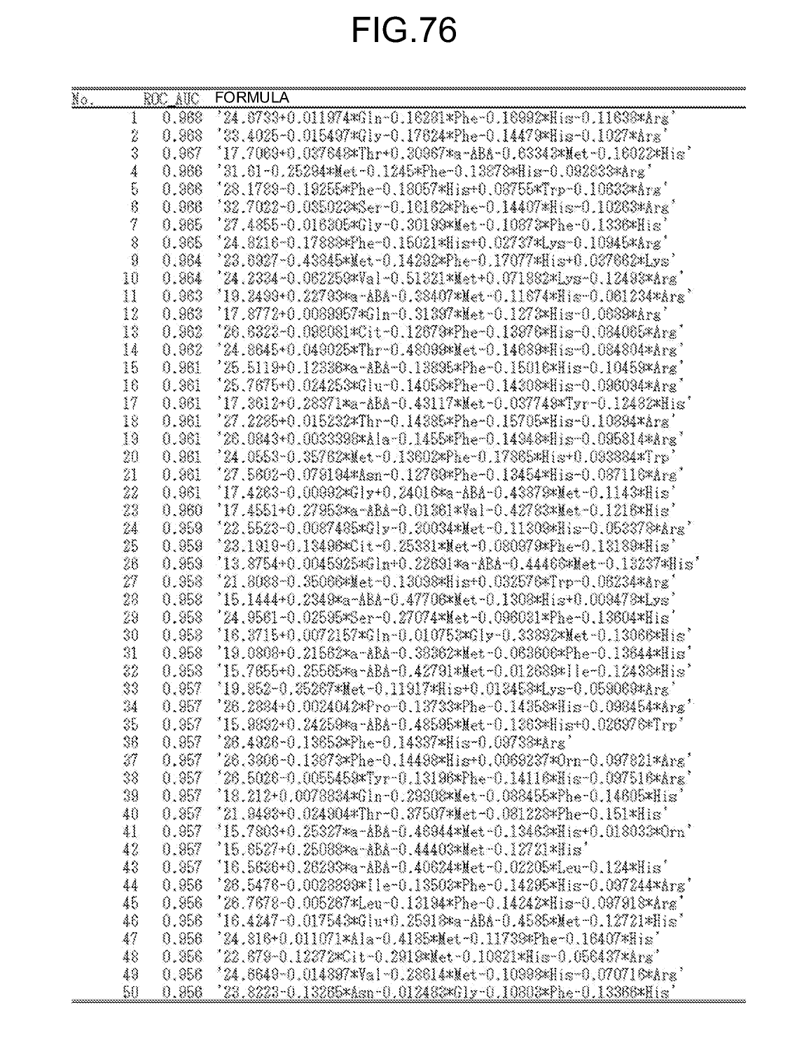

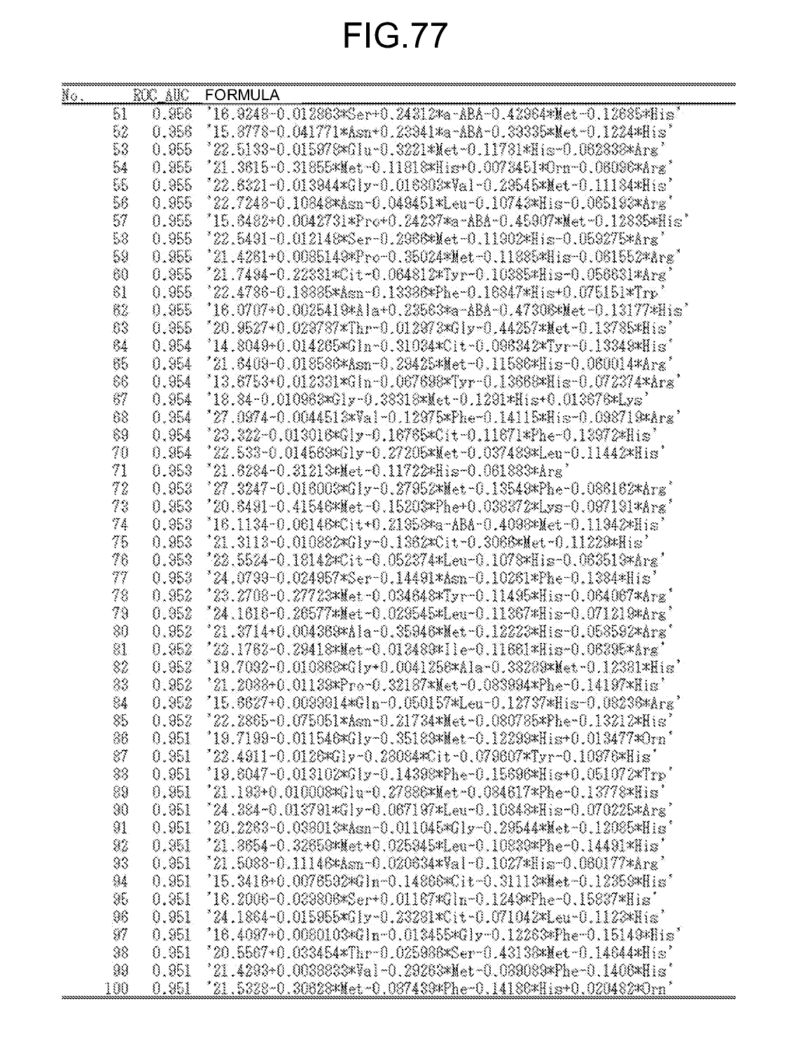

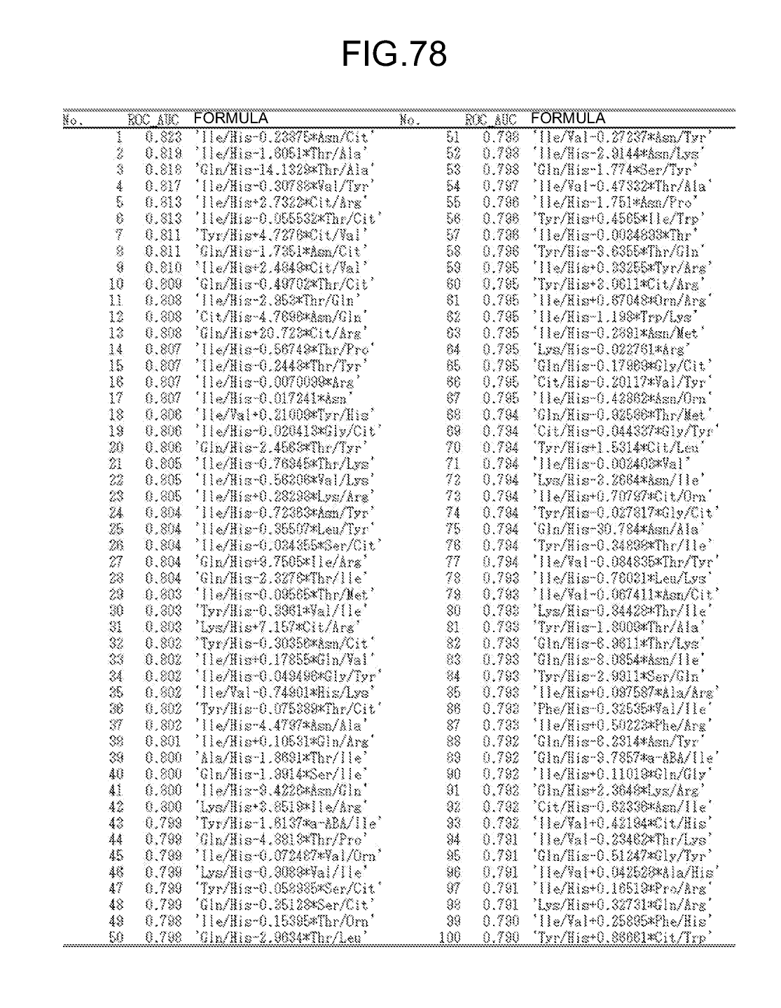

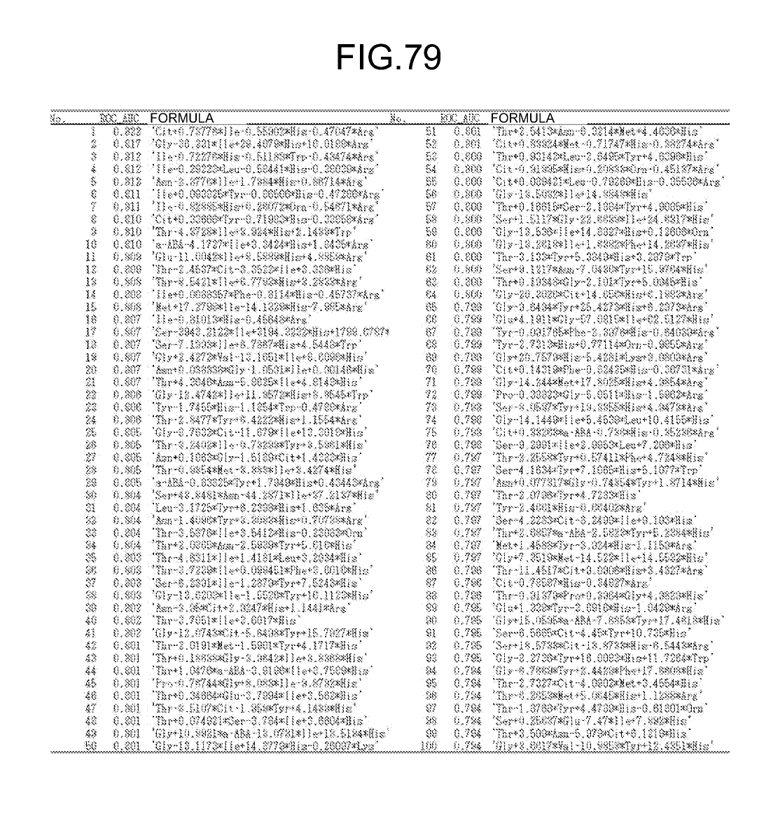

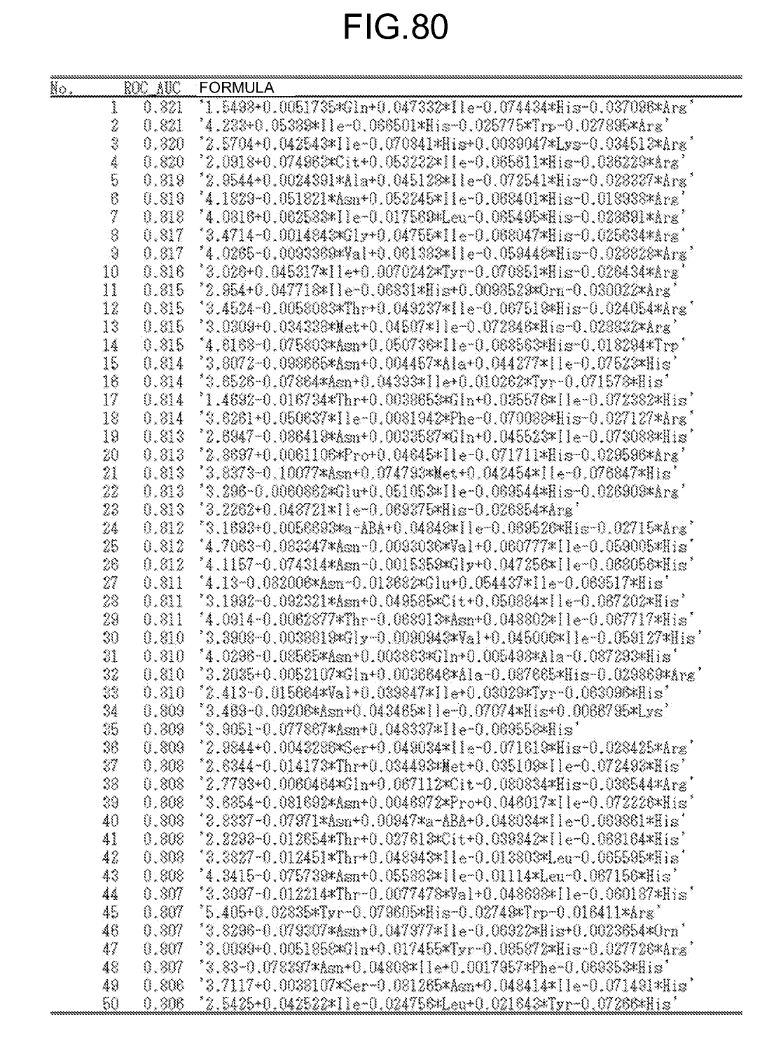

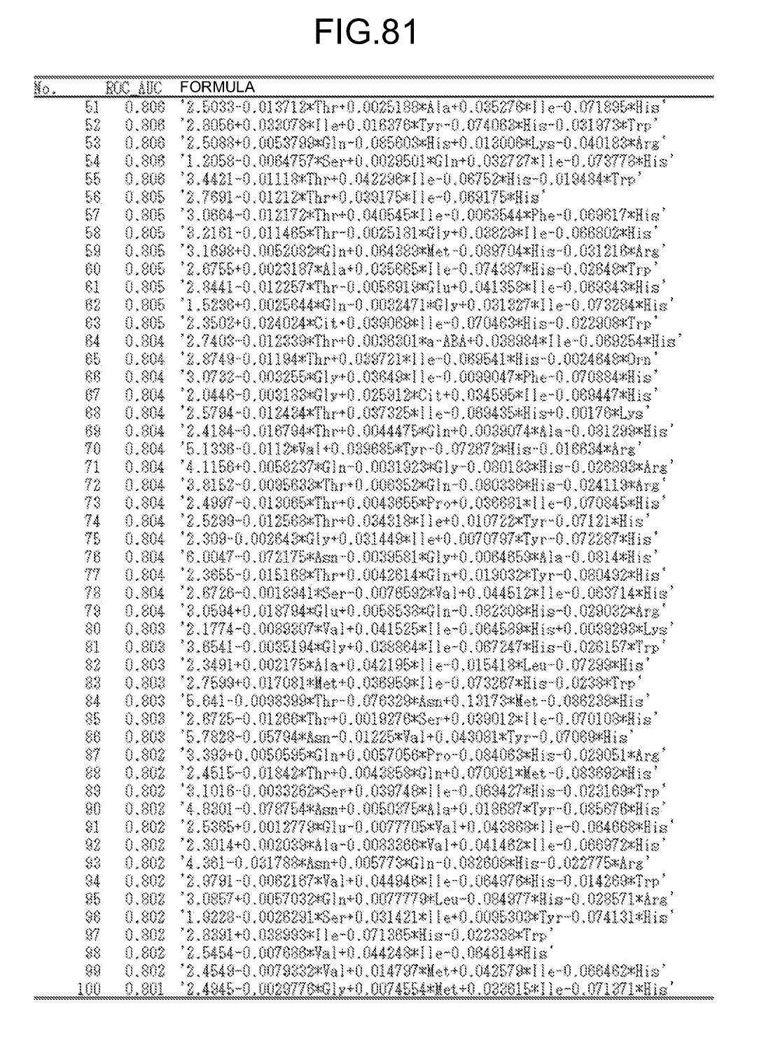

As successively shown in FIGS. 70 to 81, with respect to index formulae 31 to 39, the index formulae having equivalent discriminative ability are obtained. The value of each coefficient in the formulae shown in FIGS. 70 to 81 may be multiplied by a real number, and the value of each constant term may be subjected to addition, subtraction, multiplication, and division with an arbitrary real constant.

### Example 6

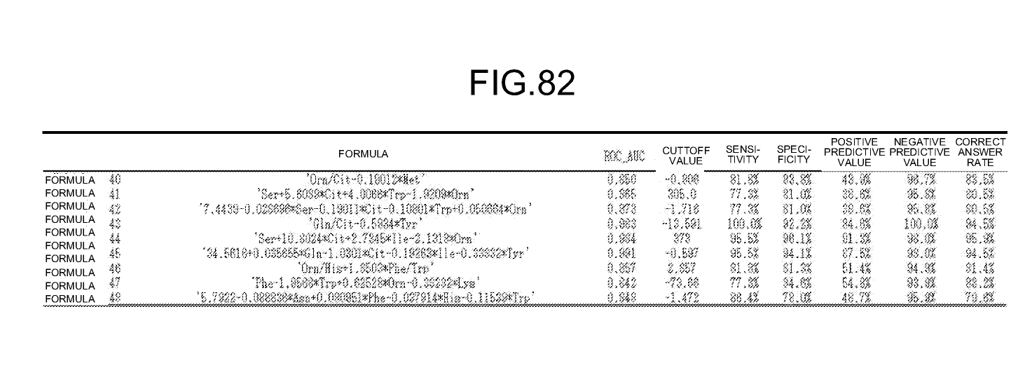

Of the sample data used in Example 1, the data of the ovarian cancer group and the cancer-free group are used. Indexes which maximize 2-group discriminative ability between the ovarian cancer group and the cancer-free group with respect to the discrimination of the cervical cancer group, the endometrial cancer group, and the ovarian cancer group are earnestly searched for, by using the method described in International publication WO 2004/052191 which is an international application by the present applicant. As a result, index formula 40 (see FIG. 82) is obtained among the index formulae having equivalent ability. Indexes which maximize 2-group discriminative ability between the ovarian cancer group and the cancer-free group with respect to the discrimination of the cervical cancer group, the endometrial cancer group, and the ovarian cancer group are searched for, by linear discriminant analysis (explanatory variable coverage method according to the minimum AIC). As a result, index formula 41 (see FIG. 82) is obtained among the index formulae having equivalent ability. Indexes which maximize 2-group discriminative ability between the ovarian cancer group and the cancer-free group with respect to the discrimination of the cervical cancer group, the endometrial cancer group, and the ovarian cancer group are searched for, by logistic regression analysis (explanatory variable coverage method according to the minimum AIC). As a result, index formula 42 (see FIG. 82) is obtained among the index formulae having equivalent ability. The value of each coefficient in index formulae 40, 41, and 42 may be multiplied by a real number, and the value of each constant term may be subjected to addition, subtraction, multiplication, and division with an arbitrary real constant.

Indexes which maximize 2-group discriminative ability between the ovarian cancer group and the healthy group with respect to the discrimination of the cervical cancer group, the endometrial cancer group, and the ovarian cancer group are earnestly searched for, by using the method described in International publication WO 2004/052191 which is an international application by the present applicant. As a result, index formula 43 (see FIG. 82) is obtained among the index formulae having equivalent ability. Indexes which maximize 2-group discriminative ability between the ovarian cancer group and the healthy group with respect to the discrimination of the cervical cancer group, the endometrial cancer group, and the ovarian cancer group are searched for, by linear discriminant analysis (explanatory variable coverage method according to the minimum AIC). As a result, index formula 44 (see FIG. 82) is obtained among the index formulae having equivalent ability. Indexes which maximize 2-group discriminative ability between the ovarian cancer group and the healthy group with respect to the discrimination of the cervical cancer group, the endometrial cancer group, and the ovarian cancer group are searched for, by logistic regression analysis (explanatory variable coverage method according to the minimum AIC). As a result, index formula 45 (see FIG. 82) is obtained among the index formulae having equivalent ability. The value of each coefficient in index formulae 43, 44, and 45 may be multiplied by a real number, and the value of each constant term may be subjected to addition, subtraction, multiplication, and division with an arbitrary real constant.

Indexes which maximize 2-group discriminative ability between the ovarian cancer group and the benign disease group with respect to the discrimination of the cervical cancer group, the endometrial cancer group, and the ovarian cancer group are earnestly searched for, by using the method described in International publication WO 2004/052191 which is an international application by the present applicant. As a result, index formula 46 (see FIG. 82) is obtained among the index formulae having equivalent ability. Indexes which maximize 2-group discriminative ability between the ovarian cancer group and the benign disease group with respect to the discrimination of the cervical cancer group, the endometrial cancer group, and the ovarian cancer group are searched for, by linear discriminant analysis (explanatory variable coverage method according to the minimum AIC). As a result, index formula 47 (see FIG. 82) is obtained among the index formulae having equivalent ability. Indexes which maximize 2-group discriminative ability between the ovarian cancer group and the benign disease group with respect to the discrimination of the cervical cancer group, the endometrial cancer group, and the ovarian cancer group are searched for, by logistic regression analysis (explanatory variable coverage method according to the minimum AIC). As a result, index formula 48 (see FIG. 82) is obtained among the index formulae having equivalent ability. The value of each coefficient in index formulae 46, 47, and 48 may be multiplied by a real number, and the value of each constant term may be subjected to addition, subtraction, multiplication, and division with an arbitrary real constant.

To examine the diagnostic ability using index formulae 40 to 42 in the discrimination of the ovarian cancer group, 2-group discrimination between the ovarian cancer group and the cancer-free group is evaluated by the ROC curve. As a result, the diagnostic ability as shown in FIG. 82 is obtained so that it is found that these index formulae are useful, with high diagnostic ability. As shown in FIG. 82, with regard to these index formulae, an optimum cutoff value, and the sensitivity, specificity, positive predictive value, negative predictive value, and correct answer rate of the used data are calculated.

To examine the diagnostic ability using index formulae 43 to 45 in the discrimination of the ovarian cancer group, 2-group discrimination between the ovarian cancer group and the healthy group is evaluated by the ROC curve. As a result, the diagnostic ability as shown in FIG. 82 is obtained so that it is found that these index formulae are useful, with high diagnostic ability. As shown in FIG. 82, with regard to these index formulae, an optimum cutoff value, and the sensitivity, specificity, positive predictive value, negative predictive value, and correct answer rate of the used data are calculated.

To examine the diagnostic ability using index formulae 46 to 48 in the discrimination of the ovarian cancer group, 2-group discrimination between the ovarian cancer group and the benign disease group is evaluated by the ROC curve. As a result, the diagnostic ability as shown in FIG. 82 is obtained so that it is found that these index formulae are useful, with high diagnostic ability. As shown in FIG. 82, with regard to these index formulae, an optimum cutoff value, and the sensitivity, specificity, positive predictive value, negative predictive value, and correct answer rate of the used data are calculated.

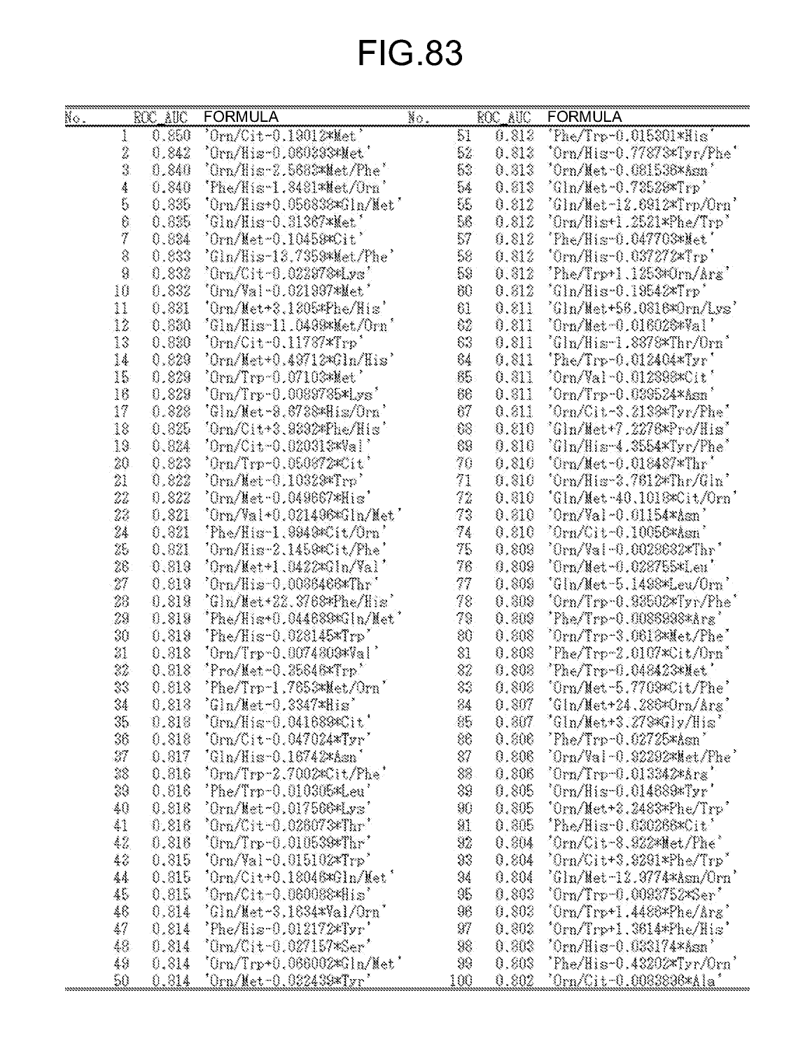

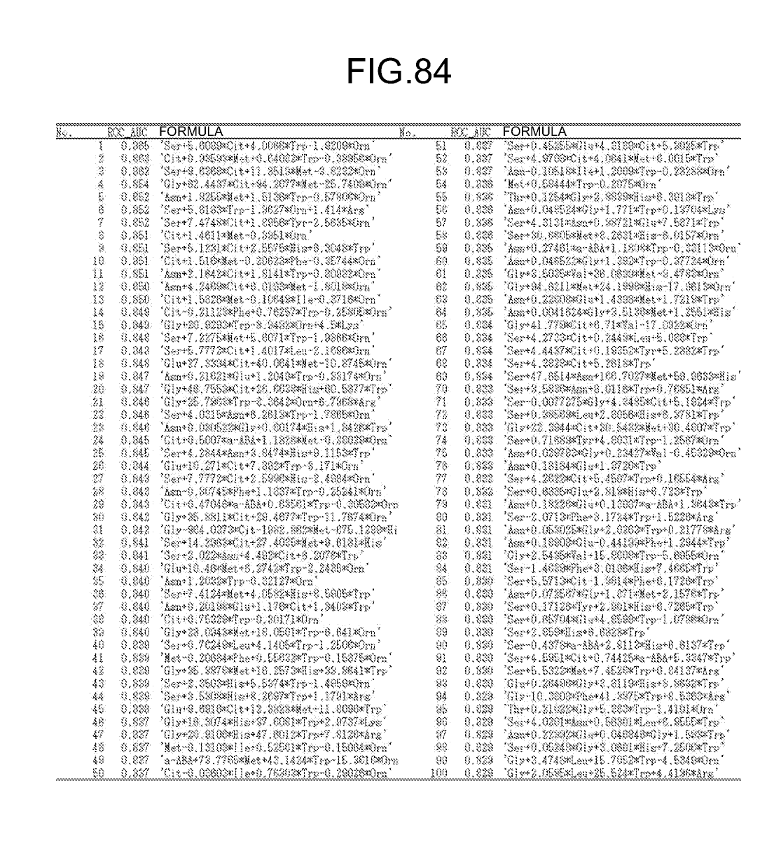

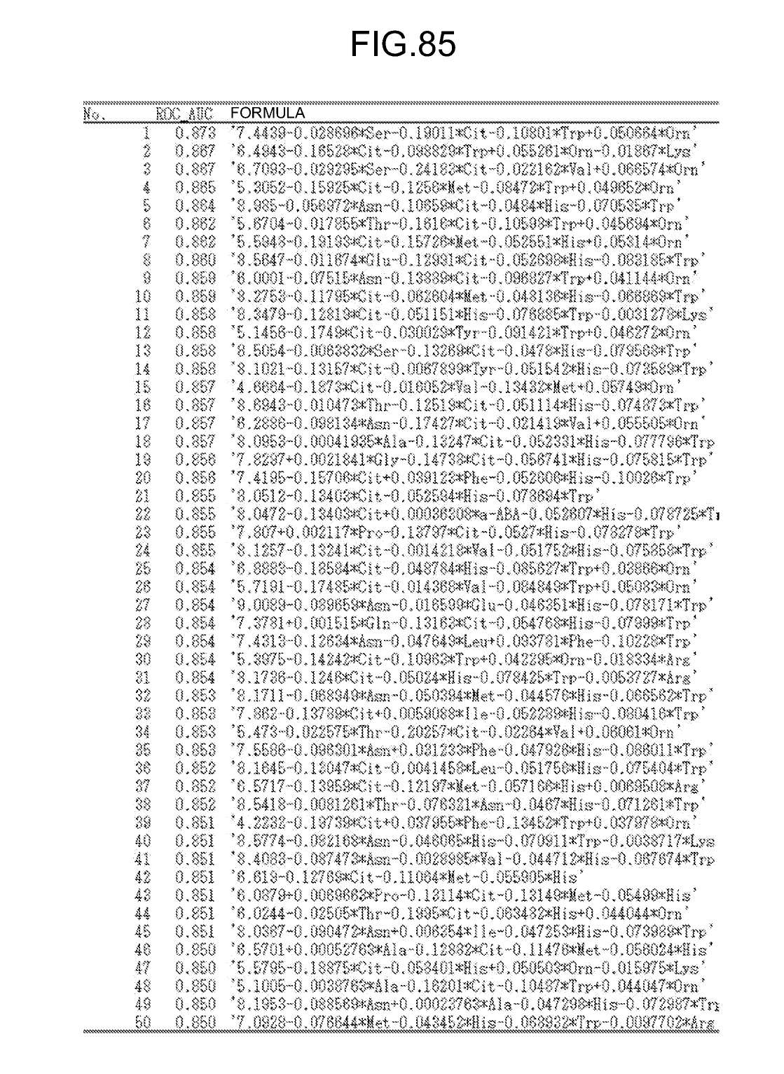

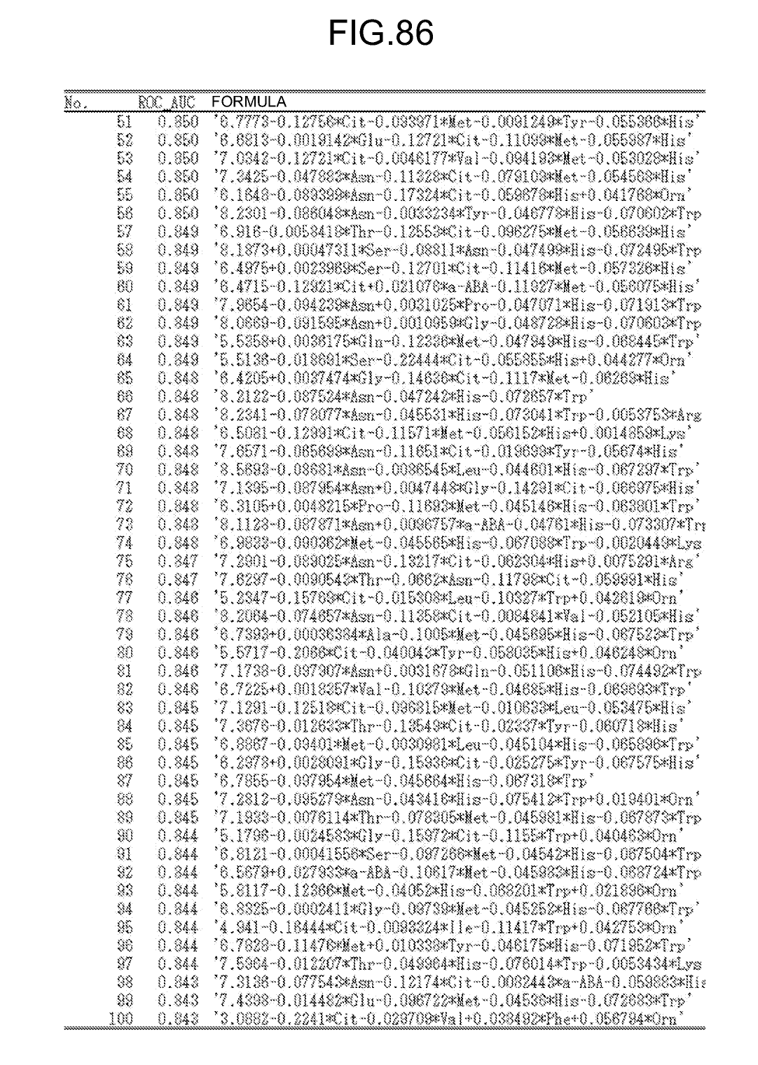

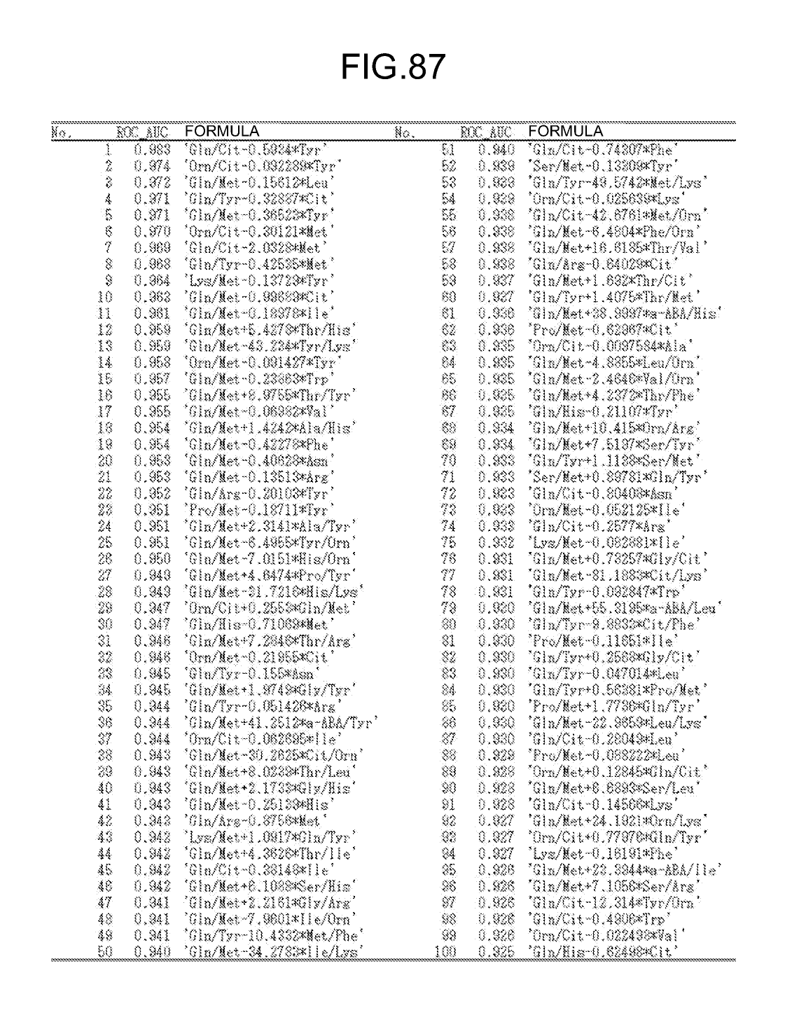

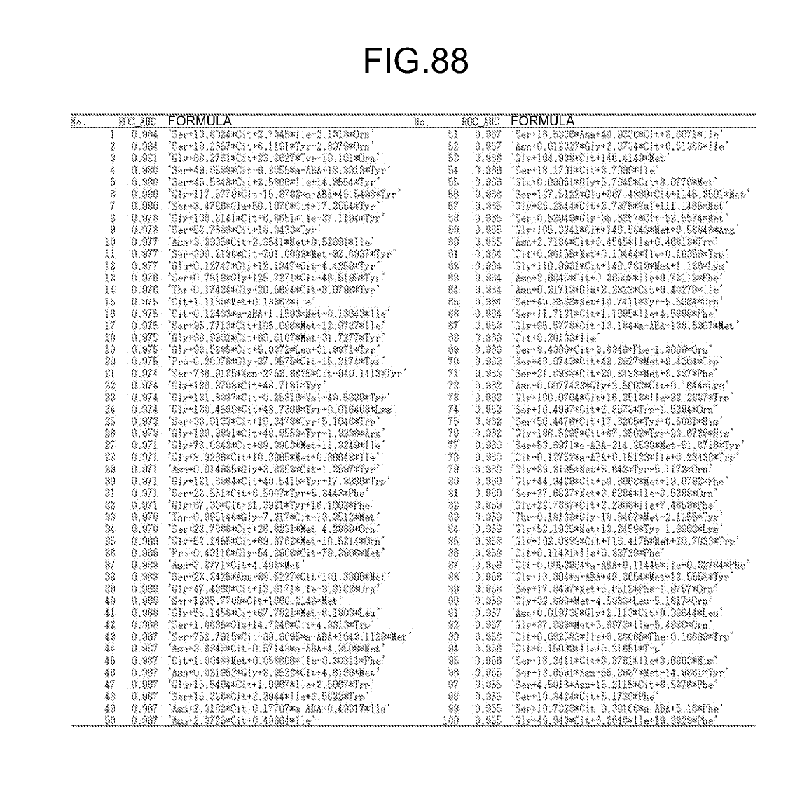

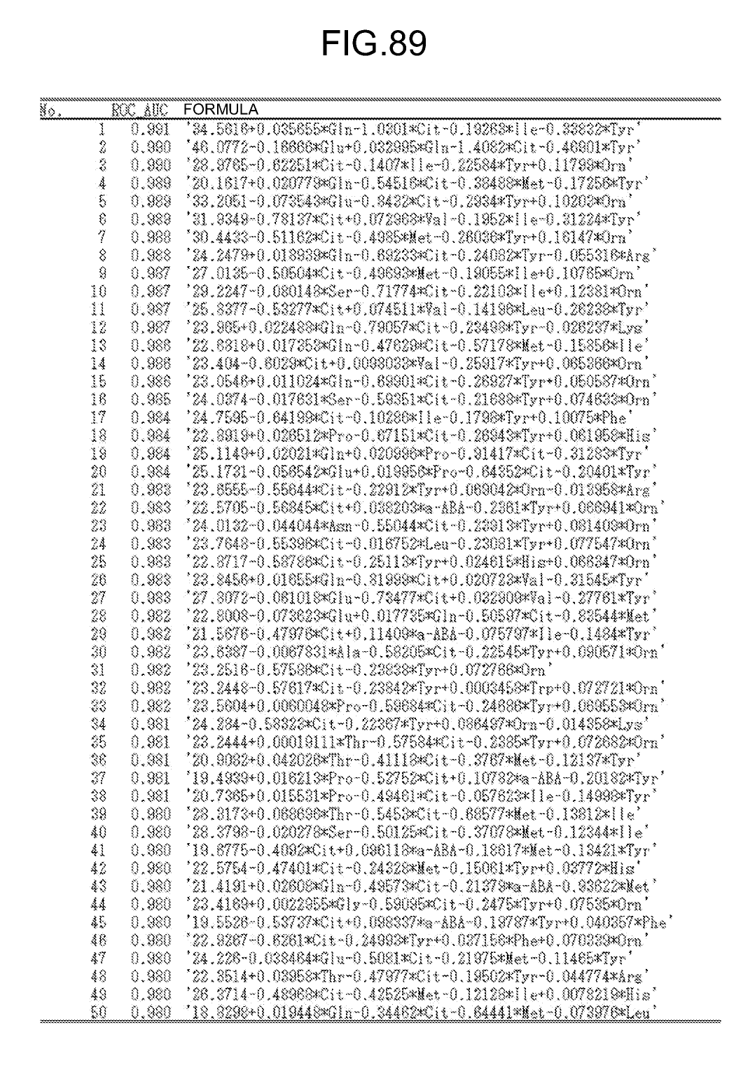

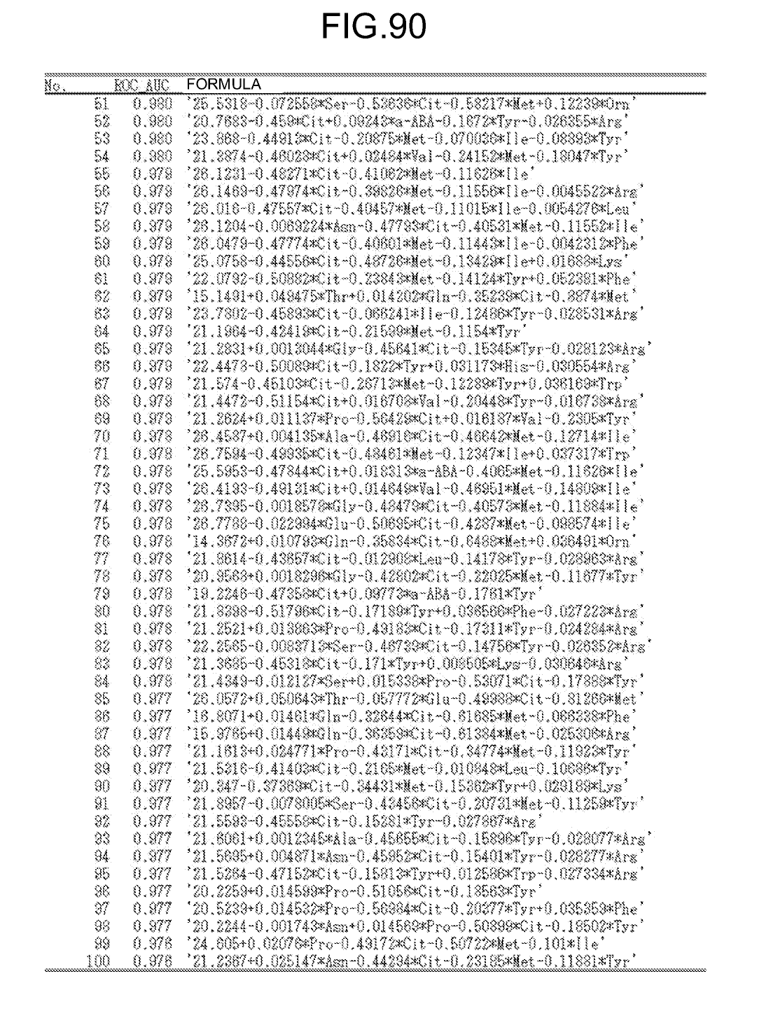

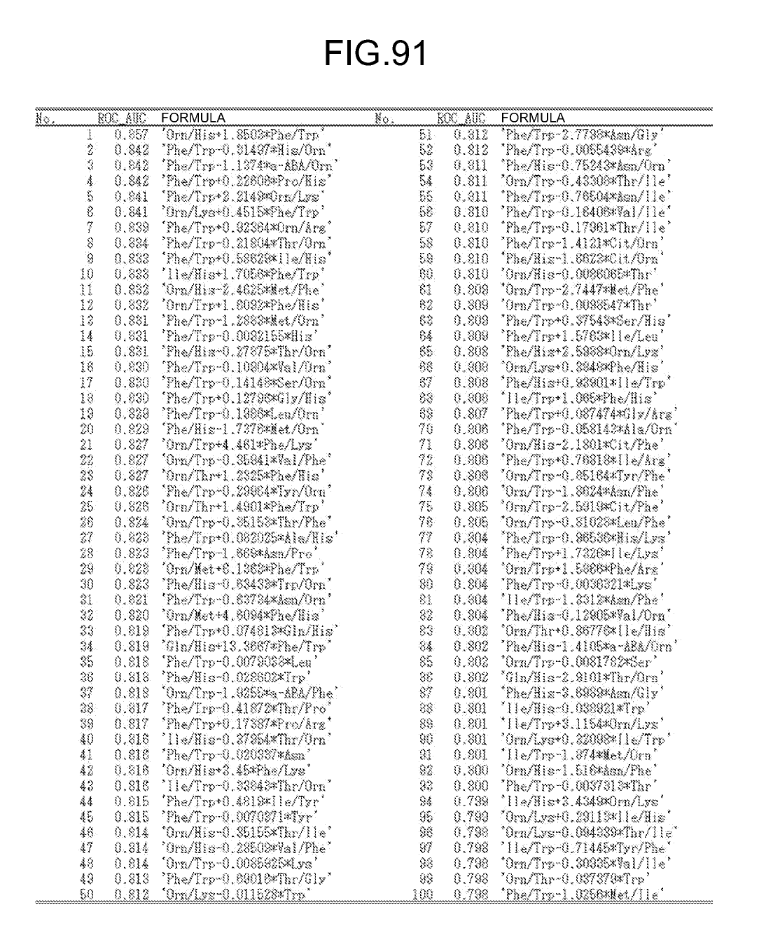

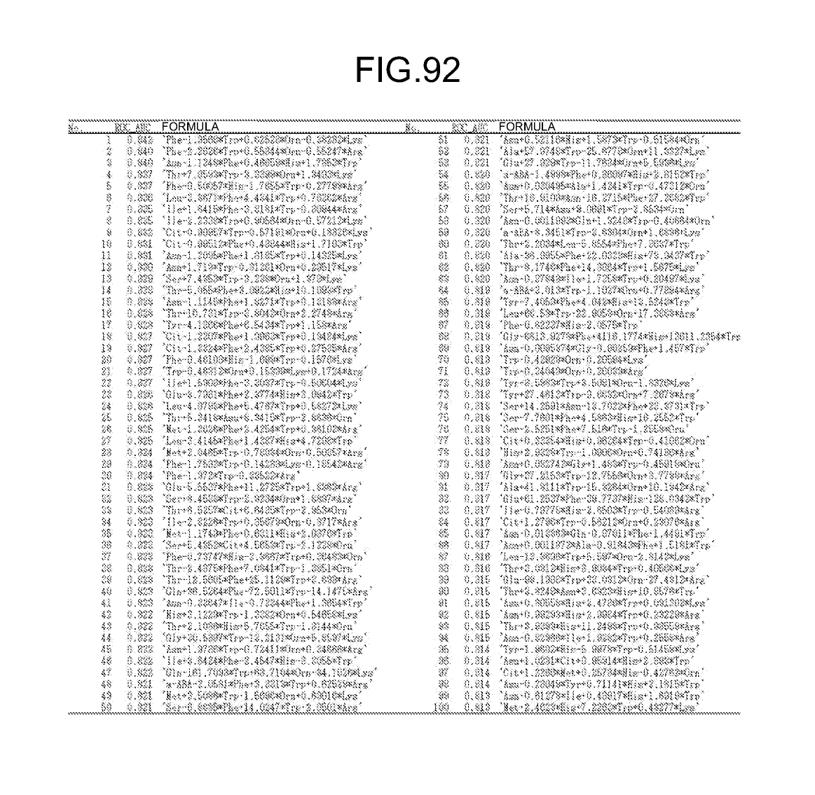

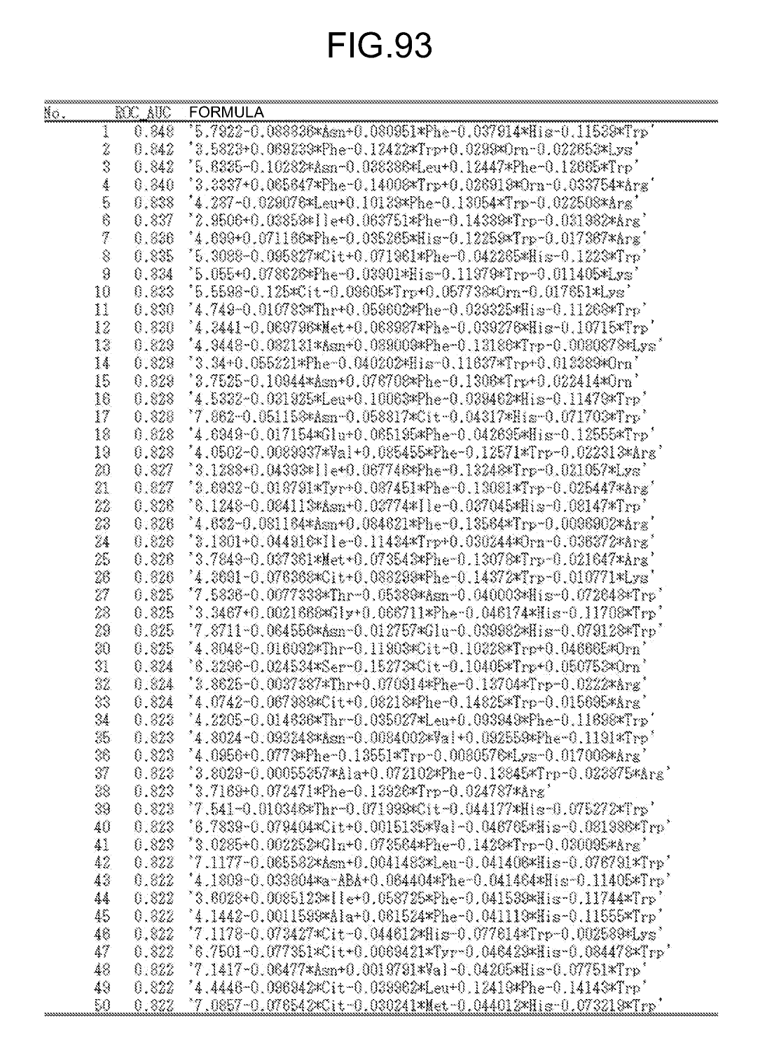

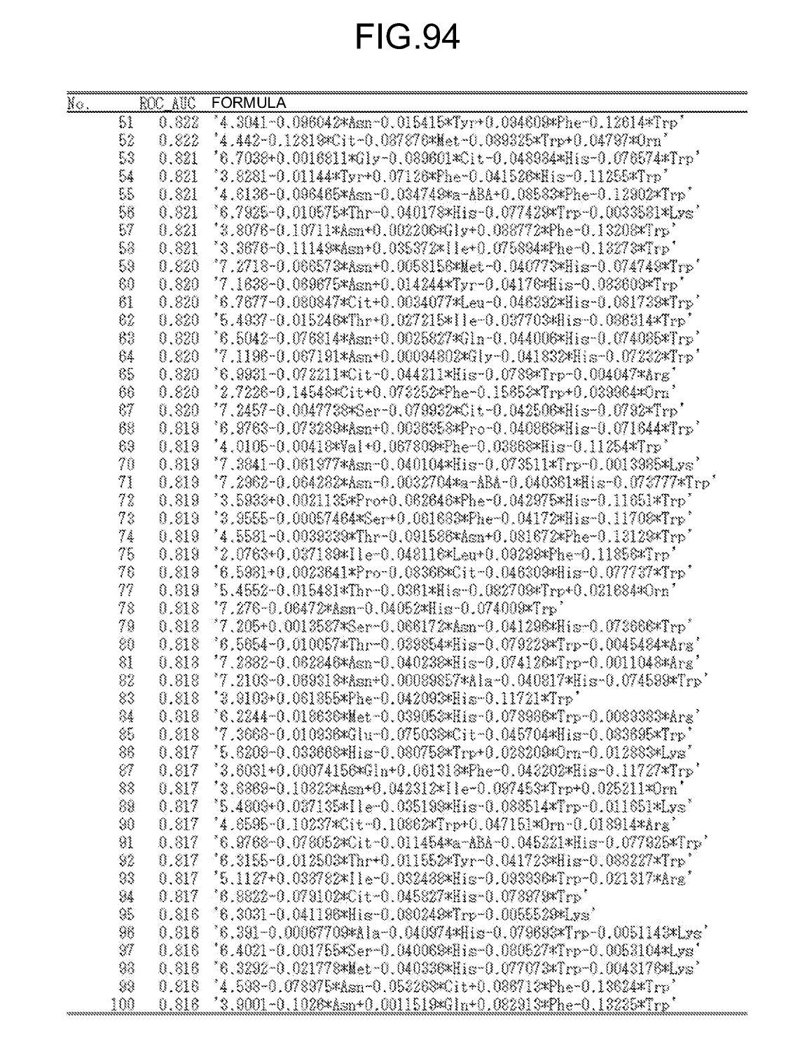

As successively shown in FIGS. 83 to 94, with respect to index formulae 40 to 48, the index formulae having equivalent discriminative ability are obtained. The value of each coefficient in the formulae shown in FIGS. 83 to 94 may be multiplied by a real number, and the value of each constant term may be subjected to addition, subtraction, multiplication, and division with an arbitrary real constant.

### Example 7

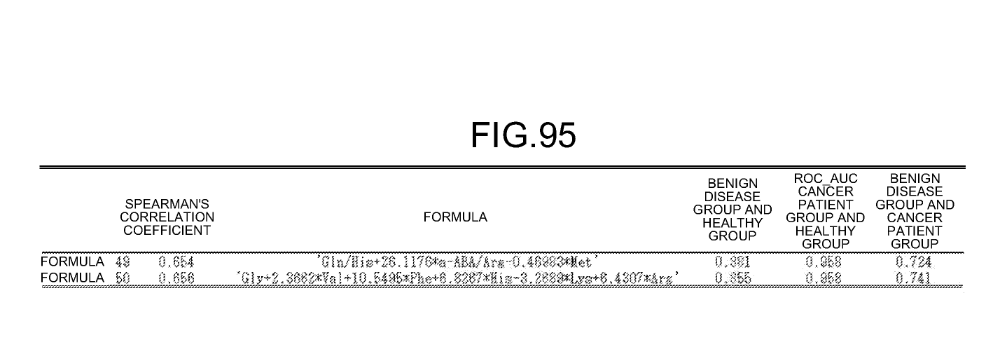

The sample data used in Example 1 is used. Indexes which maximize 3-group Spearman rank correlation coefficient between the cancer patient group, the benign disease group, and the healthy group with respect to the discrimination of the cervical cancer group, the endometrial cancer group, and the ovarian cancer group are earnestly searched for, by using the method described in International publication WO 2004/052191 which is an international application by the present applicant. As a result, index formula 49 (see FIG. 95) is obtained among the index formulae having equivalent ability. Indexes which maximize 3-group Spearman correlation coefficient between the cancer patient group, the benign disease group, and the healthy group with respect to the discrimination of the cervical cancer group, the endometrial cancer group, and the ovarian cancer group are searched for, by multiple regression analysis (explanatory variable coverage method according to the minimum AIC). As a result, index formula 50 (see FIG. 95) is obtained among the index formulae having equivalent ability. The value of each coefficient in index formulae 49 and 50 may be multiplied by a real number, and the value of each constant term may be subjected to addition, subtraction, multiplication, and division with an arbitrary real constant.

To examine the diagnostic ability using index formulae 49 and 50 in the discrimination of the cervical cancer group, the endometrial cancer group, and the ovarian cancer group, 3-group Spearman rank correlation coefficient between the cancer patient group, the benign disease group, and the healthy group, and 2-group discriminations between the cancer patient group and the healthy group, between the cancer patient group and the benign disease group, and between the benign disease group and the healthy group are evaluated by the ROC curve. As a result, the diagnostic ability as shown in FIG. 95 is obtained so that it is found that these index formulae are useful, with high diagnostic ability.

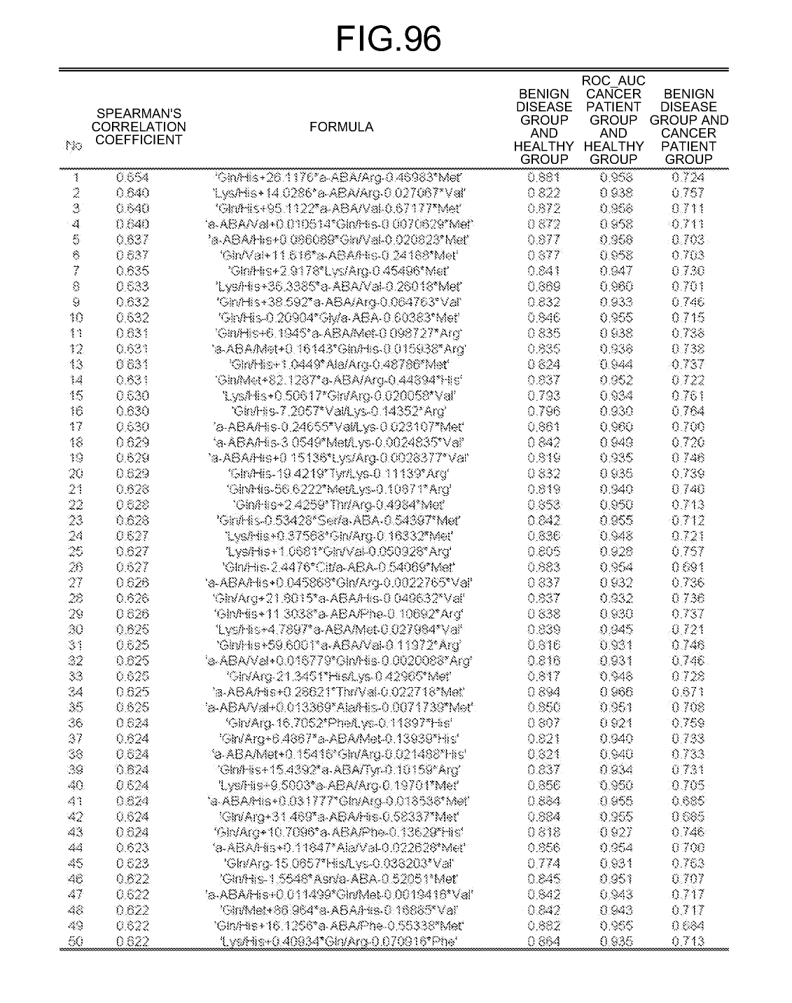

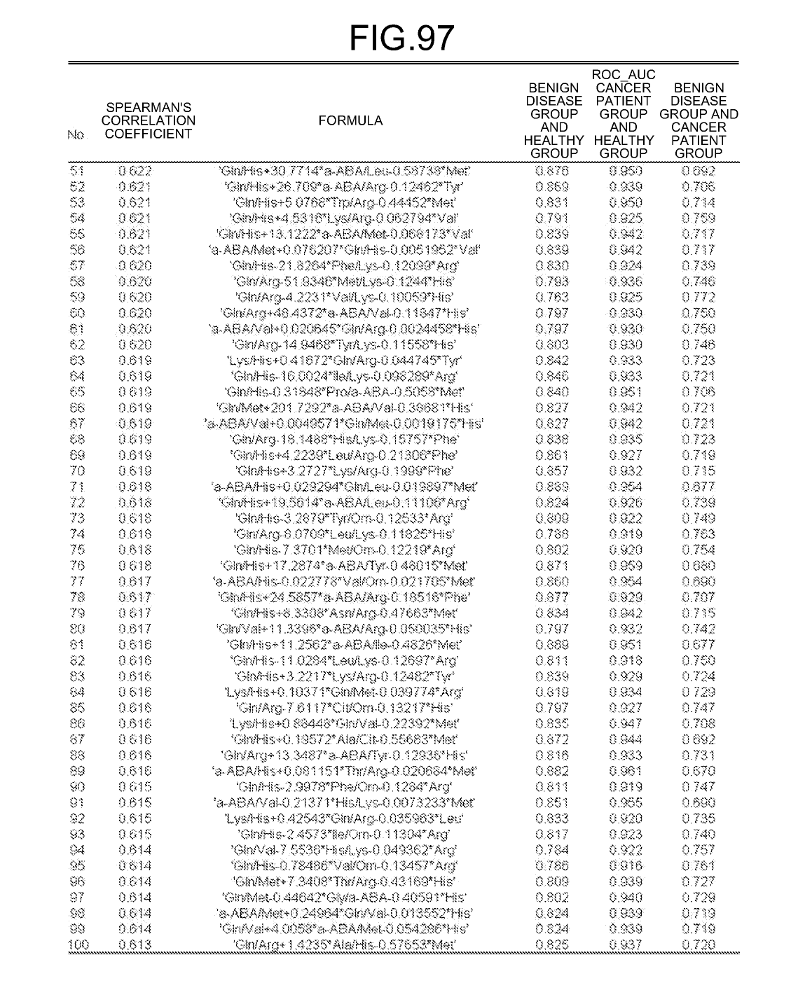

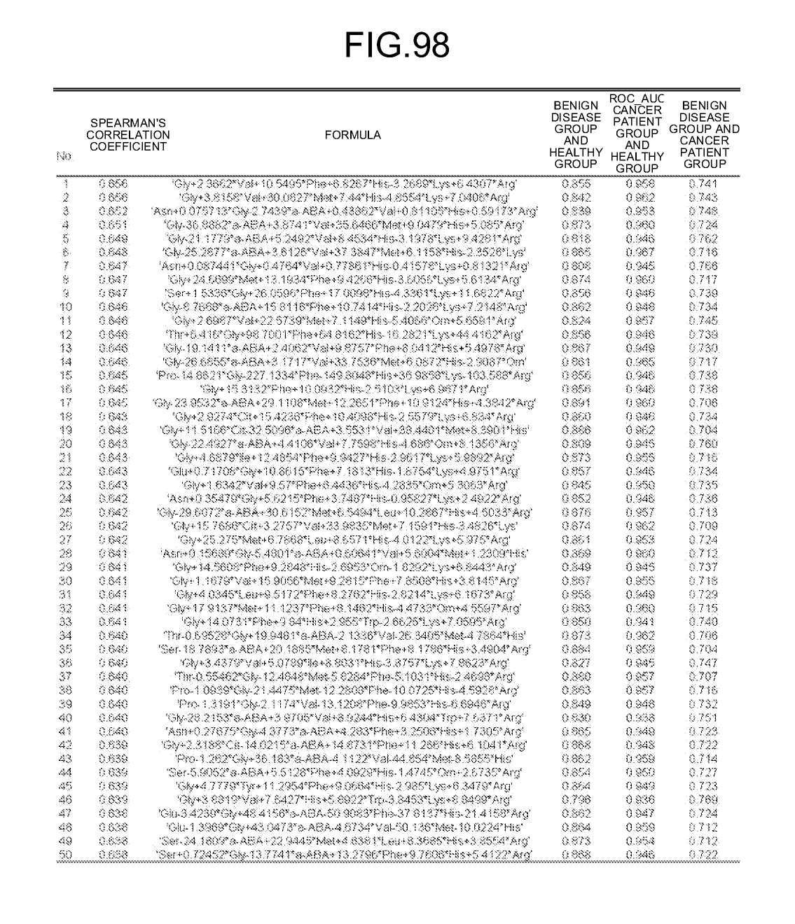

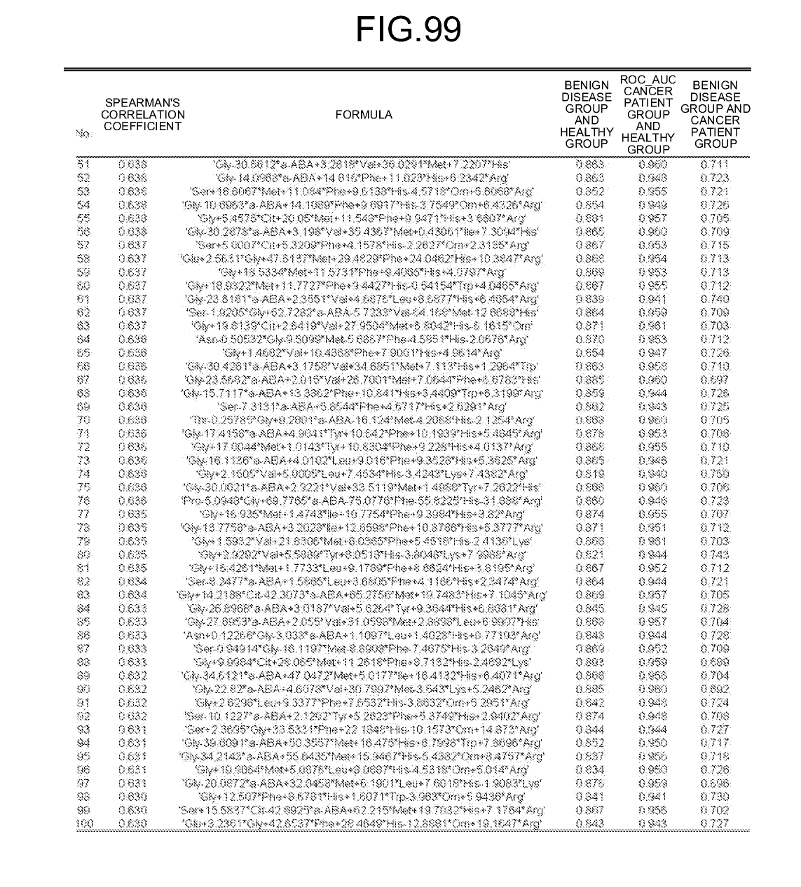

As successively shown in FIGS. 96 to 99, with respect to index formulae 49 and 50, the index formulae having equivalent discriminative ability are obtained. The value of each coefficient in the formulae shown in FIGS. 96 to 99 may be multiplied by a real number, and the value of each constant term may be subjected to addition, subtraction, multiplication, and division with an arbitrary real constant.

### Example 8

Of the sample data used in Example 1, the data of the cervical cancer group, the endometrial cancer group, and the ovarian cancer group are used. Indexes which maximize 3-group discriminative ability between the cervical cancer group, the endometrial cancer group, and the ovarian cancer group with respect to the discrimination of the cervical cancer group, the endometrial cancer group, and the ovarian cancer group are searched for, by discrimination analysis by the Mahalanobis' generalized distance by the stepwise explanatory variable selection method. As a result, as explanatory variable group 1, Cit, Met, Lys, Asn, Ala, Thr, Gln, and a-ABA are obtained.

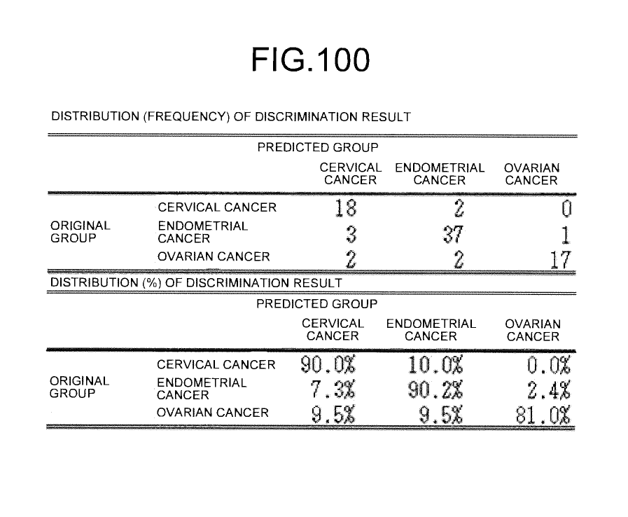

The diagnostic ability of the cervical cancer group, the endometrial cancer group, and the ovarian cancer group by the explanatory variable group 1 is evaluated by the correct answer rate of the discrimination result. As a result, as shown in FIG. 100, the correct answer rate of cervical cancer is 90.0%, the correct answer rate of endometrial cancer is 90.2%, the correct answer rate of ovarian cancer is 81.0%, and the entire correct answer rate is 87.1% when the prior probability is 33.3% and is equal in the groups, thereby showing high discriminative ability.

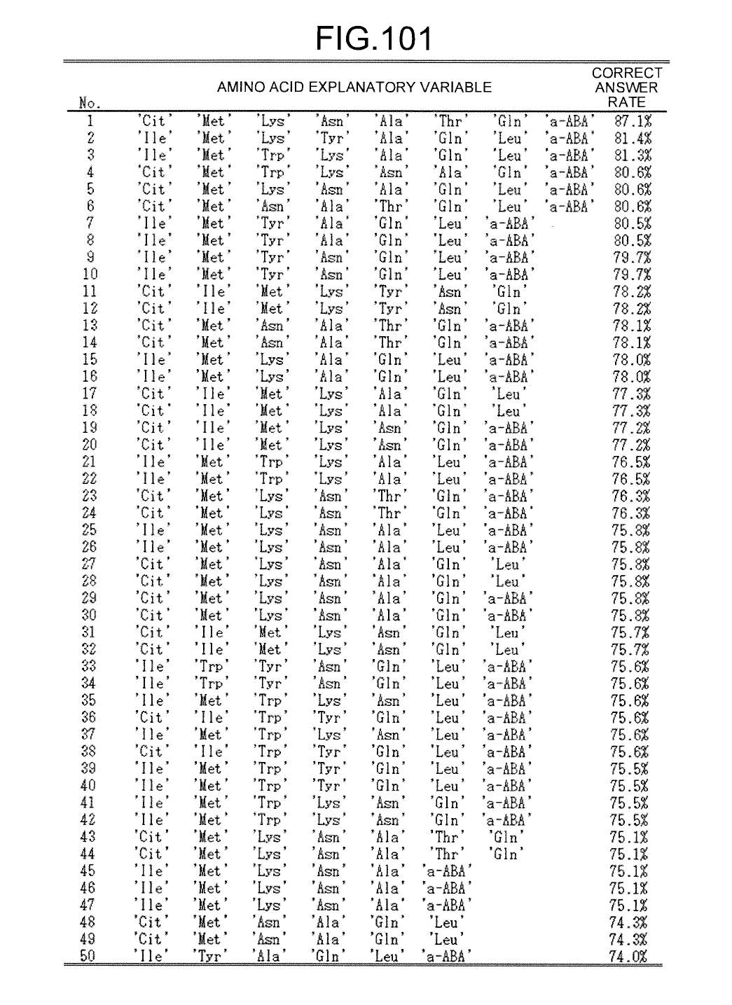

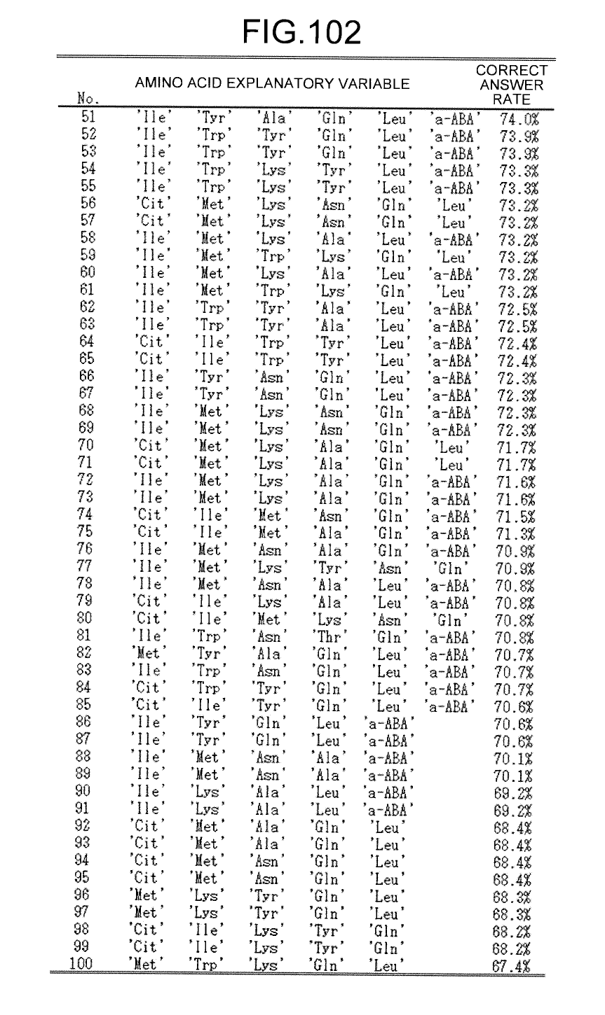

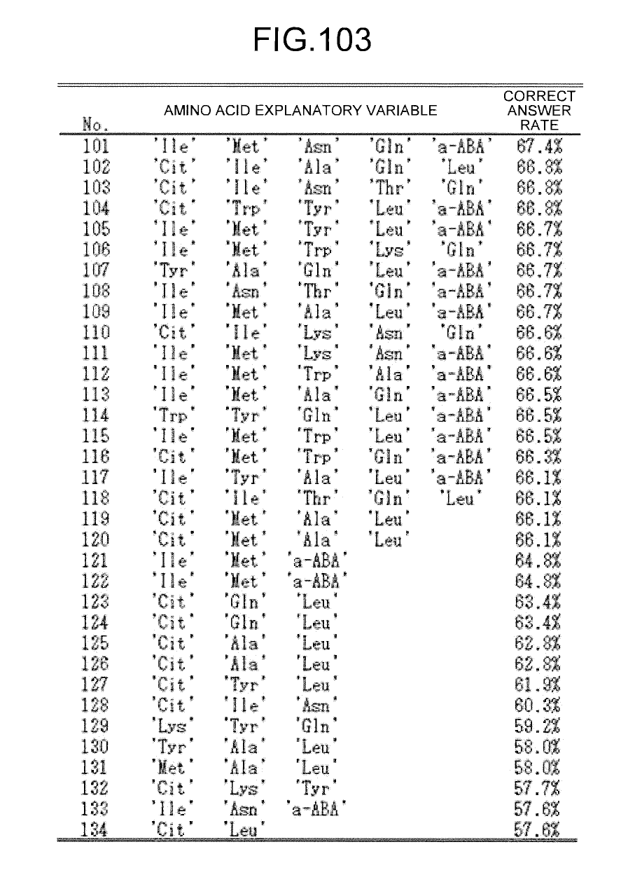

As shown in FIGS. 101 to 103, a plurality of combinations of amino acid explanatory variables having discriminative ability equivalent to the explanatory variable group 1 are obtained.

### Example 9

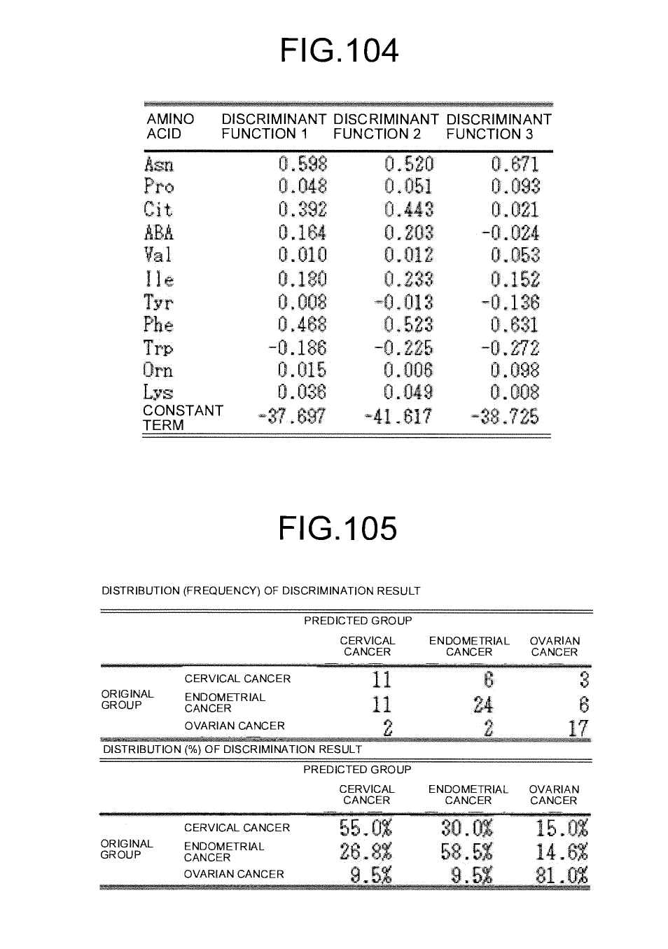

Of the sample data used in Example 1, the data of the cervical cancer group, the endometrial cancer group, and the ovarian cancer group are used. Indexes which maximize 3-group discriminative ability between the cervical cancer group, the endometrial cancer group, and the ovarian cancer group with respect to the discrimination in the cervical cancer group, the endometrial cancer group, and the ovarian cancer group are searched for, by linear discriminant analysis by the stepwise explanatory variable selection method. As a result, as index formula group 1, discriminant group (see FIG. 104) having the amino acid explanatory variables Asn, Pro, Cit, ABA, Val, Ile, Tyr, Phe, Trp, Orn, and Lys, and constant term are obtained. The value of each coefficient in the index formula group 1 may be multiplied by a real number, and the value of constant term may be subjected to addition, subtraction, multiplication, and division with an arbitrary real constant.

The diagnostic ability of the cervical cancer group, the endometrial cancer group, and the ovarian cancer group by the index formula group 1 are evaluated by the correct answer rate of the discrimination result. As a result, as shown in FIG. 105, the correct answer rate of cervical cancer is 55.0%, the correct answer rate of endometrial cancer is 58.5%, the correct answer rate of ovarian cancer is 81.0%, and the entire correct answer rate is 63.4% when the prior probability is 33.3% and is equal in the groups, thereby showing high discriminative ability.

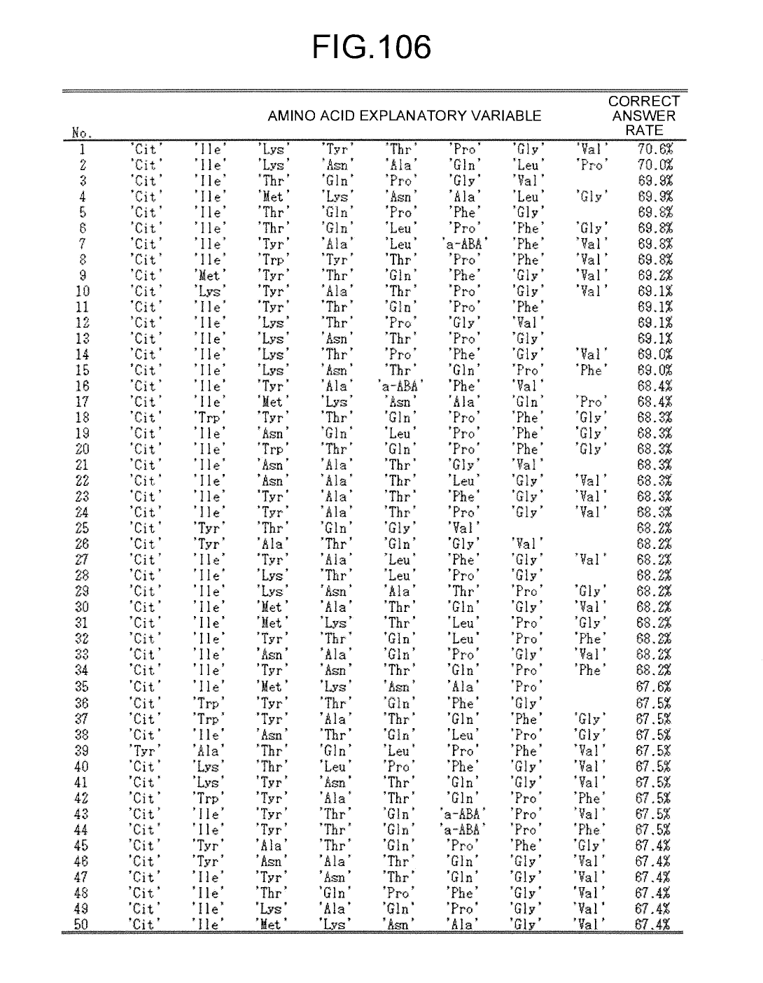

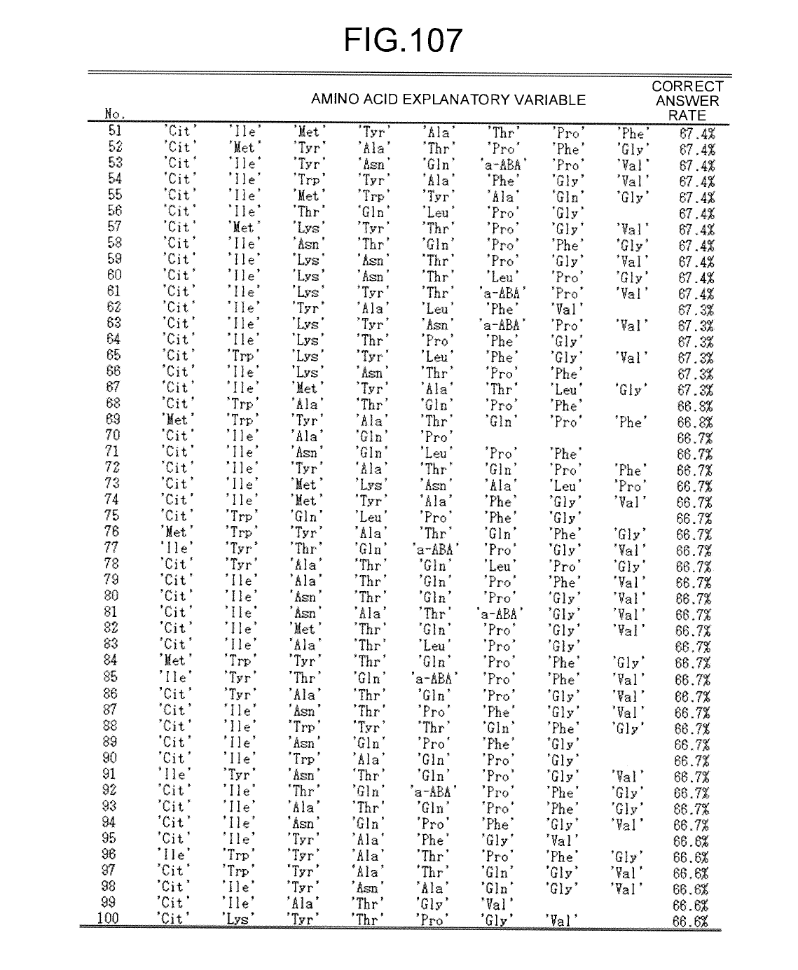

As shown in FIGS. 106 and 107, the combinations of amino acid explanatory variables having discriminative ability equivalent to the index formula group 1 are obtained.

### Example 10

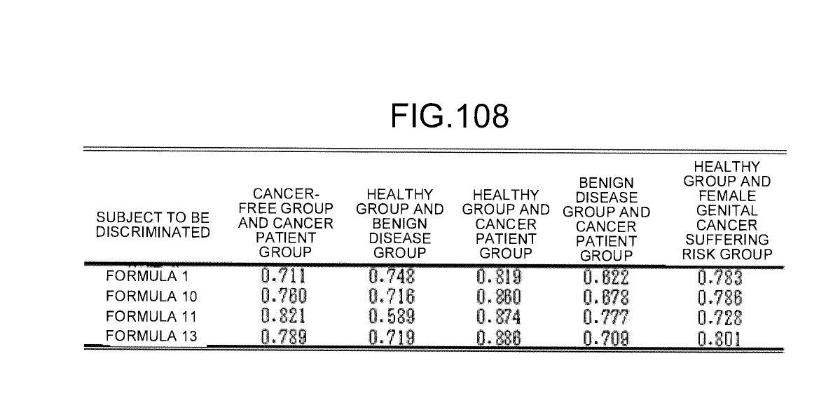

The sample data used in Example 1 is used. As a comparative example with respect to Example 2, 2-group discriminative abilities between the cancer patient group and the cancer-free group, between the healthy group and the benign disease group, between the cancer patient group and the healthy group, between the benign disease group and the cancer patient group, and between the female genital cancer suffering risk group and the healthy group with respect to the discrimination of the cervical cancer group, the endometrial cancer group, and the ovarian cancer group are examined using index formulae 1, 10, 11, and 13 described in International publication WO 2006/098192 which is an international application by the present applicant. As a result, as shown in FIG. 108, using any of the formulae for each 2-group discrimination, no ROC AUC values above ROC AUC obtained in Example 2 are obtained. From this, it is found that the multivariate discriminant in the present invention has higher discriminative ability with respect to the discrimination of the cervical cancer group, the endometrial cancer group, and the ovarian cancer group than the index formula group described in International publication WO 2006/098192 which is an international application by the present applicant.

### Example 11

Blood amino acid concentrations are measured from the blood samples of a cervical cancer patient group subjected to cervical cancer definitive diagnosis, an endometrial cancer patient group subjected to endometrial cancer definitive diagnosis, and an ovarian cancer patient group subjected to ovarian cancer definitive diagnosis and the blood samples of a cervical cancer-free group, an endometrial cancer-free group, and an ovarian cancer-free group by the amino acid analysis method. The unit of amino acid concentration is nmol/ml. In Example 11 and examples thereafter, the cervical cancer patient group, the endometrial cancer patient group, and the ovarian cancer patient group can be generically expressed as a cancer patient group, and the cervical cancer-free group, the endometrial cancer-free group, and the ovarian cancer-free group can be generically expressed as a cancer-free group. In addition, the cervical cancer patient group and the endometrial cancer patient group can be generically expressed as a uterine cancer patient group. In the cancer-free group, a group suffering from benign disease such as a myoma of a uterus can be expressed as a benign disease group, and a group other than that can be expressed as a healthy group. A group including the benign disease group and the cancer patient group can be expressed as a female genital cancer suffering risk group.

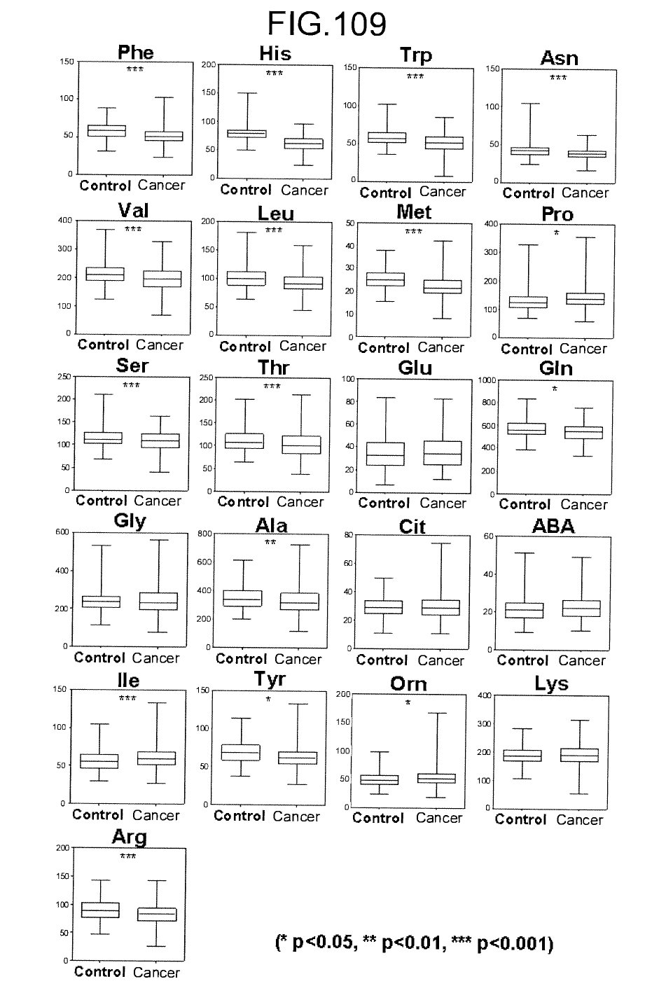

FIG. 109 is boxplots of the distribution of the amino acid explanatory variables of the cancer patient group and the cancer-free group. In FIG. 109, the horizontal axis shows the cancer-free group (Control) and the cancer patient group (Cancer), and ABA and Cys in the figure show α-ABA (α-aminobutyric acid) and cystine, respectively.

The t-test between two groups is performed for the discrimination between the cancer patient group and the cancer-free group. As a result, Pro, Ile, and Orn of the cancer patient group are increased more significantly than those of the cancer-free group (significant difference probability p<0.05), and Phe, His, Trp, Asn, Val, Leu, Met, Ser, Thr, Gln, Ala, Tyr, and Arg of the cancer patient group are decreased more significantly than those of the cancer-free group (significant difference probability p<0.05). From this, it is found that the amino acid explanatory variables Pro, Ile, Orn, Phe, His, Trp, Asn, Val, Leu, Met, Ser, Thr, Gln, Ala, Tyr, and Arg have 2-group discriminative ability between the cancer patient group and the cancer-free group.

Further, the discriminative ability of each of the amino acid explanatory variables in 2-group discrimination between the cancer patient group and the cancer-free group is evaluated by AUC of the ROC curve. As a result, AUC of the amino acid explanatory variables His, Trp, Asn, Val, Leu, and Met shows a value larger than 0.65. From this, it is found that the amino acid explanatory variables His, Trp, Asn, Val, Leu, and Met have 2-group discriminative ability between the cancer patient group and the cancer-free group.

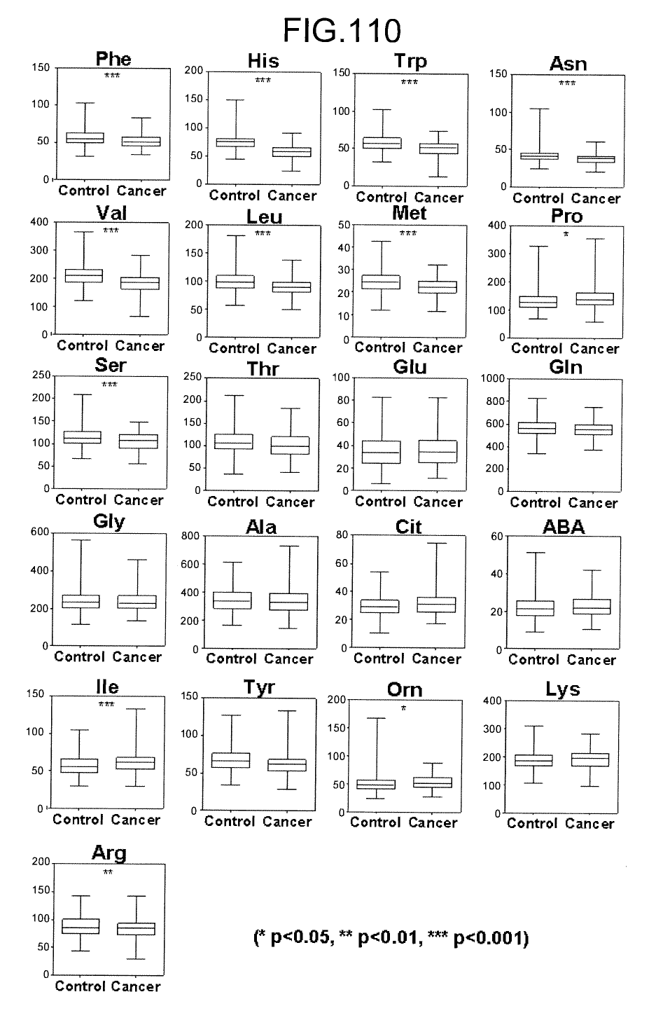

FIG. 110 is boxplots of the distribution of the amino acid explanatory variables of the uterine cancer patient group and the uterine cancer-free group. In FIG. 110, the horizontal axis shows the uterine cancer-free group (Control) and the uterine cancer patient group (Cancer), and ABA and Cys in the figure show α-ABA (α-aminobutyric acid) and cystine, respectively.

The t-test between two groups is performed for the discrimination between the uterine cancer patient group and the uterine cancer-free group. As a result, Pro, Ile, and Orn of the uterine cancer patient group are increased more significantly than those of the uterine cancer-free group (significant difference probability p<0.05), and Phe, His, Trp, Asn, Val, Leu, Met, Ser, and Arg of the uterine cancer patient group are decreased more significantly than those of the uterine cancer-free group (significant difference probability p<0.05). From this, it is found that the amino acid explanatory variables Pro, Ile, Orn, Phe, His, Trp, Asn, Val, Leu, Met, Ser, and Arg have 2-group discriminative ability between the uterine cancer patient group and the uterine cancer-free group.

Further, the discriminative ability of each of the amino acid explanatory variables in 2-group discrimination between the uterine cancer patient group and the uterine cancer-free group is evaluated by AUC of the ROC curve. As a result, AUC of the amino acid explanatory variables His, Trp, Asn, Val, Leu, and Met shows a value larger than 0.65. From this, it is found that the amino acid explanatory variables His, Trp, Asn, Val, Leu, and Met have 2-group discriminative ability between the uterine cancer patient group and the uterine cancer-free group.

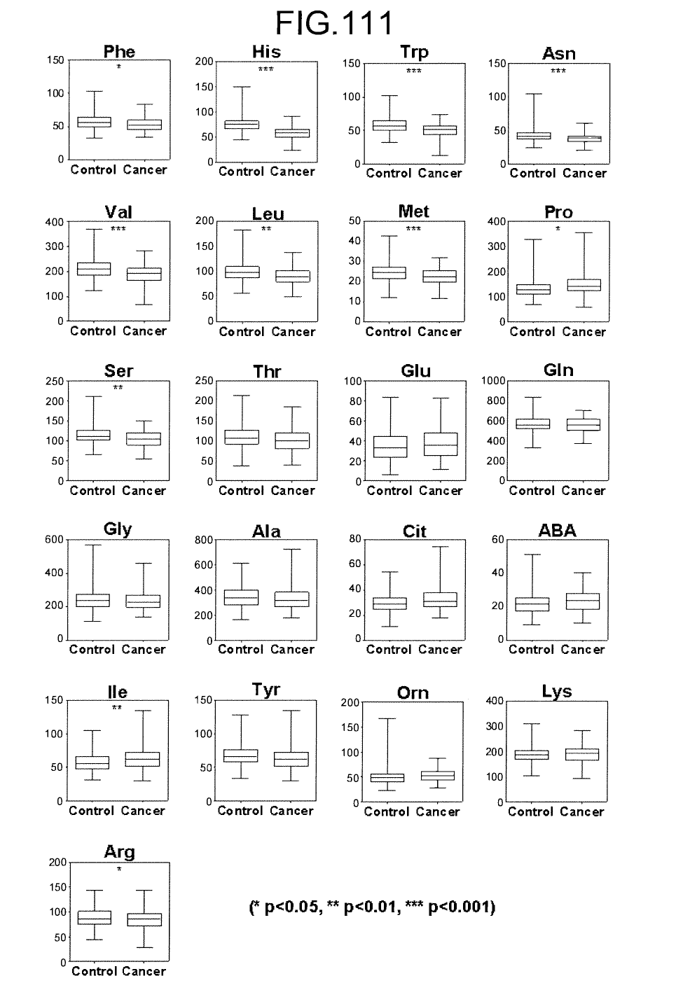

FIG. 111 is boxplots of the distribution of the amino acid explanatory variables of the endometrial cancer patient group and the endometrial cancer-free group. In FIG. 111, the horizontal axis shows the endometrial cancer-free group (Control) and the endometrial cancer patient group (Cancer), and ABA and Cys in the drawing show α-ABA (α-aminobutyric acid) and cystine, respectively.

The t-test between two groups is performed for the discrimination between the endometrial cancer patient group and the endometrial cancer-free group. As a result, Pro and Ile of the endometrial cancer patient group are increased more significantly than those of the endometrial cancer-free group (significant difference probability p<0.05), and Phe, His, Trp, Asn, Val, Leu, Met, Ser, and Arg of the endometrial cancer patient group are decreased more significantly than those of the endometrial cancer-free group (significant difference probability p<0.05). From this, it is found that the amino acid explanatory variables Pro, Ile, Phe, His, Trp, Asn, Val, Leu, Met, Ser, and Arg have 2-group discriminative ability between the endometrial cancer patient group and the endometrial cancer-free group.

Further, the discriminative ability of each of the amino acid explanatory variables in 2-group discrimination between the endometrial cancer patient group and the endometrial cancer-free group is evaluated by AUC of the ROC curve. As a result, AUC of the amino acid explanatory variables His, Trp, Asn, and Val shows a value larger than 0.65. From this, it is found that the amino acid explanatory variables His, Trp, Asn, and Val have 2-group discriminative ability between the endometrial cancer patient group and the endometrial cancer-free group.

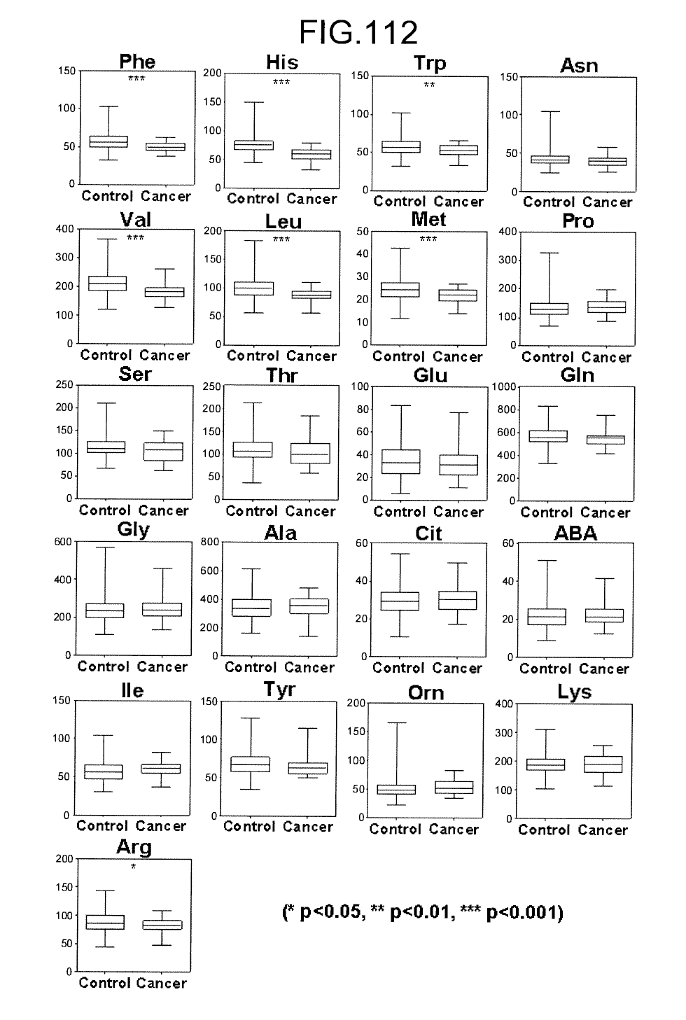

FIG. 112 is boxplots of the distribution of the amino acid explanatory variables of the cervical cancer patient group and the cervical cancer-free group. In FIG. 112, the horizontal axis shows the cervical cancer-free group (Control) and the cervical cancer patient group (Cancer), and ABA and Cys in the figure show α-ABA (α-aminobutyric acid) and cystine, respectively.

The t-text between two groups is performed for the discrimination between the cervical cancer patient group and the cervical cancer-free group. As a result, Phe, His, Trp, Val, Leu, Met, and Arg of the cervical cancer patient group are decreased more significantly than those of the cervical cancer-free group (significant difference probability p<0.05). From this, it is found that the amino acid explanatory variables Phe, His, Trp, Val, Leu, Met, and Arg have 2-group discriminative ability between the cervical cancer patient group and the cervical cancer-free group.

Further, the discriminative ability of each of the amino acid explanatory variables in 2-group discrimination between the cervical cancer patient group and the cervical cancer-free group is evaluated by AUC of the ROC curve. As a result, AUC of the amino acid explanatory variables Phe, His, Val, Leu, and Met shows a value larger than 0.65. From this, it is found that the amino acid explanatory variables Phe, His, Val, Leu, and Met have 2-group discriminative ability between the cervical cancer patient group and the cervical cancer-free group.

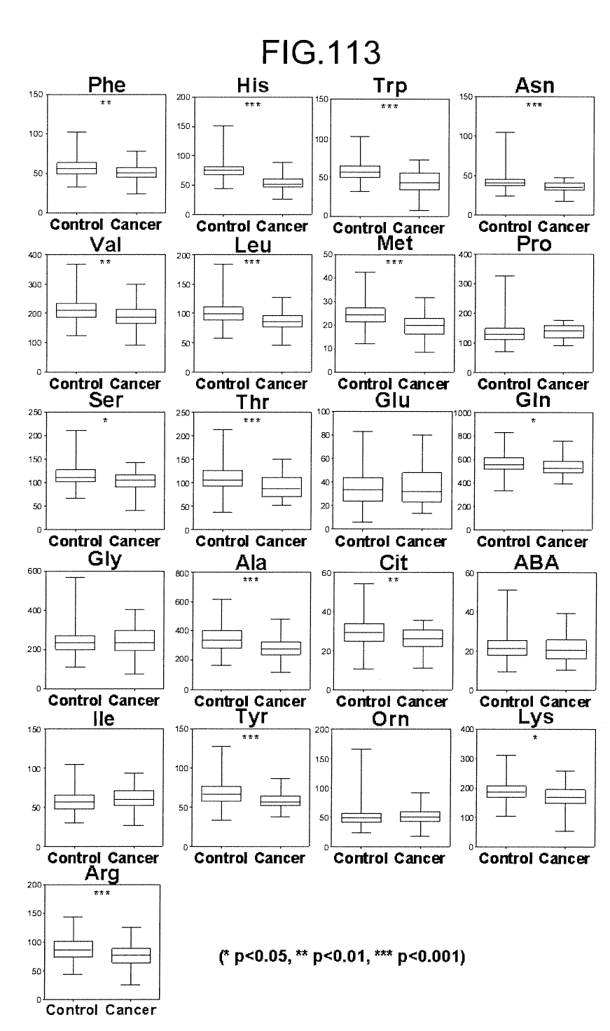

FIG. 113 is boxplots of the distribution of the amino acid explanatory variables of the ovarian cancer patient group and the ovarian cancer-free group. In FIG. 113, the horizontal axis shows the ovarian cancer-free group (Control) and the ovarian cancer patient group (Cancer), and ABA and Cys in the figure show α-ABA (α-aminobutyric acid) and cystine, respectively.

The t-text between two groups is performed for the discrimination between the ovarian cancer patient group and the ovarian cancer-free group. As a result, Cit of the ovarian cancer patient group is increased more significantly than that of the ovarian cancer-free group (significant difference probability p<0.05), and Phe, His, Trp, Asn, Val, Leu, Met, Ser, Thr, Gln, Ala, Tyr, Lys, and Arg of the ovarian cancer patient group is decreased more significantly than those of the ovarian cancer-free group (significant difference probability p<0.05). From this, it is found that the amino acid explanatory variables Cit, Phe, His, Trp, Asn, Val, Leu, Met, Ser, Thr, Gln, Ala, Tyr, Lys, and Arg have 2-group discriminative ability between the ovarian cancer patient group and the ovarian cancer-free group.

Further, the discriminative ability of each of the amino acid explanatory variables in 2-group discrimination between the ovarian cancer patient group and the ovarian cancer-free group is evaluated by AUC of the ROC curve. As a result, AUC of the amino acid explanatory variables His, Trp, Asn, Val, Leu, Met, Thr, Ala, Tyr, Lys, and Arg shows a value larger than 0.65. From this, it is found that the amino acid explanatory variables His, Trp, Asn, Val, Leu, Met, Thr, Ala, Tyr, Lys, and Arg have 2-group discriminative ability between the ovarian cancer patient group and the ovarian cancer-free group.

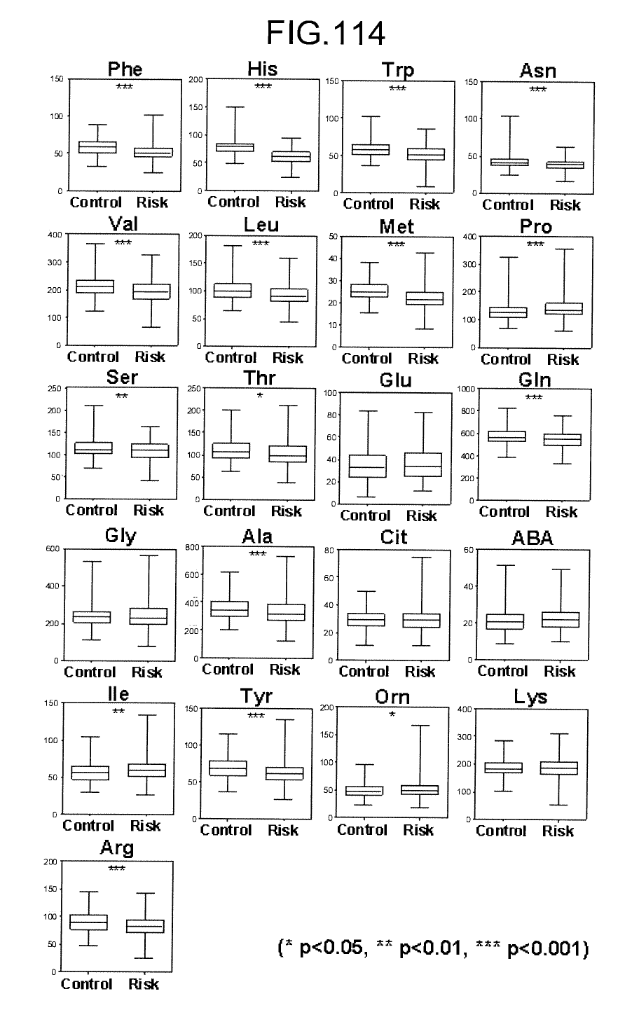

FIG. 114 is boxplots of the distribution of the amino acid explanatory variables of the female genital cancer suffering risk group and the healthy group. In FIG. 114, the horizontal axis shows the healthy group (Control) and the female genital cancer suffering risk group (Risk), and ABA and Cys in the figure show α-ABA (α-aminobutyric acid) and cystine, respectively.

The t-test between two groups is performed for the discrimination between the female genital cancer suffering risk group and the healthy group. As a result, Pro, Ile, and Orn of the female genital cancer suffering risk group are increased more significantly than those of the healthy group (significant difference probability p<0.05), and Phe, His, Trp, Asn, Val, Leu, Met, Ser, Thr, Gln, Ala, Tyr, and Arg of the female genital cancer suffering risk group are decreased more significantly than those of the healthy group (significant difference probability p<0.05). From this, it is found that the amino acid explanatory variables Pro, Ile, Orn, Phe, His, Trp, Asn, Val, Leu, Met, Ser, Thr, Gln, Ala, Tyr, and Arg have 2-group discriminative ability between the female genital cancer suffering risk group and the healthy group.

Further, the discriminative ability of each of the amino acid explanatory variables in 2-group discrimination between the female genital cancer suffering risk group and the healthy group is evaluated by AUC of the ROC curve. As a result, AUC of the amino acid explanatory variables Phe, His, Trp, and Met shows a value larger than 0.65. From this, it is found that the amino acid explanatory variables Phe, His, Trp, and Met have 2-group discriminative ability between the female genital cancer suffering risk group and the healthy group.

### Example 12

The sample data used in Example 11 is used. Indexes which maximize 2-group discriminative ability between the cancer patient group and the cancer-free group are searched for, by using logistic analysis (explanatory variable coverage method according to the maximizing criterion of area under the ROC curve). As a result, as index formula 51, a logistic regression equation having His, Leu, Met, Cit, Ile, and Tyr (the numerical coefficients of the amino acid explanatory variables His, Leu, Met, Cit, Ile, and Tyr and the constant term are −0.10000, −0.04378, −0.17879, 0.03911, 0.07852, 0.03566, and 5.86036 in order) is obtained.

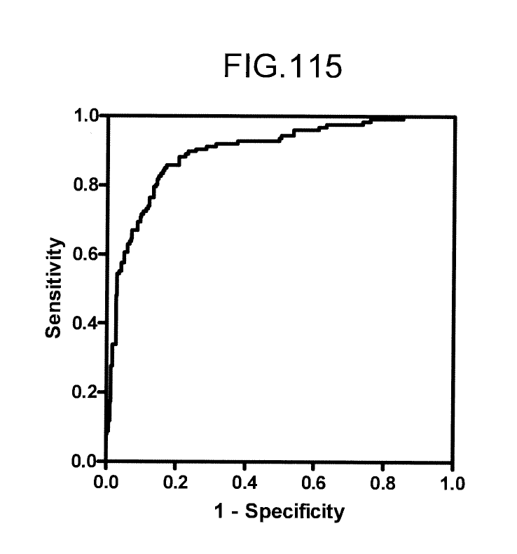

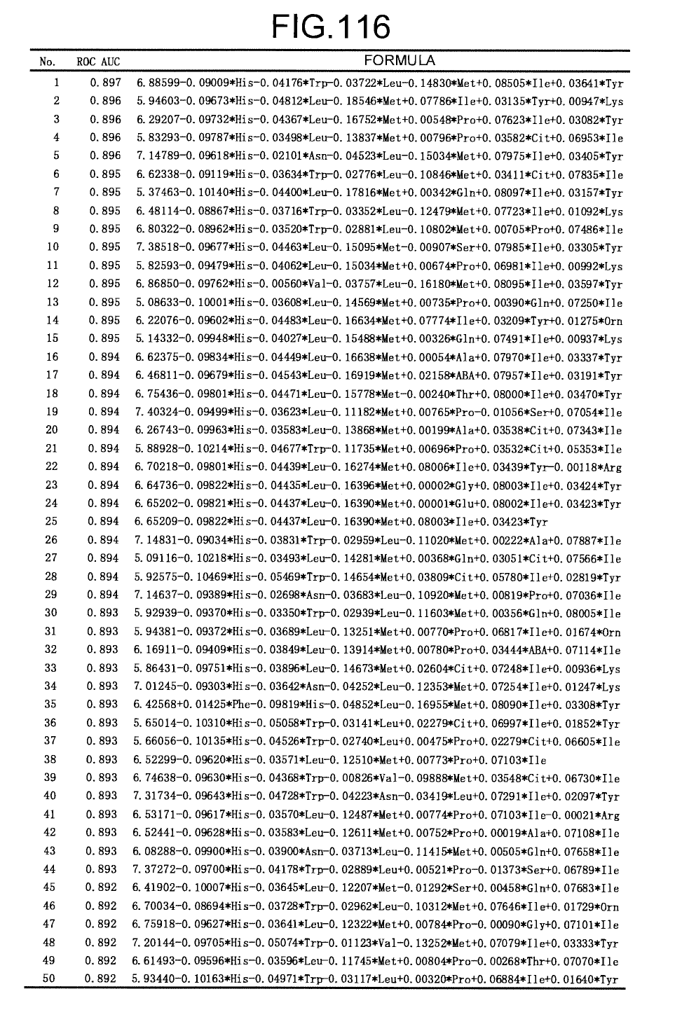

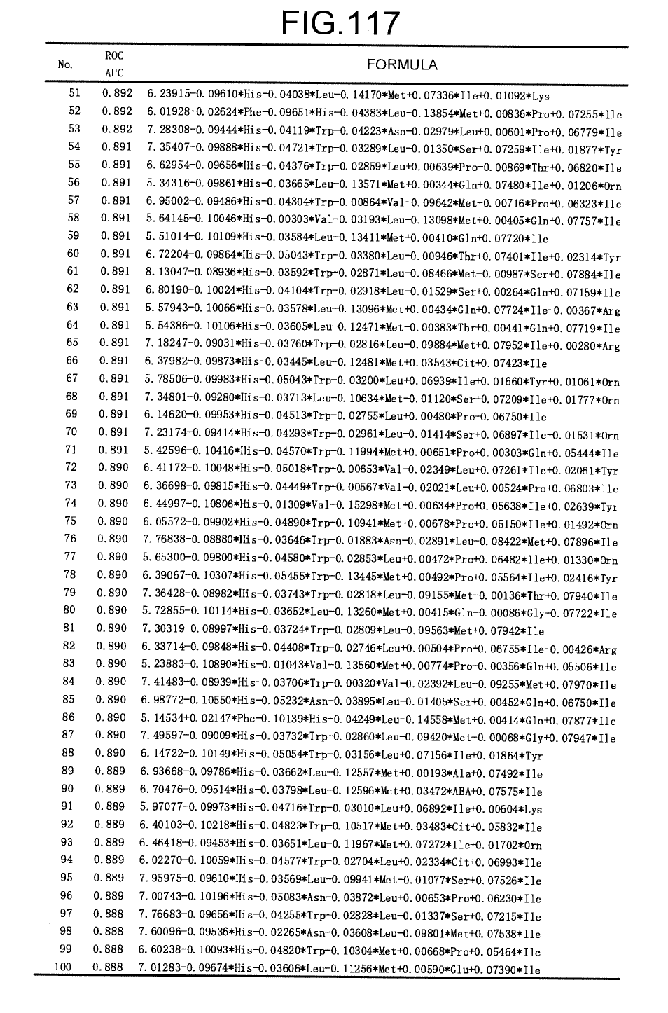

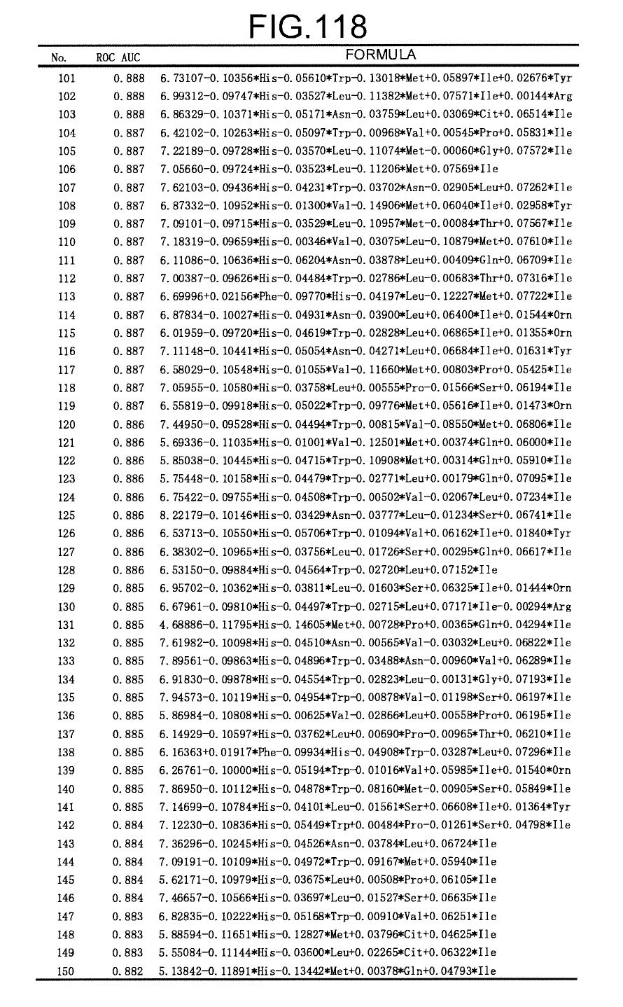

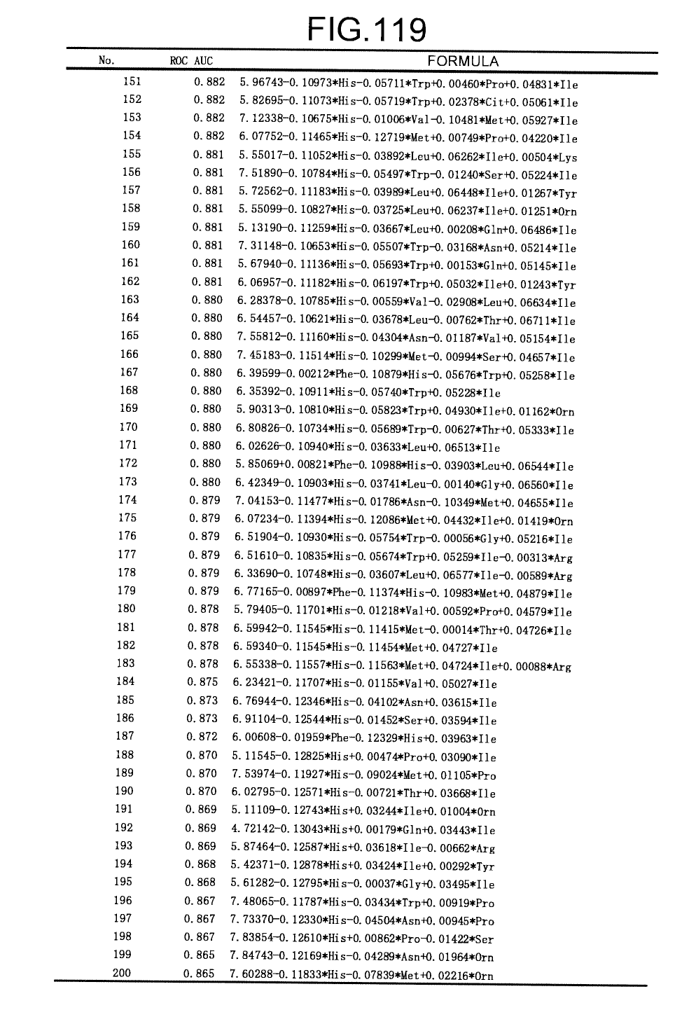

The discriminative ability of index formula 51 in 2-group discrimination between the cancer patient group and the cancer-free group is evaluated by AUC of the ROC curve (see FIG. 115). As a result, AUC of 0.898±0.017 (in 95% confidence interval, 0.865 to 0.932) is obtained. From this, it is found that index formula 51 is an index which is useful, with high diagnostic ability. In addition, when the optimum cutoff value of the average value of the sensitivity and the specificity of 2-group discrimination between the cancer patient group and the cancer-free group using index formula 51 is calculated, the cutoff value is -1.021, thereby obtaining 85.83% sensitivity and 82.74% specificity. From this, it is found that index formula 51 is an index which is useful, with high diagnostic ability. A plurality of logic regression equations having discriminative ability equivalent to index formula 51 are obtained. They are shown in FIGS. 116, 117, 118, and 119. The value of each coefficient in the equations shown in FIGS. 116, 117, 118, and 119 may be multiplied by a real number, and the value of constant term may be subjected to addition, subtraction, multiplication, and division with an arbitrary real constant.

### Example 13

The sample data used in Example 11 is used. Indexes which maximize 2-group discriminative ability between the cancer patient group and the cancer-free group are searched for, by using linear discriminant analysis (explanatory variable coverage method according to the maximizing criterion of area under the ROC curve). As a result, as index formula 52, a linear discriminant having His, Leu, Met, Cit, Ile, and Tyr (the numerical coefficients of the amino acid explanatory variables His, Leu, Met, Cit, Ile, and Tyr and the constant term are −0.09793, −0.04270, −0.17595, 0.05477, 0.07512, 0.03331, and 6.27211 in order) is obtained.

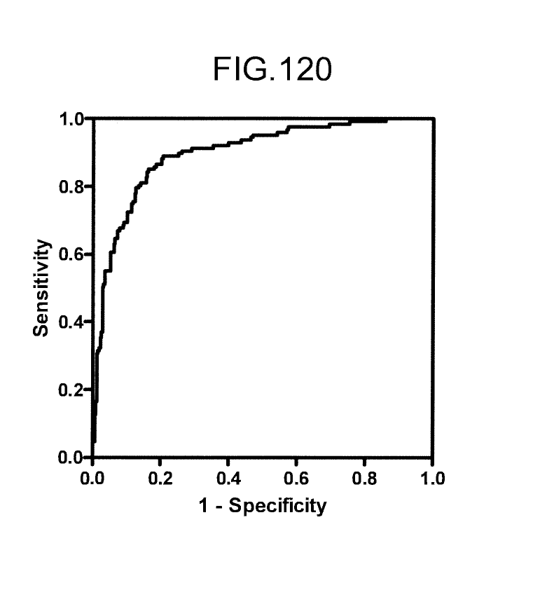

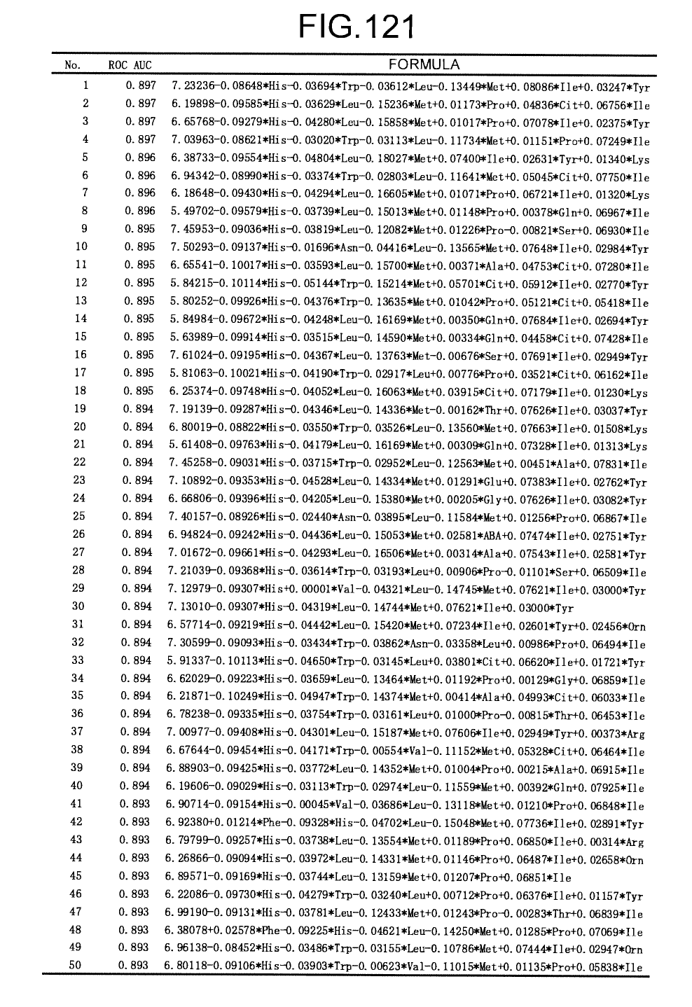

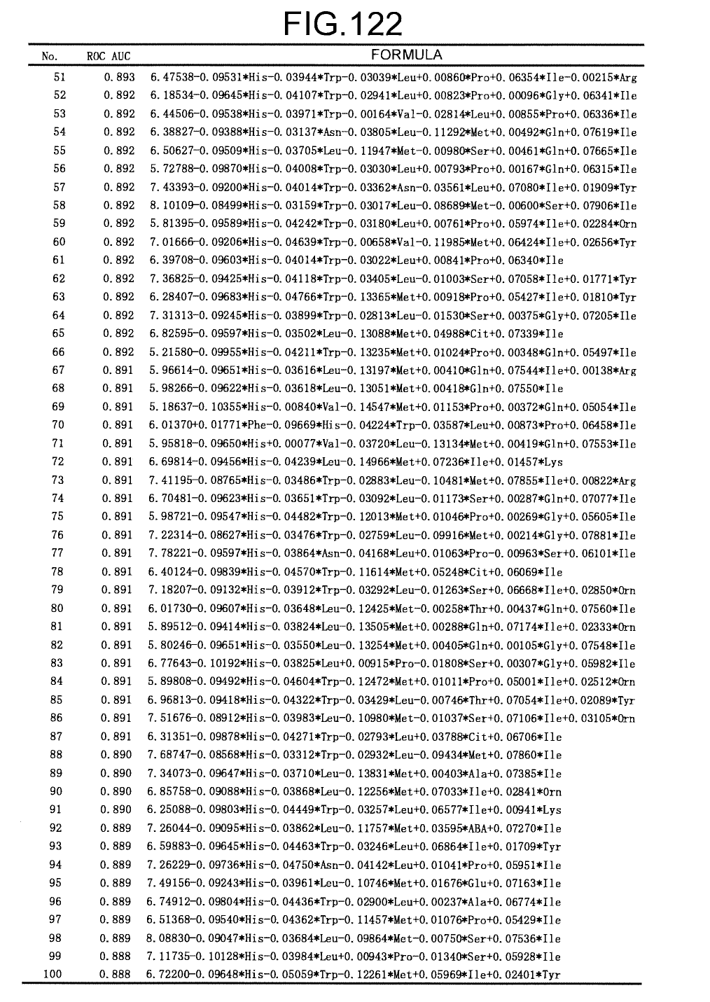

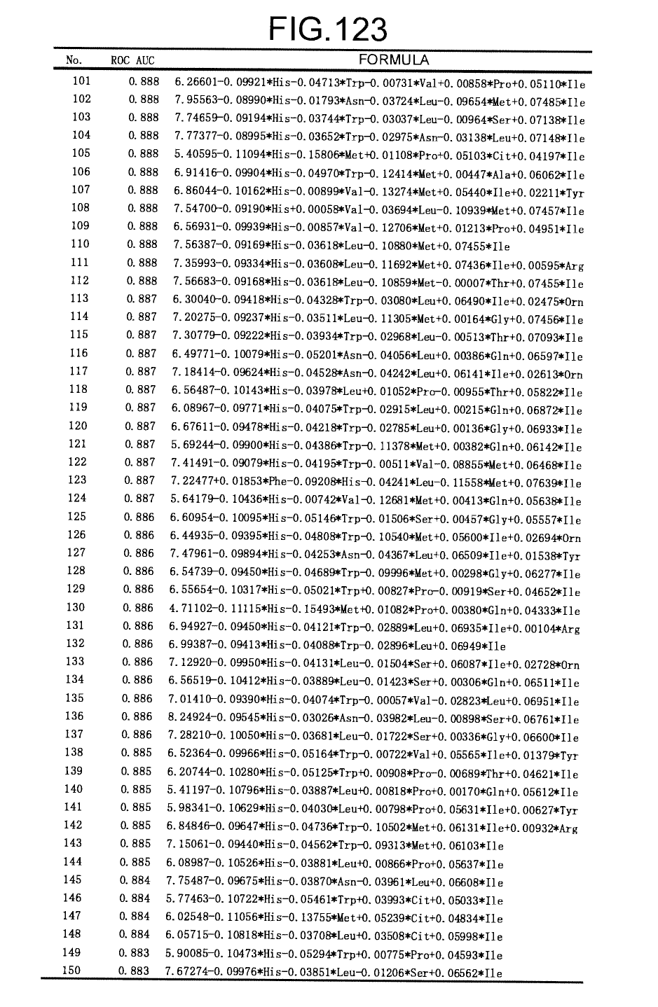

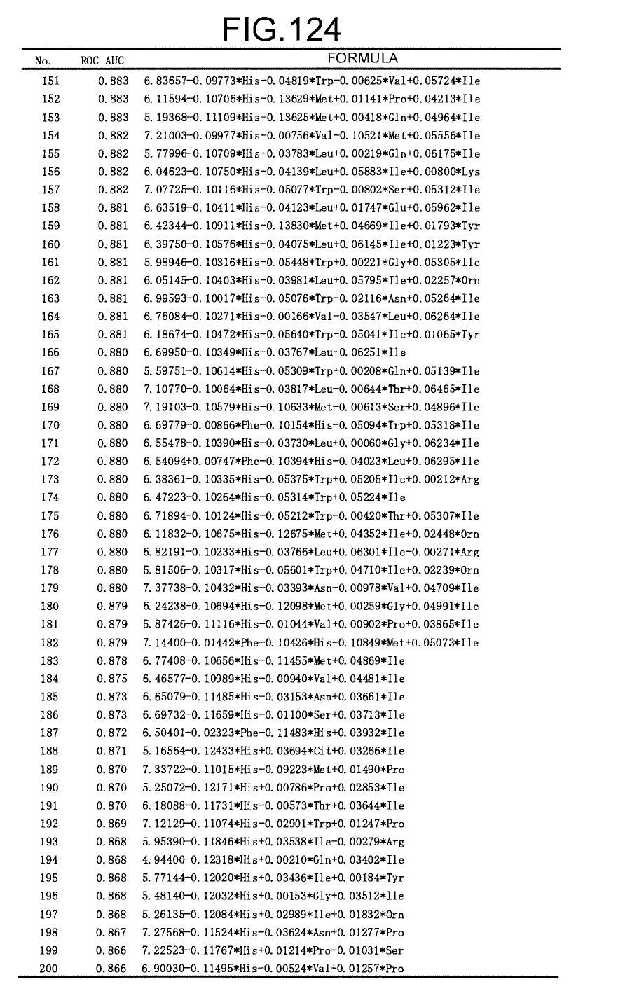

The discriminative ability of index formula 52 in 2-group discrimination between the cancer patient group and the cancer-free group is evaluated by AUC of the ROC curve (see FIG. 120). As a result, AUC of 0.899±0.017 (in 95% confidence interval, 0.866 to 0.932) is obtained. From this, it is found that index formula 52 is an index which is useful, with high diagnostic ability. In addition, when the optimum cutoff value of the average value of the sensitivity and the specificity of 2-group discrimination between the cancer patient group and the cancer-free group using index formula 52 is calculated, the cutoff value is −0.08697, thereby obtaining 85.04% sensitivity and 93.71% specificity. From this, it is found that index formula 52 is an index which is useful, with high diagnostic ability. A plurality of linear discriminants having discriminative ability equivalent to index formula 52 are obtained. They are shown in FIGS. 121, 122, 123, and 124. The value of each coefficient in the discriminants shown in FIGS. 121, 122, 123, and 124 may be multiplied by a real number, and the value of constant term may be subjected to addition, subtraction, multiplication, and division with an arbitrary real constant.

### Example 14

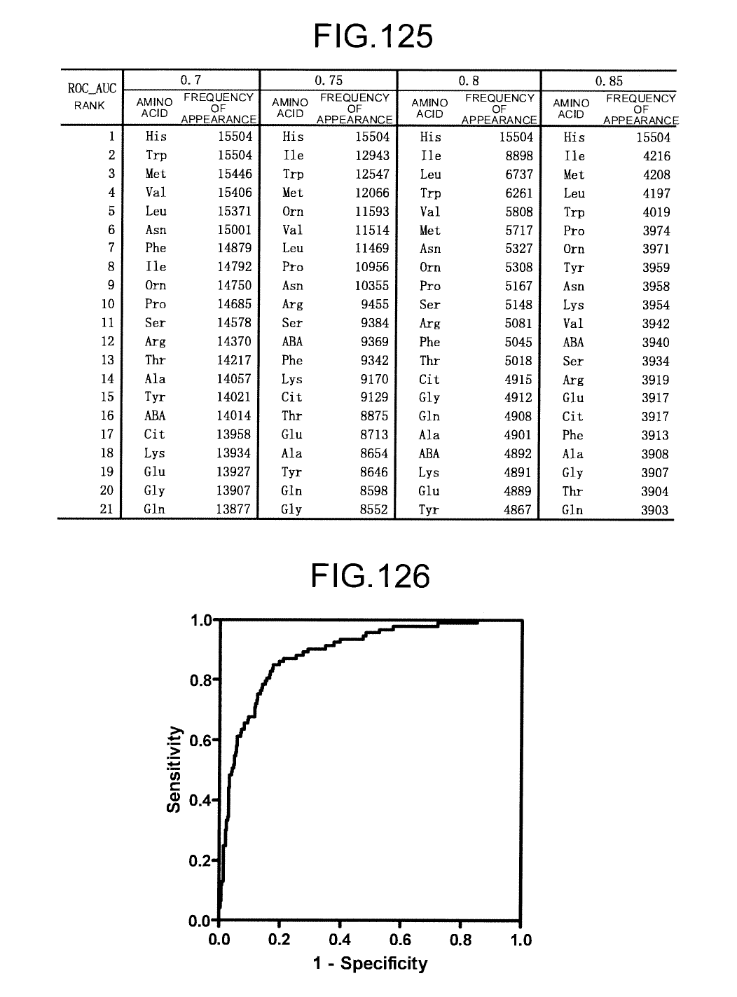

The sample data used in Example 11 is used. All the linear discriminants for performing 2-group discrimination between the cancer patient group and the cancer-free group are extracted by an explanatory variable coverage method. The areas under the ROC curve of all the discriminants satisfying the condition in which the maximum value of amino acid explanatory variables appearing in each of the discriminants is 6 are calculated. The frequencies with which the amino acids appear in the discriminants in which the areas under the ROC curve have values above certain threshold values are measured. When the areas under the ROC curve, 0.7, 0.75, 0.8, and 0.85 are threshold values, it is found that Asn, Pro, Met, Ile, Leu, His, Trp, and Orn are in the top ten among the amino acids extracted at high frequency at all times (see FIG. 125). From this, it is found that multivariate discriminants using these amino acids as explanatory variables have 2-group discriminative ability between the cancer patient group and the cancer-free group.

### Example 15

The sample data used in Example 11 is used. Indexes which maximize 2-group discriminative ability between the uterine cancer patient group and the uterine cancer-free group are searched for, by using logistic analysis (explanatory variable coverage method according to the maximizing criterion of area under the ROC curve). As a result, as index formula 53, a logistic regression equation having His, Leu, Met, Cit, Ile, and Tyr (the numerical coefficients of the amino acid explanatory variables His, Leu, Met, Cit, Ile, and Tyr and the constant term are −0.09298, −0.04434, −0.17139, 0.5732, 0.07267, 0.03790, and 4.67230 in order) is obtained.

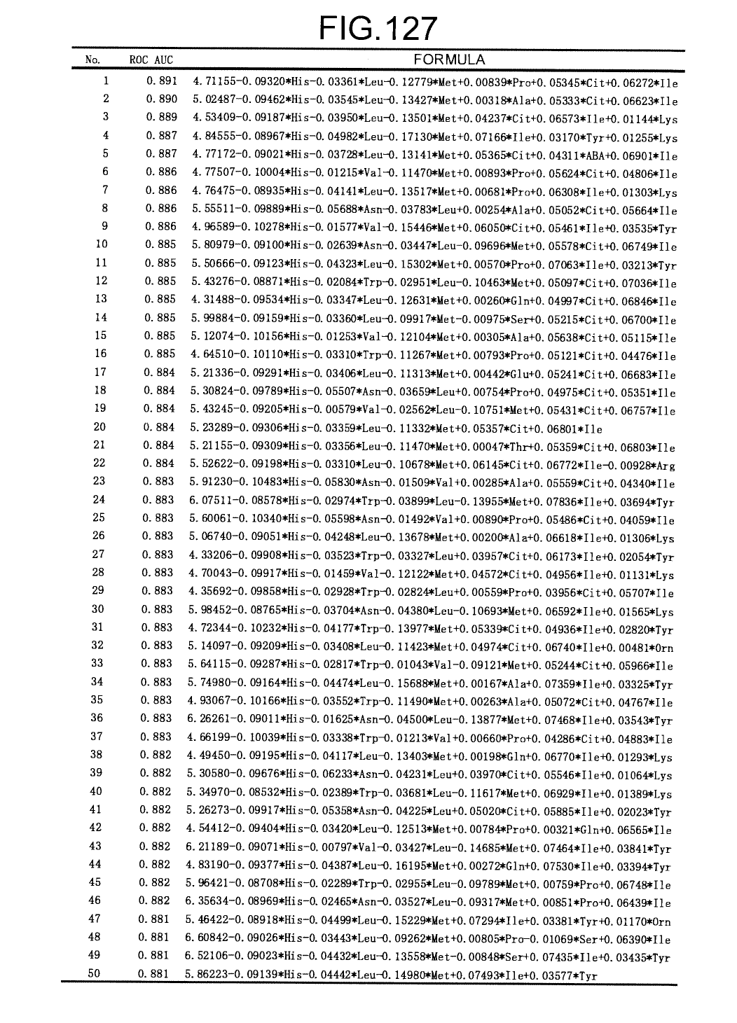

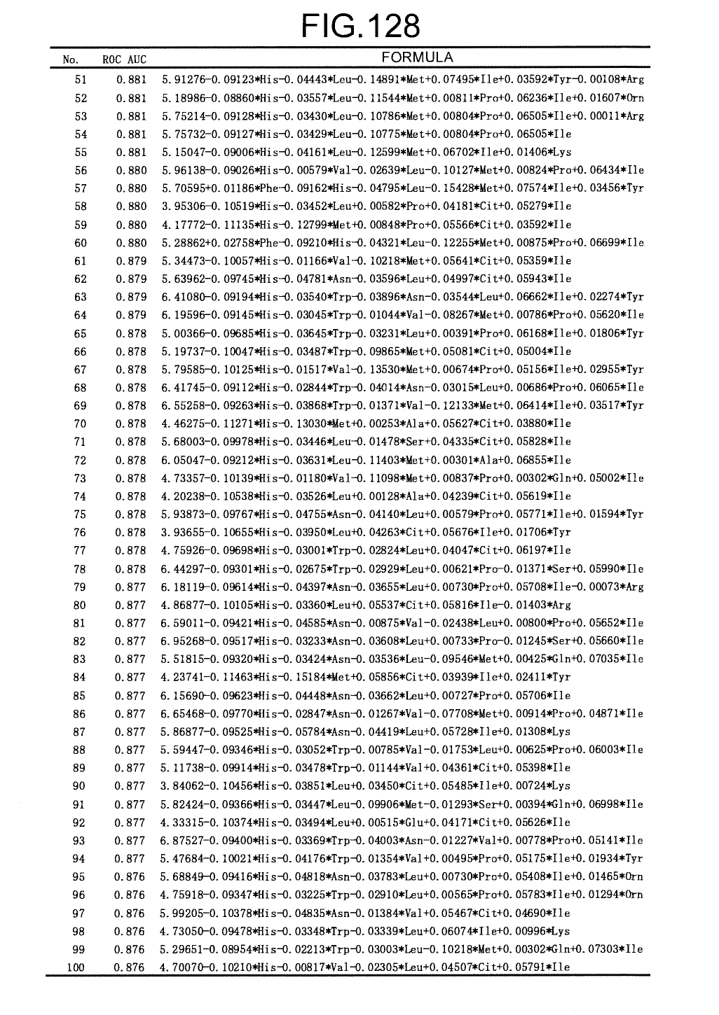

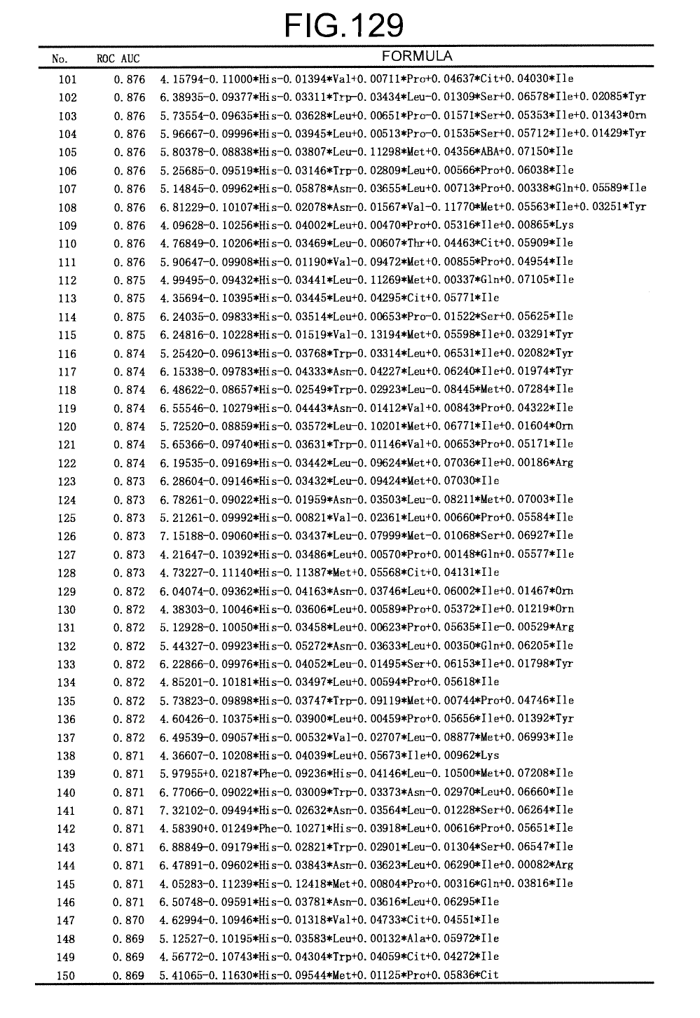

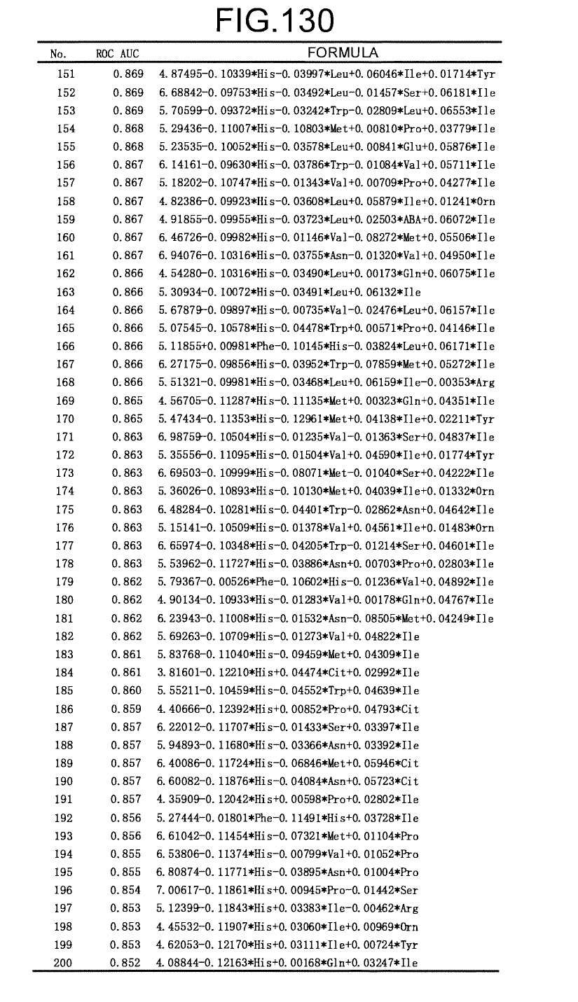

The discriminative ability of index formula 53 in 2-group discrimination between the uterine cancer patient group and the uterine cancer-free group is evaluated by AUC of the ROC curve (see FIG. 126). As a result, AUC of 0.893±0.019 (in 95% confidence interval, 0.856 to 0.930) is obtained. From this, it is found that index formula 53 is an index which is useful, with high diagnostic ability. In addition, when the optimum cutoff value of the average value of the sensitivity and the specificity of 2-group discrimination between the uterine cancer patient group and the uterine cancer-free group using index formula 53 is calculated, the cutoff value is −0.1608, thereby obtaining 87.10% sensitivity and 82.74% specificity. From this, it is found that index formula 53 is an index which is useful, with high diagnostic ability. A plurality of logistic regression equations having discriminative ability equivalent to index formula 53 are obtained. They are shown in FIGS. 127, 128, 129, and 130. The value of each coefficient in the equations shown in FIGS. 127, 128, 129, and 130 may be multiplied by a real number, and the value of constant term may be subjected to addition, subtraction, multiplication, and division with an arbitrary real constant.

### Example 16

The sample data used in Example 11 is used. Indexes which maximize 2-group discriminative ability between the uterine cancer patient group and the uterine cancer-free group are searched for, by using linear discriminant analysis (explanatory variable coverage method according to the maximizing criterion of area under the ROC curve). As a result, as index formula 54, a linear discriminant having His, Leu, Met, Cit, Ile, and Tyr (the numerical coefficients of the amino acid explanatory variables His, Leu, Met, Cit, Ile, and Tyr and the constant term are −0.09001, −0.04336, −0.17394, 0.07537, 0.06825, 0.03673, and 5.35827 in order) is obtained.

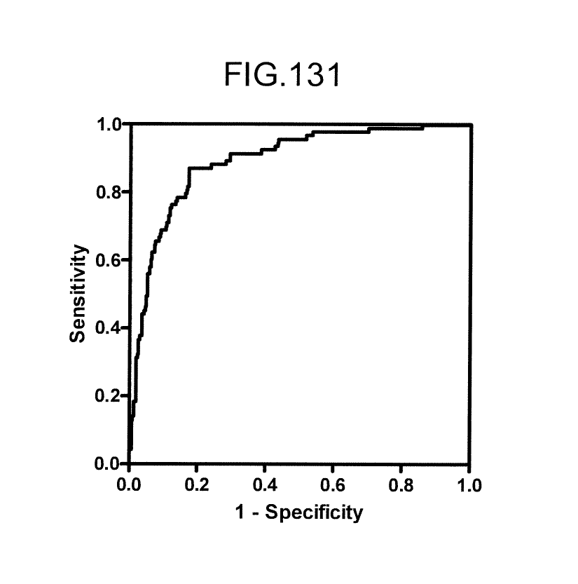

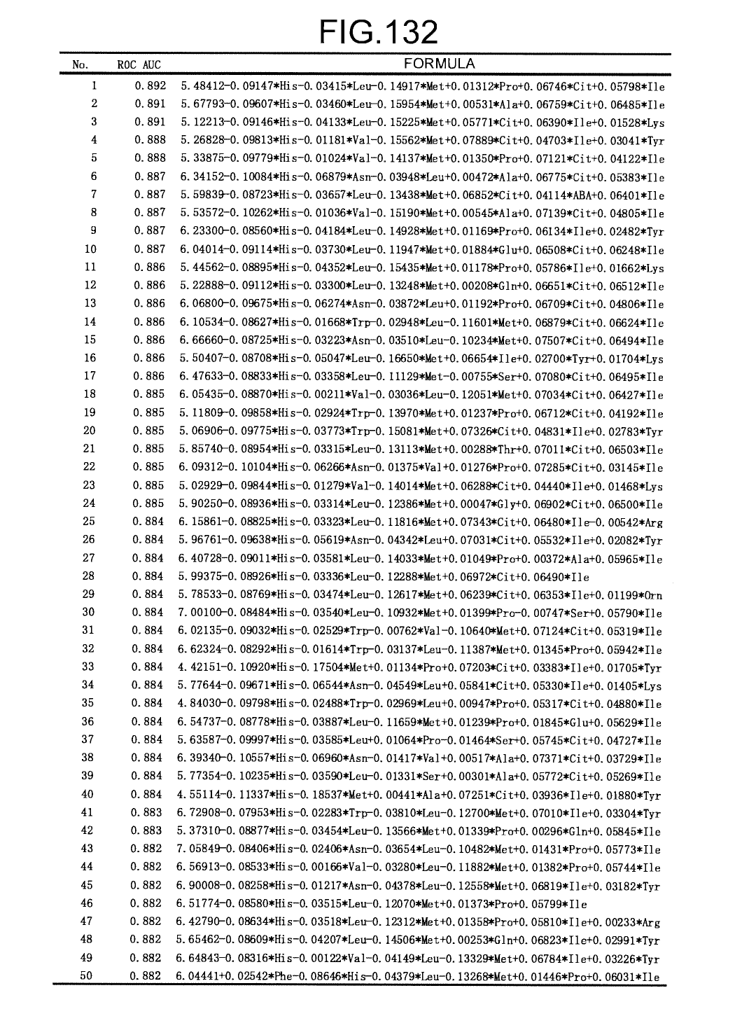

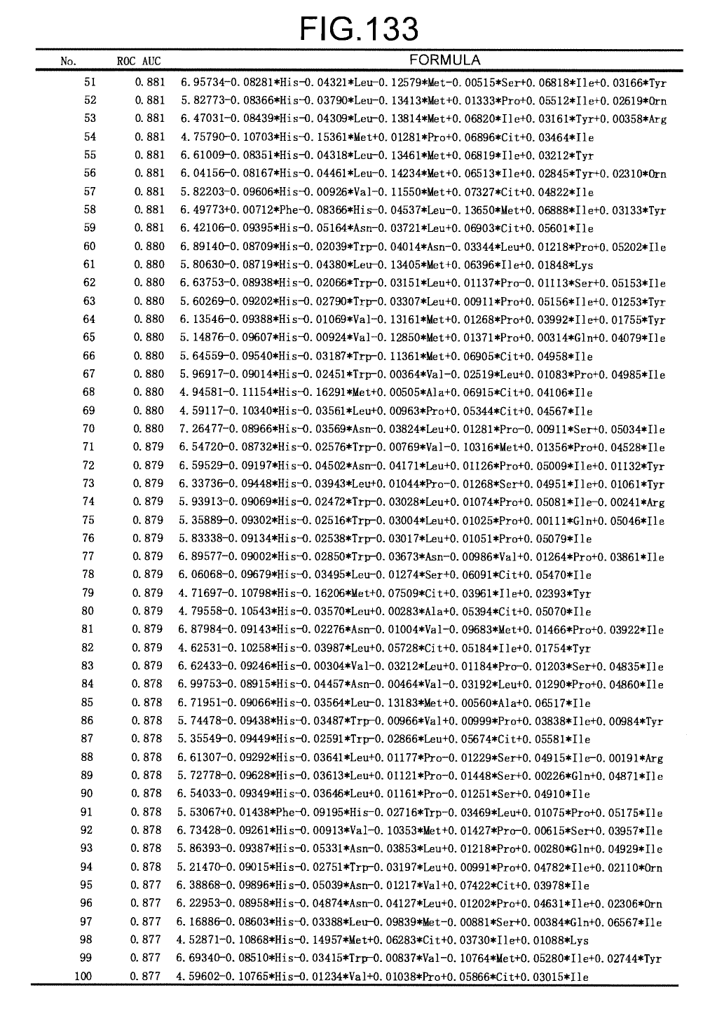

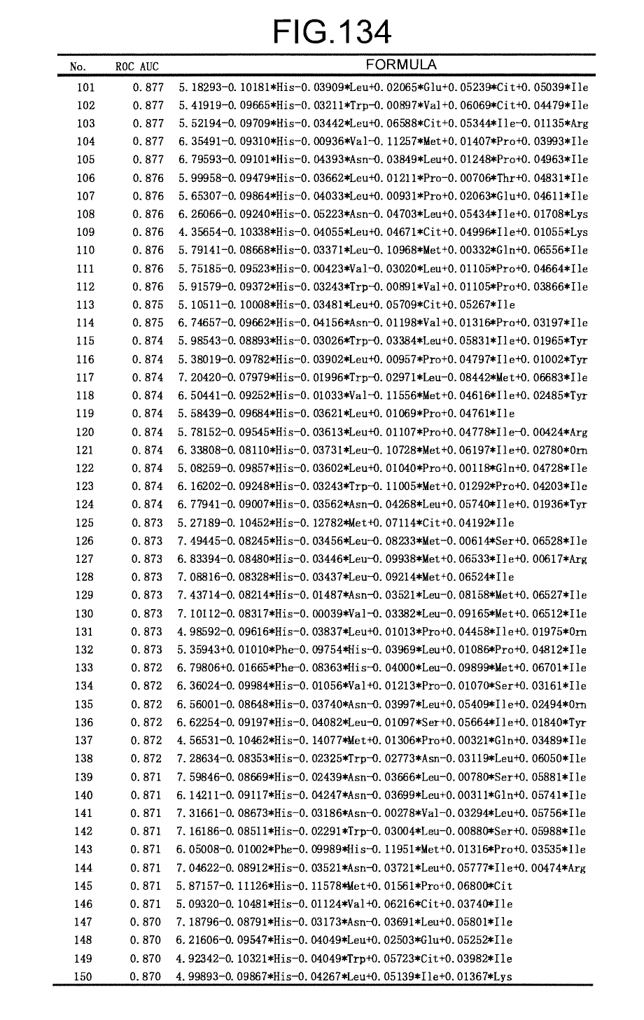

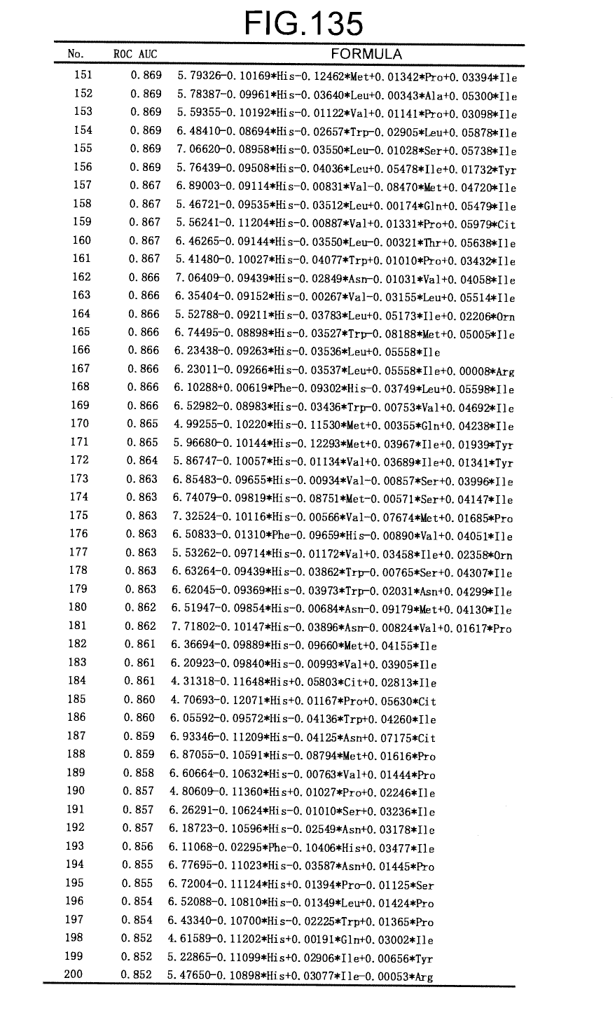

The discriminative ability of index formula 54 in 2-group discrimination between the uterine cancer patient group and the uterine cancer-free group is evaluated by AUC of the ROC curve (see FIG. 131). As a result, AUC of 0.898±0.017 (in 95% confidence interval, 0.865 to 0.932) is obtained. From this, it is found that index formula 54 is an index which is useful, with high diagnostic ability. In addition, when the optimum cutoff value of the average value of the sensitivity and the specificity of 2-group discrimination between the uterine cancer patient group and the uterine cancer-free group using index formula 54 is calculated, the cutoff value is −1.021, thereby obtaining 85.83% sensitivity and 83.06% specificity. From this, it is found that index formula 54 is an index which is useful, with high diagnostic ability. A plurality of linear discriminants having discriminative ability equivalent to index formula 54 are obtained. They are shown in FIGS. 132, 133, 134, and 135. The value of each coefficient in the discriminants shown in FIGS. 132, 133, 134, and 135 may be multiplied by a real number, and the value of constant term may be subjected to addition, subtraction, multiplication, and division with an arbitrary real constant.

### Example 17

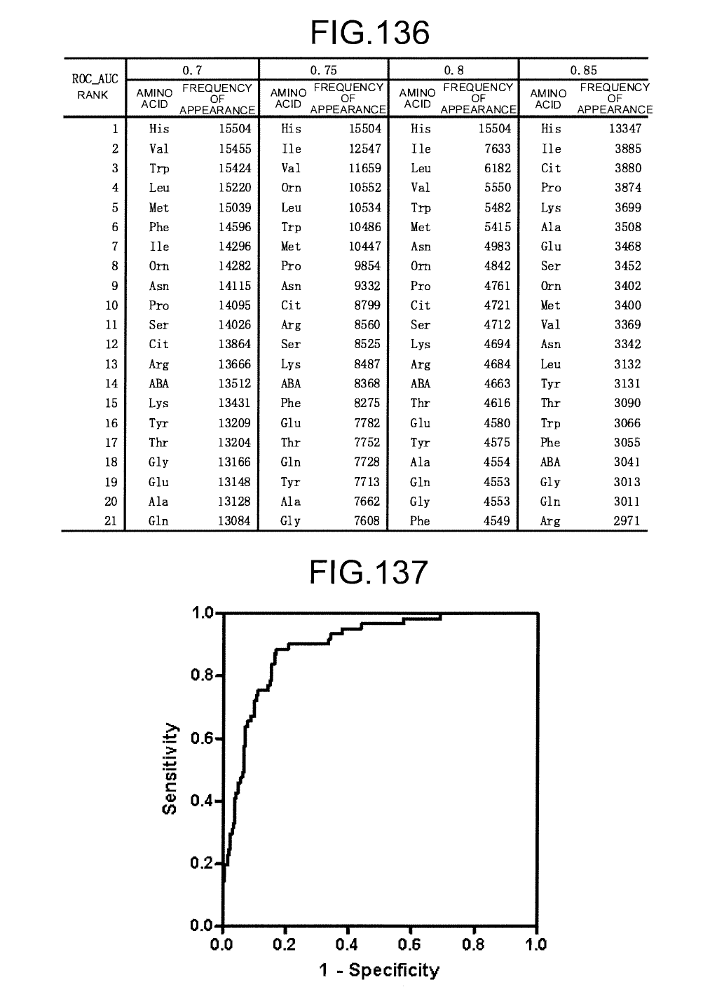

The sample data used in Example 11 is used. All the linear discriminants for performing 2-group discrimination between the uterine cancer patient group and the uterine cancer-free group are extracted by a explanatory variable coverage method. The areas under the ROC curve of all the discriminants satisfying the condition in which the maximum value of amino acid explanatory variables appearing in each of the discriminants is 6 are calculated. The frequencies with which the amino acids appear in the discriminants in which the areas under the ROC curve have values above certain threshold values are measured. When the areas under the ROC curve, 0.7, 0.75, 0.8, and 0.85 are threshold values, it is found that Pro, Met, Ile, His, and Orn are in the top ten among the amino acids extracted at high frequency at all times (see FIG. 136). From this, it is found that multivariate discriminants using these amino acids as explanatory variables have 2-group discriminative ability between the uterine cancer group and the uterine cancer-free group.

### Example 18

The sample data used in Example 11 is used. Indexes which maximize 2-group discriminative ability between the endometrial cancer patient group and the endometrial cancer-free group are searched for, by using logistic analysis (explanatory variable coverage method according to the maximizing criterion of area under the ROC curve). As a result, as index formula 55, a logistic regression equation having His, Asn, Val, Pro, Cit, and Ile (the numerical coefficients of the amino acid explanatory variables His, Asn, Val, Pro, Cit, and Ile and the constant term are −0.10149, −0.07968, −0.01336, 0.01018, 0.07129, 0.04046, and 4.92397 in order) is obtained.

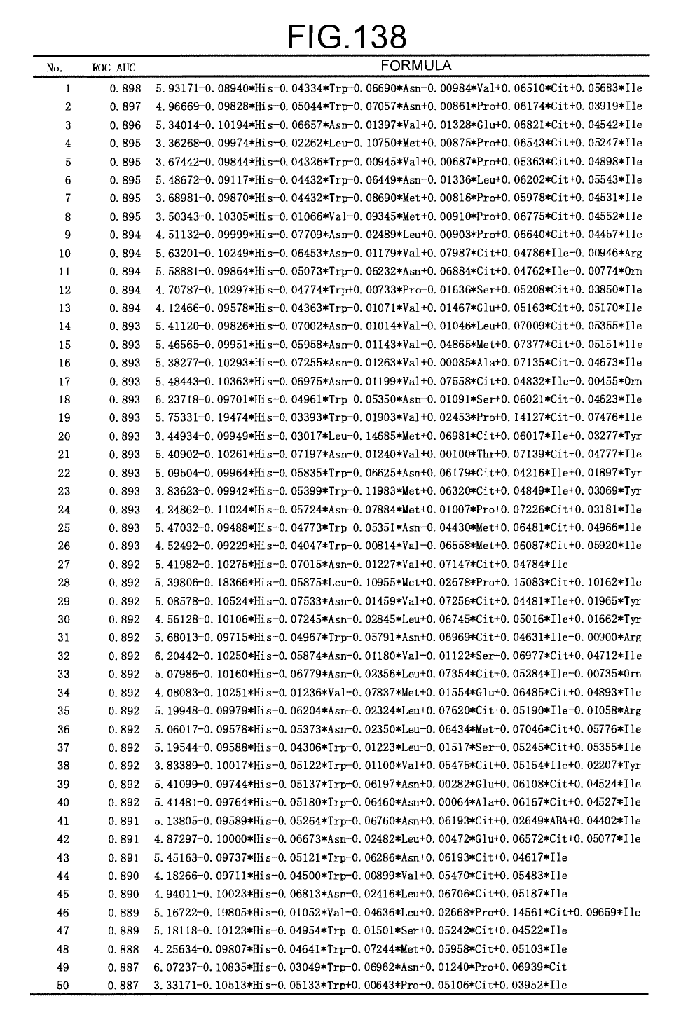

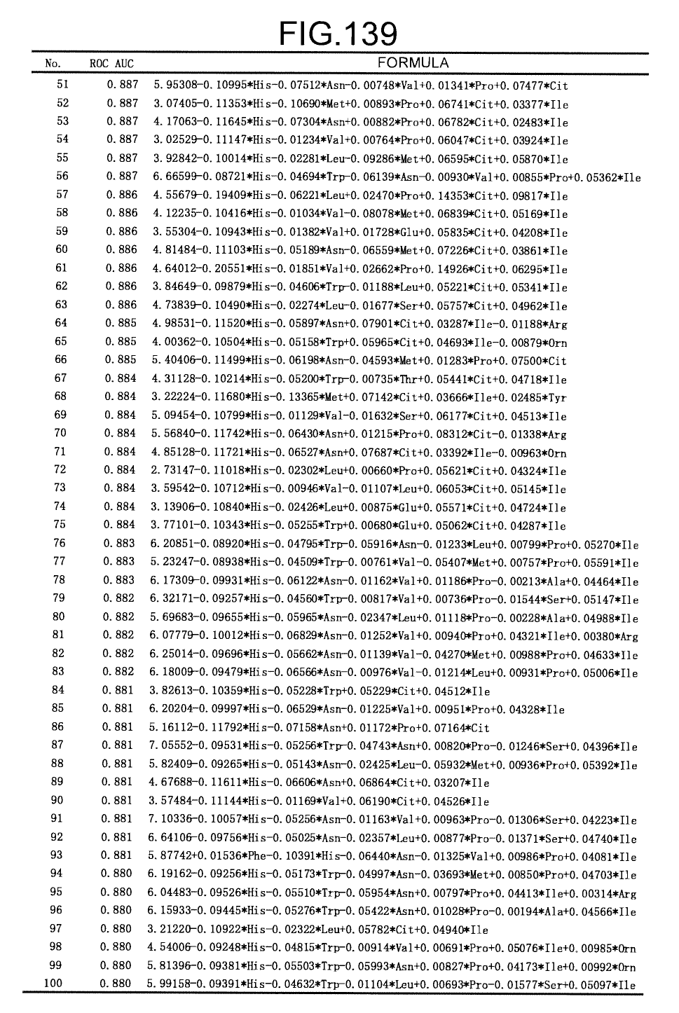

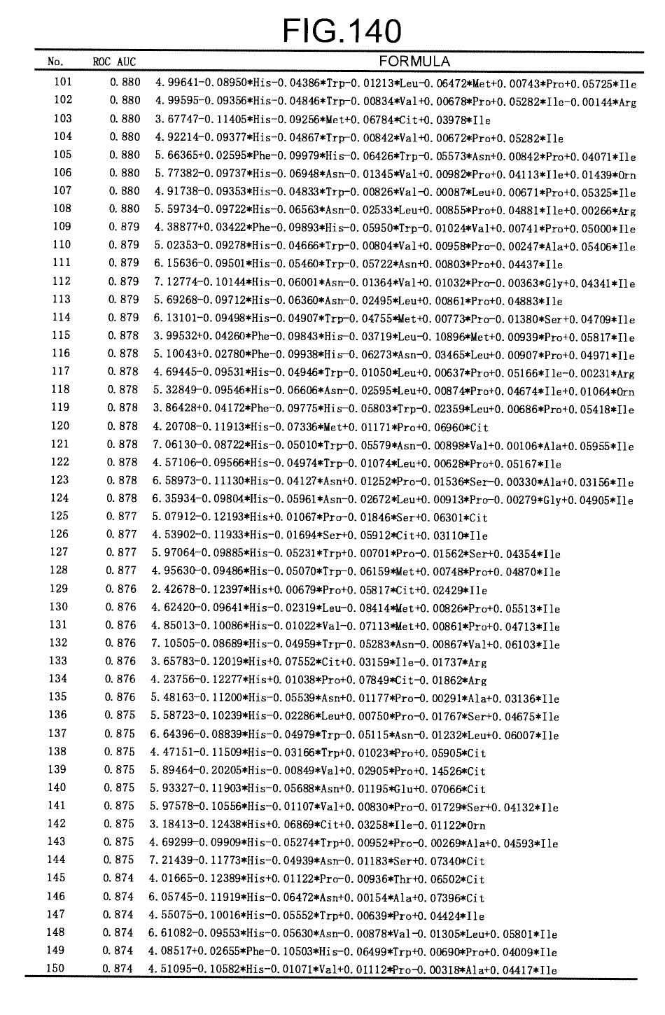

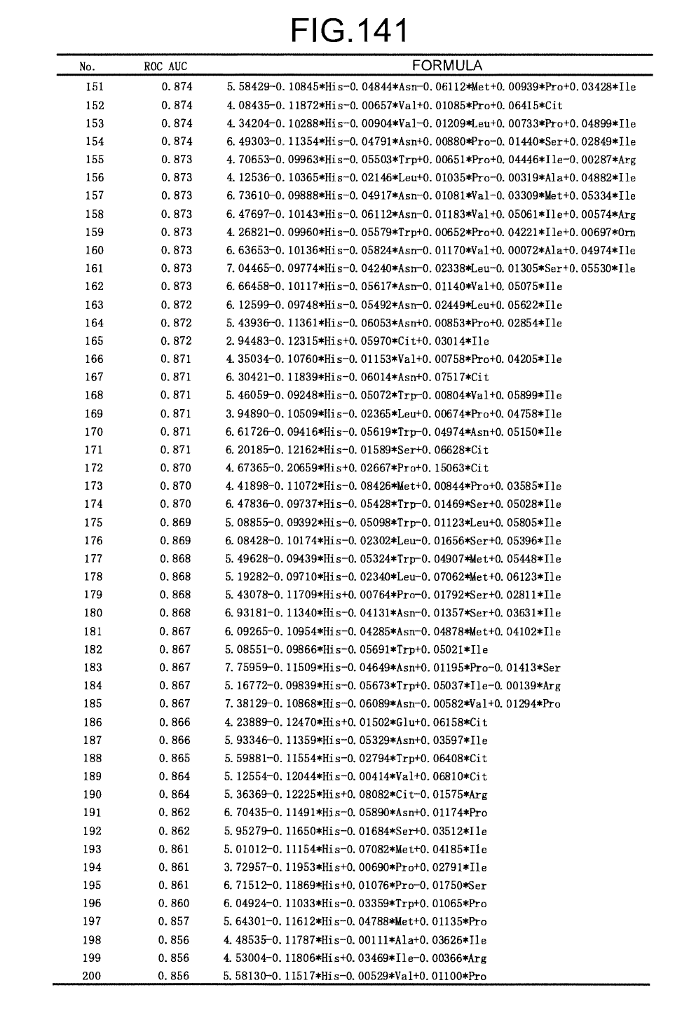

The discriminative ability of index formula 55 in 2-group discrimination between the endometrial cancer patient group and the endometrial cancer-free group is evaluated by AUC of the ROC curve (see FIG. 137). As a result, AUC of 0.8988±0.020 (in 95% confidence interval, 0.859 to 0.938) is obtained. From this, it is found that index formula 55 is an index which is useful, with high diagnostic ability. In addition, when the optimum cutoff value of the average value of the sensitivity and the specificity of 2-group discrimination between the endometrial cancer patient group and the endometrial cancer-free group using index formula 55 is calculated, the cutoff value is −1.490, thereby obtaining 88.52% sensitivity and 83.06% specificity. From this, it is found that index formula 55 is an index which is useful, with high diagnostic ability. A plurality of logistic regression equations having discriminative ability equivalent to index formula 55 are obtained. They are shown in FIGS. 138, 139, 140, and 141. The value of each coefficient in the equations shown in FIGS. 138, 139, 140, and 141 may be multiplied by a real number, and the value of constant term may be subjected to addition, subtraction, multiplication, and division with an arbitrary real constant.

### Example 19

The sample data used in Example 11 is used. Indexes which maximize 2-group discriminative ability between the endometrial cancer patient group and the endometrial cancer-free group are searched for, by using linear discriminant analysis (explanatory variable coverage method according to the maximizing criterion of area under the ROC curve). As a result, as index formula 56, a linear discriminant having His, Asn, Val, Pro, Cit, and Ile (the numerical coefficients of the amino acid explanatory variables His, Asn, Val, Pro, Cit, and Ile and the constant term are −0.10159, −0.08532, −0.01190, 0.01489, 0.09591, 0.03032, and 5.61323 in order) is obtained.

The discriminative ability of index formula 56 in 2-group discrimination between the endometrial cancer patient group and the endometrial cancer-free group is evaluated by

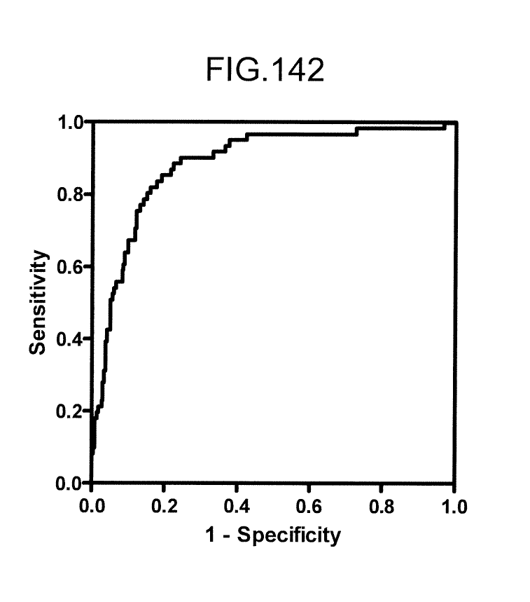

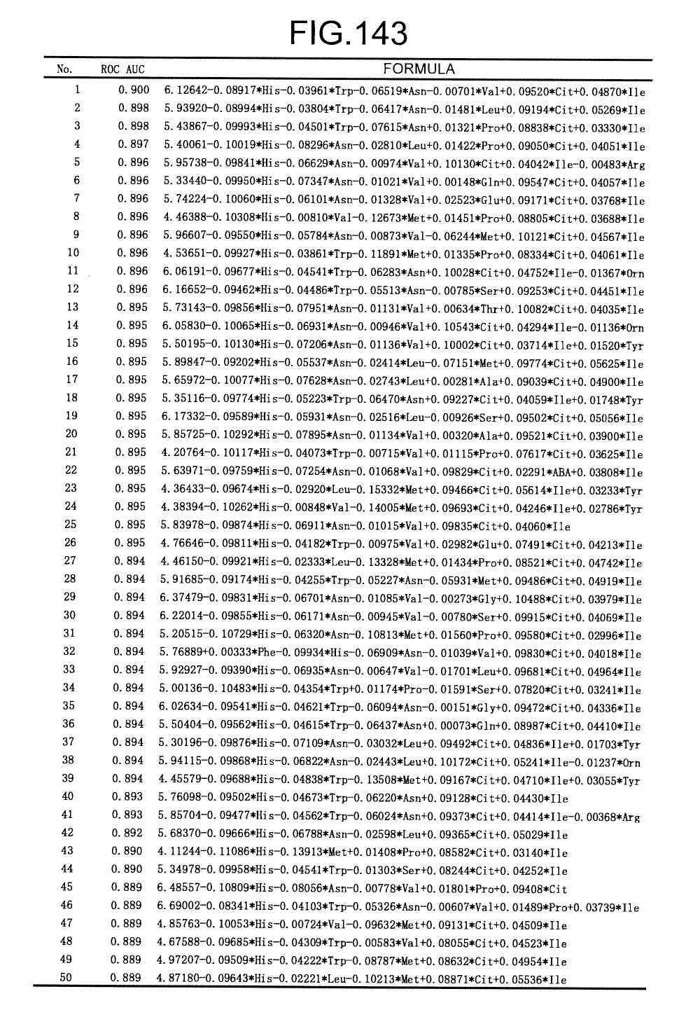

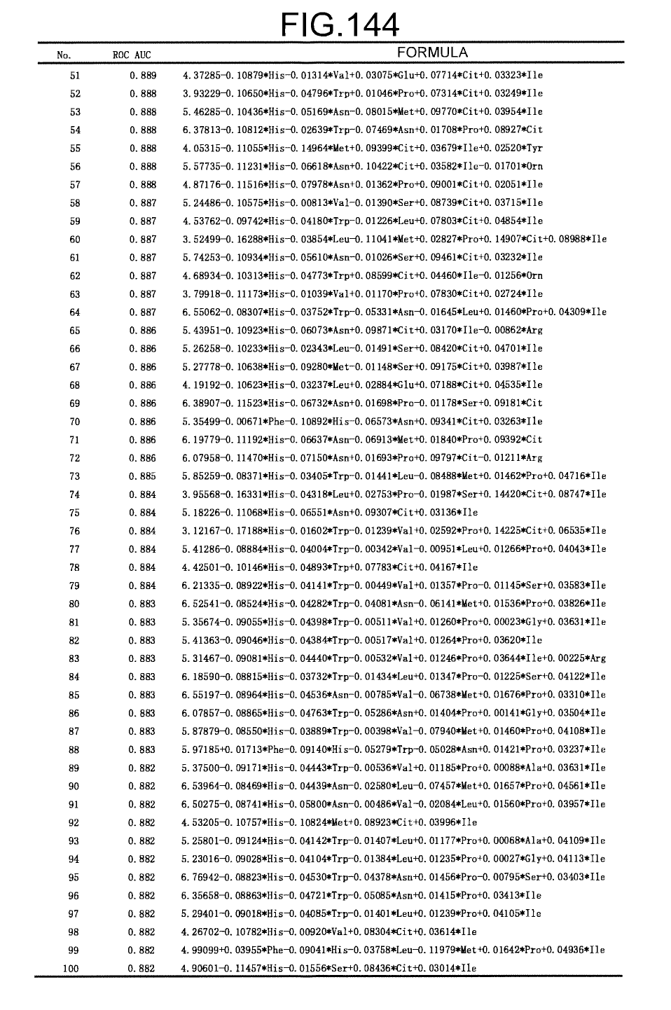

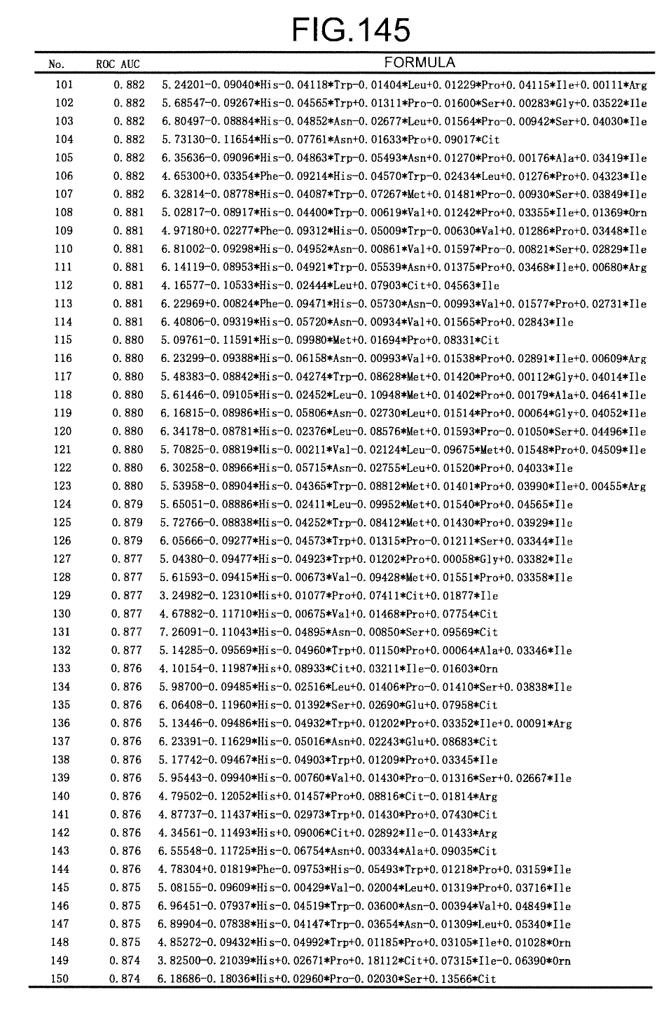

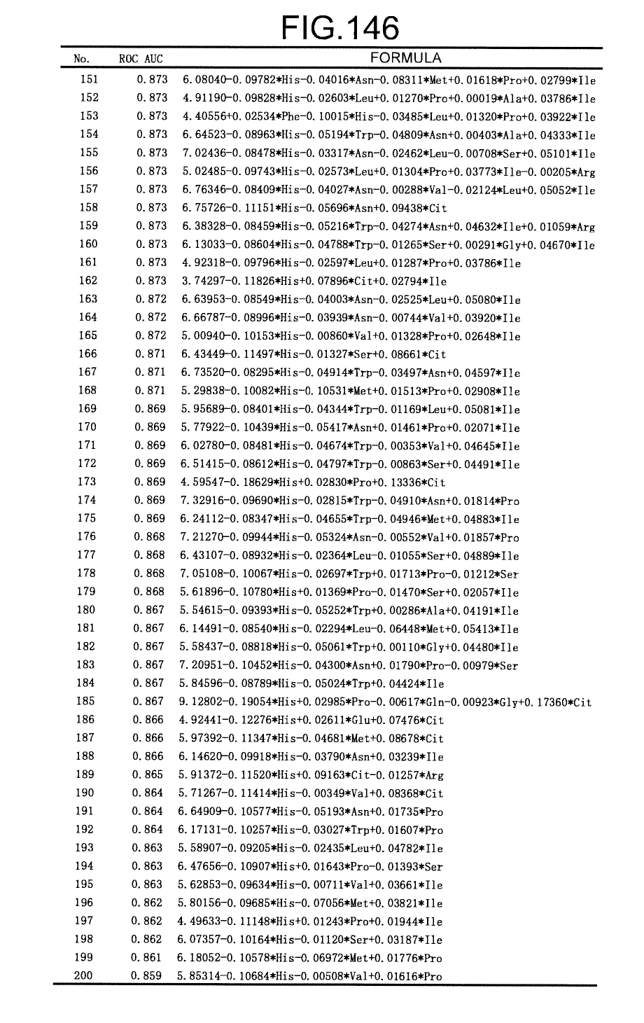

AUC of the ROC curve (see FIG. 142). As a result, AUC of 0.886±0.024 (in 95% confidence interval, 0.840 to 0.933) is obtained. From this, it is found that index formula 56 is an index which is useful, with high diagnostic ability. In addition, when the optimum cutoff value of the average value of the sensitivity and the specificity of 2-group discrimination between the endometrial cancer patient group and the endometrial cancer-free group using index formula 56 is calculated, the cutoff value is −1.356, thereby obtaining 88.52% sensitivity and 77.85% specificity. From this, it is found that index formula 56 is an index which is useful, with high diagnostic ability. A plurality of linear discriminants having discriminative ability equivalent to index formula 56 are obtained. They are shown in FIGS. 143, 144, 145, and 146. The value of each coefficient in the discriminants shown in FIGS. 143, 144, 145, and 146 may be multiplied by a real number, and the value of constant term may be subjected to addition, subtraction, multiplication, and division with an arbitrary real constant.

### Example 20

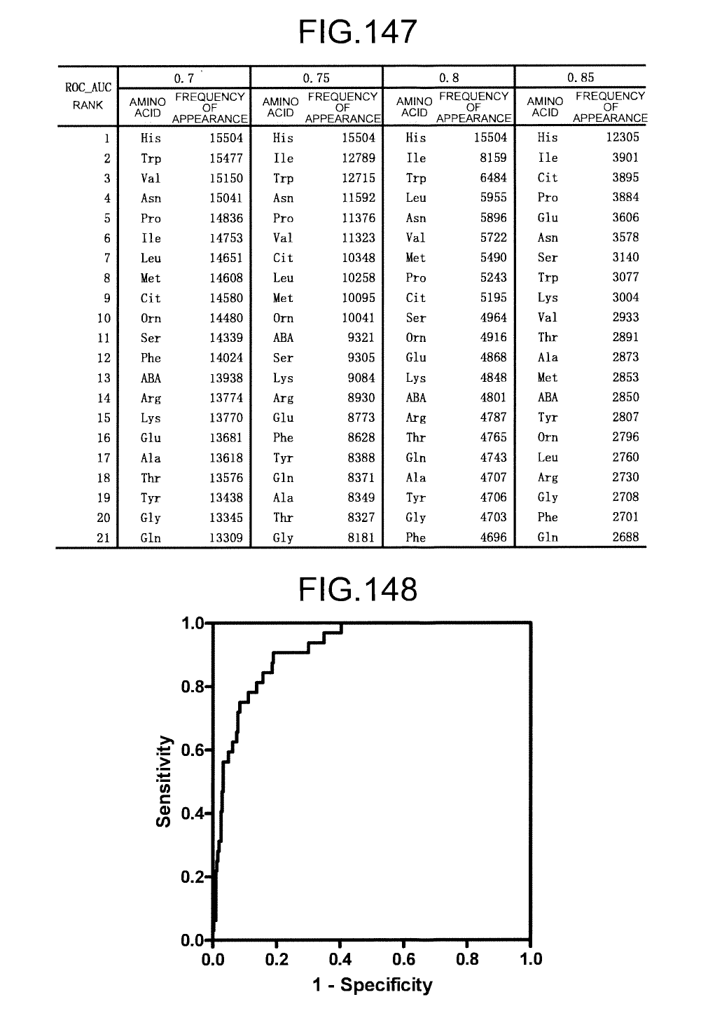

The sample data used in Example 11 is used. All the linear discriminants for performing 2-group discrimination between the endometrial cancer patient group and the endometrial cancer-free group are extracted by an explanatory variable coverage method. The areas under the ROC curve of all the discriminants satisfying the condition in which the maximum value of amino acid explanatory variables appearing in each of the discriminants is 6 are calculated. The frequencies with which the amino acids appear in the discriminants in which the areas under the ROC curve have values above certain threshold values are measured. When the areas under the ROC curve, 0.7, 0.75, 0.8, and 0.85 are threshold values, it is found that Asn, Pro, Cit, Val, Ile, His, and Trp are in the top ten among the amino acids extracted at high frequency at all times (see FIG. 147). From this, it is found that multivariate discriminants using these amino acids as explanatory variables have 2-group discriminative ability between the endometrial cancer patient group and the endometrial cancer-free group.

### Example 21

The sample data used in Example 11 is used. Indexes which maximize 2-group discriminative ability between the cervical cancer patient group and the cervical cancer-free group are searched for, by logistic analysis (explanatory variable coverage method according to the maximizing criterion of area under the ROC curve). As a result, as index formula 57, a logistic regression equation having His, Leu, Met, Ile, Tyr, and Lys (the numerical coefficients of the amino acid explanatory variables Orn, Gln, Trp, and Cit and the constant term are −0.08512, −0.07076, −0.23776, 0.07109, 0.04448, 0.01621, and 5.37165 in order) is obtained.

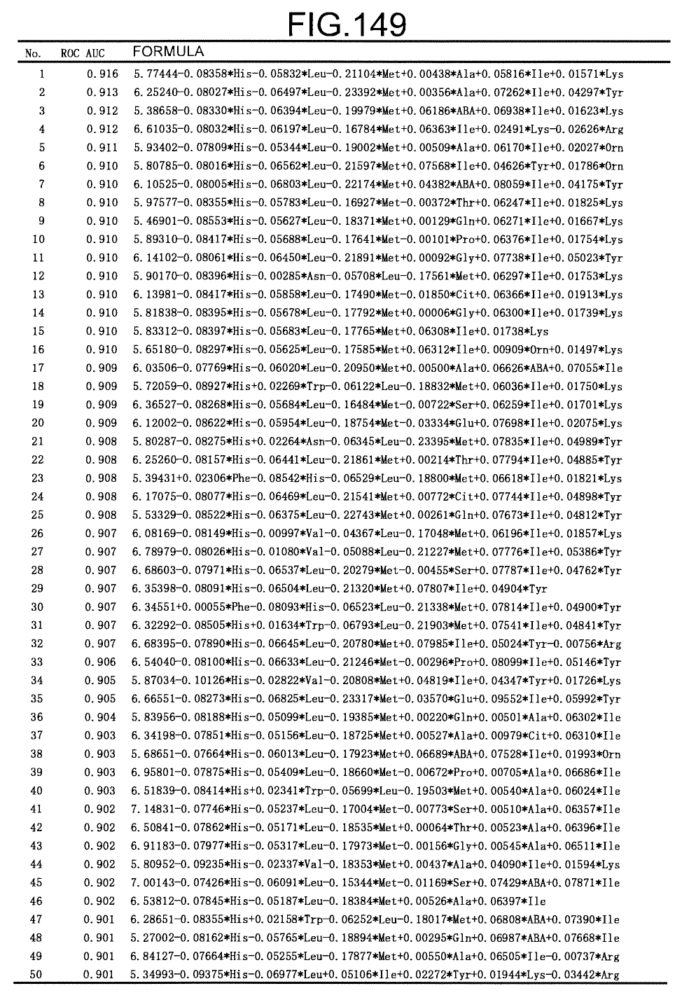

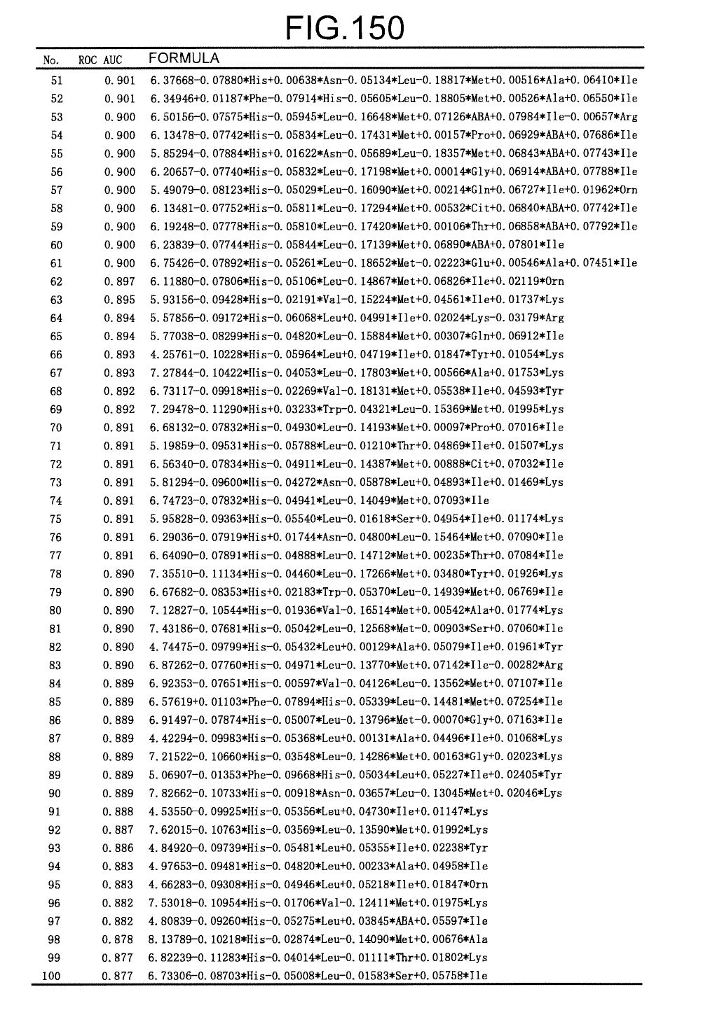

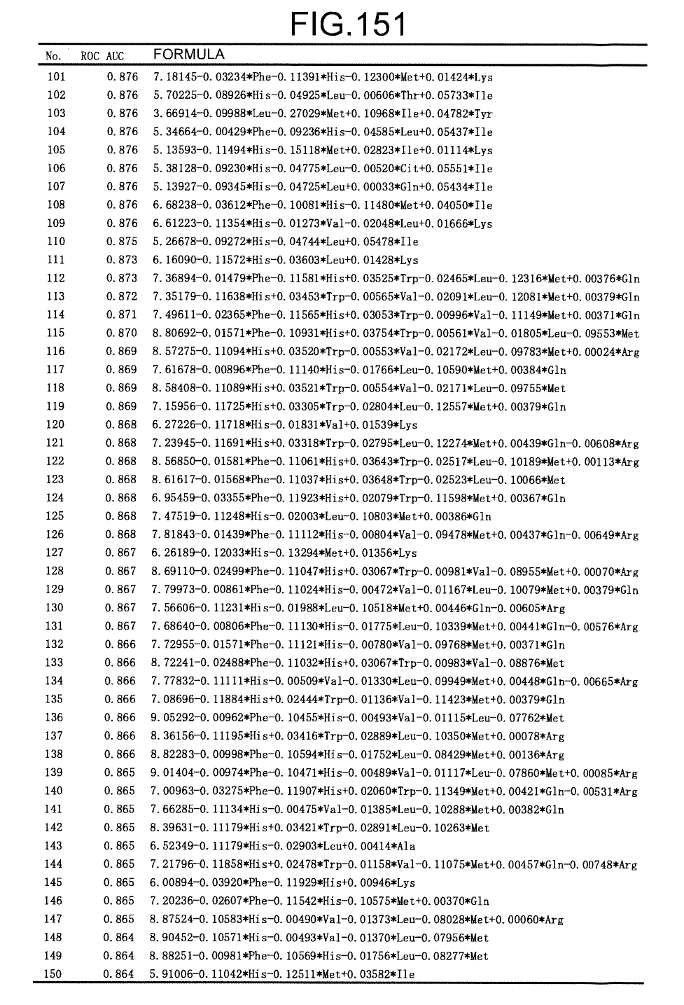

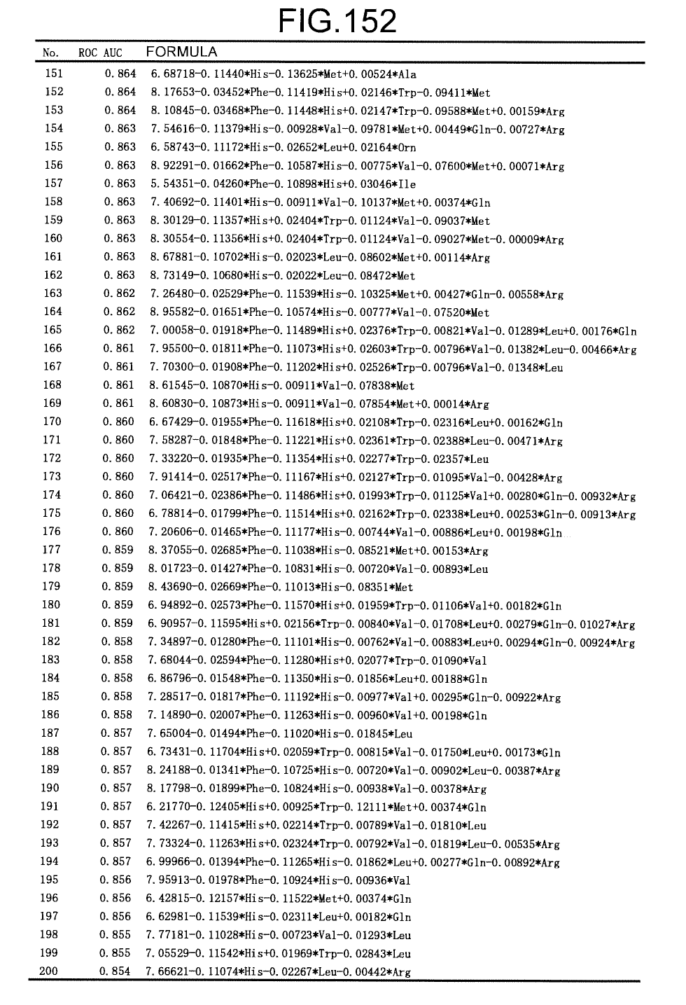

The discriminative ability of index formula 57 in 2-group discrimination between the cervical cancer patient group and the cervical cancer-free group is evaluated by AUC of the ROC curve (see FIG. 148). As a result, AUC of 0.919±0.020 (in 95% confidence interval, 0.879 to 0.959) is obtained. From this, it is found that index formula 57 is an index which is useful, with high diagnostic ability. In addition, when the optimum cutoff value of the average value of the sensitivity and the specificity of 2-group discrimination between the cervical cancer patient group and the cervical cancer-free group using index formula 57 is calculated, the cutoff value is −2.498, thereby obtaining 81.11% sensitivity and 85.87% specificity. From this, it is found that index formula 57 is an index which is useful, with high diagnostic ability. A plurality of logistic regression equations having discriminative ability equivalent to index formula 57 are obtained. They are shown in FIGS. 149, 150, 151, and 152. The value of each coefficient in the equations shown in FIGS. 149, 150, 151, and 152 may be multiplied by a real number, and the value of constant term may be subjected to addition, subtraction, multiplication, and division with an arbitrary real constant.

### Example 22

The sample data used in Example 11 is used. Indexes which maximize 2-group discriminative ability between the cervical cancer patient group and the cervical cancer-free group are searched for, by linear discriminant analysis (explanatory variable coverage method according to the maximizing criterion of area under the ROC curve). As a result, as index formula 58, a linear discriminant having His, Leu, Met, Ile, Tyr, and Lys (the numerical coefficients of the amino acid explanatory variables His, Leu, Met, Ile, Tyr, and Lys and the constant term are −0.09598, −0.08891, −0.25487, 0.09919, 0.04440, 0.02223, and 7.68576 in order) is obtained.

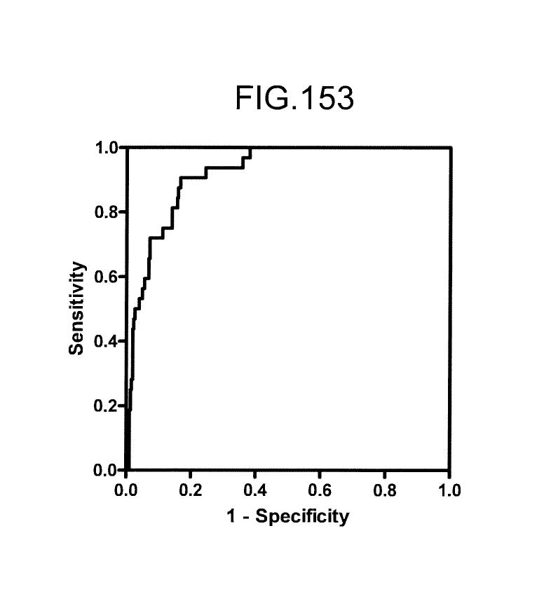

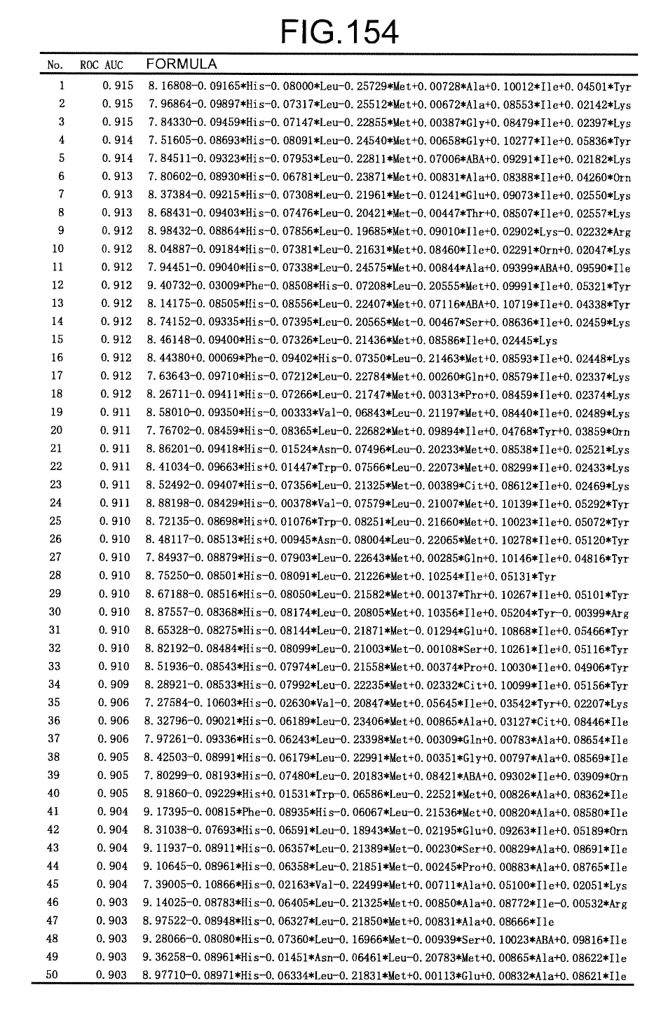

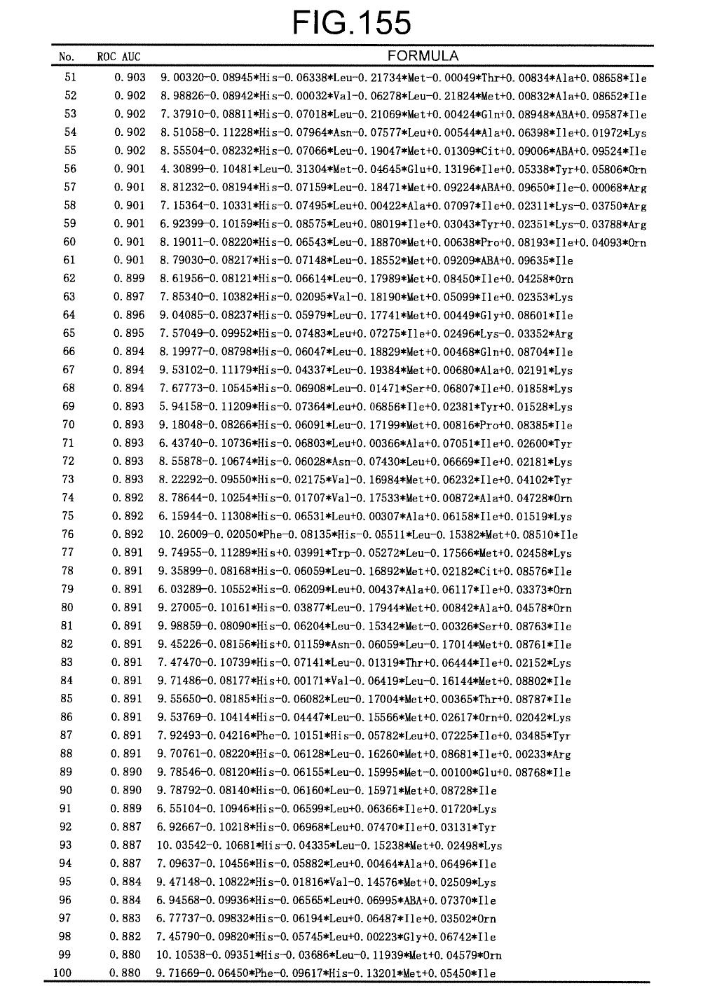

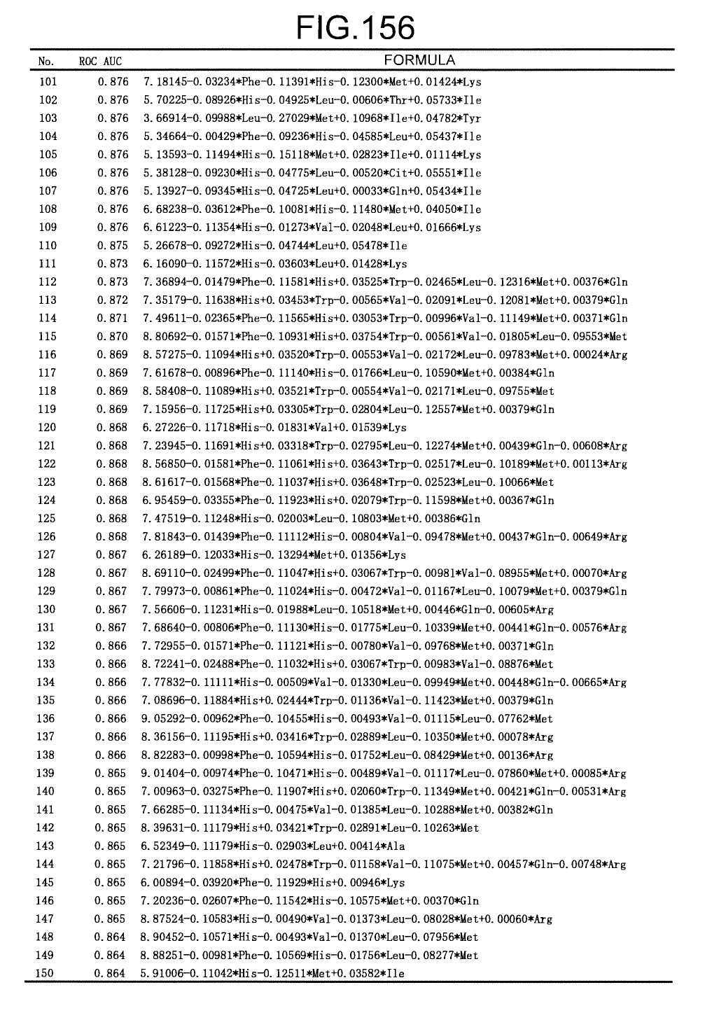

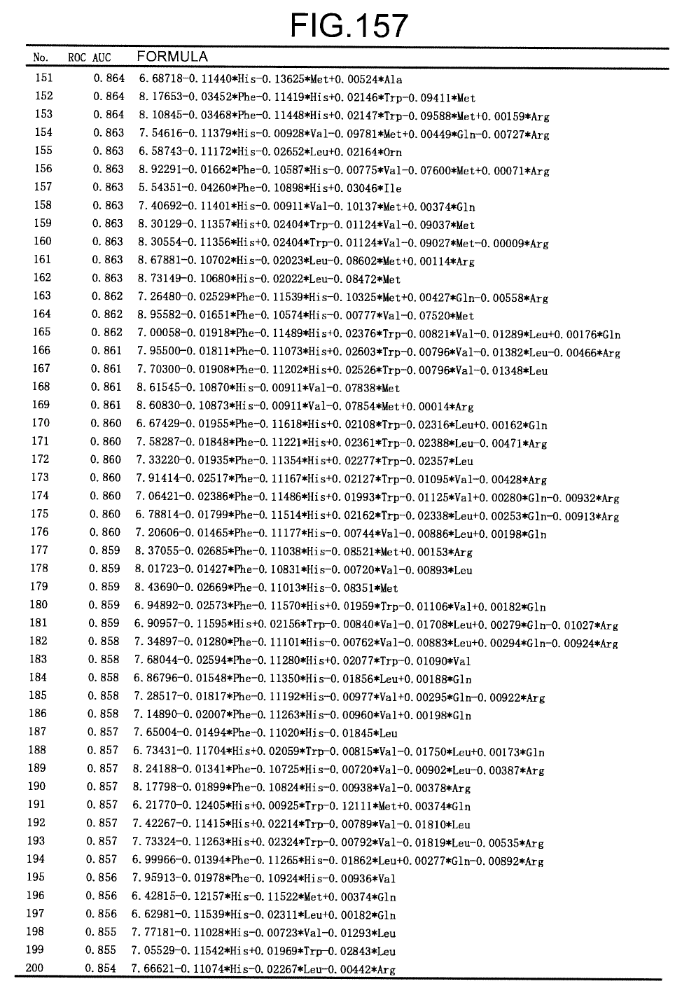

The discriminative ability of index formula 58 in 2-group discrimination between the cervical cancer patient group and the cervical cancer-free group is evaluated by AUC of the ROC curve (see FIG. 153). As a result, AUC of 0.921±0.019 (in 95% confidence interval, 0.883 to 0.959) is obtained. From this, it is found that index formula 58 is an index which is useful, with high diagnostic ability. In addition, when the optimum cutoff value of the average value of the sensitivity and the specificity of 2-group discrimination between the cervical cancer patient group and the cervical cancer-free group using index formula 58 is calculated, the cutoff value is −0.2189, thereby obtaining 90.63% sensitivity and 83.39% specificity. From this, it is found that index formula 58 is an index which is useful, with high diagnostic ability. A plurality of linear discriminants having discriminative ability equivalent to index formula 58 are obtained. They are shown in FIGS. 154, 155, 156, and 157. The value of each coefficient in the discriminants shown in FIGS. 154, 155, 156, and 157 may be multiplied by a real number, and the value of constant term may be subjected to addition, subtraction, multiplication, and division with an arbitrary real constant.

### Example 23

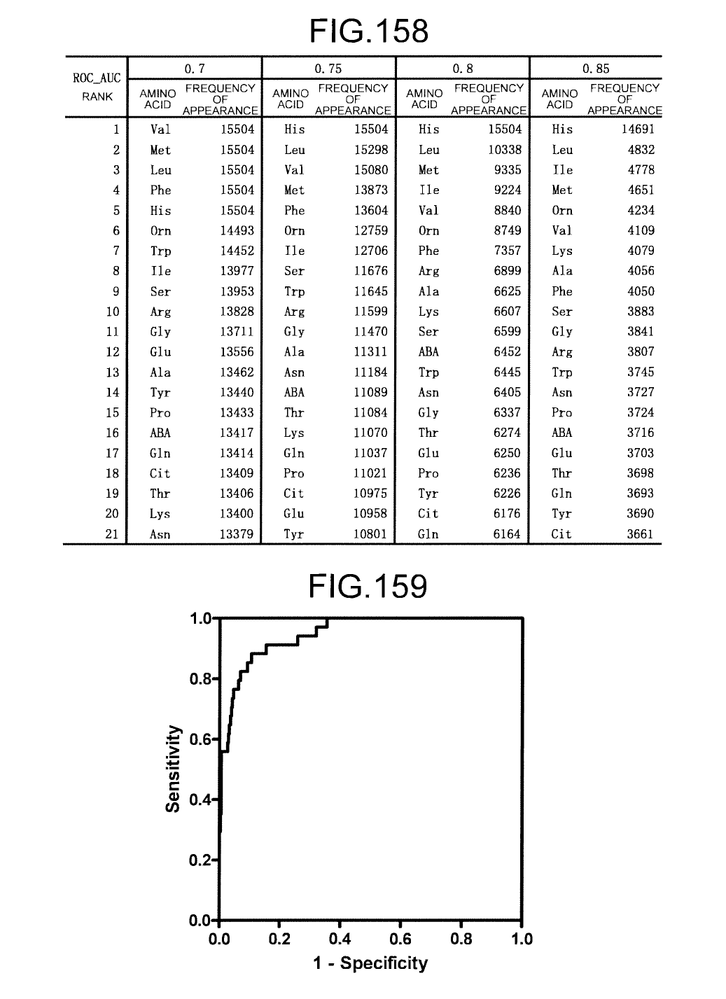

The sample data used in Example 11 is used. All the linear discriminants for performing 2-group discrimination between the cervical cancer patient group and the cervical cancer-free group are extracted by an explanatory variable coverage method. The areas under the ROC curve of all the discriminants satisfying the condition in which the maximum value of amino acid explanatory variables appearing in each of the discriminants is 6 are calculated. The frequencies with which the amino acids appear in the discriminants in which the areas under the ROC curve have values above certain threshold values are measured. When the areas under the ROC curve, 0.7, 0.75, 0.8, and 0.85 are threshold values, it is found that Val, Met, Leu, Phe, His, and Orn are in the top ten among the amino acids extracted at high frequency at all times (see FIG. 158). From this, it is found that multivariate discriminants using these amino acids as explanatory variables have 2-group discriminative ability between the cervical cancer patient group and the cervical cancer-free group.

### Example 24

The sample data used in Example 11 is used. Indexes which maximize 2-group discriminative ability between the ovarian cancer patient group and the ovarian cancer-free group are searched for, by logistic analysis (explanatory variable coverage method according to the maximizing criterion of area under the ROC curve). As a result, as index formula 59, a logistic regression equation having His, Trp, Glu, Cit, Ile, and Orn (the numerical coefficients of the amino acid explanatory variables His, Trp, Glu, Cit, Ile, and Orn and the constant term are −0.13767, −0.11457, −0.04031, −0.15449, 0.08765, 0.04631, and 10.70464 in order) is obtained.

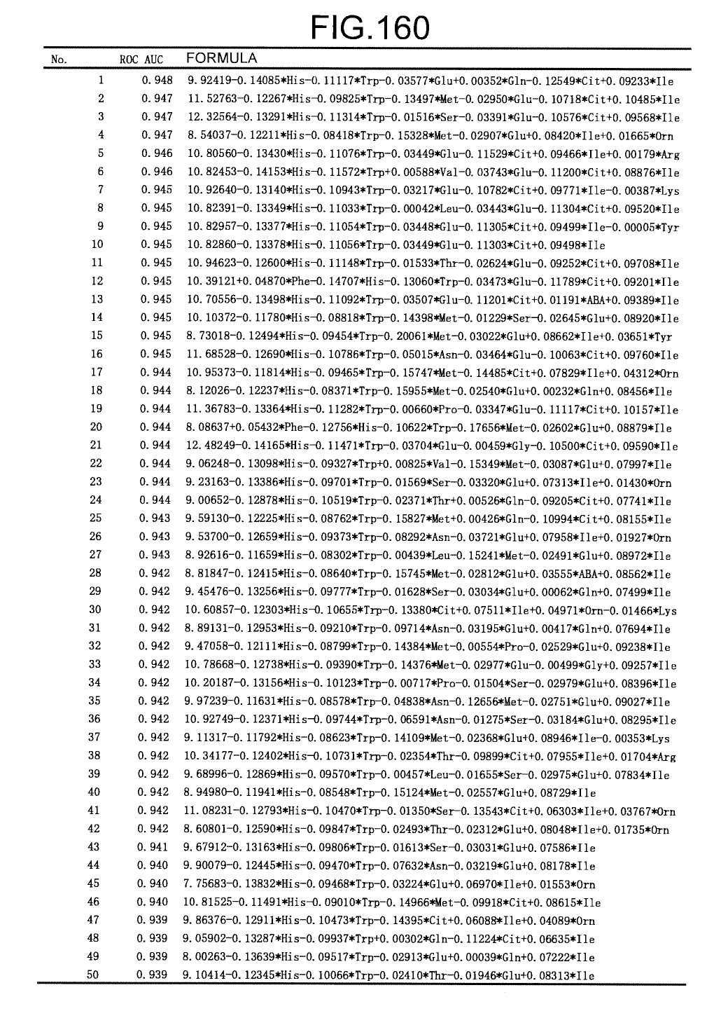

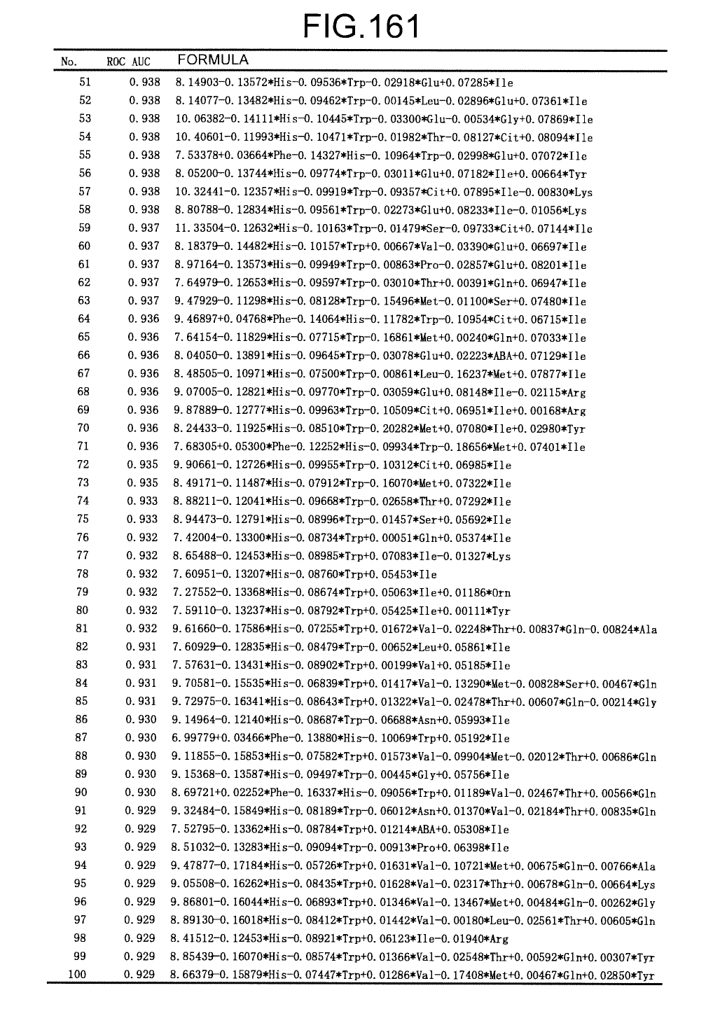

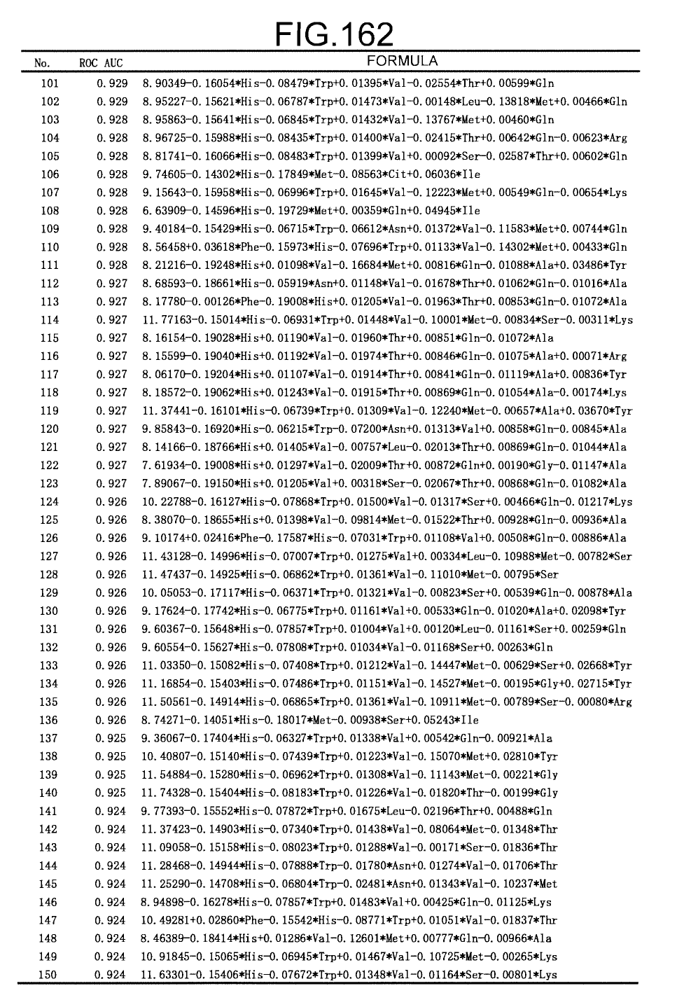

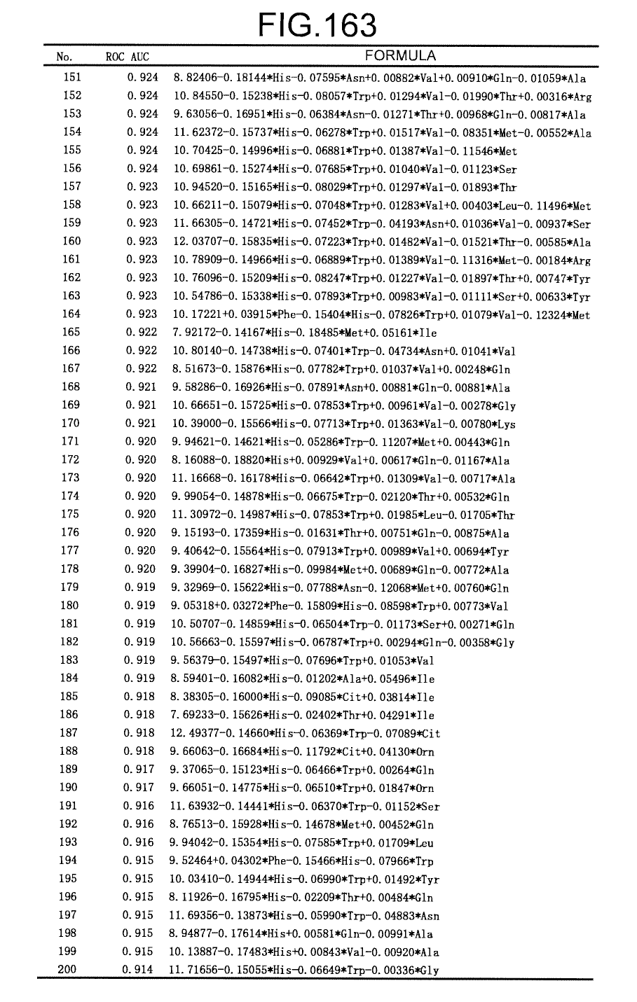

The discriminative ability of index formula 59 in 2-group discrimination between the ovarian cancer patient group and the ovarian cancer-free group is evaluated by AUC of the ROC curve (see FIG. 159). As a result, AUC of 0.950±0.016 (in 95% confidence interval, 0.917 to 0.982) is obtained. From this, it is found that index formula 59 is an index which is useful, with high diagnostic ability. In addition, when the optimum cutoff value of the average value of the sensitivity and the specificity of 2-group discrimination between the ovarian cancer patient group and the ovarian cancer-free group using index formula 59 is calculated, the cutoff value is −1.909, thereby obtaining 88.24% sensitivity and 89.58% specificity. From this, it is found that index formula 59 is an index which is useful, with high diagnostic ability. A plurality of logistic regression equations having discriminative ability equivalent to index formula 59 are obtained. They are shown in FIGS. 160, 161, 162, and 163. The value of each coefficient in the equations shown in FIGS. 160, 161, 162, and 163 may be multiplied by a real number, and the value of constant term may be subjected to addition, subtraction, multiplication, and division with an arbitrary real constant.

### Example 25

The sample data used in Example 11 is used. Indexes which maximize 2-group discriminative ability between the ovarian cancer patient group and the ovarian cancer-free group are searched for, by linear discriminant analysis (explanatory variable coverage method according to the maximizing criterion of area under the ROC curve). As a result, as index formula 60, a linear discriminant having His, Trp, Glu, Cit, Ile, and Orn (the numerical coefficients of the amino acid explanatory variables His, Trp, Glu, Cit, Ile, and Orn and the constant term are −0.13983, −0.11341, −0.04572, −0.10368, 0.12160, 0.05459, and 9.27981 in order) is obtained.

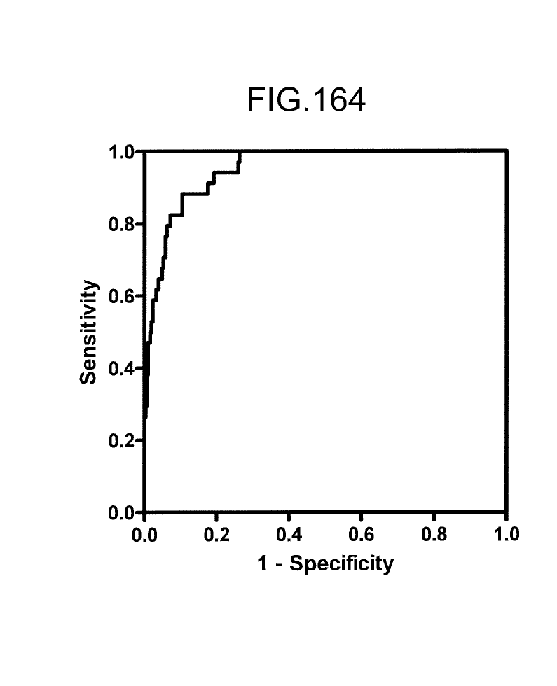

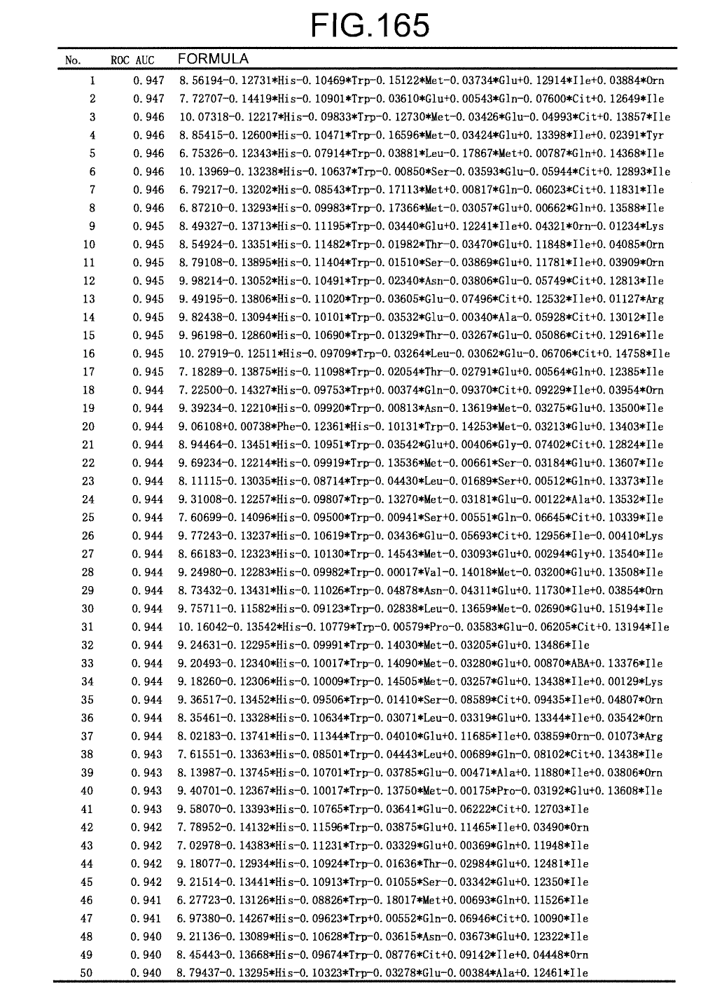

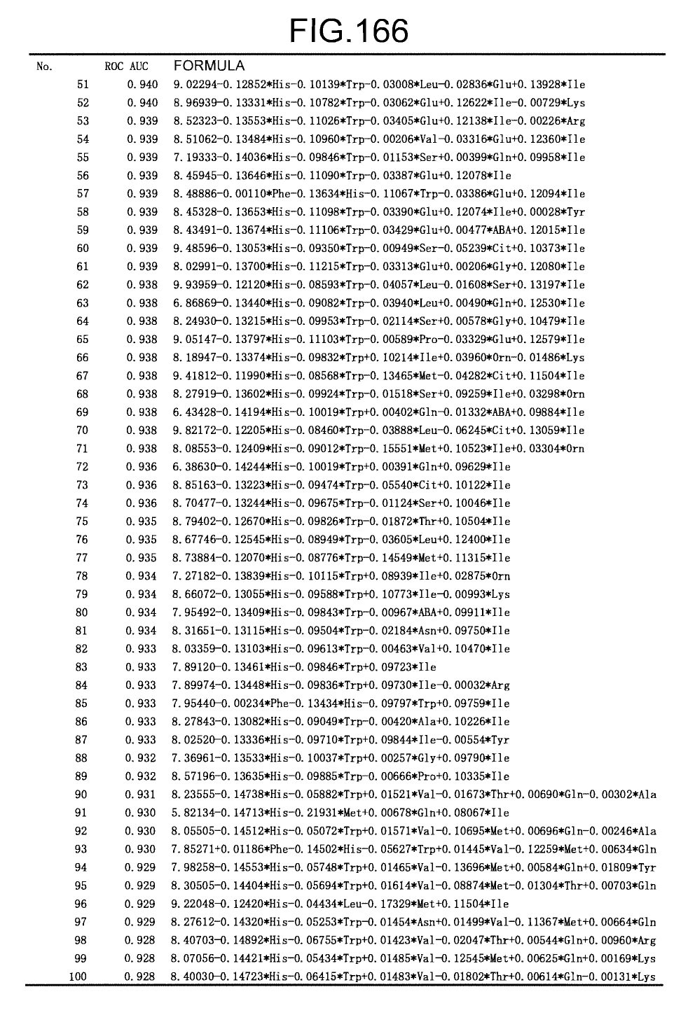

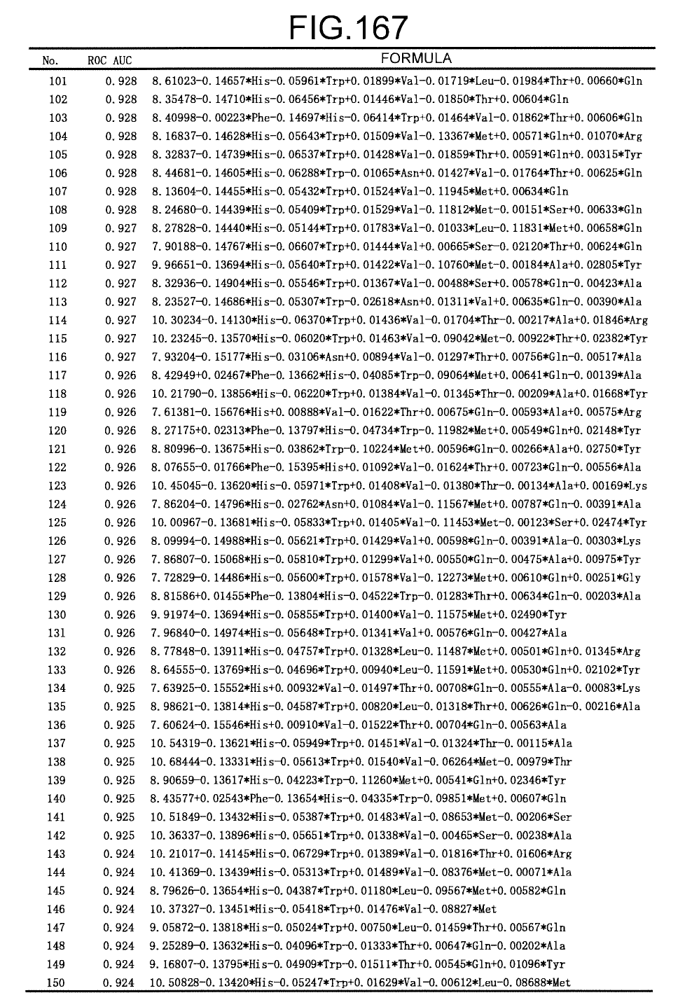

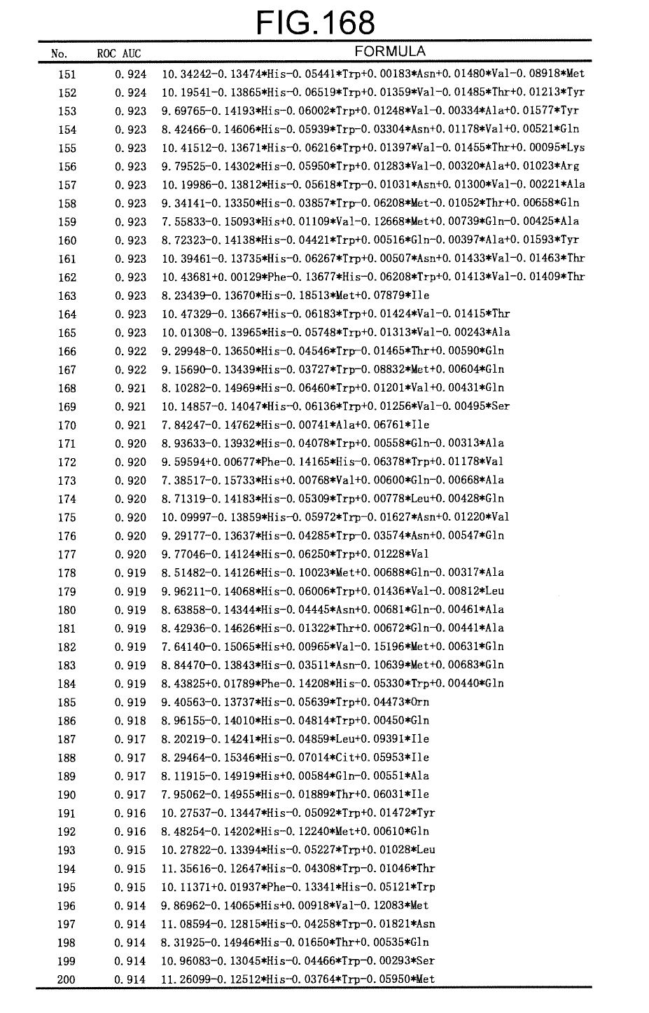

The discriminative ability of index formula 60 in 2-group discrimination between the ovarian cancer patient group and the ovarian cancer-free group is evaluated by AUC of the ROC curve (see FIG. 164). As a result, AUC of 0.951±0.014 (in 95% confidence interval, 0.924 to 0.979) is obtained. From this, it is found that index formula 60 is an index which is useful, with high diagnostic ability. In addition, when the optimum cutoff value of the average value of the sensitivity and the specificity of 2-group discrimination between the ovarian cancer patient group and the ovarian cancer-free group using index formula 60 is calculated, the cutoff value is 0.09512, thereby obtaining 88.24% sensitivity and 89.58% specificity. From this, it is found that index formula 60 is an index which is useful, with high diagnostic ability. A plurality of linear discriminants having discriminative ability equivalent to index formula 60 are obtained. They are shown in FIGS. 165, 166, 167, and 168. The value of each coefficient in the discriminants shown in FIGS. 165, 166, 167, and 168 may be multiplied by a real number, and the value of constant term may be subjected to addition, subtraction, multiplication, and division with an arbitrary real constant.

### Example 26

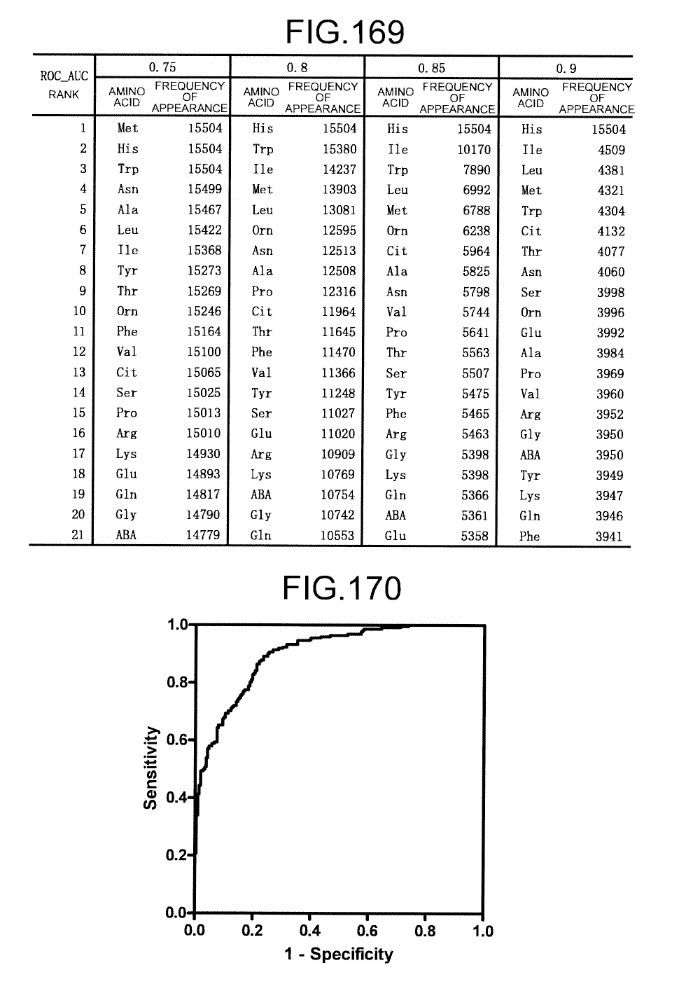

The sample data used in Example 11 is used. All the linear discriminants for performing 2-group discrimination between the ovarian cancer patient group and the ovarian cancer-free group are extracted by an explanatory variable coverage method. The areas under the ROC curve of all the discriminants satisfying the condition in which the maximum value of amino acid explanatory variables appearing in each of the discriminants is 6 are calculated. The frequencies with which the amino acids appear in the discriminants in which the areas under the ROC curve have values above certain threshold values are measured. When the areas under the ROC curve, 0.75, 0.8, 0.85, and 0.9 are threshold values, it is found that Asn, Met, Ile, Leu, His, Trp, and Orn are in the top ten among the amino acids extracted at high frequency at all times (see FIG. 169). From this, it is found that multivariate discriminants using these amino acids as explanatory variables have 2-group discriminative ability between the ovarian cancer patient group and the ovarian cancer-free group.

### Example 27

The sample data used in Example 11 is used. Indexes which maximize 2-group discriminative ability between the female genital cancer suffering risk group and the healthy group are searched for, by logistic analysis (explanatory variable coverage method according to the maximizing criterion of area under the ROC curve). As a result, as index formula 61, a logistic regression equation having Phe, His, Met, Pro, Lys, and Arg (the numerical coefficients of the amino acid explanatory variables Phe, His, Met, Pro, Lys, and Arg and the constant term are −0.06095, −0.11827, −0.14776, 0.01459, 0.03299, −0.03875, and 10.40250 in order) is obtained.

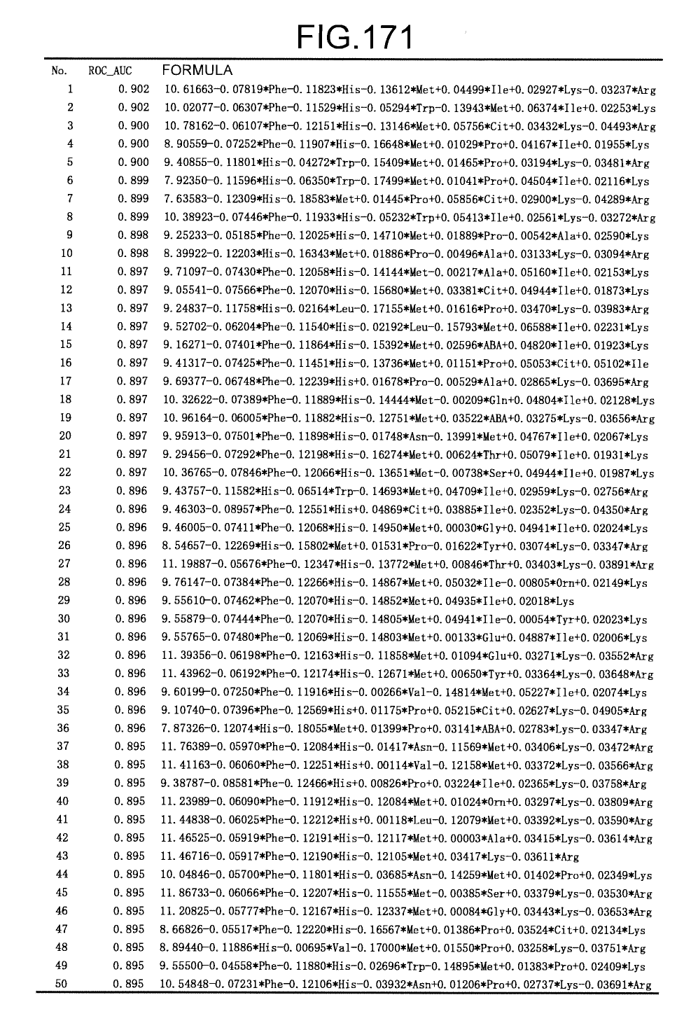

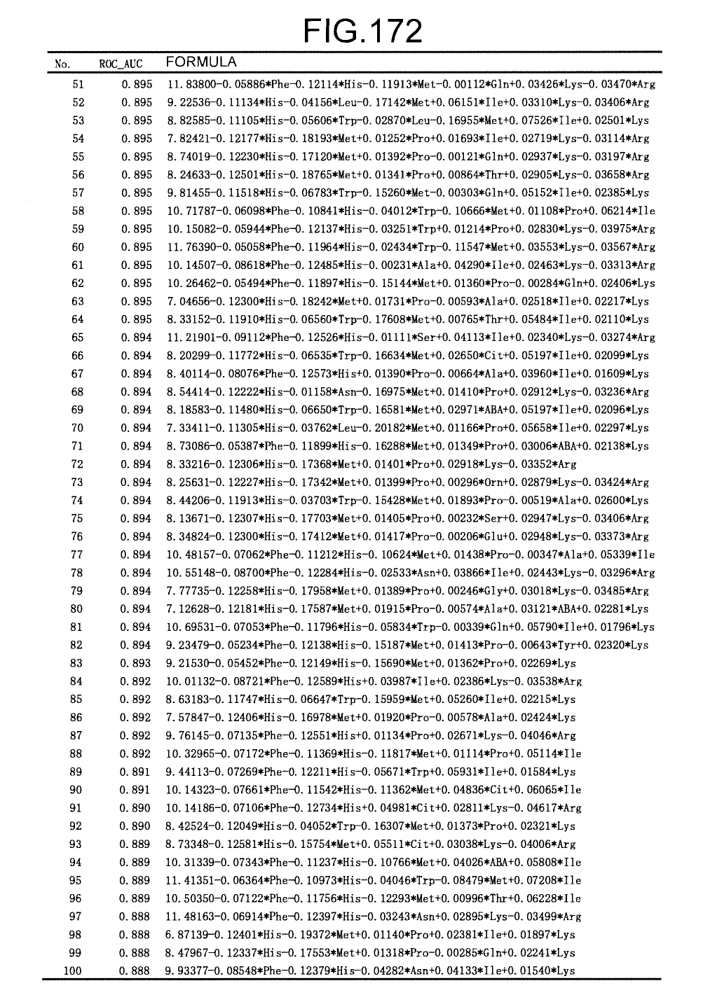

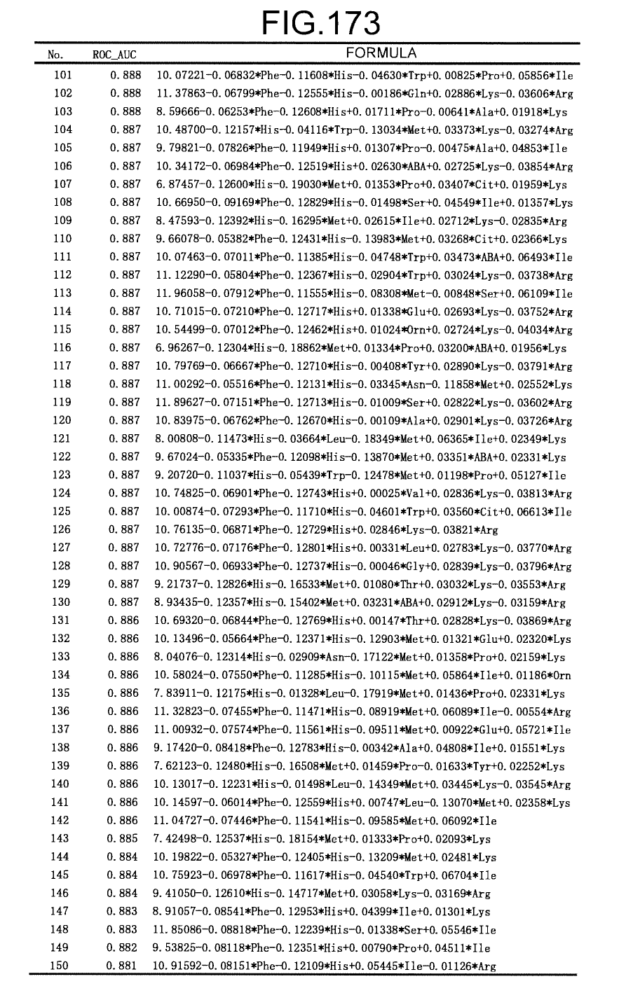

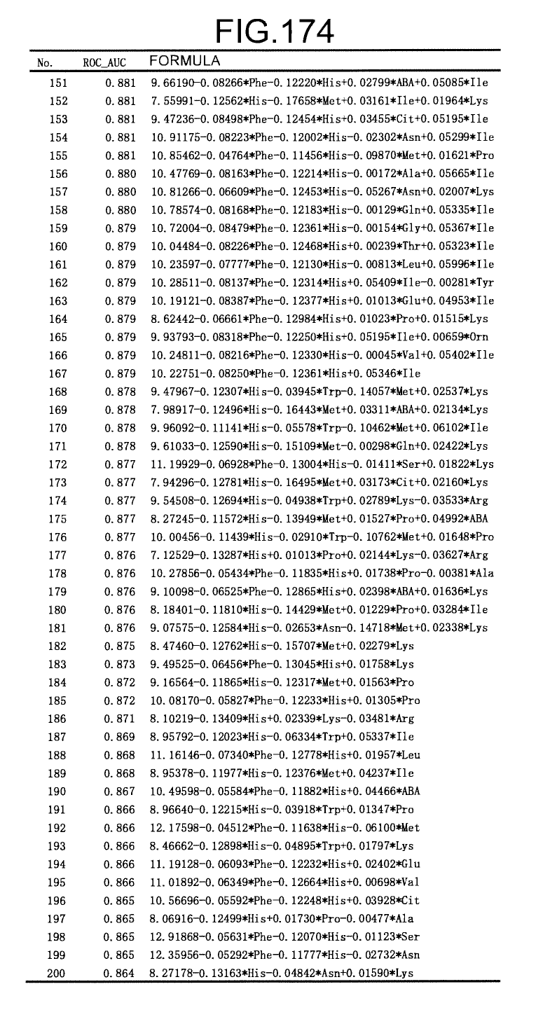

The discriminative ability of index formula 61 in 2-group discrimination between the female genital cancer suffering risk group and the healthy group is evaluated by AUC of the ROC curve (see FIG. 170). As a result, AUC of 0.903±0.014 (in 95% confidence interval, 0.876 to 0.930) is obtained. From this, it is found that index formula 61 is an index which is useful, with high diagnostic ability. In addition, when the optimum cutoff value of the average value of the sensitivity and the specificity of 2-group discrimination between the female genital cancer suffering risk group and the healthy group using index formula 61 is calculated, the cutoff value is −0.5313, thereby obtaining 89.14% sensitivity and 76.53% specificity. From this, it is found that index formula 61 is an index which is useful, with high diagnostic ability. A plurality of logistic regression equations having discriminative ability equivalent to index formula 61 are obtained. They are shown in FIGS. 171, 172, 173, and 174. The value of each coefficient in the equations shown in FIGS. 171, 172, 173, and 174 may be multiplied by a real number, and the value of constant term may be subjected to addition, subtraction, multiplication, and division with an arbitrary real constant.

### Example 28

The sample data used in Example 11 is used. Indexes which maximize 2-group discriminative ability between the female genital cancer suffering risk group and the healthy group are searched for, by linear discriminant analysis (explanatory variable coverage method according to the maximizing criterion of area under the ROC curve). As a result, as index formula 62, a linear discriminant having Phe, His, Met, Pro, Lys, and Arg (the numerical coefficients of the amino acid explanatory variables Phe, His, Met, Pro, Lys, and Arg and the constant term are −0.05213, −0.10933, −0.14686, 0.01480, 0.03207, −0.03318, and 8.84450 in order) is obtained.

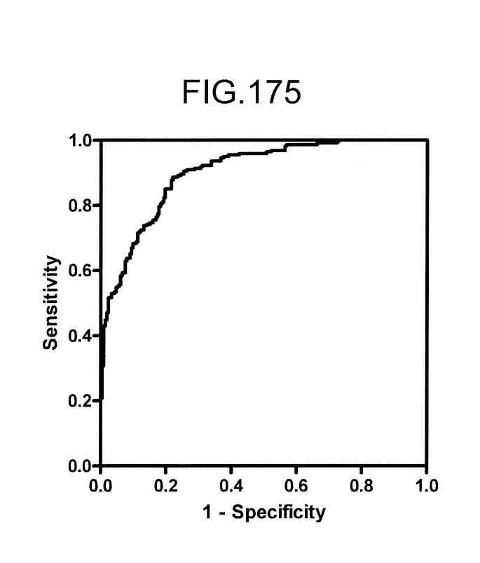

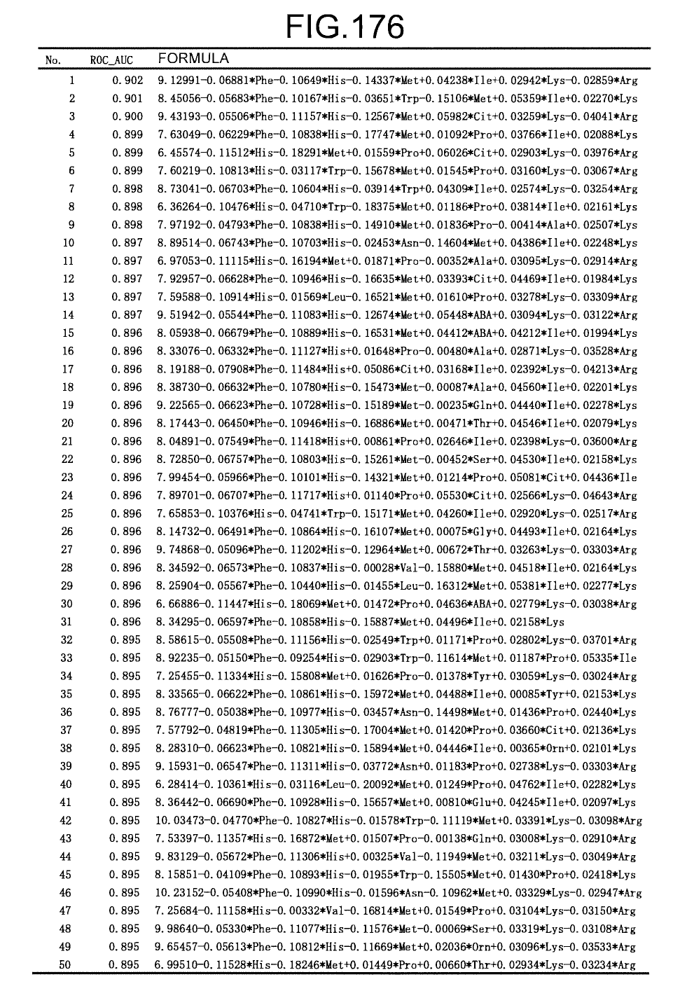

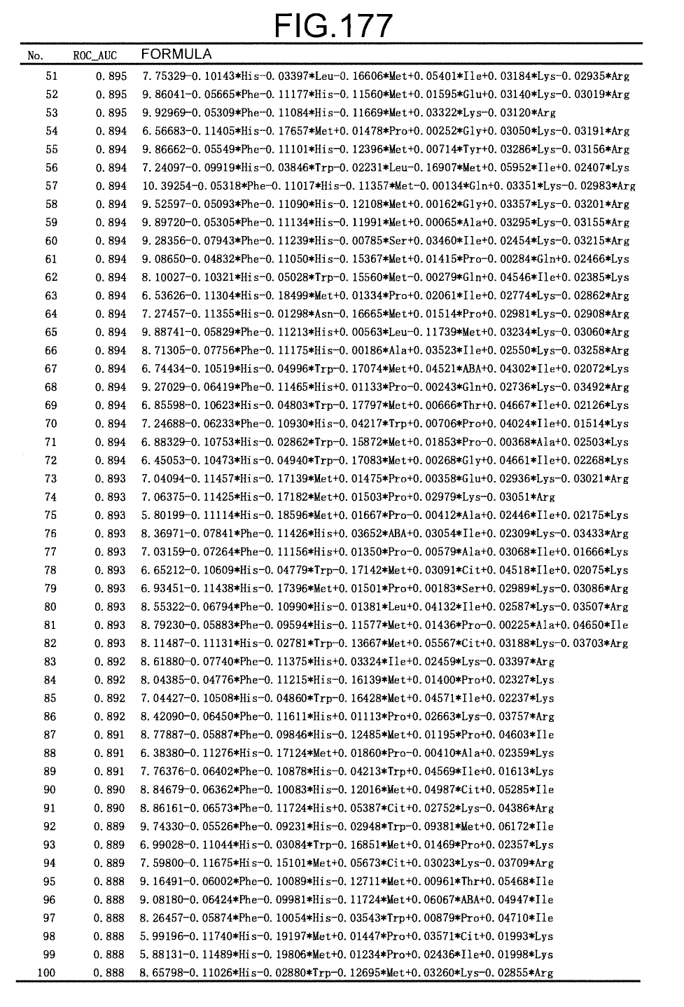

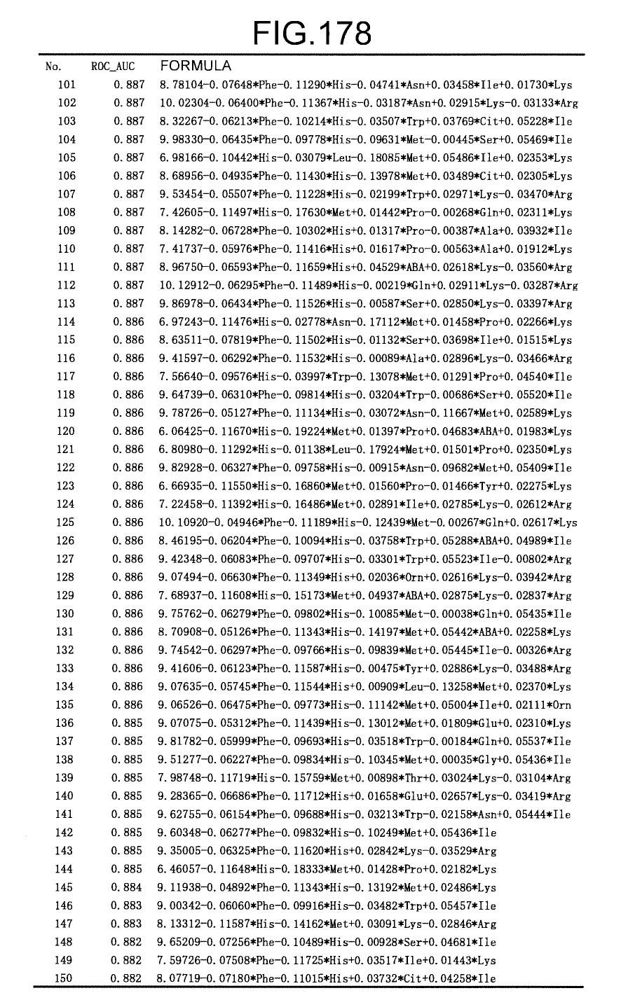

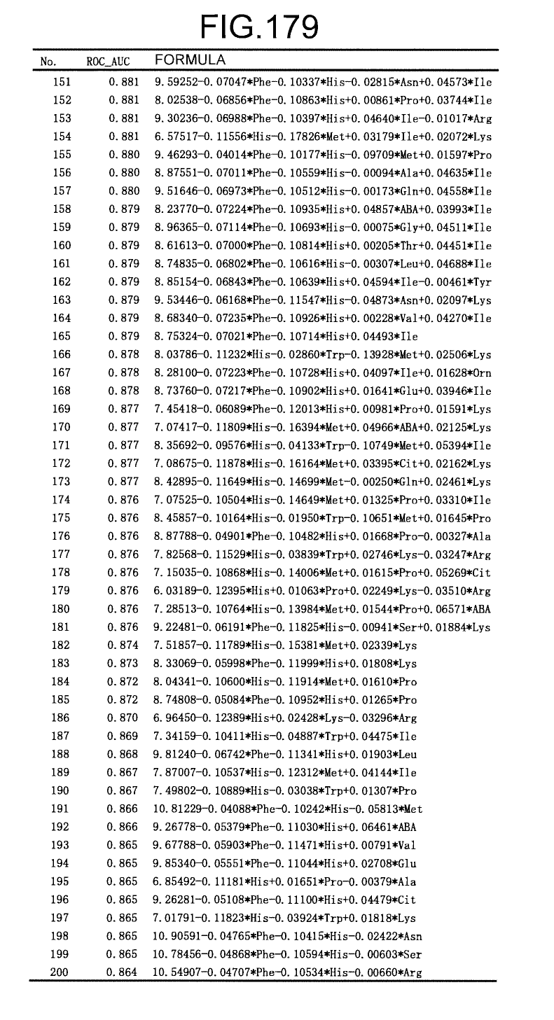

The discriminative ability of index formula 62 in 2-group discrimination between the female genital cancer suffering risk group and the healthy group is evaluated by AUC of the ROC curve (see FIG. 175). As a result, AUC of 0.903±0.014 (in 95% confidence interval, 0.876 to 0.930) is obtained. From this, it is found that index formula 62 is an index which is useful, with high diagnostic ability. In addition, when the optimum cutoff value of the average value of the sensitivity and the specificity of 2-group discrimination between the female genital cancer suffering risk group and the healthy group using index formula 62 is calculated, the cutoff value is −0.4778, thereby obtaining 88.69% sensitivity and 77.93% specificity. From this, it is found that index formula 62 is an index which is useful, with high diagnostic ability. A plurality of linear discriminants having discriminative ability equivalent to index formula 62 are obtained. They are shown in FIGS. 176, 177, 178, and 179. The value of each coefficient in the discriminants shown in FIGS. 176, 177, 178, and 179 may be multiplied by a real number, and the value of constant term may be subjected to addition, subtraction, multiplication, and division with an arbitrary real constant.

### Example 29

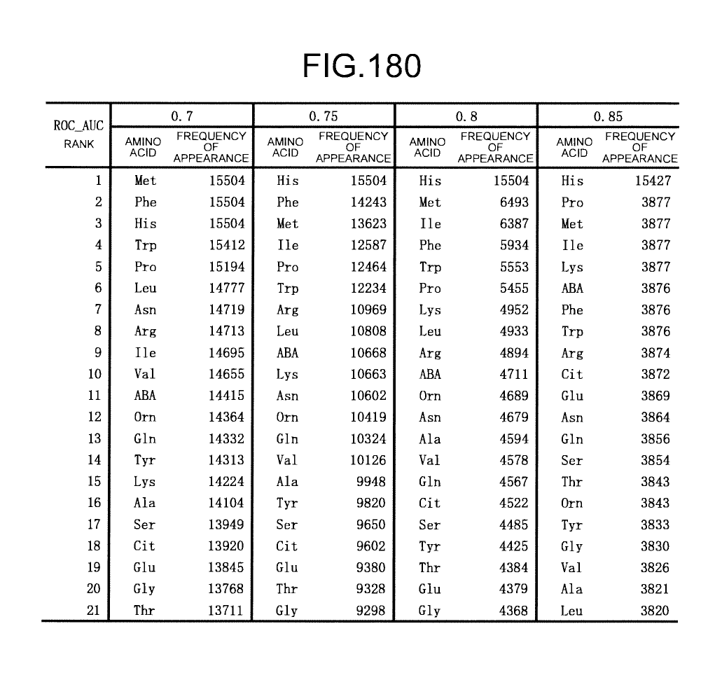

The sample data used in Example 11 is used. All the linear discriminants for performing 2-group discrimination between the female genital cancer suffering risk group and the healthy group are extracted by an explanatory variable coverage method. The areas under the ROC curve of all the discriminants satisfying the condition in which the maximum value of amino acid explanatory variables appearing in each of the discriminants is 6 are calculated. The frequencies with which the amino acids appear in the discriminants in which the areas under the ROC curve have values above certain threshold values are measured. When the areas under the ROC curve, 0.7, 0.75, 0.8, and 0.85 are threshold values, it is found that Pro, Met, Phe, His, Trp, and Arg are in the top ten among the amino acids extracted at high frequency at all times (see FIG. 180). From this, it is found that multivariate discriminants using these amino acids as explanatory variables have 2-group discriminative ability between the female genital cancer suffering risk group and the healthy group.

### Example 30

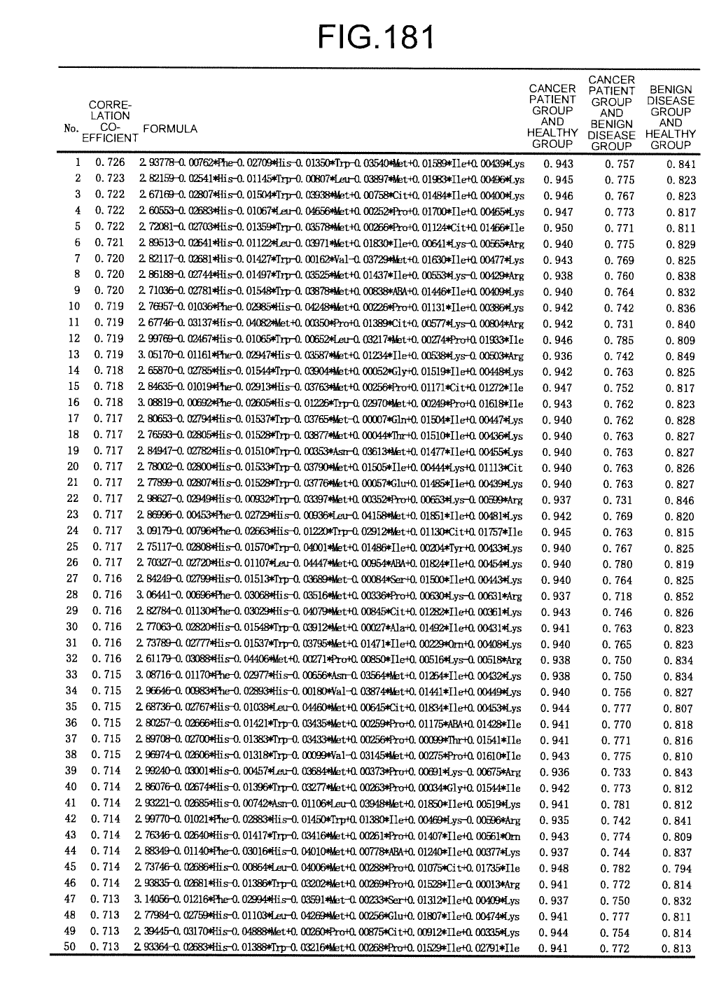

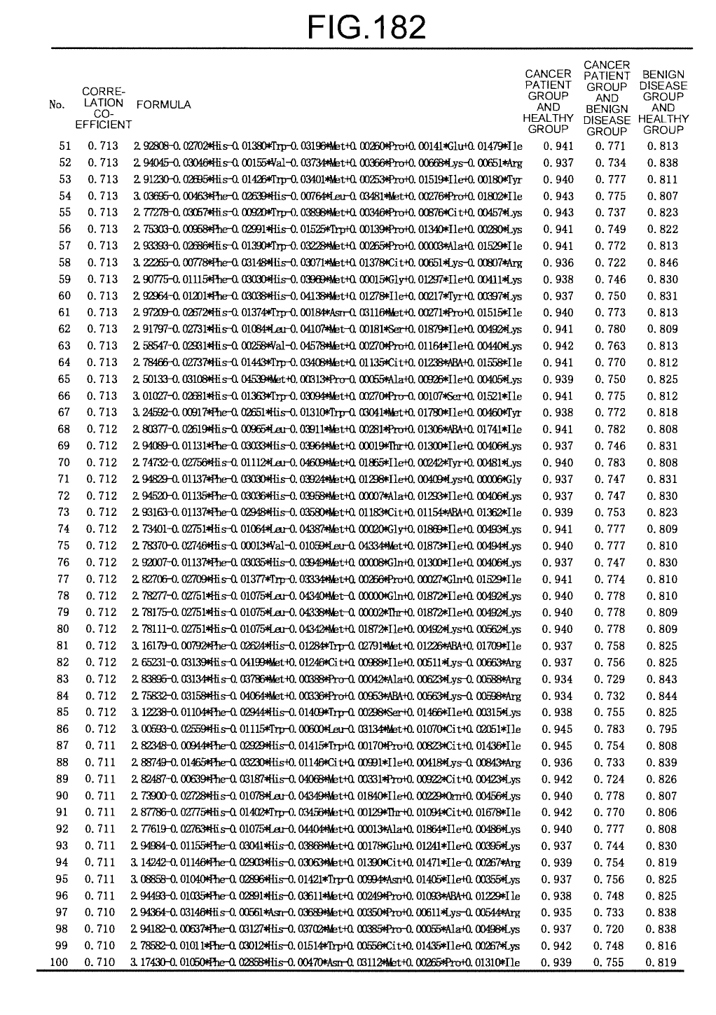

The sample data used in Example 11 is used. Indexes which maximize 3-group discriminative ability between the cancer patient group, the benign disease group, and the healthy group are searched for, by linear discriminant analysis (explanatory variable coverage method according to the maximizing criterion of the Spearman rank correlation coefficient). As a result, as index formula 63, a linear discriminant having His, Trp, Met, Pro, Ile, and Lys (the numerical coefficients of the amino acid explanatory variables His, Trp, Met, Pro, Ile, and Lys and the constant term are −0.02749, −0.01483, −0.04099, 0.00232, 0.01338, and 0.00419 in order) is obtained among a plurality of index formulae having equivalent ability. The discriminative ability of index formula 63 in 3-group discrimination between the cancer patient group, the benign disease group, and the healthy group is evaluated by the Spearman rank correlation coefficient. As a result, 0.728 is obtained. From this, it is found that index formula 63 is an index which is useful, with high diagnostic ability. The discriminative abilities of index formula 63 in 2-group discrimination between the cancer patient group and the healthy group, between the cancer patient group and the benign disease group, and between the benign disease group and the healthy group are evaluated by AUC of the ROC curve. As a result, AUCs of 0.943, 0.757, and 0.841 are obtained with respect to the respective 2-group discriminations. From this, it is found that index formula 63 is an index which is useful, with high diagnostic ability. A plurality of linear discriminants having discriminative ability equivalent to index formula 63 are obtained. They are shown in FIGS. 181 and 182. The value of each coefficient in the discriminants shown in FIGS. 181 and 182 may be multiplied by a real number, and the value of constant term may be subjected to addition, subtraction, multiplication, and division with an arbitrary real constant.

### Example 31

Of the sample data used in Example 11, the data of the cervical cancer group, the endometrial cancer group, and the ovarian cancer group are used. Amino acid explanatory variables which maximize 3-group discriminative ability between the cervical cancer group, the endometrial cancer group, and the ovarian cancer group are searched for, by discrimination analysis by the Mahalanobis' generalized distance. As a result, as explanatory variable group 1, His, Leu, Ser, Thr, Glu, Gln, Ala, and Lys are obtained.

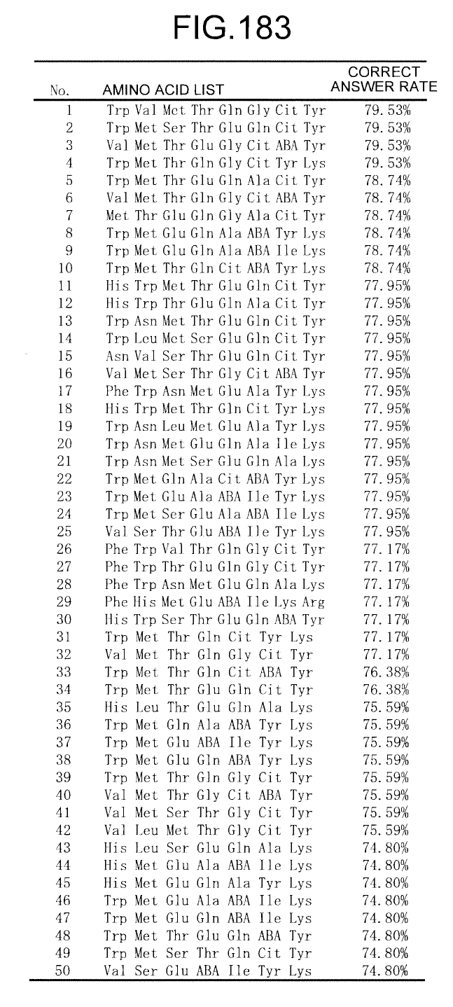

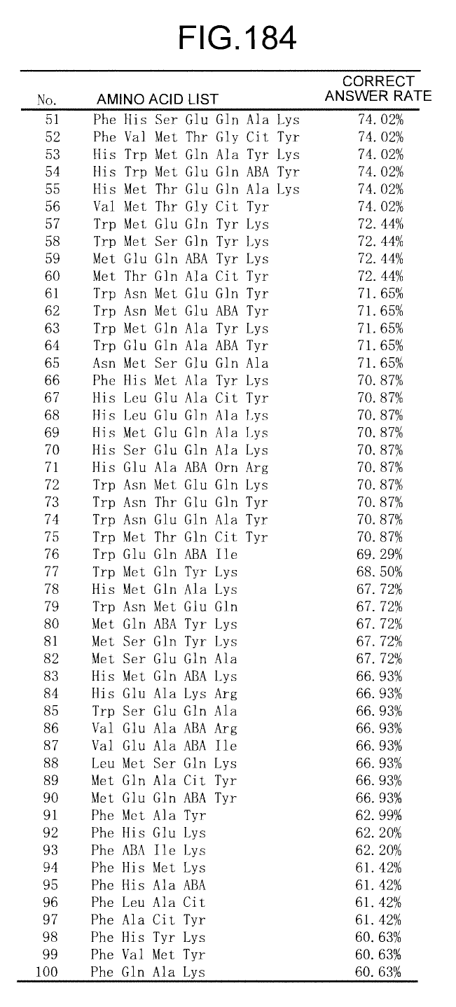

The discriminative ability of explanatory variable group 1 in 3-group discrimination between the cervical cancer group, the endometrial cancer group, and the ovarian cancer group is evaluated by the correct answer rate of the discrimination result. As a result, the entire correct answer rate is 80.3%, showing high discriminative ability. As shown in FIGS. 183 and 184, the combinations of amino acid explanatory variables having discriminative ability equivalent to explanatory variable group 1 are obtained.

### Example 32

Of the sample data used in Example 11, the data of the cervical cancer group, the endometrial cancer group, and the ovarian cancer group are used. Indexes which maximize 3-group discriminative ability between the cervical cancer group, the endometrial cancer group, and the ovarian cancer group are searched for, by linear discriminant analysis. As a result, linear discriminant group 1 having the amino acid explanatory variables Phe, Trp, Pro, Glu, Cit, Tyr, and Lys and constant term is obtained. The value of each coefficient in linear discriminant group 1 may be multiplied by a real number, and the value of constant term may be subjected to addition, subtraction, multiplication, and division with an arbitrary real constant.

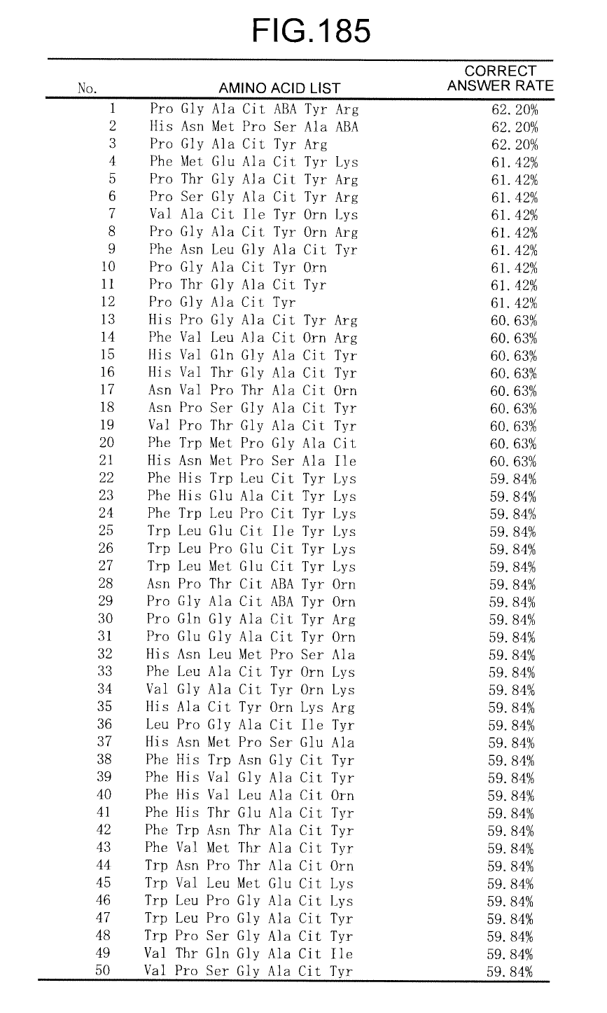

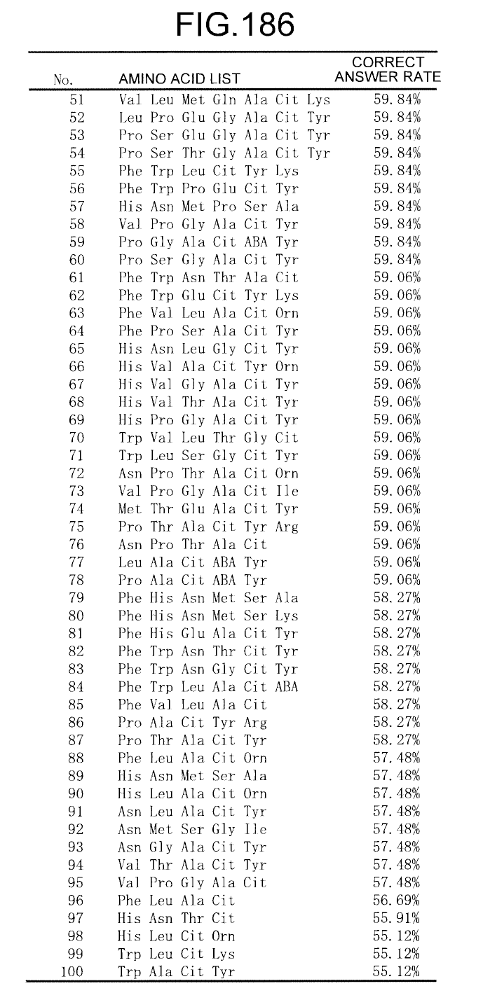

The discriminative ability of linear discriminant group 1 in 3-group discrimination between the cervical cancer group, the endometrial cancer group, and the ovarian cancer group is evaluated by the correct answer rate of the discrimination result. As a result, the entire correct answer rate is 62.2%, showing high discriminative ability. As shown in FIGS. 185 and 186, the combinations of amino acid explanatory variables including linear discriminants having discriminative ability equivalent to linear discriminant group 1 are obtained.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A method of evaluating female genital cancer, comprising:
a measuring step of measuring amino acid concentration data on a concentration value of an amino acid in blood collected from a subject to be evaluated; and
a concentration value criterion evaluating step of evaluating a state of female genital cancer including at least one of cervical cancer, endometrial cancer, and ovarian cancer in the subject, based on the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data of the subject measured at the measuring step.

2. The method of evaluating female genital cancer according to claim 1, wherein the concentration value criterion evaluating step further includes a concentration value criterion discriminating step of discriminating (i) between the female genital cancer and a female genital cancer-free, (ii) between any one of the cervical cancer, the endometrial cancer, and the ovarian cancer and the female genital cancer-free, (iii) between any one of the cervical cancer and the endometrial cancer and any one of a cervical cancer-free and an endometrial cancer-free, (iv) between the cervical cancer and the cervical cancer-free, (v) between the endometrial cancer and the endometrial cancer-free, (vi) between the ovarian cancer and a ovarian cancer-free, (vii) between a female genital cancer suffering risk group and a healthy group, or (viii) between the cervical cancer, the endometrial cancer, and the ovarian cancer in the subject, based on the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data of the subject measured at the measuring step.

3. The method of evaluating female genital cancer according to claim 1, wherein the concentration value criterion evaluating step further includes:
a discriminant value calculating step of calculating a discriminant value that is a value of a multivariate discriminant with a concentration of the amino acid as an explanatory variable, based on both (i) the concentration value of at least one of Thr, Ser, Asn, Gln, Pro, Gly, Ala, Cit, Val, Met, Ile, Leu, Tyr, Phe, His, Trp, Orn, Lys, and Arg contained in the amino acid concentration data of the subject measured at the measuring step and (ii) the previously established multivariate discriminant; and
a discriminant value criterion evaluating step of evaluating the state of female genital cancer in the subject based on the discriminant value calculated at the discriminant value calculating step, and
wherein the multivariate discriminant contains at least one of Thr, Ser, Asn, Gln, Pro, Gly, Ala, Cit, Val, Met, Ile, Leu, Tyr, Phe, His, Trp, Orn, Lys, and Arg as the explanatory variable.

4. The method of evaluating female genital cancer according to claim 3, wherein the discriminant value criterion evaluating step further includes a discriminant value criterion discriminating step of discriminating (i) between the female genital cancer and a female genital cancer-free, (ii) between any one of the cervical cancer, the endometrial cancer, and the ovarian cancer and the female genital cancer-free, (iii) between any one of the cervical cancer and the endometrial cancer and any one of a cervical cancer-free and an endometrial cancer-free, (iv) between the cervical cancer and the cervical cancer-free, (v) between the endometrial cancer and the endometrial cancer-free, (vi) between the ovarian cancer and a ovarian cancer-free, (vii) between a female genital cancer suffering risk group and a healthy group, or (viii) between the cervical cancer, the endometrial cancer, and the ovarian cancer in the subject, based on the discriminant value calculated at the discriminant value calculating step.

5. The method of evaluating female genital cancer according to claim 4, wherein the multivariate discriminant is any one of a fractional expression, the sum of a plurality of the fractional expressions, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

6. The method of evaluating female genital cancer according to claim 5, wherein (I) at the discriminant value calculating step, the discriminant value is calculated based on (i) the concentration value of at least one of Thr, Ser, Asn, Gln, Pro, Ala, Val, Met, Ile, Leu, Tyr, Phe, His, Trp, Orn, and Arg contained in the amino acid concentration data of the subject measured at the measuring step and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) at the discriminant value criterion discriminating step, the discrimination between any one of the cervical cancer, the endometrial cancer, and the ovarian cancer and the female genital cancer-free in the subject is conducted based on the discriminant value calculated at the discriminant value calculating step.

7. The method of evaluating female genital cancer according to claim 6, wherein the multivariate discriminant is (i) the fractional expression with Gln, His, and Arg as the explanatory variables, (ii) the fractional expression with a-ABA, His, and Met as the explanatory variables, (iii) the fractional expression with Ile, His, Cit, Arg, Tyr, and Trp as the explanatory variables, (iv) the fractional expression with a-ABA, Cit, and Met as the explanatory variables, (v) the linear discriminant with Gly, Val, His, and Arg as the explanatory variables, (vi) the linear discriminant with Gly, a-ABA, Met, and His as the explanatory variables, (vii) the linear discriminant with Ala, Ile, His, Trp, and Arg as the explanatory variables, (viii) the linear discriminant with Gly, Cit, Met, and Phe as the explanatory variables, (ix) the linear discriminant with His, Leu, Met, Cit, Ile, and Tyr as the explanatory variables, (x) the logistic regression equation with Val, Leu, His, and Arg as the explanatory variables, (xi) the logistic regression equation with a-ABA, Met, Tyr, and His as the explanatory variables, (xii) the logistic regression equation with Val, Ile, His, Trp, and Arg as the explanatory variables, (xiii) the logistic regression equation with Cit, a-ABA, Met, and Tyr as the explanatory variables, or (xiv) the logistic regression equation with His, Leu, Met, Cit, Ile, and Tyr as the explanatory variables.

8. The method of evaluating female genital cancer according to claim 5, wherein (I) at the discriminant value calculating step, the discriminant value is calculated based on (i) the concentration value of at least one of Thr, Ser, Asn, Pro, Gly, Cit, Val, Met, Ile, Leu, Phe, His, Trp, Orn, Lys, and Arg contained in the amino acid concentration data of the subject measured at the measuring step and (ii) the multivariate discriminant containing at least one of Thr, Ser, Asn, Pro, Gly, Cit, Val, Met, Ile, Leu, Phe, His, Trp, Orn, Lys, and Arg as the explanatory variable, and (II) at the discriminant value criterion discriminating step, the discrimination between any one of the cervical cancer and the endometrial cancer and any one of the cervical cancer-free and the endometrial cancer-free in the subject is conducted based on the discriminant value calculated at the discriminant value calculating step.

9. The method of evaluating female genital cancer according to claim 8, wherein the multivariate discriminant is (i) the fractional expression with Lys, His, and Arg as the explanatory variables, (ii) the fractional expression with a-ABA, His, and Met as the explanatory variables, (iii) the fractional expression with Ile, His, Cit, and Arg as the explanatory variables, (iv) the linear discriminant with Gly, Val, His, and Arg as the explanatory variables, (v) the linear discriminant with Gly, Phe, His, and Arg as the explanatory variables, (vi) the linear discriminant with Cit, Ile, His, and Arg as the explanatory variables, (vii) the linear discriminant with His, Leu, Met, Cit, Ile, and Tyr as the explanatory variables, (viii) the logistic regression equation with Val, His, Lys, and Arg as the explanatory variables, (ix) the logistic regression equation with Thr, a-ABA, Met, and His as the explanatory variables, (x) the logistic regression equation with Cit, Ile, His, and Arg as the explanatory variables, or (xi) the logistic regression equation with His, Leu, Met, Cit, Ile, and Tyr as the explanatory variables.

10. The method of evaluating female genital cancer according to claim 5, wherein (I) at the discriminant value calculating step, the discriminant value is calculated based on (i) the concentration value of at least one of Asn, Val, Met, Leu, Phe, His, Trp, Orn, Lys, and Arg contained in the amino acid concentration data of the subject measured at the measuring step and (ii) the multivariate discriminant containing at least one of Asn, Val, Met, Leu, Phe, His, Trp, Orn, Lys, and Arg as the explanatory variable, and (II) at the discriminant value criterion discriminating step, the discrimination between the cervical cancer and the cervical cancer-free in the subject is conducted based on the discriminant value calculated at the discriminant value calculating step.

11. The method of evaluating female genital cancer according to claim 10, wherein the multivariate discriminant is (i) the fractional expression with a-ABA, His, and Val as the explanatory variables, (ii) the fractional expression with a-ABA, Met, and Val as the explanatory variables, (iii) the fractional expression with Met, His, Cit, and Arg as the explanatory variables, (iv) the linear discriminant with Gly, Val, His, and Arg as the explanatory variables, (v) the linear discriminant with Gly, Val, Met, and Lys as the explanatory variables, (vi) the linear discriminant with Cit, Met, His, and Arg as the explanatory variables, (vii) the linear discriminant with His, Leu, Met, Ile, Tyr, and Lys as the explanatory variables, (viii) the logistic regression equation with Val, Leu, His, and Arg as the explanatory variables, (ix) the logistic regression equation with Met, His, Orn, and Arg as the explanatory variables, (x) the logistic regression equation with Val, Tyr, His, and Arg as the explanatory variables, or (xi) the logistic regression equation with His, Leu, Met, Ile, Tyr, and Lys as the explanatory variables.

12. The method of evaluating female genital cancer according to claim 5, wherein (I) at the discriminant value calculating step, the discriminant value is calculated based on (i) the concentration value of at least one of Thr, Ser, Asn, Pro, Gly, Cit, Val, Met, Ile, Leu, Phe, His, Trp, and Arg contained in the amino acid concentration data of the subject measured at the measuring step and (ii) the multivariate discriminant containing at least one of Thr, Ser, Asn, Pro, Gly, Cit, Val, Met, Ile, Leu, Phe, His, Trp, and Arg as the explanatory variable, and (II) at the discriminant value criterion discriminating step, the discrimination between the endometrial cancer and the endometrial cancer-free in the subject is conducted based on the discriminant value calculated at the discriminant value calculating step.

13. The method of evaluating female genital cancer according to claim 12, wherein the multivariate discriminant is (i) the fractional expression with Lys, His, and Arg as the explanatory variables, (ii) the fractional expression with a-ABA, His, and Met as the explanatory variables, (iii) the fractional expression with Ile, His, Asn, and Cit as the explanatory variables, (iv) the linear discriminant with Gln, His, Lys, and Arg as the explanatory variables, (v) the linear discriminant with Gly, Met, Phe, and His as the explanatory variables, (vi) the linear discriminant with Cit, Ile, His, and Arg as the explanatory variables, (vii) the linear discriminant with His, Asn, Val, Pro, Cit, and Ile as the explanatory variables, (viii) the logistic regression equation with Gln, Gly, His, and Arg as the explanatory variables, (ix) the logistic regression equation with Gln, Phe, His, and Arg as the explanatory variables, (x) the logistic regression equation with Gln, Ile, His, and Arg as the explanatory variables, or (xi) the logistic regression equation with His, Asn, Val, Pro, Cit, and Ile as the explanatory variables.

14. The method of evaluating female genital cancer according to claim 5, wherein (I) at the discriminant value calculating step, the discriminant value is calculated based on (i) the concentration value of at least one of Thr, Ser, Asn, Gln, Ala, Cit, Val, Met, Ile, Leu, Tyr, Phe, His, Trp, Orn, Lys, and Arg contained in the amino acid concentration data of the subject measured at the measuring step and (ii) the multivariate discriminant containing at least one of Thr, Ser, Asn, Gln, Ala, Cit, Val, Met, Ile, Leu, Tyr, Phe, His, Trp, Orn, Lys, and Arg as the explanatory variable, and (II) at the discriminant value criterion discriminating step, the discrimination between the ovarian cancer and the ovarian cancer-free in the subject is conducted based on the discriminant value calculated at the discriminant value calculating step.

15. The method of evaluating female genital cancer according to claim 14, wherein the multivariate discriminant is (i) the fractional expression with Orn, Cit, and Met as the explanatory variables, (ii) the fractional expression with Gln, Cit, and Tyr as the explanatory variables, (iii) the fractional expression with Orn, His, Phe, and Trp as the explanatory variables, (iv) the linear discriminant with Ser, Cit, Orn, and Trp as the explanatory variables, (v) the linear discriminant with Ser, Cit, Ile, and Orn as the explanatory variables, (vi) the linear discriminant with Phe, Trp, Orn, and Lys as the explanatory variables, (vii) the linear discriminant with His, Trp, Glu, Cit, Ile, and Orn as the explanatory variables, (viii) the logistic regression equation with Ser, Cit, Trp, and Orn as the explanatory variables, (ix) the logistic regression equation with Gln, Cit, Ile, and Tyr as the explanatory variables, (x) the logistic regression equation with Asn, Phe, His, and Trp as the explanatory variables, or (xi) the logistic regression equation with His, Trp, Glu, Cit, Ile, and Orn as the explanatory variables.

16. The method of evaluating female genital cancer according to claim 5, wherein (I) at the discriminant value calculating step, the discriminant value is calculated based on (i) the concentration value of at least one of Thr, Ser, Asn, Gln, Pro, Ala, Val, Met, Ile, Leu, Tyr, Phe, His, Trp, Orn, and Arg contained in the amino acid concentration data of the subject measured at the measuring step and (ii) the multivariate discriminant containing at least one of Thr, Ser, Asn, Gln, Pro, Ala, Val, Met, Ile, Leu, Tyr, Phe, His, Trp, Orn, and Arg as the explanatory variable, and (II) at the discriminant value criterion discriminating step, the discrimination between the female genital cancer suffering risk group and the healthy group in the subject is conducted based on the discriminant value calculated at the discriminant value calculating step.

17. The method of evaluating female genital cancer according to claim 16, wherein the multivariate discriminant is the linear discriminant with Phe, His, Met, Pro, Lys, and Arg as the explanatory variables, or the logistic regression equation with Phe, His, Met, Pro, Lys, and Arg as the explanatory variables.

18. The method of evaluating female genital cancer according to claim 5, wherein (I) at the discriminant value calculating step, the discriminant value is calculated based on (i) the concentration value of at least one of Thr, Ser, Asn, Glu, Gln, Pro, Gly, Ala, Cit, ABA, Val, Met, Ile, Leu, Tyr, Phe, His, Trp, Orn, Lys, and Arg contained in the amino acid concentration data of the subject measured at the measuring step and (ii) the multivariate discriminant containing at least one of Thr, Ser, Asn, Glu, Gln, Pro, Gly, Ala, Cit, ABA, Val, Met, Ile, Leu, Tyr, Phe, His, Trp, Orn, Lys, and Arg as the explanatory variable, and (II) at the discriminant value criterion discriminating step, the discrimination between the cervical cancer, the endometrial cancer, and the ovarian cancer in the subject is conducted based on the discriminant value calculated at the discriminant value calculating step.

19. The method of evaluating female genital cancer according to claim 18, wherein the multivariate discriminant is the discriminant with Cit, Met, Lys, Asn, Ala, Thr, Gln, and a-ABA as the explanatory variables which is prepared by the Mahalanobis' generalized distance method, or the discriminant prepared with His, Leu, Ser, Thr, Glu, Gln, Ala, and Lys as the explanatory variables which is prepared by the Mahalanobis' generalized distance method.
